(19) United States
(12) Patent Application Publication
(10) Pub. No.: US 2025/0297288 A1
(43) Pub. Date: Sep. 25, 2025

(54) GENOME EDITING OF B CELLS

(71) Applicant: Editas Medicine, Inc., Cambridge, MA (US)

(72) Inventor: John Anthony Zuris, Boston, MA (US)

(21) Appl. No.: 18/863,883

(22) PCT Filed: May 10, 2023
(86) PCT No.: PCT/US2023/021773

§ 371 Date: Nov. 7, 2024

Related U.S. Application Data
(60) Provisional application No. 63/340,222, filed on May 10, 2022.

(51) Int. Cl. C12N 15/90; A61K 35/17; C12N 5/0781; C12N 9/22; C12N 15/11

(52) U.S. Cl. CPC C12N 15/907; A61K 35/17; C12N 5/0635; C12N 9/222; C12N 15/111; C12N 2310/20; C12N 2510/00; C12N 2800/22

(57) ABSTRACT

Strategies, systems, compositions, and methods for efficient production of knock-in cellular clones without reporter genes. An essential gene is targeted using a knock-in cassette that comprises an exogenous coding sequence for a gene product of interest (or “cargo sequence”) in frame with and downstream (3′) of an exogenous coding sequence or partial coding sequence of the essential gene. Undesired targeting events create a non-functional version of the essential gene, in essence a knock-out, which is “rescued” by correct integration of the knock-in cassette, which restores the essential gene coding region so that a functional gene product is produced and positions the cargo sequence in frame with and downstream of the essential gene coding sequence.

### CROSS-REFERENCE TO RELATED APPLICATIONS

This application claims to the benefit of U.S. Provisional Application No. 63/340,222, filed May 10, 2022, the entirety of which is incorporated herein by reference.

### BACKGROUND

A problem with targeted integration strategies for the generation of genetically engineered cells is that successful targeted integration events can be rare, especially when using double-stranded DNA (dsDNA) as a template where knock-in efficiencies are often below 5%. There remains a need for methods of selecting genetically engineered cells, such as genetically engineered B cells, that include successful targeted integration events.

### SUMMARY

The present disclosure provides strategies, systems, compositions, and methods for genetically engineering B cells via targeted integration that do not require external selection markers, such as fluorescent or antibiotic resistance markers, while yielding a high frequency of correctly targeted clones. In general, the strategies, systems, compositions, and methods for genetically engineering B cells via targeted integration provided herein feature a targeted break in an essential gene mediated by a nuclease, and integration of an exogenous knock-in cassette that, if inserted correctly, results in a functional variant of the essential gene and also includes an expression construct harboring a cargo sequence.

In one aspect, the disclosure features a method of editing the genome of a B cell (e.g., a B cell in a population of B cells), the method comprising contacting the B cell (or the population of B cells) with: (i) a nuclease that causes a break within an endogenous coding sequence of an essential gene in the B cell, wherein the essential gene encodes a gene product that is required for survival and/or proliferation of the B cell, and (ii) a donor template that comprises a knock-in cassette comprising an exogenous coding sequence for a gene product of interest in frame with and downstream (3′) of an exogenous coding sequence or partial coding sequence of the essential gene, wherein the knock-in cassette is integrated into the genome of the B cell by homology-directed repair (HDR) of the break, resulting in a genome-edited B cell that expresses: (a) the gene product of interest, and (b) the gene product encoded by the essential gene that is required for survival and/or proliferation of the B cell, or a functional variant thereof.

In some embodiments, following the contacting step, at least about 60%, at least about 65%, at least about 70%, at least about 75%, at least about 80%, at least about 85%, at least about 90%, at least about 95%, or more, of the viable B cells of the population of B cells are genome-edited B cells, and/or about 40% or less, about 35% or less, about 30% or less, about 25% or less, about 20% or less, about 15% or less, about 10% or less, or about 5% or less, of the population of B cells lacking an integrated knock-in cassette are viable B cells. In some embodiments, following the contacting step, at least about 80% of the viable B cells of the population of B cells are genome-edited B cells, and about 20% or less of the population of B cells lacking an integrated knock-in cassette are viable B cells. In some embodiments, following the contacting step, at least about 60% of the viable B cells of the population of B cells are genome-edited B cells, and about 40% or less of the population of B cells lacking an integrated knock-in cassette are viable B cells. In some embodiments, following the contacting step, at least about 90% of the viable B cells of the population of B cells are genome-edited B cells, and about 10% or less of the population of B cells lacking an integrated knock-in cassette are viable B cells. In some embodiments, following the contacting step, at least about 95% of the viable B cells of the population of B cells are genome-edited B cells, and about 5% or less of the population of B cells lacking an integrated knock-in cassette are viable B cells.

In some embodiments, if the knock-in cassette is not integrated into the genome of the B cell by homology-directed repair (HDR) in the correct position or orientation, the B cell no longer expresses the gene product encoded by the essential gene, or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the essential gene. In some embodiments, the break is located within the last exon of the essential gene. In some embodiments, the break is located within the penultimate exon of the essential gene.

In some embodiments, the nuclease is highly efficient, e.g., capable of editing at least about 60%, at least about 65%, at least about 75%, at least about 80%, at least about 85%, at least about 90%, at least about 95%, or more, of B cells contacted with the nuclease. In some embodiments, the nuclease is capable of introducing indels (insertions or deletions) in at least about 60%, at least about 65%, at least about 75%, at least about 80%, at least about 85%, at least about 90%, at least about 95%, or more, of B cells contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the B cell (or the population of B cells) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the nuclease is a CRISPR/Cas nuclease selected from Table 5. In some embodiments, the guide molecule comprises a targeting domain sequence that is complementary to a portion of the endogenous coding sequence of the essential gene. In some embodiments, the guide molecule comprises a targeting domain sequence that differs by no more than 3 nucleotides from a sequence that is complementary to a portion of the endogenous coding sequence of the essential gene. In some embodiments, the guide molecule specifically binds to the portion of the endogenous coding sequence of the essential gene. In some embodiments, the guide molecule does not bind to an endogenous coding sequence of another gene, e.g., a different essential gene. In some embodiments, the guide molecule binds to and mediates CRISPR/Cas cleavage at a location within the essential gene that is necessary for function (e.g., functional gene expression or protein function). In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the B cell. In some embodiments, the donor template comprises a 3′ homology arm comprising a sequence homologous to a sequence located 3′ of the break in the genome of the B cell. In some embodiments, the donor template comprises a 5′ homology arm comprising a sequence homologous to a sequence located 5′ of the break in the genome of the B cell, and the donor template comprises a 3′ homology arm comprising a sequence homologous to a sequence located 3′ of the break in the genome of the B cell.

In some embodiments, the knock-in cassette comprises a regulatory element that enables expression of the gene product encoded by the essential gene and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the essential gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the essential gene in the knock-in cassette encodes a C-terminal fragment of a protein encoded by the essential gene. In some embodiments, the C-terminal fragment is less than about 500, 250, 150, 125, 100, 75, 50, 25, 20, 15 or 10 amino acids in length. In some embodiments, the C-terminal fragment includes an amino acid sequence that is encoded by a region of the endogenous coding sequence of the essential gene that spans the break.

In some embodiments, the exogenous coding sequence or partial coding sequence of the essential gene in the knock-in cassette is less than 100% identical to the corresponding endogenous coding sequence of the essential gene of the B cell, e.g., less than 99%, less than 95%, less than 90%, less than 85%, or less than 80% identical to the corresponding endogenous coding sequence of the essential gene of the B cell. In some embodiments, the exogenous coding sequence or partial coding sequence of the essential gene in the knock-in cassette is 80% to 99% identical to the corresponding endogenous coding sequence of the essential gene of the B cell, e.g., 85% to 95% or 90% to 99% identical to the corresponding endogenous coding sequence of the essential gene of the B cell. In some embodiments, the exogenous coding sequence or partial coding sequence of the essential gene in the knock-in cassette has been codon optimized relative to the corresponding endogenous coding sequence of the essential gene of the B cell to remove a target site of the nuclease, to reduce the likelihood of homologous recombination after integration of the knock-in cassette into the genome of the B cell, or to increase expression of the gene product of the essential gene and/or the gene product of interest after integration of the knock-in cassette into the genome of the B cell.

In some embodiments, the nuclease is a Cas (e.g., Cas9 or Cas12a), the exogenous coding sequence or partial coding sequence of the essential gene in the knock-in cassette includes at least one PAM site for the Cas, and the at least one PAM site (or all PAM sites) has been codon optimized or saturated with silent and/or missense mutations.

In some embodiments, the essential gene is GAPDH, TBP, E2F4, G6PD, or KIF11. In some embodiments, the essential gene is a gene selected from Table 3 or Table 4.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited B cell comprises knock-in cassettes at one or both alleles of the essential gene. In some embodiments, the genome-edited B cell expresses (a) the first and second gene products of interest, and (b) the gene product encoded by the essential gene that is required for survival and/or proliferation of the B cell, or a functional variant thereof. In some embodiments, the genome-edited B cell expresses (a) the first and second gene products of interest from the same allele of an essential gene, and (b) the gene product encoded by the essential gene that is required for survival and/or proliferation of the B cell, or a functional variant thereof. In some embodiments, the genome-edited B cell expresses (a) the first and second gene products of interest from different alleles of the essential gene, and (b) the gene product encoded by the essential gene that is required for survival and/or proliferation of the B cell, or a functional variant thereof.

In some embodiments, the method comprises contacting the B cell (or the population of B cells) with a first donor template that comprises a first knock-in cassette comprising a first exogenous coding sequence for a first gene product of interest in frame with and downstream (3′) of an exogenous coding sequence or partial coding sequence of the essential gene, and with a second donor template that comprises a second knock-in cassette comprising a second exogenous coding sequence for a second gene product of interest in frame with and downstream (3′) of an exogenous coding sequence or partial coding sequence of the essential gene. In some embodiments, the genome-edited B cell comprises the first knock-in cassette at a first allele of the essential gene and the second knock-in cassette at the second allele of the essential gene. In some embodiments, the genome-edited B cell expresses (a) the first and second gene products of interest, and (b) the gene product encoded by the essential gene that is required for survival and/or proliferation of the B cell, or a functional variant thereof.

In some embodiments, the method comprises contacting the B cell (or the population of B cells) with a first donor template that comprises a first knock-in cassette comprising a first exogenous coding sequence for a first gene product of interest in frame with and downstream (3′) of an exogenous coding sequence or partial coding sequence of a first essential gene, and with a second donor template that comprises a second knock-in cassette comprising a second exogenous coding sequence for a second gene product of interest in frame with and downstream (3′) of an exogenous coding sequence or partial coding sequence of a second essential gene. In some embodiments, the genome-edited B cell comprises the first knock-in cassette at one or both alleles of the first essential gene and the second knock-in cassette at one or both alleles of the second essential gene. In some embodiments, the genome-edited B cell expresses (a) the first and second gene products of interest, and (b) the gene products encoded by the first and second essential genes required for survival and/or proliferation of the B cell, or a functional variant thereof.

In another aspect, the disclosure features a genetically modified B cell comprising a genome with an exogenous coding sequence for a gene product of interest in frame with and downstream (3′) of a coding sequence of an essential gene, wherein the essential gene encodes a gene product that is required for survival and/or proliferation of the B cell, and wherein at least part of the coding sequence of the essential gene comprises an exogenous coding sequence.

In some embodiments, the exogenous coding sequence of the essential gene comprises about 2000, 1500, 1000, 750, 500, 400, 300, 200, 100, or 50 base pairs of the coding sequence of the essential gene.

In some embodiments, the exogenous coding sequence of the essential gene encodes a C-terminal fragment of a protein encoded by the essential gene. In some embodiments, the C-terminal fragment is less than about 500, 250, 150, 125, 100, 75, 50, 25, 20, 15 or 10 amino acids in length. In some embodiments, the C-terminal fragment includes an amino acid sequence that is encoded by a region of the endogenous coding sequence of the essential gene that spans the break.

In some embodiments, the exogenous coding sequence of the essential gene is less than 100% identical to the corresponding endogenous coding sequence of the essential gene of the B cell. In some embodiments, the exogenous coding sequence of the essential gene has been codon optimized relative to the corresponding endogenous coding sequence of the essential gene of the B cell to remove a target site of a nuclease, e.g., a Cas. In some embodiments, the nuclease is a Cas (e.g., Cas9 or Cas12a), the exogenous coding sequence of the essential gene includes at least one PAM site for the Cas, and the at least one PAM site (or all PAM sites) has been codon optimized or saturated with silent and/or missense mutations.

In some embodiments, the essential gene is GAPDH, TBP, E2F4, G6PD, or KIF11.

In some embodiments the B cell's genome comprises a regulatory element that enables expression of the gene product encoded by the essential gene and the gene product of interest as separate gene products, optionally, wherein at least one of the gene products is a protein and the regulatory element enables expression of that protein separate from the other gene product. In some embodiments, the B cell's genome comprises an IRES or 2A element located between the coding sequence of the essential gene and the exogenous coding sequence for the gene product of interest.

In some embodiments, the B cell's genome comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the B cell's genome does not comprise a reporter gene, e.g., a fluorescent reporter gene or an antibiotic resistance gene.

In another aspect, the disclosure features an engineered B cell comprising a genomic modification, wherein the genomic modification comprises an insertion of an exogenous knock-in cassette within an endogenous coding sequence of an essential gene in the B cell's genome, wherein the essential gene encodes a gene product that is required for survival and/or proliferation of the B cell, wherein the knock-in cassette comprises an exogenous coding sequence for a gene product of interest in frame with and downstream (3′) of an exogenous coding sequence or partial coding sequence encoding the gene product of the essential gene, or a functional variant thereof, and wherein the B cell expresses the gene product of interest and the gene product encoded by the essential gene that is required for survival and/or proliferation of the B cell, or a functional variant thereof, optionally wherein the gene product of interest and the gene product encoded by the essential gene are expressed from the endogenous promoter of the essential gene.

In some embodiments, the exogenous coding sequence or partial coding sequence encoding the gene product of the essential gene comprises about 2000, 1500, 1000, 750, 500, 400, 300, 200, 100, or 50 base pairs of the coding sequence of the essential gene.

In some embodiments, wherein the exogenous coding sequence or partial coding sequence encoding the gene product of the essential gene encodes a C-terminal fragment of a protein encoded by the essential gene. In some embodiments, the C-terminal fragment is less than about 500, 250, 150, 125, 100, 75, 50, 25, 20, 15 or 10 amino acids in length. In some embodiments, the C-terminal fragment includes an amino acid sequence that is encoded by a region of the endogenous coding sequence of the essential gene that spans the break.

In some embodiments, exogenous coding sequence or partial coding sequence encoding the gene product of the essential gene is less than 100% identical to the corresponding endogenous coding sequence of the essential gene of the B cell. In some embodiments, the exogenous coding sequence or partial coding sequence encoding the gene product of the essential gene has been codon optimized relative to the corresponding endogenous coding sequence of the essential gene of the B cell to remove a target site of a nuclease, e.g., a Cas. In some embodiments, the nuclease is a Cas (e.g., Cas9 or Cas12a), the exogenous coding sequence or partial coding sequence encoding the gene product of the essential gene includes at least one PAM site for the Cas, and the at least one PAM site (or all PAM sites) has been codon optimized or saturated with silent and/or missense mutations.

In some embodiments, the essential gene is GAPDH, TBP, E2F4, G6PD, or KIF11.

In some embodiments, the B cell's genome comprises a regulatory element that enables expression of the gene product encoded by the essential gene and the gene product of interest as separate gene products, optionally, wherein at least one of the gene products is a protein and the regulatory element enables expression of that protein separate from the other gene product. In some embodiments, the B cell's genome comprises an IRES or 2A element located between the coding sequence of the essential gene and the exogenous coding sequence for the gene product of interest.

In some embodiments, the B cell's genome comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the B cell's genome does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited B cell comprises knock-in cassettes at one or both alleles of the essential gene. In some embodiments, the genome-edited B cell expresses (a) the first and second gene products of interest, and (b) the gene product encoded by the essential gene that is required for survival and/or proliferation of the B cell, or a functional variant thereof.

In some embodiments, the engineered B cell comprises a first knock-in cassette comprising a first exogenous coding sequence for a first gene product of interest in frame with and downstream (3′) of an exogenous coding sequence or partial coding sequence of the essential gene, and with a second donor template that comprises a second knock-in cassette comprising a second exogenous coding sequence for a second gene product of interest in frame with and downstream (3′) of an exogenous coding sequence or partial coding sequence of the essential gene. In some embodiments, the engineered B cell comprises the first knock-in cassette and the second knock-in cassette at a first allele of the essential gene, optionally wherein the engineered B cell also comprises the first knock-in cassette and the second knock-in cassette at a second allele of the essential gene. In some embodiments, the engineered B cell comprises the first knock-in cassette at a first allele of the essential gene and the second knock-in cassette at the second allele of the essential gene. In some embodiments, the engineered B cell expresses (a) the first and second gene products of interest, and (b) the gene product encoded by the essential gene that is required for survival and/or proliferation of the B cell, or a functional variant thereof.

In some embodiments, the engineered B cell comprises a first knock-in cassette comprising a first exogenous coding sequence for a first gene product of interest in frame with and downstream (3′) of an exogenous coding sequence or partial coding sequence of a first essential gene, and with a second donor template that comprises a second knock-in cassette comprising a second exogenous coding sequence for a second gene product of interest in frame with and downstream (3′) of an exogenous coding sequence or partial coding sequence of a second essential gene. In some embodiments, the engineered B cell comprises the first knock-in cassette at one or both alleles of the first essential gene and the second knock-in cassette at one or both alleles of the second essential gene. In some embodiments, the genome-edited B cell expresses (a) the first and second gene products of interest, and (b) the gene products encoded by the first and second essential genes required for survival and/or proliferation of the B cell, or a functional variant thereof.

In another aspect, the disclosure features any of the B cells described herein for use as a medicament and/or for use in the treatment of a disease, disorder or condition, e.g., a disease, disorder or condition described herein, e.g., a cancer, e.g., a cancer described herein.

In another aspect, the disclosure features a B cell, or a population of B cells, produced by any of the methods described herein, or progeny thereof.

In another aspect, the disclosure features a system for editing the genome of a B cell (or a B cell in a population of B cells), the system comprising the B cell (or the population of B cells), a nuclease that causes a break within an endogenous coding sequence of an essential gene of the B cell, wherein the essential gene encodes a gene product that is required for survival and/or proliferation of the B cell, and a donor template that comprises a knock-in cassette comprising an exogenous coding sequence for a gene product of interest in frame with and downstream (3′) of an exogenous coding sequence or partial coding sequence of the essential gene.

In some embodiments, after contacting the population of B cells with the nuclease and the donor template, at least about 60%, at least about 65%, at least about 70%, at least about 75%, at least about 80%, at least about 85%, at least about 90%, at least about 95%, or more, of the viable B cells of the population of B cells are genome-edited B cells, and/or about 40% or less, about 35% or less, about 30% or less, about 25% or less, about 20% or less, about 15% or less, about 10% or less, or about 5% or less, of the population of B cells lacking an integrated knock-in cassette are viable B cells. In some embodiments, after contacting the population of B cells with the nuclease and the donor template, at least about 80% of the viable B cells of the population of B cells are genome-edited B cells, and about 20% or less of the population of B cells lacking an integrated knock-in cassette are viable B cells. In some embodiments, after contacting the population of B cells with the nuclease and the donor template, at least about 60% of the viable B cells of the population of B cells are genome-edited B cells, and about 40% or less of the population of B cells lacking an integrated knock-in cassette are viable B cells. In some embodiments, after contacting the population of B cells with the nuclease and the donor template, at least about 90% of the viable B cells of the population of B cells are genome-edited B cells, and about 10% or less of the population of B cells lacking an integrated knock-in cassette are viable B cells. In some embodiments, after contacting the population of B cells with the nuclease and the donor template, at least about 95% of the viable B cells of the population of B cells are genome-edited B cells, and about 5% or less of the population of B cells lacking an integrated knock-in cassette are viable B cells.

In some embodiments, after contacting the B cell or population of B cells with the nuclease and the donor template, if the knock-in cassette is not integrated into the genome of the B cell by homology-directed repair (HDR) in the correct position or orientation, the B cell no longer expresses the gene product encoded by the essential gene, or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the essential gene. In some embodiments, the break is located within the last exon of the essential gene.

In some embodiments, the nuclease is highly efficient, e.g., capable of editing at least about 60%, at least about 65%, at least about 75%, at least about 80%, at least about 85%, at least about 90%, at least about 95%, or more, of B cells contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the B cell (or the population of B cells) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the guide molecule comprises a targeting domain sequence that is complementary to a portion of the endogenous coding sequence of the essential gene. In some embodiments, the guide molecule comprises a targeting domain sequence that differs by no more than 3 nucleotides from a sequence that is complementary to a portion of the endogenous coding sequence of the essential gene. In some embodiments, the guide molecule specifically binds to the portion of the endogenous coding sequence of the essential gene. In some embodiments, the guide molecule does not bind to an endogenous coding sequence of another gene, e.g., a different essential gene. In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the B cell. In some embodiments, the donor template comprises a 3′ homology arm comprising a sequence homologous to a sequence located 3′ of the break in the genome of the B cell. In some embodiments, the donor template comprises a 5′ homology arm comprising a sequence homologous to a sequence located 5′ of the break in the genome of the B cell, and the donor template comprises a 3′ homology arm comprising a sequence homologous to a sequence located 3′ of the break in the genome of the B cell.

In some embodiments, the knock-in cassette comprises a regulatory element that enables expression of the gene product encoded by the essential gene and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the essential gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the essential gene in the knock-in cassette encodes a C-terminal fragment of a protein encoded by the essential gene. In some embodiments, the C-terminal fragment is less than about 500, 250, 150, 125, 100, 75, 50, 25, 20, 15 or 10 amino acids in length. In some embodiments, the C-terminal fragment includes an amino acid sequence that is encoded by a region of the endogenous coding sequence of the essential gene that spans the break.

In some embodiments, the exogenous coding sequence or partial coding sequence of the essential gene in the knock-in cassette is less than 100% identical to the corresponding endogenous coding sequence of the essential gene of the B cell. In some embodiments, the exogenous coding sequence or partial coding sequence of the essential gene in the knock-in cassette has been codon optimized relative to the corresponding endogenous coding sequence of the essential gene of the B cell to remove a target site of the nuclease, to reduce the likelihood of homologous recombination after integration of the knock-in cassette into the genome of the B cell, or to increase expression of the gene product of the essential gene and/or the gene product of interest after integration of the knock-in cassette into the genome of the B cell.

In some embodiments, the nuclease is a Cas (e.g., Cas9 or Cas12a), the exogenous coding sequence or partial coding sequence of the essential gene in the knock-in cassette includes at least one PAM site for the Cas, and the at least one PAM site (or all PAM sites) has been codon optimized or saturated with silent and/or missense mutations.

In some embodiments, the essential gene is GAPDH, TBP, E2F4, G6PD, or KIF11.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, after contacting the population of B cells with the nuclease and the donor template, the genome-edited B cell comprises knock-in cassettes at one or both alleles of the essential gene. In some embodiments, the genome-edited B cell expresses (a) the first and second gene products of interest, and (b) the gene product encoded by the essential gene that is required for survival and/or proliferation of the B cell, or a functional variant thereof.

In some embodiments, the system comprises a first a donor template that comprises a first knock-in cassette comprising a first exogenous coding sequence for a first gene product of interest in frame with and downstream (3′) of an exogenous coding sequence or partial coding sequence of the essential gene, and with a second donor template that comprises a second knock-in cassette comprising a second exogenous coding sequence for a second gene product of interest in frame with and downstream (3′) of an exogenous coding sequence or partial coding sequence of the essential gene. In some embodiments, after contacting the population of B cells with the nuclease and the donor templates, the genome-edited B cell comprises the first knock-in cassette at a first allele of the essential gene and the second knock-in cassette at the second allele of the essential gene. In some embodiments, the genome-edited B cell expresses (a) the first and second gene products of interest, and (b) the gene product encoded by the essential gene that is required for survival and/or proliferation of the B cell, or a functional variant thereof.

In some embodiments, the system comprises a first a donor template that comprises a first knock-in cassette comprising a first exogenous coding sequence for a first gene product of interest in frame with and downstream (3′) of an exogenous coding sequence or partial coding sequence of a first essential gene, and with a second donor template that comprises a second knock-in cassette comprising a second exogenous coding sequence for a second gene product of interest in frame with and downstream (3′) of an exogenous coding sequence or partial coding sequence of a second essential gene. In some embodiments, after contacting the population of B cells with the nuclease and the donor templates, the genome-edited B cell comprises the first knock-in cassette at one or both alleles of the first essential gene and the second knock-in cassette at one or both alleles of the second essential gene. In some embodiments, the genome-edited B cell expresses (a) the first and second gene products of interest, and (b) the gene products encoded by the first and second essential genes required for survival and/or proliferation of the B cell, or a functional variant thereof.

In one aspect, the disclosure features a method of producing a population of modified B cells, the method comprising contacting B cells with: (i) a nuclease that causes a break within an endogenous coding sequence of an essential gene in a plurality of the B cells, wherein the essential gene encodes a gene product that is required for survival and/or proliferation of the B cells, and (ii) a donor template that comprises a knock-in cassette comprising an exogenous coding sequence for a gene product of interest in frame with and downstream (3′) of an exogenous coding sequence or partial coding sequence of the essential gene, wherein the knock-in cassette is integrated into the genome of a plurality of the B cells by homology-directed repair (HDR) of the break, resulting in genome-edited B cells that expresses: (a) the gene product of interest, and (b) the gene product encoded by the essential gene that is required for survival and/or proliferation of the plurality of B cells, or a functional variant thereof, and wherein following the contacting step, at least about 60%, at least about 65%, at least about 70%, at least about 75%, at least about 80%, at least about 85%, at least about 90%, at least about 95%, or more, of the viable B cells are genome-edited B cells, and/or about 40% or less, about 35% or less, about 30% or less, about 25% or less, about 20% or less, about 15% or less, about 10% or less, or about 5% or less, of the B cells lacking an integrated knock-in cassette are viable B cells, thereby producing a population of modified B cells. In some embodiments, following the contacting step, at least about 80% of the viable B cells are genome-edited B cells, and about 20% or less of the B cells lacking an integrated knock-in cassette are viable B cells. In some embodiments, following the contacting step, at least about 60% of the viable B cells are genome-edited B cells, and about 40% or less of the B cells lacking an integrated knock-in cassette are viable B cells. In some embodiments, following the contacting step, at least about 90% of the viable B cells are genome-edited B cells, and about 10% or less of the B cells lacking an integrated knock-in cassette are viable B cells. In some embodiments, following the contacting step, at least about 95% of the viable B cells are genome-edited B cells, and about 5% or less of B cells lacking an integrated knock-in cassette are viable B cells.

In some embodiments, if the knock-in cassette is not integrated into the genome of the B cell by homology-directed repair (HDR) in the correct position or orientation, the B cell no longer expresses the gene product encoded by the essential gene, or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the essential gene. In some embodiments, the break is located within the last exon of the essential gene.

In some embodiments, the nuclease is highly efficient, e.g., capable of editing at least about 60%, at least about 65%, at least about 75%, at least about 80%, at least about 85%, at least about 90%, at least about 95%, or more, of B cells contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the B cell (or the population of B cells) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the guide molecule comprises a targeting domain sequence that is complementary to a portion of the endogenous coding sequence of the essential gene. In some embodiments, the guide molecule comprises a targeting domain sequence that differs by no more than 3 nucleotides from a sequence that is complementary to a portion of the endogenous coding sequence of the essential gene. In some embodiments, the guide molecule specifically binds to the portion of the endogenous coding sequence of the essential gene. In some embodiments, the guide molecule does not bind to an endogenous coding sequence of another gene, e.g., a different essential gene. In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the B cell. In some embodiments, the donor template comprises a 3′ homology arm comprising a sequence homologous to a sequence located 3′ of the break in the genome of the B cell. In some embodiments, the donor template comprises a 5′ homology arm comprising a sequence homologous to a sequence located 5′ of the break in the genome of the B cell, and the donor template comprises a 3′ homology arm comprising a sequence homologous to a sequence located 3′ of the break in the genome of the B cell.

In some embodiments, the knock-in cassette comprises a regulatory element that enables expression of the gene product encoded by the essential gene and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the essential gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the essential gene in the knock-in cassette encodes a C-terminal fragment of a protein encoded by the essential gene. In some embodiments, the C-terminal fragment is less than about 500, 250, 150, 125, 100, 75, 50, 25, 20, 15 or 10 amino acids in length. In some embodiments, the C-terminal fragment includes an amino acid sequence that is encoded by a region of the endogenous coding sequence of the essential gene that spans the break.

In some embodiments, the exogenous coding sequence or partial coding sequence of the essential gene in the knock-in cassette is less than 100% identical to the corresponding endogenous coding sequence of the essential gene of the B cell. In some embodiments, the exogenous coding sequence or partial coding sequence of the essential gene in the knock-in cassette has been codon optimized relative to the corresponding endogenous coding sequence of the essential gene of the B cell to remove a target site of the nuclease, to reduce the likelihood of homologous recombination after integration of the knock-in cassette into the genome of the B cell, or to increase expression of the gene product of the essential gene and/or the gene product of interest after integration of the knock-in cassette into the genome of the B cell.

In some embodiments, the nuclease is a Cas (e.g., Cas9 or Cas12a), the exogenous coding sequence or partial coding sequence of the essential gene in the knock-in cassette includes at least one PAM site for the Cas, and the at least one PAM site (or all PAM sites) has been codon optimized or saturated with silent and/or missense mutations.

In some embodiments, the essential gene is GAPDH, TBP, E2F4, G6PD, or KIF11.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited B cells comprise knock-in cassettes at one or both alleles of the essential gene. In some embodiments, the genome-edited B cells expresses (a) the first and second gene products of interest, and (b) the gene product encoded by the essential gene that is required for survival and/or proliferation of the B cells, or a functional variant thereof.

In some embodiments, the method comprises contacting the B cells (or the population of B cells) with a first a donor template that comprises a first knock-in cassette comprising a first exogenous coding sequence for a first gene product of interest in frame with and downstream (3′) of an exogenous coding sequence or partial coding sequence of the essential gene, and with a second donor template that comprises a second knock-in cassette comprising a second exogenous coding sequence for a second gene product of interest in frame with and downstream (3′) of an exogenous coding sequence or partial coding sequence of the essential gene. In some embodiments, the genome-edited B cells comprise the first knock-in cassette at a first allele of the essential gene and the second knock-in cassette at the second allele of the essential gene. In some embodiments, the genome-edited B cells expresses (a) the first and second gene products of interest, and (b) the gene product encoded by the essential gene that is required for survival and/or proliferation of the B cells, or a functional variant thereof.

In some embodiments, the method comprises contacting the B cells (or the population of B cells) with a first a donor template that comprises a first knock-in cassette comprising a first exogenous coding sequence for a first gene product of interest in frame with and downstream (3′) of an exogenous coding sequence or partial coding sequence of a first essential gene, and with a second donor template that comprises a second knock-in cassette comprising a second exogenous coding sequence for a second gene product of interest in frame with and downstream (3′) of an exogenous coding sequence or partial coding sequence of a second essential gene. In some embodiments, the genome-edited B cells comprise the first knock-in cassette at one or both alleles of the first essential gene and the second knock-in cassette at one or both alleles of the second essential gene. In some embodiments, the genome-edited B cells expresses (a) the first and second gene products of interest, and (b) the gene products encoded by the first and second essential genes required for survival and/or proliferation of the B cells, or a functional variant thereof.

In another aspect, the disclosure features a method of selecting and/or identifying a B cell comprising a knock-in of a gene product of interest within an endogenous coding sequence of an essential gene in the B cell, the method comprising contacting a population of B cells with: (i) a nuclease that causes a break within an endogenous coding sequence of an essential gene in a plurality of the B cells, wherein the essential gene encodes a gene product that is required for survival and/or proliferation of the B cells, and (ii) a donor template that comprises a knock-in cassette comprising an exogenous coding sequence for a gene product of interest in frame with and downstream (3′) of an exogenous coding sequence or partial coding sequence of the essential gene, wherein the knock-in cassette is integrated into the genome of a plurality of the B cells by homology-directed repair (HDR) of the break, and identifying a genome-edited B cell within the population of B cells that expresses: (a) the gene product of interest, and (b) the gene product encoded by the essential gene that is required for survival and/or proliferation of the B cell, or a functional variant thereof.

In some embodiments, following the contacting step, at least about 60%, at least about 65%, at least about 70%, at least about 75%, at least about 80%, at least about 85%, at least about 90%, at least about 95%, or more, of the viable B cells of the population of B cells are genome-edited B cells, and/or about 40% or less, about 35% or less, about 30% or less, about 25% or less, about 20% or less, about 15% or less, about 10% or less, or about 5% or less, of the population of B cells lacking an integrated knock-in cassette are viable B cells. In some embodiments, following the contacting step, at least about 80% of the viable B cells of the population of B cells are genome-edited B cells, and about 20% or less of the population of B cells lacking an integrated knock-in cassette are viable B cells. In some embodiments, following the contacting step, at least about 60% of the viable B cells of the population of B cells are genome-edited B cells, and about 40% or less of the population of B cells lacking an integrated knock-in cassette are viable B cells. In some embodiments, following the contacting step, at least about 90% of the viable B cells of the population of B cells are genome-edited B cells, and about 10% or less of the population of B cells lacking an integrated knock-in cassette are viable B cells. In some embodiments, following the contacting step, at least about 95% of the viable B cells of the population of B cells are genome-edited B cells, and about 5% or less of the population of B cells lacking an integrated knock-in cassette are viable B cells.

In some embodiments, if the knock-in cassette is not integrated into the genome of the B cell by homology-directed repair (HDR) in the correct position or orientation, the B cell no longer expresses the gene product encoded by the essential gene, or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the essential gene. In some embodiments, the break is located within the last exon of the essential gene.

In some embodiments, the nuclease is highly efficient, e.g., capable of editing at least about 60%, at least about 65%, at least about 75%, at least about 80%, at least about 85%, at least about 90%, at least about 95%, or more, of B cells contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the B cell (or the population of B cells) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the guide molecule comprises a targeting domain sequence that is complementary to a portion of the endogenous coding sequence of the essential gene. In some embodiments, the guide molecule comprises a targeting domain sequence that differs by no more than 3 nucleotides from a sequence that is complementary to a portion of the endogenous coding sequence of the essential gene. In some embodiments, the guide molecule specifically binds to the portion of the endogenous coding sequence of the essential gene. In some embodiments, the guide molecule does not bind to an endogenous coding sequence of another gene, e.g., a different essential gene. In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the B cell. In some embodiments, the donor template comprises a 3′ homology arm comprising a sequence homologous to a sequence located 3′ of the break in the genome of the B cell. In some embodiments, the donor template comprises a 5′ homology arm comprising a sequence homologous to a sequence located 5′ of the break in the genome of the B cell, and the donor template comprises a 3′ homology arm comprising a sequence homologous to a sequence located 3′ of the break in the genome of the B cell.

In some embodiments, the knock-in cassette comprises a regulatory element that enables expression of the gene product encoded by the essential gene and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the essential gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the essential gene in the knock-in cassette encodes a C-terminal fragment of a protein encoded by the essential gene. In some embodiments, the C-terminal fragment is less than about 500, 250, 150, 125, 100, 75, 50, 25, 20, 15 or 10 amino acids in length. In some embodiments, the C-terminal fragment includes an amino acid sequence that is encoded by a region of the endogenous coding sequence of the essential gene that spans the break.

In some embodiments, the exogenous coding sequence or partial coding sequence of the essential gene in the knock-in cassette is less than 100% identical to the corresponding endogenous coding sequence of the essential gene of the B cell. In some embodiments, the exogenous coding sequence or partial coding sequence of the essential gene in the knock-in cassette has been codon optimized relative to the corresponding endogenous coding sequence of the essential gene of the B cell to remove a target site of the nuclease, to reduce the likelihood of homologous recombination after integration of the knock-in cassette into the genome of the B cell, or to increase expression of the gene product of the essential gene and/or the gene product of interest after integration of the knock-in cassette into the genome of the B cell.

In some embodiments, the nuclease is a Cas (e.g., Cas9 or Cas12a), the exogenous coding sequence or partial coding sequence of the essential gene in the knock-in cassette includes at least one PAM site for the Cas, and the at least one PAM site (or all PAM sites) has been codon optimized or saturated with silent and/or missense mutations.

In some embodiments, the essential gene is GAPDH, TBP, E2F4, G6PD, or KIF11.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited B cell comprises knock-in cassettes at one or both alleles of the essential gene. In some embodiments, the genome-edited B cell expresses (a) the first and second gene products of interest, and (b) the gene product encoded by the essential gene that is required for survival and/or proliferation of the B cell, or a functional variant thereof.

In some embodiments, the method comprises contacting the population of B cells with a first a donor template that comprises a first knock-in cassette comprising a first exogenous coding sequence for a first gene product of interest in frame with and downstream (3′) of an exogenous coding sequence or partial coding sequence of the essential gene, and with a second donor template that comprises a second knock-in cassette comprising a second exogenous coding sequence for a second gene product of interest in frame with and downstream (3′) of an exogenous coding sequence or partial coding sequence of the essential gene. In some embodiments, the genome-edited B cells comprises the first knock-in cassette at a first allele of the essential gene and the second knock-in cassette at the second allele of the essential gene. In some embodiments, the genome-edited B cells expresses (a) the first and second gene products of interest, and (b) the gene product encoded by the essential gene that is required for survival and/or proliferation of the B cell, or a functional variant thereof.

In some embodiments, the method comprises contacting the population of B cells with a first a donor template that comprises a first knock-in cassette comprising a first exogenous coding sequence for a first gene product of interest in frame with and downstream (3′) of an exogenous coding sequence or partial coding sequence of a first essential gene, and with a second donor template that comprises a second knock-in cassette comprising a second exogenous coding sequence for a second gene product of interest in frame with and downstream (3′) of an exogenous coding sequence or partial coding sequence of a second essential gene. In some embodiments, the genome-edited B cells comprises the first knock-in cassette at one or both alleles of the first essential gene and the second knock-in cassette at one or both alleles of the second essential gene. In some embodiments, the genome-edited B cell expresses (a) the first and second gene products of interest, and (b) the gene products encoded by the first and second essential genes required for survival and/or proliferation of the B cell, or a functional variant thereof.

### BRIEF DESCRIPTION OF THE DRAWING

The teachings described herein will be more fully understood from the following description of various exemplary embodiments, when read together with the accompanying drawing. It should be understood that the drawing described below is for illustration purposes only and is not intended to limit the scope of the present teachings in any way.

FIG. 1 shows the locations on the GAPDH gene where exemplary AsCpf1 (AsCas12a) guide RNAs bind, and the results of screening the exemplary guide RNAs that target the GAPDH gene three days after transfection. Results are from gDNA from living cells.

FIG. 2 shows results of screening the exemplary AsCpf1 (AsCas12a) guide RNAs that target the GAPDH gene, three days after transfection. Results are from gDNA from living cells.

FIG. 3A shows an exemplary integration strategy that targets an essential gene according to certain embodiments of the present disclosure. In particular embodiments, introducing a double strand break using CRISPR gene editing (e.g., by Cas12a or Cas9) within a terminal exon (e.g., within about 500 bp upstream (5′) of the stop codon of the essential gene) and administering a donor plasmid with homology arms designed to mediate homology directed repair (HDR) at the cleavage site, results in a population of viable cells carrying a cargo of interest integrated at the essential gene locus. Those cells that were edited by the CRISPR nuclease, but failed to undergo integration of the cargo at the essential gene locus, do not survive.

FIG. 3B shows an exemplary integration strategy that targets the GAPDH gene according to certain embodiments of the present disclosure. The strategy in FIG. 3B can be applied to a variety of cell types, including primary cells, induced pluripotent stem cells (iPSCs), HSCs, and B cells.

FIG. 3C shows an exemplary integration strategy that targets the GAPDH gene according to certain embodiments of the present disclosure. The diagram shows that the only cells that should survive over time are those cells that underwent targeted integration of a cassette that restores the GAPDH locus and includes a cargo of interest, as well as unedited cells. The population of unedited cells following CRISPR editing should be small if the nuclease and guide RNA are highly effective at cleaving the essential gene target site and introduce indels that significantly reduce the function of the essential gene product.

FIG. 3D shows an exemplary integration strategy that targets an essential gene according to certain embodiments of the present disclosure. In particular embodiments, introducing a double strand break using CRISPR gene editing (e.g., by Cas12a or Cas9) to target a 5′ exon (e.g., within about 500 bp downstream (3′) of a start codon of the essential gene) and administering a donor plasmid with homology arms designed to mediate homology directed repair (HDR) at the cleavage site, results in a population of viable cells carrying a cargo of interest integrated at the essential gene locus. Those cells that were edited by the CRISPR nuclease, but failed to undergo integration of the cargo at the essential gene locus, do not survive.

FIG. 4A depicts exemplary flow cytometry data from AAV6-mediated knock-in of GFP into B cells without RNPs. Unedited cells made up 100.0% of the bulk population.

FIG. 4B depicts exemplary flow cytometry data from AAV6-mediated knock-in of GFP into B cells using RNPs comprising RSQ22337 targeting GAPDH and Cas12a (SEQ ID NO: 62). Edited GFP+ cells made up 96.8% of the bulk population. Results from three replicates are graphed in the right panel, which has a Y-axis that represents the percentage of GFP+ cells in the bulk population as measured by flow cytometry.

FIG. 5A depicts exemplary flow cytometry data from wild-type (WT) B cells. CD19+ cells made up 100% of the bulk population. GFP+ cells made up 0% of the bulk population.

FIG. 5B depicts exemplary flow cytometry data from B cells with AAV6-mediated knock-in of GFP using RNPs comprising RSQ22337 targeting GAPDH and Cas12a (SEQ ID NO: 62). CD19+GFP+ cells made up 97.0% of the bulk population.

FIG. 6 depicts exemplary flow cytometry data from B cells with (i) double knockout (DKO) of B2M and CIITA and (ii) knock-in of a HLA-E transgenic construct, using RNPs comprising gRNAs targeting B2M, CIITA, and GAPDH and Cas12a (SEQ ID NO: 62) and AAV-mediated knock-in of the HLA-E transgenic construct, as described in WO 2022/272292, at the GAPDH locus through AAV transduction. As shown in the plot at left, B2M/CIITA DKO cells made up about 95.5% of the bulk population, and as shown in the plot at center, HLA-E+ cells made up about 90.5% of the B2M/CIITA DKO cells. Unedited control cells exhibited no notable expression of HLA-E as displayed in the plot at right. The X-axis of the left plot represents CIITA expression, and the Y-axis of the left plot represents B2M expression. The X-axis of the center and right plots represents HLA-E expression, and the Y-axis of the center and right plots represents side scatter.

FIG. 7 depicts exemplary knockout and knock-in efficiency in B cells as measured by flow cytometry. RNPs comprising Cas12a (SEQ ID NO: 62) and a gRNA (SEQ ID NO: 2000) targeting B2M, either gRNA #1 (SEQ ID NO: 2001) or gRNA #2 (SEQ ID NO: 2002) targeting CIITA, and RSQ22337 targeting GAPDH were used to knockout B2M and CIITA and to knock-in a HLA-E transgenic construct, as described in WO 2022/272292, at the GAPDH locus through AAV transduction. The X axis denotes the edit (DKO=B2M and CIITA double knock-out; HLAE+=HLA-E knock-in) and the CIITA gRNA used, with each set of vertical bars per edit representing varying RNP concentration (from left to right: 4 uM, 2 uM, 1 uM). The Y axis represents the percentage of cells containing the noted edit as determined by flow cytometry.

### DETAILED DESCRIPTION

### Definitions and Abbreviations

Unless otherwise specified, each of the following terms have the meaning set forth in this section.

The indefinite articles “a” and “an” refer to at least one of the associated noun, and are used interchangeably with the terms “at least one” and “one or more.” The conjunctions “or” and “and/or” are used interchangeably as non-exclusive disjunctions.

The term “antibody” as used herein refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are composed of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fe regions for Fe receptors can be modulated through glycosylation or other modification. In some embodiments, antibodies produced and/or utilized in accordance with the present disclosure include glycosylated Fe domains, including Fe domains with modified or engineered such glycosylation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are fully human, or are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present disclosure is in a format selected from, but not limited to, intact IgG, IgE and IgM, bi- or multi-specific antibodies (e.g., Zybodies®, etc.), bi- or multi-paratopic antibodies, single chain Fys, polypeptide-Fc fusions, Fabs, camelid antibodies, masked antibodies (e.g., Probodies®), Small Modular ImmunoPharmaceuticals (“SMIPs™”), single chain or Tandem diabodies (TandAb®), VHHs, Anticalins®, Nanobodies®, minibodies, BiTE®s, ankyrin repeat proteins or DARPINs®, Avimers®, a DART, a TCR-like antibody, Adnectins®, Affilins®, Trans-Bodies®, Affibodies®, a TrimerX®, MicroProteins, Fynomers®, Centyrins®, and a KALBITOR®. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.)).

The term “cancer” (also used interchangeably with the term “neoplastic”), as used herein, refers to cells having the capacity for autonomous growth, i.e., an abnormal state or condition characterized by rapidly proliferating cell growth. Cancerous disease states may be categorized as pathologic, i.e., characterizing or constituting a disease state, e.g., malignant tumor growth, or may be categorized as non-pathologic, i.e., a deviation from normal but not associated with a disease state, e.g., cell proliferation associated with wound repair.

The terms “CRISPR/Cas nuclease” as used herein refer to any CRISPR/Cas protein with DNA nuclease activity, e.g., a Cas9 or a Cas12 protein that exhibits specific association (or “targeting”) to a DNA target site, e.g., within a genomic sequence in a cell in the presence of a guide molecule. The strategies, systems, and methods disclosed herein can use any combination of CRISPR/Cas nuclease disclosed herein, or known to those of ordinary skill in the art. Those of ordinary skill in the art will be aware of additional CRISPR/Cas nucleases and variants suitable for use in the context of the present disclosure, and it will be understood that the present disclosure is not limited in this respect.

The term “differentiation” as used herein is the process by which an unspecialized (“uncommitted”) or less specialized cell acquires the features of a specialized cell such as, for example, a blood cell. In some embodiments, a differentiated or differentiation-induced cell is one that has taken on a more specialized (“committed”) position within the lineage of a cell. For example, an iPS cell (iPSC) can be differentiated into various more differentiated cell types, for example, a hematopoietic stem cell, a lymphocyte, and other cell types, upon treatment with suitable differentiation factors in the cell culture medium. Suitable methods, differentiation factors, and cell culture media for the differentiation of pluri- and multipotent cell types into more differentiated cell types are well known to those of skill in the art. In some embodiments, the term “committed”, is applied to the process of differentiation to refer to a cell that has proceeded through a differentiation pathway to a point where, under normal circumstances, it would or will continue to differentiate into a specific cell type or subset of cell types, and cannot, under normal circumstances, differentiate into a different cell type (other than a specific cell type or subset of cell types) nor revert to a less differentiated cell type.

The term “nuclease” as used herein refers to any protein that catalyzes the cleavage of phosphodiester bonds. In some embodiments the nuclease is a DNA nuclease. In some embodiments the nuclease is a “nickase” which causes a single-strand break when it cleaves double-stranded DNA, e.g., genomic DNA in a cell. In some embodiments the nuclease causes a double-strand break when it cleaves double-stranded DNA, e.g., genomic DNA in a cell. In some embodiments the nuclease binds a specific target site within the double-stranded DNA that overlaps with or is adjacent to the location of the resulting break. In some embodiments, the nuclease causes a double-strand break that contains overhangs ranging from 0 (blunt ends) to 22 nucleotides in both 3′ and 5′ orientations. As discussed herein, CRISPR/Cas nucleases, zinc finger nucleases (ZFNs), transcription activator-like effector nucleases (TALENs) and meganucleases are exemplary nucleases that can be used in accordance with the strategies, systems, and methods of the present disclosure.

The term “embryonic stem cell” as used herein refers to pluripotent stem cells derived from the inner cell mass of the embryonic blastocyst. In some embodiments, embryonic stem cells are pluripotent and give rise during development to all derivatives of the three primary germ layers: ectoderm, endoderm and mesoderm. In some such embodiments, embryonic stem cells do not contribute to the extra-embryonic membranes or the placenta, i.e., are not totipotent.

The term “endogenous” as used herein in the context of nucleic acids refers to a native nucleic acid (e.g., a gene, a protein coding sequence) in its natural location, e.g., within the genome of a cell.

The term “essential gene” as used herein with respect to a cell refers to a gene that encodes at least one gene product that is required for survival, proliferation, development, and/or differentiation of the cell. An essential gene can be a housekeeping gene that is essential for survival of all cell types or a gene that is required to be expressed in a specific cell type for survival, proliferation, and development under particular culture conditions, e.g., for proper differentiation of iPS or ES cells or expansion of iPS- or ES-derived cells. Loss of function of an essential gene results, in some embodiments, in a significant reduction of cell survival, e.g., of the time a cell characterized by a loss of function of an essential gene survives as compared to a cell of the same cell type but without a loss of function of the same essential gene. In some embodiments, loss of function of an essential gene results in the death of the affected cell. In some embodiments, loss of function of an essential gene results in a significant reduction of cell proliferation, e.g., in the ability of a cell to divide, which can manifest in a significant time period the cell requires to complete a cell cycle, or, in some preferred embodiments, in a loss of a cell's ability to complete a cell cycle, and thus to proliferate at all.

The term “exogenous,” as used herein in the context of nucleic acids refers to a nucleic acid (whether native or non-native) that has been artificially introduced into a man-made construct (e.g., a knock-in cassette, or a donor template) or into the genome of a cell using, for example, gene editing or genetic engineering techniques, e.g., HDR based integration techniques.

The term “guide molecule” or “guide RNA” or “gRNA” when used in reference to a CRISPR/Cas system is any nucleic acid that promotes the specific association (or “targeting”) of a CRISPR/Cas nuclease, e.g., a Cas9 or a Cas12 protein to a DNA target site such as within a genomic sequence in a cell. While guide molecules are typically RNA molecules it is well known in the art that chemically modified RNA molecules including DNA/RNA hybrid molecules can be used as guide molecules.

The terms “hematopoietic stem cell,” or “definitive hematopoietic stem cell” as used herein, refer to CD34-positive (CD34+) stem cells. In some embodiments, CD34-positive stem cells are capable of giving rise to mature lymphoid cell types. In some embodiments, the lymphoid cell types include, for example, B cells.

The terms “induced pluripotent stem cell”, “iPS cell” or “iPSC” as used herein to refer to a stem cell obtained from a differentiated somatic (e.g., adult, neonatal, or fetal) cell by a process referred to as reprogramming (e.g., dedifferentiation). In some embodiments, reprogrammed cells are capable of differentiating into tissues of all three germ or dermal layers: mesoderm, endoderm, and ectoderm. iPSCs are not found in nature.

The term “multipotent stem cell” as used herein refers to a cell that has the developmental potential to differentiate into cells of one or more germ layers (ectoderm, mesoderm and endoderm), but not all three germ layers. Thus, in some embodiments, a multipotent cell may also be termed a “partially differentiated cell.” Multipotent cells are well-known in the art, and examples of multipotent cells include adult stem cells, such as for example, hematopoietic stem cells and neural stem cells. In some embodiments, “multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, in some embodiments, “multipotency” refers to a state of a cell with a degree of developmental potential that is less than totipotent and pluripotent.

The term “pluripotent” as used herein refers to ability of a cell to form all lineages of the body or soma (i.e., the embryo proper) or a given organism (e.g., human). For example, embryonic stem cells are a type of pluripotent stem cells that are able to form cells from each of the three germ layers, the ectoderm, the mesoderm, and the endoderm. Generally, pluripotency may be described as a continuum of developmental potencies ranging from an incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell or an induced pluripotent stem cell).

The term “pluripotency” as used herein refers to a cell that has the developmental potential to differentiate into cells of all three germ layers (ectoderm, mesoderm, and endoderm). In some embodiments, pluripotency can be determined, in part, by assessing pluripotency characteristics of the cells. In some embodiments, pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GC™-2, TG343, TG30, CD9, CD29, CD133/prominin, CD140a, CD56, CD73, CD90, CD105, OCT4 (also known as POU5F1), NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

The term “pluripotent stem cell morphology” as used herein refers to the classical morphological features of an embryonic stem cell. In some embodiments, normal embryonic stem cell morphology is characterized as small and round in shape, with a high nucleus-to-cytoplasm ratio, the notable presence of nucleoli, and typical intercell spacing.

The term “polycistronic” or “multicistronic” when used herein with reference to a knock-in cassette refers to the fact that the knock-in cassette can express two or more proteins from the same mRNA transcript. Similarly, a “bicistronic” knock-in cassette is a knock-in cassette that can express two proteins from the same mRNA transcript.

The term “polynucleotide” (including, but not limited to “nucleotide sequence”, “nucleic acid”, “nucleic acid molecule”, “nucleic acid sequence”, and “oligonucleotide”) as used herein refers to a series of nucleotide bases (also called “nucleotides”) and means any chain of two or more nucleotides. In some embodiments, polynucleotides, nucleotide sequences, nucleic acids, etc. can be chimeric mixtures or derivatives or modified versions thereof, single-stranded or double-stranded. In some such embodiments, modifications can occur at the base moiety, sugar moiety, or phosphate backbone, for example, to improve stability of the molecule, its hybridization parameters, etc. In general, a nucleotide sequence typically carries genetic information, including, but not limited to, the information used by cellular machinery to make proteins and enzymes. In some embodiments, a nucleotide sequence and/or genetic information comprises double- or single-stranded genomic DNA, RNA, any synthetic and genetically manipulated polynucleotide, and/or sense and/or antisense polynucleotides. In some embodiments, nucleic acids contain modified bases.

Conventional IUPAC notation is used in nucleotide sequences presented herein, as shown in Table 1, below (see also Cornish-Bowden, Nucleic Acids Res. 1985; 13(9):3021-30, incorporated by reference herein). It should be noted, however, that “T” denotes “Thymine or Uracil” in those instances where a sequence may be encoded by either DNA or RNA, for example in certain CRISPR/Cas guide molecule targeting domains.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “potency” or “developmental potency” as used herein refer to the sum of all developmental options accessible to the cell (i.e., the developmental potency), particularly, for example in the context of cellular developmental potential. In some embodiments, the continuum of cell potency includes, but is not limited to, totipotent cells, pluripotent cells, multipotent cells, oligopotent cells, unipotent cells, and terminally differentiated cells.

The terms “prevent,” “preventing,” and “prevention” as used herein with reference to a disease refer to the prevention of the disease in a mammal, e.g., in a human, including (a) avoiding or precluding the disease; (b) affecting the predisposition toward the disease; or (c) preventing or delaying the onset of at least one symptom of the disease.

The terms “protein,” “peptide” and “polypeptide” as used herein are used interchangeably to refer to a sequential chain of amino acids linked together via peptide bonds. The terms include individual proteins, groups or complexes of proteins that associate together, as well as fragments or portions, variants, derivatives and analogs of such proteins. Unless otherwise specified, peptide sequences are presented herein using conventional notation, beginning with the amino or N-terminus on the left, and proceeding to the carboxyl or C-terminus on the right. Standard one-letter or three-letter abbreviations can be used.

The term “gene product of interest” as used herein can refer to any product encoded by a gene including any polynucleotide or polypeptide. In some embodiments the gene product is a protein which is not naturally expressed by a target cell of the present disclosure. It is to be understood that the methods and cells of the present disclosure are not limited to any particular gene product of interest and that the selection of a gene product of interest will depend on the type of cell and ultimate use of the cells.

The term “reporter gene” as used herein refers to an exogenous gene that has been introduced into a cell, e.g., integrated into the genome of the cell, that confers a trait suitable for artificial selection. Common reporter genes are fluorescent reporter genes that encode a fluorescent protein, e.g., green fluorescent protein (GFP) and antibiotic resistance genes that confer antibiotic resistance to cells.

The terms “reprogramming” or “dedifferentiation” or “increasing cell potency” or “increasing developmental potency” as used herein refer to a method of increasing potency of a cell or dedifferentiating a cell to a less differentiated state. For example, in some embodiments, a cell that has an increased cell potency has more developmental plasticity (i.e., can differentiate into more cell types) compared to the same cell in the non-reprogrammed state. That is, in some embodiments, a reprogrammed cell is one that is in a less differentiated state than the same cell in a non-reprogrammed state. In some embodiments, “reprogramming” refers to de-differentiating a somatic cell, or a multipotent stem cell, into a pluripotent stem cell, also referred to as an induced pluripotent stem cell, or iPSC. Suitable methods for the generation of iPSCs from somatic or multipotent stem cells are well known to those of skill in the art.

The term “subject” as used herein means a human or non-human animal. In some embodiments a human subject can be any age (e.g., a fetus, infant, child, young adult, or adult). In some embodiments a human subject may be at risk of or suffer from a disease, or may be in need of alteration of a gene or a combination of specific genes. Alternatively, in some embodiments, a subject may be a non-human animal, which may include, but is not limited to, a mammal. In some embodiments, a non-human animal is a non-human primate, a rodent (e.g., a mouse, rat, hamster, guinea pig, etc.), a rabbit, a dog, a cat, and so on. In certain embodiments of this disclosure, the non-human animal subject is livestock, e.g., a cow, a horse, a sheep, a goat, etc. In certain embodiments, the non-human animal subject is poultry, e.g., a chicken, a turkey, a duck, etc.

The terms “treatment,” “treat,” and “treating,” as used herein refer to a clinical intervention aimed to reverse, alleviate, delay the onset of, or inhibit the progress, ameliorate, reduce severity of, prevent or delay the recurrence of a disease, disorder, or condition or one or more symptoms thereof, and/or improve one or more symptoms of a disease, disorder, or condition as described herein. In some embodiments, a condition includes an injury. In some embodiments, an injury may be acute or chronic (e.g., tissue damage from an underlying disease or disorder that causes, e.g., secondary damage such as tissue injury). In some embodiments, treatment, e.g., in the form of a B cell or a population of B cells as described herein, may be administered to a subject after one or more symptoms have developed and/or after a disease has been diagnosed. Treatment may be administered in the absence of symptoms, e.g., to prevent or delay onset of a symptom or inhibit onset or progression of a disease. For example, in some embodiments, treatment may be administered to a susceptible subject prior to the onset of symptoms (e.g., in light of genetic or other susceptibility factors). In some embodiments, treatment may also be continued after symptoms have resolved, for example to prevent or delay their recurrence. In some embodiments, treatment results in improvement and/or resolution of one or more symptoms of a disease, disorder or condition.

### Methods of Editing the Genome of a Cell

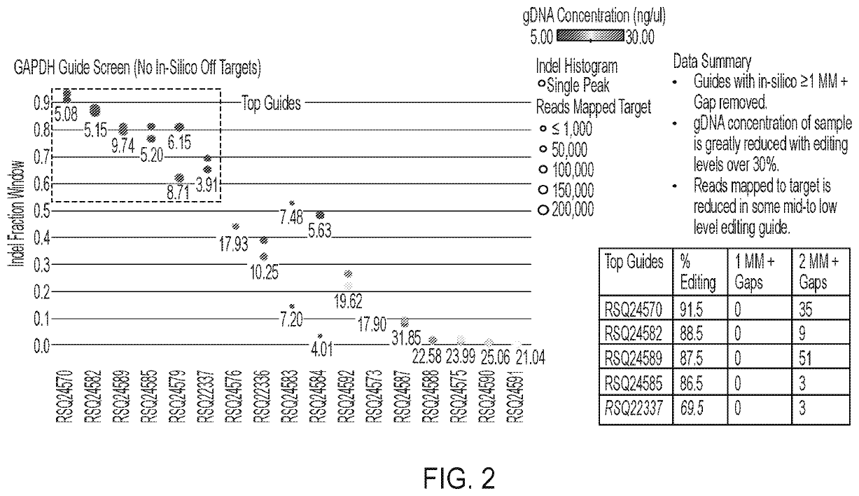

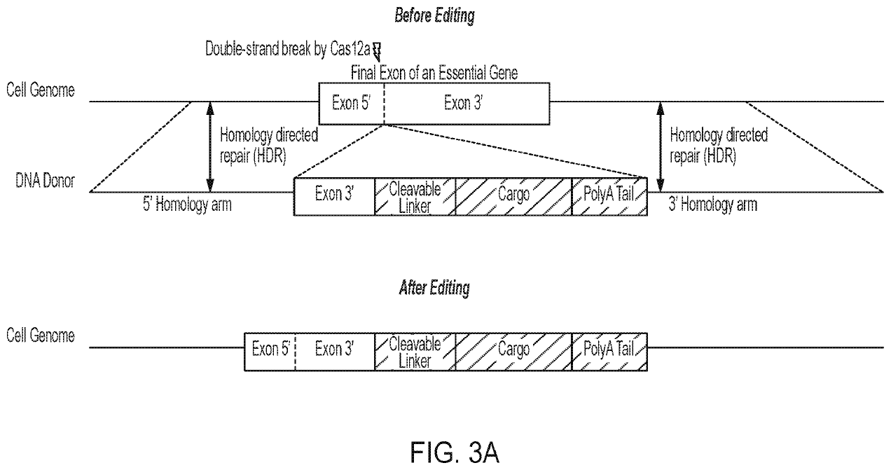

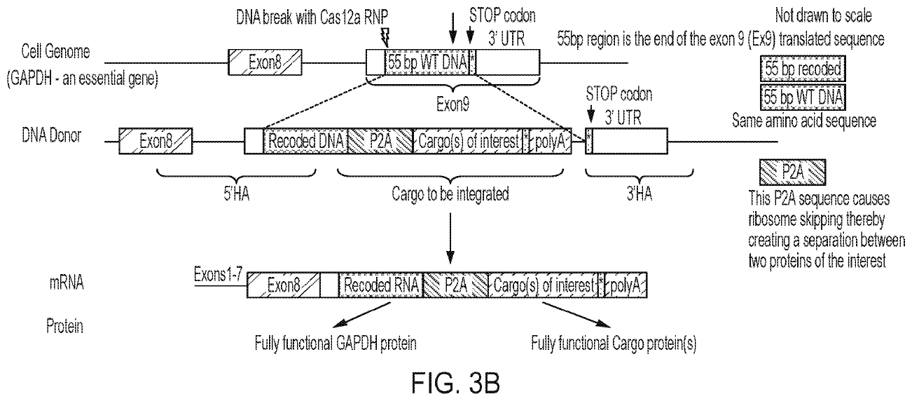

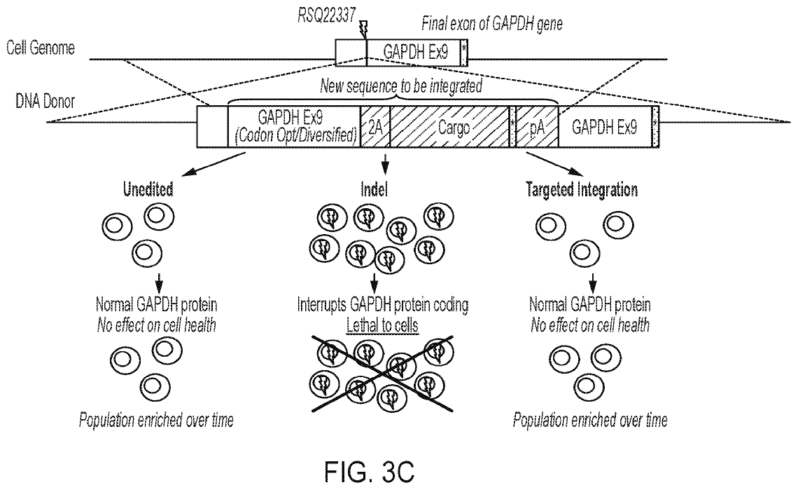

In one aspect, the present disclosure provides methods of editing the genome of a cell. In certain embodiments, the method comprises contacting the cell with a nuclease that causes a break within an endogenous coding sequence of an essential gene in the cell wherein the essential gene encodes at least one gene product that is required for survival, proliferation, and/or development of the cell. The cell is also contacted with (i) a donor template that comprises a knock-in cassette comprising an exogenous coding sequence for a gene product of interest in frame with and downstream (3′) of an exogenous coding sequence or partial coding sequence of the essential gene (FIG. 3B) and/or (ii) a donor template that comprises a knock-in cassette comprising an exogenous coding sequence for a gene product of interest in frame with and upstream (5′) of an exogenous coding sequence or partial coding sequence of the essential gene (FIG. 3D). The knock-in cassette is integrated into the genome of the cell by homology-directed repair (HDR) of the break, resulting in a genome-edited cell that expresses the gene product of interest and the gene product encoded by the essential gene that is required for survival, proliferation, and/or development of the cell, or a functional variant thereof. The genetically modified “knock-in” cell survives and proliferates to produce progeny cells with genomes that also include the exogenous coding sequence for the gene product of interest. This is illustrated in FIG. 3A for an exemplary method.

If the knock-in cassette is not properly integrated into the genome of the cell, undesired editing events that result from the break, e.g., NHEJ-mediated creation of indels, may produce a non-functional, e.g., out of frame, version of the essential gene. This produces a “knock-out” cell when the editing efficiency of the nuclease is high enough to disrupt both alleles. In certain embodiments, this produces a “knock-out” cell when the editing efficiency of the nuclease is high enough to disrupt one allele. Without sufficient functional copies of the essential gene these “knock-out” cells are unable to survive and do not produce any progeny cells.

Since the “knock-in” cells survive and the “knock-out” cells do not survive, the method automatically selects for the “knock-in” cells when it is applied to a population of starting cells. Significantly, in certain embodiments, the method does not require high knock-in efficiencies because of this automatic selection aspect. It is therefore particularly suitable for methods where the donor template is a dsDNA (e.g., a plasmid) where knock-in efficiencies are often below 5%. As noted in the exemplary method of FIG. 3C, in some embodiments some of the cells in the population of starting cells may remain unedited, i.e., unaffected by the nuclease. These cells would also survive and produce progeny with genomes that do not include the exogenous coding sequence for the gene product of interest. When the nuclease editing efficiency is high, e.g., about 60-90%, or higher the percentage of unedited cells will be relatively low as compared to the percentage of genetically modified cells. In some embodiments, high nuclease editing efficiencies (e.g., greater than 65%, greater than 70%, greater than 75%, greater than 80%, greater than 85%, greater than 90%, or greater than 95%) facilitates efficient population wide transgene integration, as the percentage of unedited cells will be relatively low as compared to the percentage of genetically modified cells. In some embodiments of the methods disclosed herein, at least about 65% of the cells (e.g., about 70%, about 75%, about 80%, about 85%, about 90%, about 91%, about 92%, about 93%, about 94%, about 95%, about 96%, about 97%, about 98%, or about 99% of the cells) are edited by a nuclease, e.g., an Cas12a or Cas9. In some embodiments, an RNP containing a CRISPR nuclease (e.g., Cas9 or Cas12a) and a guide are capable of cleaving the locus of an essential gene (e.g., a terminal exon in the locus of any essential gene provided in Table 3) in at least 65% of the cells in a population of cells (e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the cells in a population of cells). In some embodiments, editing efficiency is determined prior to target cell die off, e.g., at day 1 and/or day 2 post transfection or transduction. In some embodiments, editing efficiency measured at day 1 and/or day 2 post transfection or transduction may not capture the complete proportion of cells for which editing occurred, as in some embodiments, certain editing events may result in near immediate and/or swift cell death. In some embodiments, near immediate and/or swift cell death may be any period of time less than 48 hours post transfection or transduction, for example, less than 48 hours, less than 44 hours, less than 40 hours, less than 36 hours, less than 32 hours, less than 28 hours, less than 24 hours, less than 20 hours, less than 16 hours, less than 15 hours, less than 14 hours, less than 13 hours, less than 12 hours, less than 11 hours, less than 10 hours, less than 9 hours, less than 8 hours, less than 7 hours, less than 6 hours, less than 5 hours, less than 4 hours, less than 3 hours, less than 2 hours, or less than 1 hour after transfection or transduction.

In some embodiments, the nuclease causes a double-strand break. In some embodiments the nuclease causes a single-strand break, e.g., in some embodiments the nuclease is a nickase. In some embodiments the nuclease is a prime editor which comprises a nickase domain fused to a reverse transcriptase domain. In some embodiments the nuclease is an RNA-guided prime editor and the gRNA comprises the donor template. In some embodiments a dual-nickase system is used which causes a double-strand break via two single-strand breaks on opposing strands of a double-stranded DNA, e.g., genomic DNA of the cell.

In some embodiments, the present disclosure provides methods suitable for high-efficiency knock-in (e.g., a high proportion of a cell population comprises a knock-in allele), overcoming a major manufacturing challenge. Historically, gene of interest knock-in using plasmid vectors results in efficiencies typically between 0.1 and 5% (see e.g., Zhu et al., CRISPR/Cas-Mediated Selection-free Knockin Strategy in Human Embryonic Stem Cells. Stem Cell Reports. 2015; 4(6):1103-1111). This low knock-in efficiency can result in a need for extensive time and resources devoted to screening potentially edited clones. Additionally, gene of interest knock-in into B cells, particularly primary human B cells, has been notably inefficient even when using viral vectors as compared to other cell types—with knock-in efficiencies averaging between 10 and 25% (see e.g., Johnson et al., Engineering of Primary Human B cells with CRISPR/Cas9 Targeted Nuclease. Sci. Rep. 2018; 8(1):12144). Efficiencies seen with the usage of other template types (e.g., ssODNs) are further reduced (see e.g., Wu et al., Genetic engineering in primary human B cells with CRISPR-Cas9 ribonucleoproteins. J Immunol Methods. 2018; 457:33-40).

In some embodiments, a gene of interest knocked into a cell may have a role in effector function, specificity, stealth, persistence, homing/chemotaxis, and/or resistance to certain chemicals (see for example, Saetersmoen et al., Seminars in Immunopathology, 2019).

In certain embodiments, the present disclosure provides methods for creation of knock-in cells that maintain high levels of expression regardless of age, differentiation status, and/or exogenous conditions. For example, in some embodiments, an integrated cargo is expressed at an optimal level with a desired subcellular localization as a function of an insertion site. In some embodiments, the present disclosure provides such cells.

### Systems for Editing the Genome of a Cell

In one aspect the present disclosure provides systems for editing the genome of a cell. In some embodiments, the system comprises the cell, a nuclease that causes a break within an endogenous coding sequence of an essential gene of the cell, wherein the essential gene encodes a gene product that is required for survival, proliferation, and/or development of the cell, and a donor template that comprises a knock-in cassette comprising an exogenous coding sequence for a gene product of interest in frame with and downstream (3′) of an exogenous coding sequence or partial coding sequence of the essential gene.

In some embodiments, the nuclease causes a double-strand break. In some embodiments the nuclease causes a single-strand break, e.g., in some embodiments the nuclease is a nickase. In some embodiments the nuclease is a prime editor which comprises a nickase domain fused to a reverse transcriptase domain. In some embodiments the nuclease is an RNA-guided prime editor and the gRNA comprises the donor template. In some embodiments a dual-nickase system is used which causes a double-strand break via two single-strand breaks on opposing strand of a double-stranded DNA, e.g., genomic DNA of the cell.

Genome editing systems can be implemented (e.g., administered or delivered to a cell or a subject) in a variety of ways, and different implementations may be suitable for distinct applications. For instance, a genome editing system is implemented, in certain embodiments, as a protein/RNA complex (a ribonucleoprotein, or RNP). In certain embodiments, a genome editing system is implemented as one or more nucleic acids encoding an RNA-guided nuclease and guide RNA components described herein (optionally with one or more additional components); in certain embodiments, a genome editing system is implemented as one or more vectors comprising such nucleic acids, for instance a viral vector such as an adeno-associated virus; and in certain embodiments, a genome editing system is implemented as a combination of any of the foregoing. Additional or modified implementations that operate according to the principles set forth herein will be apparent to the skilled artisan and are within the scope of this disclosure.

In some embodiments, methods as described herein include performing certain steps in at least duplicate. For example, in some embodiments, integration of certain gene products of interest, particularly including multiple genes of interest or a large number of exogenous gene sequences, may result in an initial selection round that results in a lower than desired level of targeted integration. In certain embodiments, a lower than desirable levels of nuclease activity and/or of knock-in cassette targeted integration may result in a lower than desirable percentage of surviving cells and/or cells comprising the knock-in cassette; this may make identifying a cell with the genetic payload difficult. In some embodiments, to further enrich for the population of edited cells, cells were optionally expanded and then re-edited by providing the pool of edited cells with either both RNP and donor templates (e.g., one or more RNP particles targeting one or more loci, and one or more donor templates designed for targeted integration at one or more loci), or just RNP alone (e.g., one or more RNP that utilize residual donor template).

In some embodiments, where multiple rounds of RNP and/or donor template editing is performed, enrichment is affected by: i) removing cells that have not incorporated the genetic payload and/or ii) creating more cells with incorporated knock-in cassette. In some embodiments, the effectiveness of an additional enrichment steps, depending on the cargo, depending on whether multiple constructs are used, the target within the essential gene, or other factors, can lead to at least about two-fold, three-fold, four-fold, five-fold, or higher improvement in the percentage of cells incorporating the knock-in cassette from the donor template. In some embodiments, such enrichment can lead to uptake of the “cargo” within the essential gene of mammalian cells of greater than 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or greater than 95%.

In some embodiments, donor templates (e.g., donor nucleic acid constructs) comprise the transgene flanked by a first homologous region (HR) e.g., a homology arm, and a second HR, e.g., a second homology arm, designed to anneal to a first genomic region (GR) and a second GR within an essential gene of a cell. To be able to anneal, the HRs and GRs need not be perfectly homologous. In some embodiments, examples include a non-inhibitory small number (less than 6 and as few as 1) of mutations in the PAM 5′ of the transgene in the knock-in cassette. In some embodiments, other non-inhibitory changes include codon optimization, wherein unnecessary nucleotides in the wildtype exon are removed from the nucleotide sequence in the knock-in cassette. In some embodiments, other such silent PAM blocking mutations or codon modifications that prevent cleavage of the donor nucleic acid construct by the nuclease are further contemplated. In some embodiments, at least about 90% homology is sufficient for functional annealing for purposes of the examples herein. In some embodiments, the level of homology between the HR and GR is more than 90%, more than 92%, more than 94%, more than 96%, more than 98%, or more than 99%. Other embodiments and the concepts set forth in this paragraph are contemplated and subsumed in the term “essentially homologous.”

### Genetically Modified Cells

In one aspect the present disclosure provides genetically modified cells or engineered cells including populations of such cells and progeny of such cells.

In some embodiments, the cell is produced by a method of the present disclosure, e.g., a method that comprises contacting the cell with a nuclease that causes a break within an endogenous coding sequence of an essential gene in the cell wherein the essential gene encodes at least one gene product that is required for survival, proliferation, and/or development of the cell. The cell is also contacted with a donor template that comprises a knock-in cassette comprising an exogenous coding sequence for a gene product of interest in frame with and downstream (3′) of an exogenous coding sequence or partial coding sequence of the essential gene. The knock-in cassette is integrated into the genome of the cell by homology-directed repair (HDR) of the break, resulting in a genome-edited cell that expresses the gene product of interest and the gene product encoded by the essential gene that is required for survival, proliferation, and/or development of the cell, or a functional variant thereof. This is illustrated in FIG. 3 for an exemplary method. In some embodiments, a cell is contacted with a donor template that comprises a knock-in cassette comprising an exogenous coding sequence for a gene product of interest in frame with and upstream (5′) of an exogenous coding sequence or partial coding sequence of the essential gene.

In some embodiments, the cell comprises a genome with an exogenous coding sequence for a gene product of interest in frame with and downstream (3′) of a coding sequence of an essential gene, wherein the essential gene encodes a gene product that is required for survival, proliferation, and/or development of the cell.

In some embodiments, the cell comprises a genome with an exogenous coding sequence for a gene product of interest in frame with and upstream (5′) of a coding sequence of an essential gene, wherein the essential gene encodes a gene product that is required for survival, proliferation, and/or development of the cell.

In some embodiments, the cell comprises a genomic modification, wherein the genomic modification comprises an insertion of an exogenous knock-in cassette within an endogenous coding sequence of an essential gene in the cell's genome, wherein the essential gene encodes a gene product that is required for survival, proliferation, and/or development of the cell, wherein the knock-in cassette comprises an exogenous coding sequence for a gene product of interest in frame with and downstream (3′) of an exogenous coding sequence or partial coding sequence encoding the gene product of the essential gene, or a functional variant thereof, and wherein the cell expresses the gene product of interest and the gene product encoded by the essential gene that is required for survival, proliferation, and/or development of the cell, or a functional variant thereof. In some embodiments, the gene product of interest and the gene product encoded by the essential gene are expressed from the endogenous promoter of the essential gene.

### Donor Template

In one aspect the present disclosure provides a donor template comprising a knock-in cassette with an exogenous coding sequence for a gene product of interest in frame with and downstream (3′) of an exogenous coding sequence or partial coding sequence of an essential gene, wherein the essential gene encodes a gene product that is required for survival, proliferation, and/or development of the cell.

In another aspect the present disclosure provides a donor template comprising a knock-in cassette with an exogenous coding sequence for a gene product of interest in frame with and upstream (5′) of an exogenous coding sequence or partial coding sequence of an essential gene, wherein the essential gene encodes a gene product that is required for survival, proliferation, and/or development of the cell.

In some embodiments, the donor template is for use in editing the genome of a cell by homology-directed repair (HDR).

Donor template design is described in detail in the literature, for instance in PCT Publication No. WO2016/073990A1. Donor templates can be single-stranded or double-stranded and can be used to facilitate HDR-based repair of double-strand breaks (DSBs), and are particularly useful for inserting a new sequence into the target sequence, or replacing the target sequence altogether. In some embodiments, the donor template is a donor DNA template. In some embodiments the donor DNA template is double-stranded.

Whether single-stranded or double stranded, donor templates generally include regions that are homologous to regions of DNA within or near (e.g., flanking or adjoining) a target sequence to be cleaved. These homologous regions are referred to herein as “homology arms,” and are illustrated schematically below relative to the knock-in cassette (which may be separated from one or both of the homology arms by additional spacer sequences that are not shown):

[5′ homology arm]-[knock-in cassette]-[3′ homology arm].

The homology arms can have any suitable length (including 0 nucleotides if only one homology arm is used), and 5′ and 3′ homology arms can have the same length, or can differ in length. The selection of appropriate homology arm lengths can be influenced by a variety of factors, such as the desire to avoid homologies or microhomologies with certain sequences such as Alu repeats or other very common elements. For example, a 5′ homology arm can be shortened to avoid a sequence repeat element. In other embodiments, a 3′ homology arm can be shortened to avoid a sequence repeat element. In some embodiments, both the 5′ and the 3′ homology arms can be shortened to avoid including certain sequence repeat elements.

In some embodiments, more than one donor template can be administered to a cell population. In some embodiments, the more than one donor templates are different, for example, each donor template facilitates knock-in of “cargo” sequences encoding different gene products of interest. In some embodiments, the more than one donor templates can be provided at the same time and their payloads incorporated into the same essential gene (e.g., one incorporated at one allele, the other incorporated at the other allele). In some embodiments, this may be particularly advantageous when a particular transgene system and/or gene product of interest has functional sequences that require them to be separated into different alleles of an essential gene. Further, in some embodiments, having multiple copies of gene targets of interest that are different but accomplish a similar goal, e.g., copies of safety switches, can be helpful to assure the functionality and creation of a corresponding phenotype. In some embodiments, more than one copy of a safety switch can ensure elimination of cells when necessary. Further, in some embodiments, certain safety switches require dimerization to function as a suicide switch system (e.g., as described herein). In some embodiments, when more than one donor template is administered to a cell population, such donor templates may be designed to integrate at the same genetic locus, or at different genetic loci.

A donor template can be a nucleic acid vector, such as a viral genome or circular double-stranded DNA, e.g., a plasmid. Nucleic acid vectors comprising donor templates can include other coding or non-coding elements. For example, a donor template nucleic acid can be delivered as part of a viral genome (e.g., in an AAV, adenoviral, Sendai virus, or lentiviral genome) that includes certain genomic backbone elements (e.g., inverted terminal repeats, in the case of an AAV genome). In some embodiments, a donor template is comprised in a plasmid that has not been linearized. In some embodiments, a donor template is comprised in a plasmid that has been linearized. In some embodiments, a donor template is included within a linear dsDNA fragment. In some embodiments, a donor template nucleic acid can be delivered as part of an AAV genome. In some embodiments, a donor template nucleic acid can be delivered as a single stranded oligo donor (ssODN), for example, as a long multi-kb ssODN derived from m13 phage synthesis, or alternatively, short ssODNs, e.g., that comprise small genes of interest, tags, and/or probes. In some embodiments, a donor template nucleic acid can be delivered as a Doggybone™ DNA (dbDNA™) template. In some embodiments, a donor template nucleic acid can be delivered as a DNA minicircle. In some embodiments, a donor template nucleic acid can be delivered as an Integration-deficient Lentiviral Particle (IDLV). In some embodiments, a donor template nucleic acid can be delivered as a MMLV-derived retrovirus. In some embodiments, a donor template nucleic acid can be delivered as a piggyBac™ sequence. In some embodiments, a donor template nucleic acid can be delivered as a replicating EBNA1 episome.

In certain embodiments, the 5′ homology arm may be about 25 to about 1,000 base pairs in length, e.g., at least about 100, 200, 400, 600, or 800 base pairs in length. In certain embodiments, the 5′ homology arm comprises about 50 to 800 base pairs, e.g., 100 to 800, 200 to 800, 400 to 800, 400 to 600, or 600 to 800 base pairs. In certain embodiments, the 3′ homology arm may be about 25 to about 1,000 base pairs in length, e.g., at least about 100, 200, 400, 600, or 800 base pairs in length. In certain embodiments, the 3′ homology arm comprises about 50 to 800 base pairs, e.g., 100 to 800, 200 to 800, 400 to 800, 400 to 600, or 600 to 800 base pairs. In certain embodiments, the 5′ and 3′ homology arms are symmetrical in length. In certain embodiments, the 5′ and 3′ homology arms are asymmetrical in length.

In certain embodiments, a 5′ homology arm is less than about 3,000 base pairs, less than about 2,900 base pairs, less than about 2,800 base pairs, less than about 2,700 base pairs, less than about 2,600 base pairs, less than about 2,500 base pairs, less than about 2,400 base pairs, less than about 2,300 base pairs, less than about 2,200 base pairs, less than about 2,100 base pairs, less than about 2,000 base pairs, less than about 1,900 base pairs, less than about 1,800 base pairs, less than about 1,700 base pairs, less than about 1,600 base pairs, less than about 1,500 base pairs, less than about 1,400 base pairs, less than about 1,300 base pairs, less than about 1,200 base pairs, less than about 1,100 base pairs, less than about 1,000 base pairs, less than about 900 base pairs, less than about 800 base pairs, less than about 700 base pairs, less than about 600 base pairs, less than about 500 base pairs, or less than about 400 base pairs.

In certain embodiments, e.g., where a viral vector is utilized to introduce a knock-in cassette through a method described herein, a 5′ homology arm is less than about 1,000 base pairs, less than about 900 base pairs, less than about 800 base pairs, is less than about 700 base pairs, less than about 600 base pairs, less than about 500 base pairs, less than about 400 base pairs, or less than about 300 base pairs. In certain embodiments, e.g., where a viral vector is utilized to introduce a knock-in cassette through a method described herein, a 5′ homology arm is about 400-600 base pairs, e.g., about 500 base pairs.

In certain embodiments, a 3′ homology arm is less than about 3,000 base pairs, less than about 2,900 base pairs, less than about 2,800 base pairs, less than about 2,700 base pairs, less than about 2,600 base pairs, less than about 2,500 base pairs, less than about 2,400 base pairs, less than about 2,300 base pairs, less than about 2,200 base pairs, less than about 2,100 base pairs, less than about 2,000 base pairs, less than about 1,900 base pairs, less than about 1,800 base pairs, less than about 1,700 base pairs, less than about 1,600 base pairs, less than about 1,500 base pairs, less than about 1,400 base pairs, less than about 1,300 base pairs, less than about 1,200 base pairs, less than about 1,100 base pairs, less than 1,000 base pairs, less than about 900 base pairs, less than about 800 base pairs, less than about 700 base pairs, less than about 600 base pairs, less than about 500 base pairs, or less than about 400 base pairs.

In certain embodiments, e.g., where a viral vector is utilized to introduce a knock-in cassette through a method described herein, a 3′ homology arm is less than about 1,000 base pairs, less than about 900 base pairs, less than about 800 base pairs, less than about 700 base pairs, less than about 600 base pairs, less than about 500 base pairs, less than about 400 base pairs, or less than about 300 base pairs. In certain embodiments, e.g., where a viral vector is utilized to introduce a knock-in cassette through a method described herein, a 3′ homology arm is about 400-600 base pairs, e.g., about 500 base pairs.

In certain embodiments, the 5′ and 3′ homology arms flank the break and are less than 100, 75, 50, 25, 15, 10 or 5 base pairs away from an edge of the break. In certain embodiments, the 5′ and 3′ homology arms flank an endogenous stop codon. In certain embodiments, the 5′ and 3′ homology arms flank a break located within about 500 base pairs (e.g., about 500 base pairs, about 450 base pairs, about 400 base pairs, about 350 base pairs, about 300 base pairs, about 250 base pairs, about 200 base pairs, about 150 base pairs, about 100 base pairs, about 50 base pairs, or about 25 base pairs) upstream (5′) of an endogenous stop codon, e.g., the stop codon of an essential gene. In certain embodiments, the 5′ homology arm encompasses an edge of the break.

Certain donor templates are also described in, e.g., WO2021/226151 and/or WO2022/272292, each of which is herein incorporated by reference in its entirety.

### Knock-In Cassette

In some embodiments, a knock-in cassette within the donor template comprises an exogenous coding sequence for the gene product of interest in frame with and downstream (3′) of an exogenous coding sequence or partial coding sequence of the essential gene. In some embodiments, a knock-in cassette within a donor template comprises an exogenous coding sequence for the gene product of interest in frame with and upstream (5′) of an exogenous coding sequence or partial coding sequence of an essential gene. In some embodiments, the knock-in cassette is a polycistronic knock-in cassette. In some embodiments, the knock-in cassette is a bicistronic knock-in cassette. In some embodiment the knock-in cassette does not comprise a reporter gene, e.g., a fluorescent reporter gene or an antibiotic resistance gene.

In some embodiments, a single essential gene locus will be targeted by two knock-in cassettes comprising different “cargo” sequences. In some embodiments, one allele will incorporate one knock-in cassette, while the other allele will incorporate the other knock-in cassette. In some embodiments, a gRNA utilized to generate an appropriate DNA break may be the same for each of the two different knock-in cassettes. In some embodiments, gRNAs utilized to generate appropriate DNA breaks for each of the two different knock-in cassettes may be different, such that the “cargo” sequence is incorporated at a different position for each allele. In some embodiments, such a different position for each allele may still be within the ultimate exons coding region. In some embodiments, such a different position for each allele may be within the penultimate exon (second to last), and/or ultimate (last) exons coding region. In some embodiments, such a different position for at least one of the alleles may be within the first exon. In some embodiments, such a different position for at least one of the alleles may be within the first or second exon.

In order to properly restore the essential gene coding region in the genetically modified cell (so that a functioning gene product is produced) the knock-in cassette does not need to comprise an exogenous coding sequence that corresponds to the entire coding sequence of the essential gene. Indeed, depending on the location of the break in the endogenous coding sequence of the essential gene it may be possible to restore the essential gene by providing a knock-in cassette that comprises a partial coding sequence of the essential gene, e.g., that corresponds to a portion of the endogenous coding sequence of the essential gene that spans the break and the entire region downstream of the break (minus the stop codon), and/or that corresponds to a portion of the endogenous coding sequence of the essential gene that spans the break and the entire region upstream of the break (up to and optionally including the start codon).

In order to minimize the size of the knock-in cassette it may in fact be advantageous, in some embodiments, to have the break located within the last 1500, 1000, 750, 500, 400, 300, 200, 100, or 50 base pairs of the endogenous coding sequence of the essential gene, i.e., towards the 3′ end of the coding sequence. In some embodiments, a base pair's location in a coding sequence may be defined 3′-to-5′ from an endogenous translational stop signal (e.g., a stop codon). In some embodiments, as used herein, an “endogenous coding sequence” can include both exonic and intronic base pairs, and refers to gene sequence occurring 5′ to an endogenous functional translational stop signal. In some embodiments, a break within an endogenous coding sequence comprises a break within one DNA strand. In some embodiments, a break within an endogenous coding sequence comprises a break within both DNA strands. In some embodiments, a break is located within the last 1000 base pairs of the endogenous coding sequence. In some embodiments, a break is located within the last 750 base pairs of the endogenous coding sequence. In some embodiments, a break is located within the last 600 base pairs of the endogenous coding sequence. In some embodiments, a break is located within the last 500 base pairs of the endogenous coding sequence. In some embodiments, a break is located within the last 400 base pairs of the endogenous coding sequence. In some embodiments, a break is located within the last 300 base pairs of the endogenous coding sequence. In some embodiments, a break is located within the last 250 base pairs of the endogenous coding sequence. In some embodiments, a break is located within the last 200 base pairs of the endogenous coding sequence. In some embodiments, a break is located within the last 150 base pairs of the endogenous coding sequence. In some embodiments, a break is located within the last 100 base pairs of the endogenous coding sequence. In some embodiments, a break is located within the last 75 base pairs of the endogenous coding sequence. In some embodiments, a break is located within the last 50 base pairs of the endogenous coding sequence. In some embodiments, a break is located within the last 21 base pairs of the endogenous coding sequence.

In some embodiments, the exogenous partial coding sequence of the essential gene in the knock-in cassette encodes a C-terminal fragment of a protein encoded by the essential gene, e.g., a fragment that is less than 500, 250, 150, 125, 100, 75, 50, 25, 20, 15 or 10 amino acids in length. In some embodiments, the exogenous partial coding sequence of the essential gene in the knock-in cassette is codon optimized. In some embodiments, the exogenous partial coding sequence of the essential gene in the knock-in cassette is codon optimized to eliminate at least one PAM site. In some embodiments, the exogenous partial coding sequence of the essential gene in the knock-in cassette is codon optimized to eliminate more than one PAM site. In some embodiments, the exogenous partial coding sequence of the essential gene in the knock-in cassette is codon optimized to eliminate all relevant nuclease specific PAM sites. In some embodiments, a C-terminal fragment of a protein encoded by the essential gene is about 140 amino acids in length. In some embodiments, a C-terminal fragment of a protein encoded by the essential gene is about 130 amino acids in length. In some embodiments, a C-terminal fragment of a protein encoded by the essential gene is about 120 amino acids in length. In some embodiments, the C-terminal fragment includes an amino acid sequence that is encoded by a region of the endogenous coding sequence of the essential gene that spans the break. In some embodiments, a C-terminal fragment includes an amino acid sequence that is encoded by a region of the endogenous coding sequence within 1 exon of the essential gene. In some embodiments, a C-terminal fragment includes an amino acid sequence that is encoded by a region of the endogenous coding sequence within 2 exons of the essential gene. In some embodiments, a C-terminal fragment includes an amino acid sequence that is encoded by a region of the endogenous coding sequence within 3 exons of the essential gene. In some embodiments, a C-terminal fragment includes an amino acid sequence that is encoded by a region of the endogenous coding sequence within 4 exons of the essential gene. In some embodiments, a C-terminal fragment includes an amino acid sequence that is encoded by a region of the endogenous coding sequence within 5 exons of the essential gene.

In some embodiments, the exogenous partial coding sequence of an essential gene in a knock-in cassette encodes a C-terminal fragment of a protein encoded by an essential gene, e.g., a fragment that is less than 500, 250, 150, 125, 100, 75, 50, 25, 20, 19, 18, 17, 16, 15, 14, 13, 12, 11, 10, 9, 8, or 7 amino acids in length. In some embodiments, the exogenous partial coding sequence of an essential gene in a knock-in cassette encodes a 20 amino acid C-terminal fragment of a protein encoded by an essential gene. In some embodiments, the exogenous partial coding sequence of an essential gene in a knock-in cassette encodes a 19 amino acid C-terminal fragment of a protein encoded by an essential gene. In some embodiments, the exogenous partial coding sequence of an essential gene in a knock-in cassette encodes an 18 amino acid C-terminal fragment of a protein encoded by an essential gene. In some embodiments, the exogenous partial coding sequence of an essential gene in a knock-in cassette encodes a 17 amino acid C-terminal fragment of a protein encoded by an essential gene. In some embodiments, the exogenous partial coding sequence of an essential gene in a knock-in cassette encodes a 16 amino acid C-terminal fragment of a protein encoded by an essential gene. In some embodiments, the exogenous partial coding sequence of an essential gene in a knock-in cassette encodes a 1 amino acid C-terminal fragment of a protein encoded by an essential gene.

In some embodiments, e.g., when the essential gene includes many exons as shown in the exemplary method of FIG. 3A, it may be advantageous to have the break within the last exon of the essential gene. In some embodiments, e.g., when the essential gene includes many exons as shown in the exemplary method of FIG. 3A, it may be advantageous to have the break within the penultimate exon of the essential gene. It is to be understood however that the present disclosure is not limited to any particular location for the break and that the available positions will vary depending on the nature and length of the essential gene and the length of the exogenous coding sequence for the gene product of interest. For example, for essential genes that include a few exons or when the gene product of interest is small it may be possible to locate the break in an upstream exon.

In order to minimize the size of the knock-in cassette it may in fact be advantageous, in some embodiments, to have the break located within the first 1500, 1000, 750, 500, 400, 300, 200, 100, or 50 base pairs of an endogenous coding sequence of the essential gene, i.e., starting from the 5′ end of a coding sequence. In some embodiments, a base pair's location in a coding sequence may be defined 5′-to-3′ from an endogenous translational start signal (e.g., a start codon). In some embodiments, as used herein, an “endogenous coding sequence” can include both exonic and intronic base pairs, and refers to gene sequence occurring 3′ to an endogenous functional translational start signal. In some embodiments, a break within an endogenous coding sequence comprises a break within one DNA strand. In some embodiments, a break within an endogenous coding sequence comprises a break within both DNA strands. In some embodiments, a break is located within the first 1000 base pairs of the endogenous coding sequence. In some embodiments, a break is located within the first 750 base pairs of an endogenous coding sequence. In some embodiments, a break is located within the first 600 base pairs of the endogenous coding sequence. In some embodiments, a break is located within the first 500 base pairs of the endogenous coding sequence. In some embodiments, a break is located within the first 400 base pairs of the endogenous coding sequence. In some embodiments, a break is located within the first 300 base pairs of the endogenous coding sequence. In some embodiments, a break is located within the first 250 base pairs of the endogenous coding sequence. In some embodiments, a break is located within the first 200 base pairs of the endogenous coding sequence. In some embodiments, a break is located within the first 150 base pairs of the endogenous coding sequence. In some embodiments, a break is located within the first 100 base pairs of the endogenous coding sequence. In some embodiments, a break is located within the first 75 base pairs of the endogenous coding sequence. In some embodiments, a break is located within the first 50 base pairs of the endogenous coding sequence. In some embodiments, a break is located within the first 21 base pairs of the endogenous coding sequence.

In some embodiments, the exogenous partial coding sequence of the essential gene in the knock-in cassette encodes an N-terminal fragment of a protein encoded by the essential gene, e.g., a fragment that is less than 500, 250, 150, 125, 100, 75, 50, 25, 20, 15 or 10 amino acids in length. In some embodiments, an N-terminal fragment of a protein encoded by the essential gene is about 140 amino acids in length. In some embodiments, an N-terminal fragment of a protein encoded by the essential gene is about 130 amino acids in length. In some embodiments, an N-terminal fragment of a protein encoded by the essential gene is about 120 amino acids in length. In some embodiments, an N-terminal fragment includes an amino acid sequence that is encoded by a region of the endogenous coding sequence of the essential gene that spans the break. In some embodiments, an N-terminal fragment includes an amino acid sequence that is encoded by a region of the endogenous coding sequence within 1 exon of the essential gene. In some embodiments, an N-terminal fragment includes an amino acid sequence that is encoded by a region of the endogenous coding sequence within 2 exons of the essential gene. In some embodiments, an N-terminal fragment includes an amino acid sequence that is encoded by a region of the endogenous coding sequence within 3 exons of the essential gene. In some embodiments, an N-terminal fragment includes an amino acid sequence that is encoded by a region of the endogenous coding sequence within 4 exons of the essential gene. In some embodiments, an N-terminal fragment includes an amino acid sequence that is encoded by a region of the endogenous coding sequence within 5 exons of the essential gene.

In some embodiments, the exogenous partial coding sequence of an essential gene in a knock-in cassette encodes an N-terminal fragment of a protein encoded by an essential gene, e.g., a fragment that is less than 500, 250, 150, 125, 100, 75, 50, 25, 20, 19, 18, 17, 16, 15, 14, 13, 12, 11, 10, 9, 8, or 7 amino acids in length. In some embodiments, the exogenous partial coding sequence of an essential gene in a knock-in cassette encodes a 20 amino acid N-terminal fragment of a protein encoded by an essential gene. In some embodiments, the exogenous partial coding sequence of an essential gene in a knock-in cassette encodes a 19 amino acid N-terminal fragment of a protein encoded by an essential gene. In some embodiments, the exogenous partial coding sequence of an essential gene in a knock-in cassette encodes an 18 amino acid N-terminal fragment of a protein encoded by an essential gene. In some embodiments, the exogenous partial coding sequence of an essential gene in a knock-in cassette encodes a 17 amino acid N-terminal fragment of a protein encoded by an essential gene. In some embodiments, the exogenous partial coding sequence of an essential gene in a knock-in cassette encodes a 16 amino acid N-terminal fragment of a protein encoded by an essential gene. In some embodiments, the exogenous partial coding sequence of an essential gene in a knock-in cassette encodes a 1 amino acid N-terminal fragment of a protein encoded by an essential gene.

In some embodiments, the exogenous coding sequence or partial coding sequence of the essential gene in the knock-in cassette is less than 100% identical to the corresponding endogenous coding sequence of the essential gene of the cell, e.g., less than 99%, 98%, 97%, 96%, 95%, 94%, 93%, 92%, 91%, 90%, 85%, 80%, 75%, 70%, 65%, 60%, 55% or less than 50% (i.e., when the two sequences are aligned using a standard pairwise sequence alignment tool that maximizes the alignment between the corresponding sequences). For example, in some embodiments, the exogenous coding sequence or partial coding sequence of the essential gene in the knock-in cassette is codon optimized relative to the corresponding endogenous coding sequence of the essential gene of the cell, e.g., to prevent further binding of a nuclease to the target site. Alternatively or additionally it may be codon optimized to reduce the likelihood of recombination after integration of the knock-in cassette into the genome of the cell and/or to increase expression of the gene product of the essential gene and/or the gene product of interest after integration of the knock-in cassette into the genome of the cell.

In some embodiments, a knock-in cassette comprises one or more nucleotides or base pairs that differ (e.g., are mutations) relative to an endogenous knock-in site. In some embodiments, such mutations in a knock-in cassette provide resistance to cutting by a nuclease. In some embodiments, such mutations in a knock-in cassette prevent a nuclease from cutting the target loci following homologous recombination. In some embodiments, such mutations in a knock-in cassette occur within one or more coding and/or non-coding regions of a target gene. In some embodiments, such mutations in a knock-in cassette are silent mutations. In some embodiments, such mutations in a knock-in cassette are silent and/or missense mutations.

In some embodiments, such mutations in a knock-in cassette occur within a target protospacer motif and/or a target protospacer adjacent motif (PAM) site. In some embodiments, a knock-in cassette includes a target protospacer motif and/or a PAM site that are saturated with silent mutations. In some embodiments, a knock-in cassette includes a target protospacer motif and/or a PAM site that are approximately 30%, 40%, 50%, 60%, 70%, 80%, or 90% saturated with silent mutations. In some embodiments, a knock-in cassette includes a target protospacer motif and/or a PAM site that are saturated with silent and/or missense mutations. In some embodiments, a knock-in cassette includes a target protospacer motif and/or a PAM site that comprise at least one mutation, at least 2 mutations, at least 3 mutations, at least 4 mutations, at least 5 mutations, at least 6 mutations, at least 7 mutations, at least 8 mutations, at least 9 mutations, at least 10 mutations, at least 11 mutations, at least 12 mutations, at least 13 mutations, at least 14 mutations, or at least 15 mutations.

In some embodiments, certain codons encoding certain amino acids in a target site cannot be mutated through codon-optimization without losing some portion of an endogenous proteins natural function. In some embodiments, certain codons encoding certain amino acids in a target site cannot be mutated through codon-optimization.

In some embodiments, the knock-in cassette is codon optimized in only a portion of the coding sequence. For example, in some embodiments, a knock-in cassette encodes a C-terminal fragment of a protein encoded by an essential gene, e.g., a fragment that is less than 500, 250, 150, 125, 100, 75, 50, 25, 20, 19, 18, 17, 16, 15, 14, 13, 12, 11, 10, 9, 8, or 7 amino acids in length. In some embodiments, the exogenous partial coding sequence of an essential gene in a knock-in cassette that has been codon optimized encodes a 20 amino acid C-terminal fragment of a protein encoded by an essential gene. In some embodiments, the exogenous partial coding sequence of an essential gene in a knock-in cassette that has been codon optimized encodes a 19 amino acid C-terminal fragment of a protein encoded by an essential gene. In some embodiments, the exogenous partial coding sequence of an essential gene in a knock-in cassette that has been codon optimized encodes an 18 amino acid C-terminal fragment of a protein encoded by an essential gene. In some embodiments, the exogenous partial coding sequence of an essential gene in a knock-in cassette that has been codon optimized encodes a 17 amino acid C-terminal fragment of a protein encoded by an essential gene. In some embodiments, the exogenous partial coding sequence of an essential gene in a knock-in cassette that has been codon optimized encodes a 16 amino acid C-terminal fragment of a protein encoded by an essential gene. In some embodiments, the exogenous partial coding sequence of an essential gene in a knock-in cassette that has been codon optimized encodes a 15 amino acid C-terminal fragment of a protein encoded by an essential gene. In some embodiments, the exogenous partial coding sequence of an essential gene in a knock-in cassette that has been codon optimized encodes a 14 amino acid C-terminal fragment of a protein encoded by an essential gene. In some embodiments, the exogenous partial coding sequence of an essential gene in a knock-in cassette that has been codon optimized encodes a 13 amino acid C-terminal fragment of a protein encoded by an essential gene. In some embodiments, the exogenous partial coding sequence of an essential gene in a knock-in cassette that has been codon optimized encodes a 12 amino acid C-terminal fragment of a protein encoded by an essential gene. In some embodiments, the exogenous partial coding sequence of an essential gene in a knock-in cassette that has been codon optimized encodes a 11 amino acid C-terminal fragment of a protein encoded by an essential gene. In some embodiments, the exogenous partial coding sequence of an essential gene in a knock-in cassette that has been codon optimized encodes a 10 amino acid C-terminal fragment of a protein encoded by an essential gene. In some embodiments, the exogenous partial coding sequence of an essential gene in a knock-in cassette that has been codon optimized encodes a 9 amino acid C-terminal fragment of a protein encoded by an essential gene. In some embodiments, the exogenous partial coding sequence of an essential gene in a knock-in cassette that has been codon optimized encodes an 8 amino acid C-terminal fragment of a protein encoded by an essential gene. In some embodiments, the exogenous partial coding sequence of an essential gene in a knock-in cassette that has been codon optimized encodes a 7 amino acid C-terminal fragment of a protein encoded by an essential gene. In some embodiments, the exogenous partial coding sequence of an essential gene in a knock-in cassette that has been codon optimized encodes a 6 amino acid C-terminal fragment of a protein encoded by an essential gene. In some embodiments, the exogenous partial coding sequence of an essential gene in a knock-in cassette that has been codon optimized encodes a 5 amino acid C-terminal fragment of a protein encoded by an essential gene. In some embodiments, the exogenous partial coding sequence of an essential gene in a knock-in cassette that has been codon optimized encodes an amino acid C-terminal fragment that is less than 5 amino acids of a protein encoded by an essential gene.

In some embodiments, the knock-in cassette is codon optimized in only a portion of the coding sequence. For example, in some embodiments, a knock-in cassette encodes an N-terminal fragment of a protein encoded by an essential gene, e.g., a fragment that is less than 500, 250, 150, 125, 100, 75, 50, 25, 20, 19, 18, 17, 16, 15, 14, 13, 12, 11, 10, 9, 8, or 7 amino acids in length. In some embodiments, the exogenous partial coding sequence of an essential gene in a knock-in cassette that has been codon optimized encodes a 20 amino acid N-terminal fragment of a protein encoded by an essential gene. In some embodiments, the exogenous partial coding sequence of an essential gene in a knock-in cassette that has been codon optimized encodes a 19 amino acid N-terminal fragment of a protein encoded by an essential gene. In some embodiments, the exogenous partial coding sequence of an essential gene in a knock-in cassette that has been codon optimized encodes an 18 amino acid N-terminal fragment of a protein encoded by an essential gene. In some embodiments, the exogenous partial coding sequence of an essential gene in a knock-in cassette that has been codon optimized encodes a 17 amino acid N-terminal fragment of a protein encoded by an essential gene. In some embodiments, the exogenous partial coding sequence of an essential gene in a knock-in cassette that has been codon optimized encodes a 16 amino acid N-terminal fragment of a protein encoded by an essential gene. In some embodiments, the exogenous partial coding sequence of an essential gene in a knock-in cassette that has been codon optimized encodes a 15 amino acid N-terminal fragment of a protein encoded by an essential gene. In some embodiments, the exogenous partial coding sequence of an essential gene in a knock-in cassette that has been codon optimized encodes a 14 amino acid N-terminal fragment of a protein encoded by an essential gene. In some embodiments, the exogenous partial coding sequence of an essential gene in a knock-in cassette that has been codon optimized encodes a 13 amino acid N-terminal fragment of a protein encoded by an essential gene. In some embodiments, the exogenous partial coding sequence of an essential gene in a knock-in cassette that has been codon optimized encodes a 12 amino acid N-terminal fragment of a protein encoded by an essential gene. In some embodiments, the exogenous partial coding sequence of an essential gene in a knock-in cassette that has been codon optimized encodes a 11 amino acid N-terminal fragment of a protein encoded by an essential gene. In some embodiments, the exogenous partial coding sequence of an essential gene in a knock-in cassette that has been codon optimized encodes a 10 amino acid N-terminal fragment of a protein encoded by an essential gene. In some embodiments, the exogenous partial coding sequence of an essential gene in a knock-in cassette that has been codon optimized encodes a 9 amino acid N-terminal fragment of a protein encoded by an essential gene. In some embodiments, the exogenous partial coding sequence of an essential gene in a knock-in cassette that has been codon optimized encodes an 8 amino acid N-terminal fragment of a protein encoded by an essential gene. In some embodiments, the exogenous partial coding sequence of an essential gene in a knock-in cassette that has been codon optimized encodes a 7 amino acid N-terminal fragment of a protein encoded by an essential gene. In some embodiments, the exogenous partial coding sequence of an essential gene in a knock-in cassette that has been codon optimized encodes a 6 amino acid N-terminal fragment of a protein encoded by an essential gene. In some embodiments, the exogenous partial coding sequence of an essential gene in a knock-in cassette that has been codon optimized encodes a 5 amino acid N-terminal fragment of a protein encoded by an essential gene. In some embodiments, the exogenous partial coding sequence of an essential gene in a knock-in cassette that has been codon optimized encodes an amino acid N-terminal fragment that is less than 5 amino acids of a protein encoded by an essential gene.

In some embodiments, the knock-in cassette comprises one or more sequences encoding a linker peptide, e.g., between an exogenous coding sequence or partial coding sequence of the essential gene and a “cargo” sequence and/or a regulatory element described herein. Such linker peptides are known in the art, any of which can be included in a knock-in cassette described herein. In some embodiments, the linker peptide comprises the amino acid sequence GSG.

In some embodiments, the knock-in cassette comprises other regulatory elements such as a polyadenylation sequence, and optionally a 3′ UTR sequence, downstream of the exogenous coding sequence for the gene product of interest. If a 3′UTR sequence is present, the 3′UTR sequence is positioned 3′ of the exogenous coding sequence and 5′ of the polyadenylation sequence.

In some embodiments, the knock-in cassette comprises other regulatory elements such as a 5′ UTR and a start codon, upstream of the exogenous coding sequence for the gene product of interest. If a 5′UTR sequence is present, the 5′UTR sequence is positioned 5′ of the “cargo” sequence and/or exogenous coding sequence.

Certain knock-in cassettes are also described in, e.g., WO2021/226151 and/or WO2022/272292, each of which is herein incorporated by reference in its entirety.

### Exemplary Homology Arms (HA)

In certain embodiments, a donor template comprises a 5′ and/or 3′ homology arm homologous to region of a GAPDH locus. In some embodiments, a donor template comprises a 5′ homology arm comprising or consisting of the sequence of SEQ ID NO:1, 2, or 3. In some embodiments, a 5′ homology arm comprises or consists of a sequence that is at least 85%, 90%, 95%, 98% or 99% identical to the sequence of SEQ ID NO: 1, 2, or 3. In some embodiments, a donor template comprises a 3′ homology arm comprising or consisting of the sequence of SEQ ID NO:4 or 5. In certain embodiments, a 3′ homology arm comprises or consists of a sequence that is at least 85%, 90%, 95%, 98% or 99% identical to the sequence of SEQ ID NO: 4 or 5.

In some embodiments, a donor template comprises a 5′ homology arm comprising SEQ ID NO: 1, and a 3′ homology arm comprising SEQ ID NO: 4. In some embodiments, a donor template comprises a 5′ homology arm comprising SEQ ID NO: 2, and a 3′ homology arm comprising SEQ ID NO: 4. In some embodiments, a donor template comprises a 5′ homology arm comprising SEQ ID NO: 3, and a 3′ homology arm comprising SEQ ID NO:5.

In some embodiments, a stretch of sequence flanking a nuclease cleavage site may be duplicated in both a 5′ and 3′ homology arm. In some embodiments, such a duplication is designed to optimize HDR efficiency. In some embodiments, one of the duplicated sequences may be codon optimized, while the other sequence is not codon optimized. In some embodiments, both of the duplicated sequences may be codon optimized. In some embodiments, codon optimization may remove a target PAM site. In some embodiments, a duplicated sequence may be no more than: 100 bp in length, 90 bp in length, 80 bp in length, 70 bp in length, 60 bp in length, 50 bp in length, 40 bp in length, 30 bp in length, or 20 bp in length.

exemplary 5′ HA for knock-in cassette insertion at GAPDH locus
SEQ ID NO: 1
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAG
exemplary 5′ HA for knock-in cassette insertion at GAPDH locus
SEQ ID NO: 2
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCT
exemplary 5′ HA for knock-in cassette insertion at GAPDH locus
SEQ ID NO: 3
GGCTTTCCCATAATTTCCTTTCAAGGTGGGGAGGGAGGTAGAGGGGTGATGTGGGGAGTACGCT
GCAGGGCCTCACTCCTTTTGCAGACCACAGTCCATGCCATCACTGCCACCCAGAAGACTGTGGA
TGGCCCCTCCGGGAAACTGTGGCGTGATGGCCGCGGGGCTCTCCAGAACATCATCCCTGCCTCT
ACTGGCGCTGCCAAGGCTGTGGGCAAGGTCATCCCTGAGCTGAACGGGAAGCTCACTGGCATGG
CCTTCCGTGTCCCCACTGCCAACGTGTCAGTGGTGGACCTGACCTGCCGTCTAGAAAAACCTGC
CAAATATGATGACATCAAGAAGGTGGTGAAGCAGGCGTCGGAGGGCCCCCTCAAGGGCATCCTG
GGCTACACTGAGCACCAGGTGGTCTCCTCTGACTTCAACAGCGACACCCACTCCTCCACCTTTG
ACGCTGGGGCTGGCATTGCCCTCAACGACCACTTTGTCAAGCTCATCTCTTGGTACGACAATGA
GTTCGGATATAGCAATAGAGTGGTCGATCTGATGGCTCATATGGCTAGCAAAGAG
exemplary 3′ HA for knock-in cassette insertion at GAPDH locus
SEQ ID NO: 4
ATTTGGCTACAGCAACAGGGTGGTGGACCTCATGGCCCACATGGCCTCCAAGGAGTAAGACCCC
TGGACCACCAGCCCCAGCAAGAGCACAAGAGGAAGAGAGAGACCCTCACTGCTGGGGAGTCCCT
GCCACACTCAGTCCCCCACCACACTGAATCTCCCCTCCTCACAGTTGCCATGTAGACCCCTTGA
AGAGGGGAGGGGCCTAGGGAGCCGCACCTTGTCATGTACCATCAATAAAGTACCCTGTGCTCAA
CCAGTTACTTGTCCTGTCTTATTCTAGGGTCTGGGGCAGAGGGGAGGGAAGCTGGGCTTGTGTC
AAGGTGAGACATTCTTGCTGGGGAGGGACCTGGTATGTTCTCCTCAGACTGAGGGTAGGGCCTC
CAAACAGCCTTGCTTGCTTCGAGAACCATTTGCTTCCCGCTCAGACGTCTTGAGTGCTACAGGA
AGCTGGCACCACTACTTCAGAGAACAAGGCCTTTTCCTCTCCTCGCTCCAGT
exemplary 3′ HA for knock-in cassette insertion at GAPDH locus
SEQ ID NO: 5
AGACTGGCTCTTAAAAAGTGCAGGGTCTGGCGCCCTCTGGTGGCTGGCTCAGAAAAAGGGCCCT
GACAACTCTTTTCATCTTCTAGGTATGACAACGAATTTGGCTACAGCAACAGGGTGGTGGACCT
CATGGCCCACATGGCCTCCAAGGAGTAAGACCCCTGGACCACCAGCCCCAGCAAGAGCACAAGA
GGAAGAGAGAGACCCTCACTGCTGGGGAGTCCCTGCCACACTCAGTCCCCCACCACACTGAATC
TCCCCTCCTCACAGTTGCCATGTAGACCCCTTGAAGAGGGGAGGGGCCTAGGGAGCCGCACCTT
GTCATGTACCATCAATAAAGTACCCTGTGCTCAACCAGTTACTTGTCCTGTCTTATTCTAGGGT
CTGGGGCAGAGGGGAGGGAAGCTGGGCTTGTGTCAAGGTGAGACATTCTTGCTGGGGAGGGACC
TGGTATGTTCTCCTCAGACTGAGGGTAGGGCCTCCAAACAGCCTTGCTTGCT

In some embodiments, a donor template comprises a 5′ and/or 3′ homology arm homologous to a region of a TBP locus. In some embodiments, a donor template comprises a 5′ homology arm comprising or consisting of the sequence of SEQ ID NO:6, 7, or 8. In some embodiments, a 5′ homology arm comprises or consists of a sequence that is at least 85%, 90%, 95%, 98% or 99% identical to the sequence of SEQ ID NO: 6, 7, or 8. In some embodiments, a donor template comprises a 3′ homology arm comprising or consisting of the sequence of SEQ ID NO:9, 10, or 11. In certain embodiments, a 3′ homology arm comprises or consists of a sequence that is at least 85%, 90%, 95%, 98% or 99% identical to the sequence of SEQ ID NO: 9, 10, or 11.

In some embodiments, a donor template comprises a 5′ homology arm comprising SEQ ID NO: 6, and a 3′ homology arm comprising SEQ ID NO: 9. In some embodiments, a donor template comprises a 5′ homology arm comprising SEQ ID NO: 7, and a 3′ homology arm comprising SEQ ID NO: 10. In some embodiments, a donor template comprises a 5′ homology arm comprising SEQ ID NO: 8, and a 3′ homology arm comprising SEQ ID NO: 11.

exemplary 5′ HA for knock-in cassette insertion at TBP locus
SEQ ID NO: 6
GCAGACTTCCATTTACAGTGAGGAGGTGAGCATTGCATTGAACAAAAGATGGCGTTTTCACTTG
GAATTAGTTATCTGAAGCTTTAGGATTCCTCAGCAATATGATTATGAGACAAGAAAGGAAGATT
CAGAAATGAGTCTAGTTGAAGGCAGCAATTCAGAGAAGAAGATTCAGTTGTTATCATTGCCGTC
CTGCTTGGTTTATGGCCTGGTTCAGGACCAAGGAGAGAAGTGTGAATACATGCCTCTTGAGCTA
TAGAATGAGACGCTGGAGTCACTAAGATGATTTTTTAAAAGTATTGTTTTATAAACAAAAATAA
GATTGTGACAAGGGATTCCACTATTAATGTTTTCATGCCTGTGCCTTAATCTGACTGGGTATGG
TGAGAATTGTGCTTGCAGCTTTAAGGTAAGAATTTTACCATCTTAATATGTTAAGAAGTGCCAT
TTCAGTCTCTCATCTCTACTCCAACTTGTCTTCTTAGGTGCTAAAGTCAGAGCCGAAATCTACG
AGGCCTTCGAGAACATCTACCCCATCCTGAAGGGCTTCAGAAAGACCACC
exemplary 5′ HA for knock-in cassette insertion at TBP locus
SEQ ID NO: 7
CTGACCACAGCTCTGCAAGCAGACTTCCATTTACAGTGAGGAGGTGAGCATTGCATTGAACAAA
AGATGGCGTTTTCACTTGGAATTAGTTATCTGAAGCTTTAGGATTCCTCAGCAATATGATTATG
AGACAAGAAAGGAAGATTCAGAAATGAGTCTAGTTGAAGGCAGCAATTCAGAGAAGAAGATTCA
GTTGTTATCATTGCCGTCCTGCTTGGTTTATGGCCTGGTTCAGGACCAAGGAGAGAAGTGTGAA
TACATGCCTCTTGAGCTATAGAATGAGACGCTGGAGTCACTAAGATGATTTTTTAAAAGTATTG
TTTTATAAACAAAAATAAGATTGTGACAAGGGATTCCACTATTAATGTTTTCATGCCTGTGCCT
TAATCTGACTGGGTATGGTGAGAATTGTGCTTGCAGCTTTAAGGTAAGAATTTTACCATCTTAA
TATGTTAAGAAGTGCCATTTCAGTCTCTCATCTCTACTCCAACTTGTCTTCTTAGGGGCTAAAG
TGCGGGCCGAGATCTACGAGGCCTTCGAGAATATCTACCCCATCCTGAAGGGCTTCAGAAAGAC
CACC
exemplary 5′ HA for knock-in cassette insertion at TBP locus
SEQ ID NO: 8
ACAAAAGATGGCGTTTTCACTTGGAATTAGTTATCTGAAGCTTTAGGATTCCTCAGCAATATGA
TTATGAGACAAGAAAGGAAGATTCAGAAATGAGTCTAGTTGAAGGCAGCAATTCAGAGAAGAAG
ATTCAGTTGTTATCATTGCCGTCCTGCTTGGTTTATGGCCTGGTTCAGGACCAAGGAGAGAAGT
GTGAATACATGCCTCTTGAGCTATAGAATGAGACGCTGGAGTCACTAAGATGATTTTTTAAAAG
TATTGTTTTATAAACAAAAATAAGATTGTGACAAGGGATTCCACTATTAATGTTTTCATGCCTG
TGCCTTAATCTGACTGGGTATGGTGAGAATTGTGCTTGCAGCTTTAAGGTAAGAATTTTACCAT
CTTAATATGTTAAGAAGTGCCATTTCAGTCTCTCATCTCTACTCCAACTTGTCTTCTTAGGTGC
TAAAGTCAGAGCAGAAATTTATGAAGCATTCGAGAACATCTACCCTATTCTAAAGGGATTCAGG
AAGACGACG
exemplary 3′ HA for knock-in cassette insertion at TBP locus
SEQ ID NO: 9
CAGAAATTTATGAAGCATTTGAAAACATCTACCCTATTCTAAAGGGATTCAGGAAGACGACGTA
ATGGCTCTCATGTACCCTTGCCTCCCCCACCCCCTTCTTTTTTTTTTTTTAAACAAATCAGTTT
GTTTTGGTACCTTTAAATGGTGGTGTTGTGAGAAGATGGATGTTGAGTTGCAGGGTGTGGCACC
AGGTGATGCCCTTCTGTAAGTGCCCACCGCGGGATGCCGGGAAGGGGCATTATTTGTGCACTGA
GAACACCGCGCAGCGTGACTGTGAGTTGCTCATACCGTGCTGCTATCTGGGCAGCGCTGCCCAT
TTATTTATATGTAGATTTTAAACACTGCTGTTGACAAGTTGGTTTGAGGGAGAAAACTTTAAGT
GTTAAAGCCACCTCTATAATTGATTGGACTTTTTAATTTTAATGTTTTTCCCCATGAACCACAG
TTTTTATATTTCTACCAGAAAAGTAAAAATCTTTTTTAAAAGTGTTGTTTTT
exemplary 3′ HA for knock-in cassette insertion at TBP locus
SEQ ID NO: 10
TAGGTGCTAAAGTCAGAGCAGAAATTTATGAAGCATTTGAAAACATCTACCCTATTCTAAAGGG
ATTCAGGAAGACGACGTAATGGCTCTCATGTACCCTTGCCTCCCCCACCCCCTTCTTTTTTTTT
TTTTAAACAAATCAGTTTGTTTTGGTACCTTTAAATGGTGGTGTTGTGAGAAGATGGATGTTGA
GTTGCAGGGTGTGGCACCAGGTGATGCCCTTCTGTAAGTGCCCACCGCGGGATGCCGGGAAGGG
GCATTATTTGTGCACTGAGAACACCGCGCAGCGTGACTGTGAGTTGCTCATACCGTGCTGCTAT
CTGGGCAGCGCTGCCCATTTATTTATATGTAGATTTTAAACACTGCTGTTGACAAGTTGGTTTG
AGGGAGAAAACTTTAAGTGTTAAAGCCACCTCTATAATTGATTGGACTTTTTAATTTTAATGTT
TTTCCCCATGAACCACAGTTTTTATATTTCTACCAGAAAAGTAAAAATCTTT
exemplary 3′ HA for knock-in cassette insertion at TBP locus
SEQ ID NO: 11
AAGGGATTCAGGAAGACGACGTAATGGCTCTCATGTACCCTTGCCTCCCCCACCCCCTTCTTTT
TTTTTTTTTAAACAAATCAGTTTGTTTTGGTACCTTTAAATGGTGGTGTTGTGAGAAGATGGAT
GTTGAGTTGCAGGGTGTGGCACCAGGTGATGCCCTTCTGTAAGTGCCCACCGCGGGATGCCGGG
AAGGGGCATTATTTGTGCACTGAGAACACCGCGCAGCGTGACTGTGAGTTGCTCATACCGTGCT
GCTATCTGGGCAGCGCTGCCCATTTATTTATATGTAGATTTTAAACACTGCTGTTGACAAGTTG
GTTTGAGGGAGAAAACTTTAAGTGTTAAAGCCACCTCTATAATTGATTGGACTTTTTAATTTTA
ATGTTTTTCCCCATGAACCACAGTTTTTATATTTCTACCAGAAAAGTAAAAATCTTTTTTAAAA
GTGTTGTTTTTCTAATTTATAACTCCTAGGGGTTATTTCTGTGCCAGACACA

In some embodiments, a donor template comprises a 5′ and/or 3′ homology arm homologous to a region of a G6PD locus. In some embodiments, a donor template comprises a 5′ homology arm comprising or consisting of the sequence of SEQ ID NO:12. In some embodiments, a 5′ homology arm comprises or consists of a sequence that is at least 85%, 90%, 95%, 98% or 99% identical to the sequence of SEQ ID NO: 12. In some embodiments, a donor template comprises a 3′ homology arm comprising or consisting of the sequence of SEQ ID NO:13. In certain embodiments, a 3′ homology arm comprises or consists of a sequence that is at least 85%, 90%, 95%, 98% or 99% identical to the sequence of SEQ ID NO:13.

In some embodiments, a donor template comprises a 5′ homology arm comprising SEQ ID NO: 12, and a 3′ homology arm comprising SEQ ID NO: 13.

exemplary 5′ HA for knock-in cassette insertion at G6PD locus
SEQ ID NO: 12
GGCCCGGGGGACTCCACATGGTGGCAGGCAGTGGCATCAGCAAGACACTCTCTCCCTCACAGAA
CGTGAAGCTCCCTGACGCCTATGAGCGCCTCATCCTGGACGTCTTCTGCGGGAGCCAGATGCAC
TTCGTGCGCAGGTGAGGCCCAGCTGCCGGCCCCTGCATACCTGTGGGCTATGGGGTGGCCTTTG
CCCTCCCTCCCTGTGTGCCACCGGCCTCCCAAGCCATACCATGTCCCCTCAGCGACGAGCTCCG
TGAGGCCTGGCGTATTTTCACCCCACTGCTGCACCAGATTGAGCTGGAGAAGCCCAAGCCCATC
CCCTATATTTATGGCAGGTGAGGAAAGGGTGGGGGCTGGGGACAGAGCCCAGCGGGCAGGGGCG
GGGTGAGGGTGGAGCTACCTCATGCCTCTCCTCCACCCGTCACTCTCCAGCCGAGGCCCCACGG
AGGCAGACGAGCTGATGAAGAGAGTGGGCTTCCAGTACGAGGGAACCTACAAATGGGTCAACCC
TCACAAGCTG
exemplary 3′ HA for knock-in cassette insertion at G6PD locus
SEQ ID NO: 13
GTGGGTGAACCCCCACAAGCTCTGAGCCCTGGGCACCCACCTCCACCCCCGCCACGGCCACCCT
CCTTCCCGCCGCCCGACCCCGAGTCGGGAGGACTCCGGGACCATTGACCTCAGCTGCACATTCC
TGGCCCCGGGCTCTGGCCACCCTGGCCCGCCCCTCGCTGCTGCTACTACCCGAGCCCAGCTACA
TTCCTCAGCTGCCAAGCACTCGAGACCATCCTGGCCCCTCCAGACCCTGCCTGAGCCCAGGAGC
TGAGTCACCTCCTCCACTCACTCCAGCCCAACAGAAGGAAGGAGGAGGGCGCCCATTCGTCTGT
CCCAGAGCTTATTGGCCACTGGGTCTCACTCCTGAGTGGGGCCAGGGTGGGAGGGAGGGACGAG
GGGGAGGAAAGGGGCGAGCACCCACGTGAGAGAATCTGCCTGTGGCCTTGCCCGCCAGCCTCAG
TGCCACTTGACATTCCTTGTCACCAGCAACATCTCGAGCCCCCTGGATGTCC

In some embodiments, a donor template comprises a 5′ and/or 3′ homology arm homologous to a region of a E2F4 locus. In some embodiments, a donor template comprises a 5′ homology arm comprising or consisting of the sequence of SEQ ID NO: 14, 15, or 16. In some embodiments, a 5′ homology arm comprises or consists of a sequence that is at least 85%, 90%, 95%, 98% or 99% identical to the sequence of SEQ ID NO: 14, 15, or 16. In some embodiments, a donor template comprises a 3′ homology arm comprising or consisting of the sequence of SEQ ID NO: 17, 18, or 19. In certain embodiments, a 3′ homology arm comprises or consists of a sequence that is at least 85%, 90%, 95%, 98% or 99% identical to the sequence of SEQ ID NO: 17, 18, or 19.

In some embodiments, a donor template comprises a 5′ homology arm comprising SEQ ID NO: 14, and a 3′ homology arm comprising SEQ ID NO: 17. In some embodiments, a donor template comprises a 5′ homology arm comprising SEQ ID NO: 15, and a 3′ homology arm comprising SEQ ID NO: 18. In some embodiments, a donor template comprises a 5′ homology arm comprising SEQ ID NO: 16, and a 3′ homology arm comprising SEQ ID NO: 19.

exemplary 5′ HA for knock-in cassette insertion at E2F4 locus
SEQ ID NO: 14
CCAGGGGGCTGTAGTGGGGCCAGGCTGGACCTCTGTGCCCTGAGCATGGCTTTCTTGTTTTTCA
GTTTTGGAACTCCCCAAAGAGCTGTCAGAAATCTTTGATCCCACACGAGGTAGGCTGCTGCATT
CCTCCCTGAGGCTAGGGGTAAGGGACACAGCTCATTGGGTCCTATGGCTGTTTTCTTGCCCTTT
TGAGGACCTTGTTGTGGCGCTTATGGTAACTGGGGCAAAGGGTGAAGTTCCTGATGGGCAGGTG
GGGTTCCCTTTCCTGGGCTTTGGTGGGTGGAGAGGTGGGAGCTGGAATGTTAGTAACTGAGCTC
CCTCCATTCCCAGAGTGCATGAGCTCGGAGCTGCTGGAGGAGTTGATGTCCTCAGAAGGTGGGT
GGCCCTGGAAGGTGGGAGTGGGTGTGGGCAGGGGTTGGGCTGCTGCTAGGGGAGCCCTGGCCCA
GGGCCTGAGACTAGTGCTCTCTGCAGTGTTCGCCCCTCTGCTGAGACTTTCTCCTCCTCCTGGC
GACCACGACTACATCTACAACCTGGACGAGAGCGAGGGCGTGTGCGACCTGTTTGATGTGCCCG
TGCTGAACCTG
exemplary 5′ HA for knock-in cassette insertion at E2F4 locus
SEQ ID NO: 15
CCAGGCTGGACCTCTGTGCCCTGAGCATGGCTTTCTTGTTTTTCAGTTTTGGAACTCCCCAAAG
AGCTGTCAGAAATCTTTGATCCCACACGAGGTAGGCTGCTGCATTCCTCCCTGAGGCTAGGGGT
AAGGGACACAGCTCATTGGGTCCTATGGCTGTTTTCTTGCCCTTTTGAGGACCTTGTTGTGGCG
CTTATGGTAACTGGGGCAAAGGGTGAAGTTCCTGATGGGCAGGTGGGGTTCCCTTTCCTGGGCT
TTGGTGGGTGGAGAGGTGGGAGCTGGAATGTTAGTAACTGAGCTCCCTCCATTCCCAGAGTGCA
TGAGCTCGGAGCTGCTGGAGGAGTTGATGTCCTCAGAAGGTGGGTGGCCCTGGAAGGTGGGAGT
GGGTGTGGGCAGGGGTTGGGCTGCTGCTAGGGGAGCCCTGGCCCAGGGCCTGAGACTAGTGCTC
TCTGCAGTGTTTGCCCCTCTGCTTCGTCTTAGTCCTCCTCCGGGCGACCACGACTACATCTACA
ACCTGGACGAGAGCGAGGGCGTGTGCGACCTGTTTGATGTGCCCGTGCTGAACCTG
exemplary 5′ HA for knock-in cassette insertion at E2F4 locus
SEQ ID NO: 16
GTCAGAAATCTTTGATCCCACACGAGGTAGGCTGCTGCATTCCTCCCTGAGGCTAGGGGTAAGG
GACACAGCTCATTGGGTCCTATGGCTGTTTTCTTGCCCTTTTGAGGACCTTGTTGTGGCGCTTA
TGGTAACTGGGGCAAAGGGTGAAGTTCCTGATGGGCAGGTGGGGTTCCCTTTCCTGGGCTTTGG
TGGGTGGAGAGGTGGGAGCTGGAATGTTAGTAACTGAGCTCCCTCCATTCCCAGAGTGCATGAG
CTCGGAGCTGCTGGAGGAGTTGATGTCCTCAGAAGGTGGGTGGCCCTGGAAGGTGGGAGTGGGT
GTGGGCAGGGGTTGGGCTGCTGCTAGGGGAGCCCTGGCCCAGGGCCTGAGACTAGTGCTCTCTG
CAGTGTTTGCCCCTCTGCTTCGTCTTTCTCCACCCCCGGGAGACCACGATTATATCTACAACCT
GGACGAGAGTGAAGGTGTCTGTGACCTCTTCGACGTGCCCGTGCTCAACCTC
exemplary 3′ HA for knock-in cassette insertion at E2F4 locus
SEQ ID NO: 17
CCACCCCCGGGAGACCACGATTATATCTACAACCTGGACGAGAGTGAAGGTGTCTGTGACCTCT
TTGATGTGCCTGTTCTCAACCTCTGACTGACAGGGACATGCCCTGTGTGGCTGGGACCCAGACT
GTCTGACCTGGGGGTTGCCTGGGGACCTCTCCCACCCGACCCCTACAGAGCTTGAGAGCCACAG
ACGCCTGGCTTCTCCGGCCTCCCCTCACCGCACAGTTCTGGCCACAGCTCCCGCTCCTGTGCTG
GCACTTCTGTGCTCGCAGAGCAGGGGAACAGGACTCAGCCCCCATCACCGTGGAGCCAAAGTGT
TTGCTTCTCCCTTTCTGCGGCCTTCGCCAGCCCAGGCTCGGCTGCCACCCAGTGGCACAGAACC
GAGGAGCTGCCATTACCCCCCATAGGGGGCAGTGTCTTGTTCCTGCCAGCCTCAGTGTCTTGCT
TCTGCCAGCTCCTTCCCCTAGGAGGGAAGGGTGGGGTGGAACTGGGCACATG
exemplary 3′ HA for knock-in cassette insertion at E2F4 locus
SEQ ID NO: 18
ATTATATCTACAACCTGGACGAGAGTGAAGGTGTCTGTGACCTCTTTGATGTGCCTGTTCTCAA
CCTCTGACTGACAGGGACATGCCCTGTGTGGCTGGGACCCAGACTGTCTGACCTGGGGGTTGCC
TGGGGACCTCTCCCACCCGACCCCTACAGAGCTTGAGAGCCACAGACGCCTGGCTTCTCCGGCC
TCCCCTCACCGCACAGTTCTGGCCACAGCTCCCGCTCCTGTGCTGGCACTTCTGTGCTCGCAGA
GCAGGGGAACAGGACTCAGCCCCCATCACCGTGGAGCCAAAGTGTTTGCTTCTCCCTTTCTGCG
GCCTTCGCCAGCCCAGGCTCGGCTGCCACCCAGTGGCACAGAACCGAGGAGCTGCCATTACCCC
CCATAGGGGGCAGTGTCTTGTTCCTGCCAGCCTCAGTGTCTTGCTTCTGCCAGCTCCTTCCCCT
AGGAGGGAAGGGTGGGGTGGAACTGGGCACATGCCAGCACCACTTCTAGCTT
exemplary 3′ HA for knock-in cassette insertion at E2F4 locus
SEQ ID NO: 19
TGACTGACAGGGACATGCCCTGTGTGGCTGGGACCCAGACTGTCTGACCTGGGGGTTGCCTGGG
GACCTCTCCCACCCGACCCCTACAGAGCTTGAGAGCCACAGACGCCTGGCTTCTCCGGCCTCCC
CTCACCGCACAGTTCTGGCCACAGCTCCCGCTCCTGTGCTGGCACTTCTGTGCTCGCAGAGCAG
GGGAACAGGACTCAGCCCCCATCACCGTGGAGCCAAAGTGTTTGCTTCTCCCTTTCTGCGGCCT
TCGCCAGCCCAGGCTCGGCTGCCACCCAGTGGCACAGAACCGAGGAGCTGCCATTACCCCCCAT
AGGGGGCAGTGTCTTGTTCCTGCCAGCCTCAGTGTCTTGCTTCTGCCAGCTCCTTCCCCTAGGA
GGGAAGGGTGGGGTGGAACTGGGCACATGCCAGCACCACTTCTAGCTTCCTTCGCTATCCCCCA
CCCCCTGACCCTCCAGCTCCTCCTGGCCCTCTCACGTGCCCACTTCTGCTGG

In some embodiments, a donor template comprises a 5′ and/or 3′ homology arm homologous to a region of a KIF11 locus. In some embodiments, a donor template comprises a 5′ homology arm comprising or consisting of the sequence of SEQ ID NO: 20, 21, or 22. In some embodiments, a 5′ homology arm comprises or consists of a sequence that is at least 85%, 90%, 95%, 98% or 99% identical to the sequence of SEQ ID NO: 20, 21, or 22. In some embodiments, a donor template comprises a 3′ homology arm comprising or consisting of the sequence of SEQ ID NO: 23, 24, or 25. In certain embodiments, a 3′ homology arm comprises or consists of a sequence that is at least 85%, 90%, 95%, 98% or 99% identical to the sequence of SEQ ID NO: 23, 24, or 25.

In some embodiments, a donor template comprises a 5′ homology arm comprising SEQ ID NO: 20, and a 3′ homology arm comprising SEQ ID NO: 23. In some embodiments, a donor template comprises a 5′ homology arm comprising SEQ ID NO: 21, and a 3′ homology arm comprising SEQ ID NO: 24. In some embodiments, a donor template comprises a 5′ homology arm comprising SEQ ID NO: 22, and a 3′ homology arm comprising SEQ ID NO: 25.

exemplary 5′ HA for knock-in cassette insertion at KIF11 locus
SEQ ID NO: 20
AGAGCAGGGTTTCTTGACAGCAGTGCTATTGGCATTTTAAACTGGATAATTCTTTGTTGTGATG
GGCTTTCCTGTGGACTGTACTATGTTGGTACACAAGAAAAACAGTGTACTATGTGAATACTCAC
TCAAAGCCAGTAGCACTCCCTGATTGTAACACCAAAAAAGTCTCTCAGCATTGCCAAATGTCCC
CTGTGGCAGCAGAATCACTCCCTGATGAGAACCACTACCCTGGAGTAAAATCTATAACTATGTC
TTAGAAAATAACACAGAAAATTAATATTTCTTTCACTCTACTCCTTCCATTAGTGATCAAATAA
AGAAGGCATTTGGCGCTACTTGCCAAATTGTTGGCTCAAACTTGTGCTGAACCTTTTTTGGTTT
TCTACACTTAAGTTTTTTTGCCTATAACCCAGAGAACTTTGAAAATAGAGTGTAGTTAATGTGT
ATCTAATGTTACTTTGTATTGACTTAATTTACCGGCCTTTAATCCACAGCATAAGAAGTCCCAC
GGCAAGGACAAAGAGAACCGGGGCATCAACACACTGGAACGGTCCAAGGTCGAGGAAACAACCG
AGCACCTGGTCACCAAGAGCAGACTGCCTCTGAGAGCCCAGATCAACCTG
exemplary 5′ HA for knock-in cassette insertion at KIF11 locus
SEQ ID NO: 21
TTCCTGTGGACTGTACTATGTTGGTACACAAGAAAAACAGTGTACTATGTGAATACTCACTCAA
AGCCAGTAGCACTCCCTGATTGTAACACCAAAAAAGTCTCTCAGCATTGCCAAATGTCCCCTGT
GGCAGCAGAATCACTCCCTGATGAGAACCACTACCCTGGAGTAAAATCTATAACTATGTCTTAG
AAAATAACACAGAAAATTAATATTTCTTTCACTCTACTCCTTCCATTAGTGATCAAATAAAGAA
GGCATTTGGCGCTACTTGCCAAATTGTTGGCTCAAACTTGTGCTGAACCTTTTTTGGTTTTCTA
CACTTAAGTTTTTTTGCCTATAACCCAGAGAACTTTGAAAATAGAGTGTAGTTAATGTGTATCT
AATGTTACTTTGTATTGACTTAATTTTCCCGCCTTAAATCCACAGCATAAAAAATCACATGGAA
AAGACAAAGAAAACAGAGGCATTAACACACTGGAGAGGTCTAAAGTGGAAGAAACAACCGAGCA
CCTGGTCACCAAGAGCAGACTGCCTCTGAGAGCCCAGATCAACCTG
exemplary 5′ HA for knock-in cassette insertion at KIF11 locus
SEQ ID NO: 22
TTAAACTGGATAATTCTTTGTTGTGATGGGCTTTCCTGTGGACTGTACTATGTTGGTACACAAG
AAAAACAGTGTACTATGTGAATACTCACTCAAAGCCAGTAGCACTCCCTGATTGTAACACCAAA
AAAGTCTCTCAGCATTGCCAAATGTCCCCTGTGGCAGCAGAATCACTCCCTGATGAGAACCACT
ACCCTGGAGTAAAATCTATAACTATGTCTTAGAAAATAACACAGAAAATTAATATTTCTTTCAC
TCTACTCCTTCCATTAGTGATCAAATAAAGAAGGCATTTGGCGCTACTTGCCAAATTGTTGGCT
CAAACTTGTGCTGAACCTTTTTTGGTTTTCTACACTTAAGTTTTTTTGCCTATAACCCAGAGAA
CTTTGAAAATAGAGTGTAGTTAATGTGTATCTAATGTTACTTTGTATTGACTTAATTTTCCCGC
CTTAAATCCACAGCATAAAAAATCACATGGAAAAGACAAAGAAAACAGAGGCATCAACACACTG
GAACGGTCCAAGGTCGAGGAAACAACCGAGCACCTGGTCACCAAGAGCAGACTGCCTCTGAGAG
CCCAGATCAACCTG
exemplary 3′ HA for knock-in cassette insertion at KIF11 locus
SEQ ID NO: 23
AAAAAATCACATGGAAAAGACAAAGAAAACAGAGGCATTAACACACTGGAGAGGTCTAAAGTGG
AAGAAACTACAGAGCACTTGGTTACAAAGAGCAGATTACCTCTGCGAGCCCAGATCAACCTTTA
ATTCACTTGGGGGTTGGCAATTTTATTTTTAAAGAAAACTTAAAAATAAAACCTGAAACCCCAG
AACTTGAGCCTTGTGTATAGATTTTAAAAGAATATATATATCAGCCGGGCGCGGTGGCTCATGC
CTGTAATCCCAGCACTTTGGGAGGCTGAGGCGGGTGGATTGCTTGAGCCCAGGAGTTTGAGACC
AGCCTGGCCAACGTGGCAAAACCTCGTCTCTGTTAAAAATTAGCCGGGCGTGGTGGCACACTCC
TGTAATCCCAGCTACTGGGGAGGCTGAGGCACGAGAATCACTTGAACCCAGGAAGCGGGGTTGC
AGTGAGCCAAAGGTACACCACTACACTCCAGCCTGGGCAACAGAGCAAGACT
exemplary 3′ HA for knock-in cassette insertion at KIF11 locus
SEQ ID NO: 24
AACTACAGAGCACTTGGCTACATAGAGCAGATTACCTCTGCGAGCCCAGATCAACCTTTAATTC
ACTTGGGGGTTGGCAATTTTATTTTTAAAGAAAACTTAAAAATAAAACCTGAAACCCCAGAACT
TGAGCCTTGTGTATAGATTTTAAAAGAATATATATATCAGCCGGGCGCGGTGGCTCATGCCTGT
AATCCCAGCACTTTGGGAGGCTGAGGCGGGTGGATTGCTTGAGCCCAGGAGTTTGAGACCAGCC
TGGCCAACGTGGCAAAACCTCGTCTCTGTTAAAAATTAGCCGGGCGTGGTGGCACACTCCTGTA
ATCCCAGCTACTGGGGAGGCTGAGGCACGAGAATCACTTGAACCCAGGAAGCGGGGTTGCAGTG
AGCCAAAGGTACACCACTACACTCCAGCCTGGGCAACAGAGCAAGACTCGGTCTCAAAAACAAA
ATTTAAAAAAGATATAAGGCAGTACTGTAAATTCAGTTGAATTTTGATATCT
exemplary 3′ HA for knock-in cassette insertion at KIF11 locus
SEQ ID NO: 25
ATTAACACACTGGAGAGTTCTGAAGTGGAAGAAACTACAGAGCACTTGGTTACAAAGAGCAGAT
TACCTCTGCGAGCCCAGATCAACCTTTAATTCACTTGGGGGTTGGCAATTTTATTTTTAAAGAA
AACTTAAAAATAAAACCTGAAACCCCAGAACTTGAGCCTTGTGTATAGATTTTAAAAGAATATA
TATATCAGCCGGGCGCGGTGGCTCATGCCTGTAATCCCAGCACTTTGGGAGGCTGAGGCGGGTG
GATTGCTTGAGCCCAGGAGTTTGAGACCAGCCTGGCCAACGTGGCAAAACCTCGTCTCTGTTAA
AAATTAGCCGGGCGTGGTGGCACACTCCTGTAATCCCAGCTACTGGGGAGGCTGAGGCACGAGA
ATCACTTGAACCCAGGAAGCGGGGTTGCAGTGAGCCAAAGGTACACCACTACACTCCAGCCTGG
GCAACAGAGCAAGACTCGGTCTCAAAAACAAAATTTAAAAAAGATATAAGGC

### Inverted Terminal Repeats (ITRs)

In certain embodiments, a donor template comprises an AAV derived sequence. In certain embodiments, a donor template comprises AAV derived sequences that are typical of an AAV construct, such as cis-acting 5′ and 3′ inverted terminal repeats (ITRs) (See, e.g., B. J. Carter, in “Handbook of Parvoviruses”, ed., P. Tijsser, CRC Press, pp. 155 168 (1990), which is incorporated in its entirety herein by reference). Generally, ITRs are able to form a hairpin. The ability to form a hairpin can contribute to an ITRs ability to self-prime, allowing primase-independent synthesis of a second DNA strand. ITRs also play a role in integration of AAV construct (e.g., a coding sequence) into a genome of a target cell. ITRs can also aid in efficient encapsidation of an AAV construct in an AAV particle.

In some embodiments, a donor template described herein is included within an rAAV particle (e.g., an AAV6 particle). In some embodiments, an ITR is or comprises about 145 nucleic acids. In some embodiments, all or substantially all of a sequence encoding an ITR is used. In some embodiments, an AAV ITR sequence may be obtained from any known AAV, including presently identified mammalian AAV types. In some embodiments an ITR is an AAV6 ITR.

An example of an AAV construct employed in the present disclosure is a “cis-acting” construct containing a cargo sequence (e.g., a donor template described herein), in which the donor template is flanked by 5′ or “left” and 3′ or “right” AAV ITR sequences. 5′ and left designations refer to a position of an ITR sequence relative to an entire construct, read left to right, in a sense direction. For example, in some embodiments, a 5′ or left ITR is an ITR that is closest to a target loci promoter (as opposed to a polyadenylation sequence) for a given construct, when a construct is depicted in a sense orientation, linearly. Concurrently, 3′ and right designations refer to a position of an ITR sequence relative to an entire construct, read left to right, in a sense direction. For example, in some embodiments, a 3′ or right ITR is an ITR that is closest to a polyadenylation sequence in a target loci (as opposed to a promoter sequence) for a given construct, when a construct is depicted in a sense orientation, linearly. ITRs as provided herein are depicted in 5′ to 3′ order in accordance with a sense strand. Accordingly, one of skill in the art will appreciate that a 5′ or “left” orientation ITR can also be depicted as a 3′ or “right” ITR when converting from sense to antisense direction. Further, it is well within the ability of one of skill in the art to transform a given sense ITR sequence (e.g., a 5′/left AAV ITR) into an antisense sequence (e.g., 3/right ITR sequence). One of ordinary skill in the art would understand how to modify a given ITR sequence for use as either a 5′/left or 3/right ITR, or an antisense version thereof.

For example, in some embodiments an ITR (e.g., a 5′ ITR) can have a sequence according to SEQ ID NO: 158. In some embodiments, an ITR (e.g., a 3′ ITR) can have a sequence according to SEQ ID NO: 159. In some embodiments, an ITR includes one or more modifications, e.g., truncations, deletions, substitutions or insertions, as is known in the art. In some embodiments, an ITR comprises fewer than 145 nucleotides, e.g., 127, 130, 134 or 141 nucleotides. For example, in some embodiments, an ITR comprises 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143 144, or 145 nucleotides.

A non-limiting example of 5′ AAV ITR sequences includes SEQ ID NO: 158. A non-limiting example of 3′ AAV ITR sequences includes SEQ ID NO: 159. In some embodiments, the 5′ and a 3′ AAV ITRs (e.g., SEQ ID NO: 158 and 159) flank a donor template described herein (e.g., a donor template comprising a 5′HA, a knock-in cassette, and a 3′ HA). The ability to modify ITR sequences is within the skill of the art. (See, e.g., texts such as Sambrook et al. “Molecular Cloning. A Laboratory Manual”, 2d ed., Cold Spring Harbor Laboratory, New York (1989); and K. Fisher et al., J Virol., 70:520 532 (1996), each of which is incorporated in its entirety herein by reference). In some embodiments, a 5′ ITR sequence is at least 85%, 90%, 95%, 98% or 99% identical to a 5′ ITR sequence represented by SEQ ID NO: 158. In some embodiments, a 3′ ITR sequence is at least 85%, 90%, 95%, 98% or 99% identical to a 3′ ITR sequence represented by SEQ ID NO: 159.

exemplary 5′ ITR for knock-in cassette insertion
SEQ ID NO: 158
CCTGCAGGCAGCTGCGCGCTCGCTCGCTCACTGAGGCCGCCCGGG
CAAAGCCCGGGCGTCGGGCGACCTTTGGTCGCCCGGCCTCAGTGA
GCGAGCGAGCGCGCAGAGAGGGAGTGGCCAACTCCATCACTAGGG
GTTCCT
exemplary 3′ ITR for knock-in cassette insertion
SEQ ID NO: 159
AGGAACCCCTAGTGATGGAGTTGGCCACTCCCTCTCTGCGCGCTC
GCTCGCTCACTGAGGCCGGGCGACCAAAGGTCGCCCGACGCCCGG
GCTTTGCCCGGGCGGCCTCAGTGAGCGAGCGAGCGCGCAGCTGCC
TGCAGG

### Flanking Untranslated Regions, 5′ UTRs and 3′ UTRs

In some embodiments, a knock-in cassette described herein includes all or a portion of an untranslated region (UTR), such as a 5′ UTR and/or a 3′ UTR. UTRs of a gene are transcribed but not translated. A 5′ UTR starts at a transcription start site and continues to the start codon but does not include the start codon. A 3′ UTR starts immediately following the stop codon and continues until the transcriptional termination signal. The regulatory and/or control features of a UTR can be incorporated into any of the knock-in cassettes described herein to enhance or otherwise modulate the expression of an essential target gene loci and/or a cargo sequence.

Natural 5′ UTRs include a sequence that plays a role in translation initiation. In some embodiments, a 5′ UTR comprises sequences, like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. The 5′ UTRs have also been known to form secondary structures that are involved in elongation factor binding. Non-limiting examples of 5′ UTRs include those from the following genes: albumin, serum amyloid A, Apolipoprotein A/B/E, transferrin, alpha fetoprotein, erythropoietin, and Factor VIII.

In some embodiments, a UTR may comprise a non-endogenous regulatory region. In some embodiments, a UTR that comprises a non-endogenous regulatory region is a 3′ UTR. In some embodiments, a UTR that comprises a non-endogenous regulatory region is a 5′ UTR. In some embodiments, a non-endogenous regulatory region may be a target of at least one inhibitory nucleic acid. In some embodiments, an inhibitory nucleic acid inhibits expression and/or activity of a target gene. In some embodiments, an inhibitory nucleic acid is a short interfering RNA (siRNA), a short hairpin RNA (shRNA), a microRNA (miRNA), an antisense oligonucleotide, a guide RNA (gRNA), or a ribozyme. In some embodiments, an inhibitory nucleic acid is an endogenous molecule. In some embodiments, an inhibitory nucleic acid is a non-endogenous molecule. In some embodiments, an inhibitory nucleic acid displays a tissue specific expression pattern. In some embodiments, an inhibitory nucleic acid displays a cell specific expression pattern.

In some embodiments, a knock-in cassette may comprise more than one non-endogenous regulatory regions, e.g., two, three, four, five, six, seven, eight, nine, or ten regulatory regions. In some embodiments, a knock-in cassette may comprise four non-endogenous regulatory regions. In some embodiments, a construct may comprise more than one non-endogenous regulatory regions, wherein at least one of the more than one non-endogenous regulatory regions are not the same as at least one of the other non-endogenous regulatory regions.

In some embodiments, a 3′ UTR is found immediately 3′ to the stop codon of a gene of interest. In some embodiments, a 3′ UTR from an mRNA that is transcribed by a target cell can be included in any knock-in cassette described herein. In some embodiments, a 3′ UTR is derived from an endogenous target loci and may include all or part of the endogenous sequence. In some embodiments, a 3′ UTR sequence is at least 85%, 90%, 95% or 98% identical to the sequence of SEQ ID NO: 26.

exemplary 3′ UTR for knock-in cassette insertion
SEQ ID NO: 26
GCGGCCGCGTCGAGTCTAGAGGGCCCGTTTAAACCCGCTGATCAG
CCTCGA

### Polyadenylation Sequences

In some embodiments, a knock-in cassette construct provided herein can include a polyadenylation (poly(A)) signal sequence. Most nascent eukaryotic mRNAs possess a poly(A) tail at their 3′ end, which is added during a complex process that includes cleavage of the primary transcript and a coupled polyadenylation reaction driven by the poly(A) signal sequence (see, e.g., Proudfoot et al., Cell 108:501-512, 2002, which is incorporated herein by reference in its entirety). A poly(A) tail confers mRNA stability and transferability (Molecular Biology of the Cell, Third Edition by B. Alberts et al., Garland Publishing, 1994, which is incorporated herein by reference in its entirety). In some embodiments, a poly(A) signal sequence is positioned 3′ to a coding sequence.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. A 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In some embodiments, a poly(A) tail is added onto transcripts that contain a specific sequence, e.g., a polyadenylation (or poly(A)) signal. A poly(A) tail and associated proteins aid in protecting mRNA from degradation by exonucleases. Polyadenylation also plays a role in transcription termination, export of the mRNA from the nucleus, and translation. Polyadenylation typically occurs in the nucleus immediately after transcription of DNA into RNA, but also can occur later in the cytoplasm. After transcription has been terminated, an mRNA chain is cleaved through the action of an endonuclease complex associated with RNA polymerase. A cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

There are several poly(A) signal sequences that can be used, including those derived from bovine growth hormone (bGH) (Woychik et al., Proc. Natl. Acad Sci. US.A. 81(13):3944-3948, 1984; U.S. Pat. No. 5,122,458, each of which is incorporated herein by reference in its entirety), mouse-β-globin, mouse-α-globin (Orkin et al., EMBO J 4(2):453-456, 1985; Thein et al., Blood 71(2):313-319, 1988, each of which is incorporated herein by reference in its entirety), human collagen, polyoma virus (Batt et al., Mol. Cell Biol. 15(9):4783-4790, 1995, which is incorporated herein by reference in its entirety), the Herpes simplex virus thymidine kinase gene (HSV TK), IgG heavy-chain gene polyadenylation signal (US 2006/0040354, which is incorporated herein by reference in its entirety), human growth hormone (hGH) (Szymanski et al., Mol. Therapy 15(7):1340-1347, 2007, which is incorporated herein by reference in its entirety), the group comprising a SV40 poly(A) site, such as the SV40 late and early poly(A) site (Schek et al., Mol. Cell Biol. 12(12):5386-5393, 1992, which is incorporated herein by reference in its entirety).

The poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414, which is incorporated herein by reference in its entirety).

In some embodiments, a poly(A) signal sequence can be a synthetic polyadenylation site (see, e.g., the pCl-neo expression construct of Promega that is based on Levitt el al., Genes Dev. 3(7):1019-1025, 1989, which is incorporated herein by reference in its entirety). In some embodiments, a poly(A) signal sequence is the polyadenylation signal of soluble neuropilin-1 (sNRP) (AAATAAAATACGAAATG) (see, e.g., WO 05/073384, which is incorporated herein by reference in its entirety). In some embodiments, a poly(A) signal sequence comprises or consists of the SV40 poly(A) site. In some embodiments, a poly(A) signal sequence comprises or consists of SEQ ID NO: 27. In some embodiments, a poly(A) signal sequence comprises or consists of bGHpA. In some embodiments, a poly(A) signal sequence comprises or consists of SEQ ID NO: 28. Additional examples of poly(A) signal sequences are known in the art. In some embodiments, a poly(A) sequence is at least 85%, 90%, 95%, 98% or 99% identical to the sequence of SEQ ID NOs: 27 or 28.

exemplary SV40 poly(A) signal sequence
SEQ ID NO: 27
AACTTGTTTATTGCAGCTTATAATGGTTACAAATAAAGCAATAGC
ATCACAAATTTCACAAATAAAGCATTTTTTTCACTGCATTCTAGT
TGTGGTTTGTCCAAACTCATCAATGTATCTTA
exemplary bGH poly(A) signal sequence
SEQ ID NO: 28
CTGTGCCTTCTAGTTGCCAGCCATCTGTTGTTTGCCCCTCCCCCG
TGCCTTCCTTGACCCTGGAAGGTGCCACTCCCACTGTCCTTTCCT
AATAAAATGAGGAAATTGCATCGCATTGTCTGAGTAGGTGTCATT
CTATTCTGGGGGGTGGGGTGGGGCAGGACAGCAAGGGGGAGGATT
GGGAAGACAATAGCAGGCATGCTGGGGATGCGGTGGGCTCTATGG

### IRES and 2A Elements

In some embodiments, the knock-in cassette comprises a regulatory element that enables expression of the gene product encoded by the essential gene and the gene product of interest as separate gene products, e.g., an IRES or 2A element located between the exogenous coding sequence or partial coding sequence of the essential gene and the exogenous coding sequence for the gene product of interest.

In some embodiments, a knock-in cassette may comprise multiple gene products of interest (e.g., at least two gene products of interest). In some embodiments, gene products of interest may be separated by a regulatory element that enables expression of the at least two gene products of interest as more than one gene product, e.g., an IRES or 2A element located between the at least two coding sequences, facilitating creation of at least two peptide products.

Internal Ribosome Entry Site (IRES) elements are one type of regulatory element that are commonly used for this purpose. As is well known in the art, IRES elements allow for initiation of translation from an internal region of the mRNA and hence expression of two separate proteins from the same mRNA transcript. IRES was originally discovered in poliovirus RNA, where it promotes translation of the viral genome in eukaryotic cells. Since then, a variety of IRES sequences have been discovered—many from viruses, but also some from cellular mRNAs, e.g., see Mokrejs et al., Nucleic Acids Res. 2006; 34(Database issue):D125-D130.

2A elements are another type of regulatory element that are commonly used for this purpose. These 2A elements encode so-called “self-cleaving” 2A peptides which are short peptides (about 20 amino acids) that were first discovered in picornaviruses. The term “self-cleaving” is not entirely accurate, as these peptides are thought to function by making the ribosome skip the synthesis of a peptide bond at the C-terminus of a 2A element, leading to separation between the end of the 2A sequence and the next peptide downstream. The “cleavage” occurs between the Glycine (G) and Proline (P) residues found on the C-terminus meaning the upstream cistron, i.e., protein encoded by the essential gene will have a few additional residues from the 2A peptide added to the end, while the downstream cistron, i.e., gene product of interest will start with the Proline (P).

Table 2 below lists the four commonly used 2A peptides (an optional GSG sequence is sometimes added to the N-terminal end of the peptide to improve cleavage efficiency). There are many potential 2A peptides that may be suitable for methods and compositions described herein (see e.g., Luke et al., Occurrence, function and evolutionary origins of ‘2A-like’ sequences in virus genomes. J Gen Virol. 2008). Those skilled in the art know that the choice of specific 2A peptide for a particular knock-in cassette will ultimately depend on a number of factors such as cell type or experimental conditions. Those skilled in the art will recognize that nucleotide sequences encoding specific 2A peptides can vary while still encoding a peptide suitable for inducing a desired cleavage event.

TABLE 2
Exemplary 2A peptide sequences

| SEQ | | |
|---|---|---|
| ID | 2A | |
| NO: | peptide | Sequence |
| 29 | T2A | EGRGSLLTCGDVEENPGP |
| 30 | P2A | ATNFSLLKQAGDVEENPGP |
| 31 | E2A | QCTNYALLKLAGDVESNPGP |
| 32 | F2A | VKQTLNFDLLKLAGDVESNPGP |
| 33 | T2A | GAGGGCAGAGGAAGTCTTCTAACATGOGGT |
| | | GACGTGGAGGAGAATCCTGGCCCG |
| 34 | P2A | GGAAGCGGAGCTACTAACTTCAGCCTGCTG |
| | | AAGCAGGCTGGAGACGTGGAGGAGAACCCT |
| | | GGACCT |
| 35 | E2A | CAGTGTACTAATTATGCTCTCTTGAAATTG |
| | | GCTGGAGATGTTGAGAGCAACCCTGGACCT |
| 36 | F2A | GTGAAACAGACTTTGAATTTTGACCTTCTC |
| | | AAGTTGGCGGGAGACGTGGAGTCCAACCCT |
| | | GGACCT |
| 37 | IRES | CCCCTCTCCCTCCCCCCCCCCTAACGTTAC |
| | | TGGCCGAAGCCGCTTGGAATAAGGCCGGTG |
| | | TGCGTTTGTCTATATGTTATTTTCCACCAT |
| | | ATTGCCGTCTTTTGGCAATGTGAGGGCCCG |
| | | GAAACCTGGCCCTGTCTTCTTGACGAGCAT |
| | | TCCTAGGGGTCTTTCCCCTCTCGCCAAAGG |
| | | AATGCAAGGTCTGTTGAATGTCGTGAAGGA |
| | | AGCAGTTCCTCTGGAAGCTTCTTGAAGACA |
| | | AACAACGTCTGTAGCGACCCTTTGCAGGCA |
| | | GCGGAACCCCCCACCTGGCGACAGGTGCCT |
| | | CTGCGGCCAAAAGCCACGTGTATAAGATAC |
| | | ACCTGCAAAGGCGGCACAACCCCAGTGCCA |
| | | CGTTGTGAGTTGGATAGTTGTGGAAAGAGT |
| | | CAAATGGCTCTCCTCAAGCGTATTCAACAA |
| | | GGGGCTGAAGGATGCCCAGAAGGTACCCCA |
| | | TTGTATGGGATCTGATCTGGGGCCTCGGTG |
| | | CACATGCTTTACATGTGTTTAGTCGAGGTT |
| | | AAAAAAACGTCTAGGCCCCCCGAACCACGG |
| | | GGACGTGGTTTTCCTTTGAAAAACACGATG |
| | | ATAA |

### Essential Genes

An essential gene can be any gene that is essential for the survival, the proliferation, and/or the development of the cell.

In some embodiments, an essential gene is a housekeeping gene that is essential for survival of all cell types, e.g., a gene listed in Table 3. See also other housekeeping genes discussed in Eisenberg, Trends in Gen. 2014; 30(3):119-20 and Mocin et al., Adv. Biomed Res. 2017; 6:15. In some embodiments, an essential gene is a housekeeping gene that is essential for survival of a B cell. In some embodiments, an essential gene is a housekeeping gene that is essential for survival of an iPSC/ESC. Additional genes that are essential for various cell types, including iPSCs/ESCs, are listed in Table 4 (see also the essential genes discussed in Yilmaz et al., Nat. Cell Biol. 2018; 20:610-619 the entire contents of which are incorporated herein by reference).

In some embodiments, an essential gene is a gene that is essential for development of a B cell, e.g., a gene that is essential for differentiation of a B cell from an iPSC, ESC, or HSC to a B cell.

In some embodiments the essential gene is GAPDH and the DNA nuclease causes a break in exon 9, e.g., a double-strand break. In some embodiments the essential gene is TBP and the DNA nuclease causes a break in exon 7, or exon 8, e.g., a double-strand break. In some embodiments the essential gene is E2F4 and the DNA nuclease causes a break in exon 10, e.g., a double-strand break. In some embodiments the essential gene is G6PD and the DNA nuclease causes a break in exon 13, e.g., a double-strand break. In some embodiments the essential gene is KIF11 and the DNA nuclease causes a break in exon 22, e.g., a double-strand break.

TABLE 3
Exemplary housekeeping genes

| Ensembl | Gene | Ensembl | Gene |
|---|---|---|---|
| ID | Symbol | ID | Symbol |
| ENSG00000075624 | ACTB | ENSG00000231500 | RPS18 |
| ENSG00000116459 | ATP5F1 | ENSG00000112592 | TBP |
| ENSG00000166710 | B2M | ENSG00000072274 | TFRC |
| ENSG00000111640 | GAPDH | ENSG00000164924 | YWHAZ |
| ENSG00000169919 | GUSB | ENSG00000089157 | RPLP0 |
| ENSG00000165704 | HPRT1 | ENSG00000142541 | RPL13A |
| ENSG00000102144 | PGK1 | ENSG00000147604 | RPL7 |
| ENSG00000196262 | PPIA | ENSG00000205250 | E2F4 |
| ENSG00000138160 | KIF11 | ENSG00000160211 | G6PD |

TABLE 4
Additional exemplary essential genes

| Ensembl ID | Gene Symbol | Ensembl ID | Gene Symbol |
|---|---|---|---|
| ENSG00000111704 | NANOG | ENSG00000181449 | SOX2 |
| ENSG00000179059 | ZFP42 | ENSG00000136997 | MYC |
| ENSG00000136826 | KLF4 | ENSG00000175166 | PSMD2 |
| ENSG00000118655 | DCLRE1B | ENSG00000070614 | NDST1 |
| ENSG00000172409 | CLP1 | ENSG00000115484 | CCT4 |
| ENSG00000082898 | XPO1 | ENSG00000100890 | KIAA0391 |
| ENSG00000114867 | EIF4G1 | ENSG00000149474 | CSRP2BP |
| ENSG00000115866 | DARS | ENSG00000102738 | MRPS31 |
| ENSG00000204628 | GNB2L1 | ENSG00000136104 | RNASEH2B |
| ENSG00000198242 | RPL23A | ENSG00000106246 | PTCD1 |
| ENSG00000158526 | TSR2 | ENSG00000248919 | ATP5J2-PTCD1 |
| ENSG00000125450 | NUP85 | ENSG00000138663 | COPS4 |
| ENSG00000134371 | CDC73 | ENSG00000115368 | WDR75 |
| ENSG00000164941 | INTS8 | ENSG00000128564 | VGF |
| ENSG00000055483 | USP36 | ENSG00000128191 | DGCR8 |
| ENSG00000258366 | RTEL1 | ENSG00000008294 | SPAG9 |
| ENSG00000188846 | RPL14 | ENSG00000131475 | VPS25 |
| ENSG00000247626 | MARS2 | ENSG00000105523 | FAM83E |
| ENSG00000095787 | WAC | ENSG00000172269 | DPAGT1 |
| ENSG00000108094 | CUL2 | ENSG00000170312 | CDK1 |
| ENSG00000185946 | RNPC3 | ENSG00000104131 | EIF3J |
| ENSG00000154473 | BUB3 | ENSG00000150753 | CCT5 |
| ENSG00000204394 | VARS | ENSG00000140443 | IGF1R |
| ENSG00000103051 | COG4 | ENSG00000010292 | NCAPD2 |
| ENSG00000104738 | MCM4 | ENSG00000171763 | SPATA5L1 |
| ENSG00000117222 | RBBP5 | ENSG00000180098 | TRNAU1AP |
| ENSG00000082516 | GEMIN5 | ENSG00000168374 | ARF4 |
| ENSG00000100162 | CENPM | ENSG00000173812 | EIF1 |
| ENSG00000141456 | PELP1 | ENSG00000100554 | ATP6V1D |
| ENSG00000137807 | KIF23 | ENSG00000072756 | TRNT1 |
| ENSG00000112685 | EXOC2 | ENSG00000135372 | NAT10 |
| ENSG00000125995 | ROMO1 | ENSG00000178394 | HTR1A |
| ENSG00000136891 | TEX10 | ENSG00000128272 | ATF4 |
| ENSG00000173113 | TRMT112 | ENSG00000204070 | SYS1 |
| ENSG00000075914 | EXOSC7 | ENSG00000137815 | RTF1 |
| ENSG00000119523 | ALG2 | ENSG00000198026 | ZNF335 |
| ENSG00000244038 | DDOST | ENSG00000117410 | ATP6V0B |
| ENSG00000108175 | ZMIZ1 | ENSG00000112739 | PRPF4B |
| ENSG00000129691 | ASH2L | ENSG00000129347 | KRI1 |
| ENSG00000183207 | RUVBL2 | ENSG00000221818 | EBF2 |
| ENSG00000055044 | NOP58 | ENSG00000198431 | TXNRD1 |
| ENSG00000204315 | FKBPL | ENSG00000104979 | C19orf53 |
| ENSG00000187522 | HSPA14 | ENSG00000136709 | WDR33 |
| ENSG00000169375 | SIN3A | ENSG00000149100 | EIF3M |
| ENSG00000143748 | NVL | ENSG00000125835 | SNRPB |
| ENSG00000021776 | AQR | ENSG00000116698 | SMG7 |
| ENSG00000132467 | UTP3 | ENSG00000087586 | AURKA |
| ENSG00000087470 | DNM1L | ENSG00000169230 | PRELID1 |
| ENSG00000130811 | EIF3G | ENSG00000143799 | PARP1 |
| ENSG00000180198 | RCC1 | ENSG00000146731 | CCT6A |
| ENSG00000101407 | TTI1 | ENSG00000163877 | SNIP1 |
| ENSG00000116455 | WDR77 | ENSG00000215421 | ZNF407 |
| ENSG00000135763 | URB2 | ENSG00000197724 | PHF2 |
| ENSG00000133316 | WDR74 | ENSG00000172590 | MRPL52 |
| ENSG00000189091 | SF3B3 | ENSG00000175203 | DCTN2 |
| ENSG00000109917 | ZNF259 | ENSG00000149273 | RPS3 |
| ENSG00000130640 | TUBGCP2 | ENSG00000204822 | MRPL53 |
| ENSG00000011376 | LARS2 | ENSG00000109775 | UFSP2 |
| ENSG00000135249 | RINT1 | ENSG00000165733 | BMS1 |
| ENSG00000126883 | NUP214 | ENSG00000104671 | DCTN6 |
| ENSG00000163510 | CWC22 | ENSG00000175224 | ATG13 |
| ENSG00000101138 | CSTF1 | ENSG00000142541 | RPL13A |
| ENSG00000104221 | BRF2 | ENSG00000173805 | HAP1 |
| ENSG00000125630 | POLR1B | ENSG00000115750 | TAF1B |
| ENSG00000083896 | YTHDC1 | ENSG00000165688 | PMPCA |
| ENSG00000105726 | ATP13A1 | ENSG00000159720 | ATP6V0D1 |
| ENSG00000105618 | PRPF31 | ENSG00000074201 | CLNS1A |
| ENSG00000117748 | RPA2 | ENSG00000158417 | EIF5B |
| ENSG00000143294 | PRCC | ENSG00000196588 | MKL1 |
| ENSG00000156239 | N6AMT1 | ENSG00000138614 | VWA9 |
| ENSG00000143384 | MCL1 | ENSG00000124571 | XPO5 |
| ENSG00000113407 | TARS | ENSG00000198000 | NOL8 |
| ENSG00000086589 | RBM22 | ENSG00000181991 | MRPS11 |
| ENSG00000133119 | RFC3 | ENSG00000149823 | VPS51 |
| ENSG00000052749 | RRP12 | ENSG00000151348 | EXT2 |
| ENSG00000103047 | TANGO6 | ENSG00000162396 | PARS2 |
| ENSG00000142751 | GPN2 | ENSG00000204843 | DCTN1 |
| ENSG00000101057 | MYBL2 | ENSG00000177302 | TOP3A |
| ENSG00000176915 | ANKLE2 | ENSG00000142684 | ZNF593 |
| ENSG00000071127 | WDR1 | ENSG00000074800 | ENO1 |
| ENSG00000106344 | RBM28 | ENSG00000167513 | CDT1 |
| ENSG00000100316 | RPL3 | ENSG00000141101 | NOB1 |
| ENSG00000139131 | YARS2 | ENSG00000047315 | POLR2B |
| ENSG00000182831 | C16orf72 | ENSG00000131966 | ACTR10 |
| ENSG00000167325 | RRM1 | ENSG00000115875 | SRSF7 |
| ENSG00000172262 | ZNF131 | ENSG00000186141 | POLR3C |
| ENSG00000007168 | PAFAH1B1 | ENSG00000108424 | KPNB1 |
| ENSG00000117174 | ZNHIT6 | ENSG00000111845 | PAK1IP1 |
| ENSG00000196497 | IPO4 | ENSG00000148832 | PAOX |
| ENSG00000188566 | NDOR1 | ENSG00000156017 | C9orf41 |
| ENSG00000183091 | NEB | ENSG00000198901 | PRC1 |
| ENSG00000011304 | PTBP1 | ENSG00000134001 | EIF2S1 |
| ENSG00000109805 | NCAPG | ENSG00000146918 | NCAPG2 |
| ENSG00000123154 | WDR83 | ENSG00000144713 | RPL32 |
| ENSG00000147416 | ATP6V1B2 | ENSG00000185122 | HSF1 |
| ENSG00000163961 | RNF168 | ENSG00000167658 | EEF2 |
| ENSG00000163811 | WDR43 | ENSG00000164190 | NIPBL |
| ENSG00000143624 | INTS3 | ENSG00000163902 | RPN1 |
| ENSG00000101161 | PRPF6 | ENSG00000244045 | TMEM199 |
| ENSG00000130726 | TRIM28 | ENSG00000143476 | DTL |
| ENSG00000165494 | PCF11 | ENSG00000149503 | INCENP |
| ENSG00000053900 | ANAPC4 | ENSG00000071243 | ING3 |
| ENSG00000168255 | POLR2J3 | ENSG00000186073 | C15orf41 |
| ENSG00000129534 | MIS18BP1 | ENSG00000088836 | SLC4A11 |
| ENSG00000164754 | RAD21 | ENSG00000136273 | HUS1 |
| ENSG00000120158 | RCL1 | ENSG00000005007 | UPF1 |
| ENSG00000161016 | RPL8 | ENSG00000070010 | UFD1L |
| ENSG00000030066 | NUP160 | ENSG00000106263 | EIF3B |
| ENSG00000099624 | ATP5D | ENSG00000213024 | NUP62 |
| ENSG00000116120 | FARSB | ENSG00000067191 | CACNB1 |
| ENSG00000115233 | PSMD14 | ENSG00000179091 | CYC1 |
| ENSG00000086504 | MRPL28 | ENSG00000113312 | TTC1 |
| ENSG00000160752 | FDPS | ENSG00000085831 | TTC39A |
| ENSG00000049541 | RFC2 | ENSG00000118197 | DDX59 |
| ENSG00000148688 | RPP30 | ENSG00000134871 | COL4A2 |
| ENSG00000114573 | ATP6V1A | ENSG00000088986 | DYNLL1 |
| ENSG00000086200 | IPO11 | ENSG00000138778 | CENPE |
| ENSG00000119720 | NRDE2 | ENSG00000106244 | PDAP1 |
| ENSG00000058262 | SEC61A1 | ENSG00000177600 | RPLP2 |
| ENSG00000073111 | MCM2 | ENSG00000112081 | SRSF3 |
| ENSG00000138160 | KIF11 | ENSG00000100413 | POLR3H |
| ENSG00000215193 | PEX26 | ENSG00000172508 | CARNS1 |
| ENSG00000161057 | PSMC2 | ENSG00000147123 | NDUFB11 |
| ENSG00000187514 | PTMA | ENSG00000119953 | SMNDC1 |
| ENSG00000135829 | DHX9 | ENSG00000111640 | GAPDH |
| ENSG00000058729 | RIOK2 | ENSG00000117899 | MESDC2 |
| ENSG00000110330 | BIRC2 | ENSG00000075624 | ACTB |
| ENSG00000141759 | TXNL4A | ENSG00000163166 | IWS1 |
| ENSG00000166986 | MARS | ENSG00000114503 | NCBP2 |
| ENSG00000153774 | CFDP1 | ENSG00000198522 | GPN1 |
| ENSG00000130177 | CDC16 | ENSG00000099899 | TRMT2A |
| ENSG00000241553 | ARPC4 | ENSG00000181544 | FANCB |
| ENSG00000132604 | TERF2 | ENSG00000136982 | DSCC1 |
| ENSG00000114982 | KANSL3 | ENSG00000068366 | ACSL4 |
| ENSG00000213780 | GTF2H4 | ENSG00000062716 | VMP1 |
| ENSG00000139343 | SNRPF | ENSG00000111802 | TDP2 |
| ENSG00000101189 | MRGBP | ENSG00000185627 | PSMD13 |
| ENSG00000079246 | XRCC5 | ENSG00000020426 | MNAT1 |
| ENSG00000196943 | NOP9 | ENSG00000113734 | BNIP1 |
| ENSG00000122965 | RBM19 | ENSG00000102241 | HTATSF1 |
| ENSG00000132383 | RPA1 | ENSG00000160789 | LMNA |
| ENSG00000094880 | CDC23 | ENSG00000062822 | POLD1 |
| ENSG00000213639 | PPP1CB | ENSG00000168944 | CEP120 |
| ENSG00000109911 | ELP4 | ENSG00000139718 | SETD1B |
| ENSG00000180957 | PITPNB | ENSG00000132792 | CTNNBL1 |
| ENSG00000122257 | RBBP6 | ENSG00000173540 | GMPPB |
| ENSG00000173145 | NOC3L | ENSG00000128789 | PSMG2 |
| ENSG00000179115 | FARSA | ENSG00000196365 | LONP1 |
| ENSG00000105171 | POP4 | ENSG00000160214 | RRP1 |
| ENSG00000148303 | RPL7A | ENSG00000179041 | RRS1 |
| ENSG00000167508 | MVD | ENSG00000143106 | PSMA5 |
| ENSG00000115541 | HSPE1 | ENSG00000168411 | RFWD3 |
| ENSG00000170445 | HARS | ENSG00000073584 | SMARCE1 |
| ENSG00000168496 | FEN1 | ENSG00000175334 | BANF1 |
| ENSG00000141367 | CLTC | ENSG00000077152 | UBE2T |
| ENSG00000087191 | PSMC5 | ENSG00000173611 | SCAI |
| ENSG00000163159 | VPS72 | ENSG00000171720 | HDAC3 |
| ENSG00000130741 | EIF2S3 | ENSG00000182197 | EXT1 |
| ENSG00000168495 | POLR3D | ENSG00000114346 | ECT2 |
| ENSG00000071894 | CPSF1 | ENSG00000124214 | STAU1 |
| ENSG00000058600 | POLR3E | ENSG00000126254 | RBM42 |
| ENSG00000100726 | TELO2 | ENSG00000127184 | COX7C |
| ENSG00000165501 | LRR1 | ENSG00000174276 | ZNHIT2 |
| ENSG00000113575 | PPP2CA | ENSG00000177971 | IMP3 |
| ENSG00000116922 | C1orf109 | ENSG00000104872 | PIH1D1 |
| ENSG00000073712 | FERMT2 | ENSG00000132155 | RAF1 |
| ENSG00000174437 | ATP2A2 | ENSG00000163872 | YEATS2 |
| ENSG00000176407 | KCMF1 | ENSG00000119906 | FAM178A |
| ENSG00000140525 | FANCI | ENSG00000217930 | PAM16 |
| ENSG00000101182 | PSMA7 | ENSG00000197498 | RPF2 |
| ENSG00000130204 | TOMM40 | ENSG00000130348 | QRSL1 |
| ENSG00000239306 | RBM14 | ENSG00000147536 | GINS4 |
| ENSG00000248643 | RBM14-RBM4 | ENSG00000174748 | RPL15 |
| ENSG00000172113 | NME6 | ENSG00000159147 | DONSON |
| ENSG00000136448 | NMT1 | ENSG00000157593 | SLC35B2 |
| ENSG00000186166 | CCDC84 | ENSG00000181938 | GINS3 |
| ENSG00000166233 | ARIH1 | ENSG00000187446 | CHP1 |
| ENSG00000111877 | MCM9 | ENSG00000070371 | CLTCL1 |
| ENSG00000204316 | MRPL38 | ENSG00000096063 | SRPK1 |
| ENSG00000101868 | POLA1 | ENSG00000141564 | RPTOR |
| ENSG00000107951 | MTPAP | ENSG00000108474 | PIGL |
| ENSG00000039650 | PNKP | ENSG00000187741 | FANCA |
| ENSG00000123064 | DDX54 | ENSG00000213465 | ARL2 |
| ENSG00000183955 | SETD8 | ENSG00000117593 | DARS2 |
| ENSG00000138107 | ACTR1A | ENSG00000171863 | RPS7 |
| ENSG00000244005 | NFS1 | ENSG00000117395 | EBNA1BP2 |
| ENSG00000188986 | NELFB | ENSG00000111142 | METAP2 |
| ENSG00000018699 | TTC27 | ENSG00000113272 | THG1L |
| ENSG00000167112 | TRUB2 | ENSG00000117360 | PRPF3 |
| ENSG00000100393 | EP300 | ENSG00000221978 | CCNL2 |
| ENSG00000101639 | CEP192 | ENSG00000163832 | ELP6 |
| ENSG00000126461 | SCAF1 | ENSG00000108852 | MPP2 |
| ENSG00000172171 | TEFM | ENSG00000175832 | ETV4 |
| ENSG00000135913 | USP37 | ENSG00000185359 | HGS |
| ENSG00000135624 | CCT7 | ENSG00000120705 | ETF1 |
| ENSG00000100804 | PSMB5 | ENSG00000108384 | RAD51C |
| ENSG00000175792 | RUVBL1 | ENSG00000036257 | CUL3 |
| ENSG00000183431 | SF3A3 | ENSG00000152382 | TADA1 |
| ENSG00000108773 | KAT2A | ENSG00000114742 | WDR48 |
| ENSG00000100949 | RABGGTA | ENSG00000214026 | MRPL23 |
| ENSG00000151503 | NCAPD3 | ENSG00000105671 | DDX49 |
| ENSG00000111880 | RNGTT | ENSG00000104731 | KLHDC4 |
| ENSG00000168883 | USP39 | ENSG00000010256 | UQCRC1 |
| ENSG00000151461 | UPF2 | ENSG00000154743 | TSEN2 |
| ENSG00000105486 | LIG1 | ENSG00000178896 | EXOSC4 |
| ENSG00000111300 | NAA25 | ENSG00000168393 | DTYMK |
| ENSG00000144559 | TAMM41 | ENSG00000035928 | RFC1 |
| ENSG00000137574 | TGS1 | ENSG00000048707 | VPS13D |
| ENSG00000172273 | HINFP | ENSG00000154832 | CXXC1 |
| ENSG00000133112 | TPT1 | ENSG00000130985 | UBA1 |
| ENSG00000167986 | DDB1 | ENSG00000065150 | IPO5 |
| ENSG00000125319 | C17orf53 | ENSG00000161800 | RACGAP1 |
| ENSG00000113161 | HMGCR | ENSG00000142534 | RPS11 |
| ENSG00000100941 | PNN | ENSG00000136003 | ISCU |
| ENSG00000139697 | SBNO1 | ENSG00000065000 | AP3D1 |
| ENSG00000135336 | ORC3 | ENSG00000100401 | RANGAP1 |
| ENSG00000101115 | SALL4 | ENSG00000196230 | TUBB |
| ENSG00000100902 | PSMA6 | ENSG00000181555 | SETD2 |
| ENSG00000141141 | DDX52 | ENSG00000055950 | MRPL43 |
| ENSG00000254093 | PINX1 | ENSG00000188389 | PDCD1 |
| ENSG00000184445 | KNTC1 | ENSG00000165684 | SNAPC4 |
| ENSG00000089053 | ANAPC5 | ENSG00000147533 | GOLGA7 |
| ENSG00000111602 | TIMELESS | ENSG00000064313 | TAF2 |
| ENSG00000145592 | RPL37 | ENSG00000137154 | RPS6 |
| ENSG00000106615 | RHEB | ENSG00000104886 | PLEKHJ1 |
| ENSG00000180817 | PPA1 | ENSG00000122882 | ECD |
| ENSG00000110172 | CHORDC1 | ENSG00000184967 | NOC4L |
| ENSG00000137876 | RSL24D1 | ENSG00000088325 | TPX2 |
| ENSG00000104408 | EIF3E | ENSG00000183520 | UTP11L |
| ENSG00000143436 | MRPL9 | ENSG00000179051 | RCC2 |
| ENSG00000108883 | EFTUD2 | ENSG00000157510 | AFAP1L1 |
| ENSG00000140740 | UQCRC2 | ENSG00000066379 | ZNRD1 |
| ENSG00000211456 | SACM1L | ENSG00000172115 | CYCS |
| ENSG00000131051 | RBM39 | ENSG00000086827 | ZW10 |
| ENSG00000136758 | YME1L1 | ENSG00000109534 | GAR1 |
| ENSG00000112578 | BYSL | ENSG00000175387 | SMAD2 |
| ENSG00000163781 | TOPBP1 | ENSG00000115947 | ORC4 |
| ENSG00000106628 | POLD2 | ENSG00000010072 | SPRTN |
| ENSG00000132952 | USPL1 | ENSG00000185163 | DDX51 |
| ENSG00000168538 | TRAPPC11 | ENSG00000177370 | TIMM22 |
| ENSG00000168488 | ATXN2L | ENSG00000076924 | XAB2 |
| ENSG00000022277 | RTFDC1 | ENSG00000124562 | SNRPC |
| ENSG00000179988 | PSTK | ENSG00000127586 | CHTF18 |
| ENSG00000092199 | HNRNPC | ENSG00000066117 | SMARCD1 |
| ENSG00000156831 | NSMCE2 | ENSG00000177494 | ZBED2 |
| ENSG00000125691 | RPL23 | ENSG00000133401 | PDZD2 |
| ENSG00000083520 | DIS3 | ENSG00000127554 | GFER |
| ENSG00000115761 | NOL10 | ENSG00000117697 | NSL1 |
| ENSG00000173894 | CBX2 | ENSG00000184659 | FOXD4L4 |
| ENSG00000243147 | MRPL33 | ENSG00000204828 | FOXD4L2 |
| ENSG00000139618 | BRCA2 | ENSG00000110200 | ANAPC15 |
| ENSG00000109519 | GRPEL1 | ENSG00000169291 | SHE |
| ENSG00000203760 | CENPW | ENSG00000132313 | MRPL35 |
| ENSG00000166851 | PLK1 | ENSG00000115816 | CEBPZ |
| ENSG00000121579 | NAA50 | ENSG00000243667 | WDR92 |
| ENSG00000163608 | C3orf17 | ENSG00000107959 | PITRM1 |
| ENSG00000005075 | POLR2J | ENSG00000103035 | PSMD7 |
| ENSG00000148606 | POLR3A | ENSG00000163946 | FAM208A |
| ENSG00000160949 | TONSL | ENSG00000178057 | NDUFAF3 |
| ENSG00000128159 | TUBGCP6 | ENSG00000170540 | ARL6IP1 |
| ENSG00000125449 | ARMC7 | ENSG00000091009 | RBM27 |
| ENSG00000122406 | RPL5 | ENSG00000205609 | EIF3CL |
| ENSG00000126226 | PCID2 | ENSG00000165526 | RPUSD4 |
| ENSG00000159377 | PSMB4 | ENSG00000120314 | WDR55 |
| ENSG00000167967 | E4F1 | ENSG00000013275 | PSMC4 |
| ENSG00000141076 | CIRH1A | ENSG00000131931 | THAP1 |
| ENSG00000069248 | NUP133 | ENSG00000155660 | PDIA4 |
| ENSG00000242372 | EIF6 | ENSG00000162607 | USP1 |
| ENSG00000087269 | NOP14 | ENSG00000109606 | DHX15 |
| ENSG00000163468 | CCT3 | ENSG00000261949 | LOC100507003 |
| ENSG00000140326 | CDAN1 | ENSG00000130589 | HELZ2 |
| ENSG00000146834 | MEPCE | ENSG00000145734 | BDP1 |
| ENSG00000143222 | UFC1 | ENSG00000103194 | USP10 |
| ENSG00000110871 | COQ5 | ENSG00000076201 | PTPN23 |
| ENSG00000119285 | HEATR1 | ENSG00000140854 | KATNB1 |
| ENSG00000145386 | CCNA2 | ENSG00000164053 | ATRIP |
| ENSG00000164109 | MAD2L1 | ENSG00000167088 | SNRPD1 |
| ENSG00000185347 | C14orf80 | ENSG00000154781 | CCDC174 |
| ENSG00000134748 | PRPF38A | ENSG00000115446 | UNC50 |
| ENSG00000070061 | IKBKAP | ENSG00000177700 | POLR2L |
| ENSG00000099995 | SF3A1 | ENSG00000162063 | CCNF |
| ENSG00000100029 | PES1 | ENSG00000152904 | GGPS1 |
| ENSG00000130255 | RPL36 | ENSG00000151657 | KIN |
| ENSG00000085231 | AK6 | ENSG00000182810 | DDX28 |
| ENSG00000187145 | MRPS21 | ENSG00000006744 | ELAC2 |
| ENSG00000062650 | WAPAL | ENSG00000116898 | MRPS15 |
| ENSG00000122484 | RPAP2 | ENSG00000255072 | PIGY |
| ENSG00000090861 | AARS | ENSG00000130332 | LSM7 |
| ENSG00000161888 | SPC24 | ENSG00000051180 | RAD51 |
| ENSG00000087087 | SRRT | ENSG00000178171 | AMER3 |
| ENSG00000134910 | STT3A | ENSG00000254901 | MEF2BNB |
| ENSG00000161526 | SAP30BP | ENSG00000149925 | ALDOA |
| ENSG00000068654 | POLR1A | ENSG00000100604 | CHGA |
| ENSG00000140983 | RHOT2 | ENSG00000172602 | RND1 |
| ENSG00000184708 | EIF4ENIF1 | ENSG00000138592 | USP8 |
| ENSG00000100479 | POLE2 | ENSG00000172613 | RAD9A |
| ENSG00000134440 | NARS | ENSG00000132196 | HSD17B7 |
| ENSG00000014164 | ZC3H3 | ENSG00000151849 | CENPJ |
| ENSG00000113812 | ACTR8 | ENSG00000105221 | AKT2 |
| ENSG00000145331 | TRMT10A | ENSG00000185504 | C17orf70 |
| ENSG00000110104 | CCDC86 | ENSG00000025796 | SEC63 |
| ENSG00000164163 | ABCE1 | ENSG00000168438 | CDC40 |
| ENSG00000167863 | ATP5H | ENSG00000163918 | RFC4 |
| ENSG00000176946 | THAP4 | ENSG00000152147 | GEMIN6 |
| ENSG00000169251 | NMD3 | ENSG00000166887 | VPS39 |
| ENSG00000166226 | CCT2 | ENSG00000018625 | ATP1A2 |
| ENSG00000131747 | TOP2A | ENSG00000163346 | PBXIP1 |
| ENSG00000267673 | FDX1L | ENSG00000135966 | TGFBRAP1 |
| ENSG00000108559 | NUP88 | ENSG00000099901 | RANBP1 |
| ENSG00000104957 | CCDC130 | ENSG00000010327 | STAB1 |
| ENSG00000167522 | ANKRD11 | ENSG00000163344 | PMVK |
| ENSG00000130706 | ADRM1 | ENSG00000102921 | N4BP1 |
| ENSG00000048162 | NOP16 | ENSG00000177150 | FAM210A |
| ENSG00000159210 | SNF8 | ENSG00000158042 | MRPL17 |
| ENSG00000113360 | DROSHA | ENSG00000124659 | TBCC |
| ENSG00000108296 | CWC25 | ENSG00000113593 | PPWD1 |
| ENSG00000161395 | PGAP3 | ENSG00000188306 | LRRIQ4 |
| ENSG00000089195 | TRMT6 | ENSG00000074966 | TXK |
| ENSG00000185838 | GNB1L | ENSG00000228049 | POLR2J2 |
| ENSG00000101146 | RAE1 | ENSG00000133226 | SRRM1 |
| ENSG00000092853 | CLSPN | ENSG00000121577 | POPDC2 |
| ENSG00000107949 | BCCIP | ENSG00000130876 | SLC7A10 |
| ENSG00000159079 | C21orf59 | ENSG00000130810 | PPAN |
| ENSG00000137947 | GTF2B | ENSG00000243207 | PPAN-P2RY11 |
| ENSG00000160948 | VPS28 | ENSG00000081248 | CACNA1S |
| ENSG00000065427 | KARS | ENSG00000153201 | RANBP2 |
| ENSG00000102978 | POLR2C | ENSG00000126698 | DNAJC8 |
| ENSG00000182154 | MRPL41 | ENSG00000103018 | CYB5B |
| ENSG00000139168 | ZCRB1 | ENSG00000130816 | DNMT1 |
| ENSG00000175110 | MRPS22 | ENSG00000102103 | PQBP1 |
| ENSG00000177084 | POLE | ENSG00000120253 | NUP43 |
| ENSG00000197681 | TBC1D3 | ENSG00000164327 | RICTOR |
| ENSG00000053501 | USE1 | ENSG00000139719 | VPS33A |
| ENSG00000121879 | PIK3CA | ENSG00000168566 | SNRNP48 |
| ENSG00000108278 | ZNHIT3 | ENSG00000063244 | U2AF2 |
| ENSG00000161547 | SRSF2 | ENSG00000108423 | TUBD1 |
| ENSG00000129083 | COPB1 | ENSG00000164880 | INTS1 |
| ENSG00000012048 | BRCA1 | ENSG00000148297 | MED22 |
| ENSG00000171314 | PGAM1 | ENSG00000185825 | BCAP31 |
| ENSG00000112159 | MDN1 | ENSG00000084623 | EIF3I |
| ENSG00000174243 | DDX23 | ENSG00000066422 | ZBTB11 |
| ENSG00000096401 | CDC5L | ENSG00000119041 | GTF3C3 |
| ENSG00000128513 | POT1 | ENSG00000083093 | PALB2 |
| ENSG00000071859 | FAM50A | ENSG00000120699 | EXOSC8 |
| ENSG00000100084 | HIRA | ENSG00000166135 | HIF1AN |
| ENSG00000100813 | ACIN1 | ENSG00000188976 | NOC2L |
| ENSG00000005100 | DHX33 | ENSG00000102974 | CTCF |
| ENSG00000101158 | NELFCD | ENSG00000148229 | POLE3 |
| ENSG00000115946 | PNO1 | ENSG00000167118 | URM1 |
| ENSG00000188647 | PTAR1 | ENSG00000176386 | CDC26 |
| ENSG00000146007 | ZMAT2 | ENSG00000110063 | DCPS |
| ENSG00000241837 | ATP5O | ENSG00000089737 | DDX24 |
| ENSG00000113643 | RARS | ENSG00000119383 | PPP2R4 |
| ENSG00000162521 | RBBP4 | ENSG00000143319 | ISG20L2 |
| ENSG00000116830 | TTF2 | ENSG00000141552 | ANAPC11 |
| ENSG00000187555 | USP7 | ENSG00000155506 | LARP1 |
| ENSG00000137216 | TMEM63B | ENSG00000144867 | SRPRB |
| ENSG00000161904 | LEMD2 | ENSG00000093000 | NUP50 |
| ENSG00000241945 | PWP2 | ENSG00000107937 | GTPBP4 |
| ENSG00000134982 | APC | ENSG00000083635 | NUFIP1 |
| ENSG00000156983 | BRPF1 | ENSG00000174527 | MYO1H |
| ENSG00000164346 | NSA2 | ENSG00000124641 | MED20 |
| ENSG00000223496 | EXOSC6 | ENSG00000240694 | PNMA2 |
| ENSG00000113569 | NUP155 | ENSG00000122012 | SV2C |
| ENSG00000080986 | NDC80 | ENSG00000017260 | ATP2C1 |
| ENSG00000143374 | TARS2 | ENSG00000179965 | ZNF771 |
| ENSG00000104835 | SARS2 | ENSG00000126216 | TUBGCP3 |
| ENSG00000152253 | SPC25 | ENSG00000126814 | TRMT5 |
| ENSG00000088356 | PDRG1 | ENSG00000101945 | SUV39H1 |
| ENSG00000044574 | HSPA5 | ENSG00000182185 | RAD51B |
| ENSG00000116874 | WARS2 | ENSG00000163681 | SLMAP |
| ENSG00000204531 | POU5F1 | ENSG00000179295 | PTPN11 |
| ENSG00000004779 | NDUFAB1 | ENSG00000004487 | KDM1A |
| ENSG00000161981 | SNRNP25 | ENSG00000136100 | VPS36 |
| ENSG00000126457 | PRMT1 | ENSG00000168066 | SF1 |
| ENSG00000142507 | PSMB6 | ENSG00000197181 | PIWIL2 |
| ENSG00000164808 | SPIDR | ENSG00000128908 | INO80 |
| ENSG00000234972 | TBC1D3C | ENSG00000102144 | PGK1 |
| ENSG00000144554 | FANCD2 | ENSG00000007923 | DNAJC11 |
| ENSG00000147383 | NSDHL | ENSG00000143514 | TP53BP2 |
| ENSG00000165732 | DDX21 | ENSG00000076650 | GPATCH1 |
| ENSG00000155975 | VPS37A | ENSG00000130749 | ZC3H4 |
| ENSG00000002822 | MAD1L1 | ENSG00000062582 | MRPS24 |
| ENSG00000179271 | GADD45GIP1 | ENSG00000087085 | ACHE |
| ENSG00000101452 | DHX35 | ENSG00000197976 | AKAP17A |
| ENSG00000074071 | MRPS34 | ENSG00000100028 | SNRPD3 |
| ENSG00000169045 | HNRNPH1 | ENSG00000128731 | HERC2 |
| ENSG00000087510 | TFAP2C | ENSG00000134014 | ELP3 |
| ENSG00000105819 | PMPCB | ENSG00000181163 | NPM1 |
| ENSG00000204351 | SKIV2L | ENSG00000148444 | COMMD3 |
| ENSG00000160783 | PMF1 | ENSG00000095319 | NUP188 |
| ENSG00000152234 | ATP5A1 | ENSG00000169564 | PCBP1 |
| ENSG00000127463 | EMC1 | ENSG00000182208 | MOB2 |
| ENSG00000124228 | DDX27 | ENSG00000055070 | SZRD1 |
| ENSG00000100319 | ZMAT5 | ENSG00000182473 | EXOC7 |
| ENSG00000065183 | WDR3 | ENSG00000136930 | PSMB7 |
| ENSG00000058272 | PPP1R12A | ENSG00000107863 | ARHGAP21 |
| ENSG00000136628 | EPRS | ENSG00000197223 | C1D |
| ENSG00000163017 | ACTG2 | ENSG00000184270 | HIST2H2AB |
| ENSG00000104884 | ERCC2 | ENSG00000161036 | LRWD1 |
| ENSG00000166483 | WEE1 | ENSG00000144736 | SHQ1 |
| ENSG00000135837 | CEP350 | ENSG00000137100 | DCTN3 |
| ENSG00000104897 | SF3A2 | ENSG00000131149 | GSE1 |
| ENSG00000140598 | EFTUD1 | ENSG00000214753 | HNRNPUL2 |
| ENSG00000143774 | GUK1 | ENSG00000111358 | GTF2H3 |
| ENSG00000085721 | RRN3 | ENSG00000147677 | EIF3H |
| ENSG00000172053 | QARS | ENSG00000125676 | THOC2 |
| ENSG00000165934 | CPSF2 | ENSG00000149554 | CHEK1 |
| ENSG00000052802 | MSMO1 | ENSG00000176476 | CCDC101 |
| ENSG00000135476 | ESPL1 | ENSG00000147596 | PRDM14 |
| ENSG00000174177 | CTU2 | ENSG00000092094 | OSGEP |
| ENSG00000120438 | TCP1 | ENSG00000155393 | HEATR3 |
| ENSG00000170892 | TSEN34 | ENSG00000083845 | RPS5 |
| ENSG00000204574 | ABCF1 | ENSG00000148296 | SURF6 |
| ENSG00000175376 | EIF1AD | ENSG00000162613 | FUBP1 |
| ENSG00000146263 | MMS22L | ENSG00000182220 | ATP6AP2 |
| ENSG00000121022 | COPS5 | ENSG00000115163 | CENPA |
| ENSG00000168090 | COPS6 | ENSG00000176225 | RTTN |
| ENSG00000167491 | GATAD2A | ENSG00000176208 | ATAD5 |
| ENSG00000084072 | PPIE | ENSG00000254827 | SLC22A18AS |
| ENSG00000115268 | RPS15 | ENSG00000128708 | HAT1 |
| ENSG00000163938 | GNL3 | ENSG00000106400 | ZNHIT1 |
| ENSG00000151665 | PIGF | ENSG00000123219 | CENPK |
| ENSG00000148843 | PDCD11 | ENSG00000264424 | MYH4 |
| ENSG00000141736 | ERBB2 | ENSG00000066468 | FGFR2 |
| ENSG00000103168 | TAF1C | ENSG00000095059 | DHPS |
| ENSG00000105401 | CDC37 | ENSG00000110921 | MVK |
| ENSG00000163933 | RFT1 | ENSG00000141556 | TBCD |
| ENSG00000122085 | MTERFD2 | ENSG00000196305 | IARS |
| ENSG00000164032 | H2AFZ | ENSG00000131055 | COX4I2 |
| ENSG00000140943 | MBTPS1 | ENSG00000153789 | FAM92B |
| ENSG00000198952 | SMG5 | ENSG00000088930 | XRN2 |
| ENSG00000169021 | UQCRFS1 | ENSG00000145220 | LYAR |
| ENSG00000013810 | TACC3 | ENSG00000172809 | RPL38 |
| ENSG00000105258 | POLR2I | ENSG00000108788 | MLX |
| ENSG00000167978 | SRRM2 | ENSG00000197170 | PSMD12 |
| ENSG00000095564 | BTAF1 | ENSG00000225899 | FRG2B |
| ENSG00000138095 | LRPPRC | ENSG00000174886 | NDUFA11 |
| ENSG00000063978 | RNF4 | ENSG00000172058 | SERF1A |
| ENSG00000162368 | CMPK1 | ENSG00000205572 | SERF1B |
| ENSG00000140829 | DHX38 | ENSG00000242485 | MRPL20 |
| ENSG00000158169 | FANCC | ENSG00000089225 | TBX5 |
| ENSG00000161960 | EIF4A1 | ENSG00000149428 | HYOU1 |
| ENSG00000181222 | POLR2A | ENSG00000166595 | FAM96B |
| ENSG00000165916 | PSMC3 | ENSG00000131462 | TUBG1 |
| ENSG00000198060 | MARCH5 | ENSG00000185990 | F8A3 |
| ENSG00000149923 | PPP4C | ENSG00000197932 | F8A1 |
| ENSG00000111667 | USP5 | ENSG00000198444 | F8A2 |
| ENSG00000198755 | RPL10A | ENSG00000031823 | RANBP3 |
| ENSG00000141499 | WRAP53 | ENSG00000100353 | EIF3D |
| ENSG00000093009 | CDC45 | ENSG00000163605 | PPP4R2 |
| ENSG00000105732 | ZNF574 | ENSG00000164162 | ANAPC10 |
| ENSG00000104064 | GABPB1 | ENSG00000132153 | DHX30 |
| ENSG00000108294 | PSMB3 | ENSG00000154723 | ATP5J |
| ENSG00000130856 | ZNF236 | ENSG00000182256 | GABRG3 |
| ENSG00000133980 | VRTN | ENSG00000119487 | MAPKAP1 |
| ENSG00000149308 | NPAT | ENSG00000132394 | EEFSEC |
| ENSG00000120071 | KANSL1 | ENSG00000122952 | ZWINT |
| ENSG00000129084 | PSMA1 | ENSG00000131042 | LILRB2 |
| ENSG00000117877 | CD3EAP | ENSG00000222004 | C7orf71 |
| ENSG00000127616 | SMARCA4 | ENSG00000168802 | CHTF8 |
| ENSG00000163882 | POLR2H | ENSG00000069849 | ATP1B3 |
| ENSG00000183718 | TRIM52 | ENSG00000074582 | BCS1L |
| ENSG00000106803 | SEC61B | ENSG00000103126 | AXIN1 |
| ENSG00000114942 | EEF1B2 | ENSG00000187144 | SPATA21 |
| ENSG00000067704 | IARS2 | ENSG00000221914 | PPP2R2A |
| ENSG00000114686 | MRPL3 | ENSG00000163386 | NBPF10 |
| ENSG00000172315 | TP53RK | ENSG00000134987 | WDR36 |
| ENSG00000173120 | KDM2A | ENSG00000132300 | PTCD3 |
| ENSG00000138442 | WDR12 | ENSG00000156931 | VPS8 |
| ENSG00000145982 | FARS2 | ENSG00000165632 | TAF3 |
| ENSG00000117481 | NSUN4 | ENSG00000044115 | CTNNA1 |
| ENSG00000142676 | RPL11 | ENSG00000035403 | VCL |
| ENSG00000164615 | CAMLG | ENSG00000088256 | GNA11 |
| ENSG00000138073 | PREB | ENSG00000164334 | FAM170A |
| ENSG00000136888 | ATP6V1G1 | ENSG00000166225 | FRS2 |
| ENSG00000221829 | FANCG | ENSG00000241186 | TDGF1 |
| ENSG00000198887 | SMC5 | ENSG00000196374 | HIST1H2BM |
| ENSG00000102900 | NUP93 | ENSG00000117614 | SYF2 |
| ENSG00000108344 | PSMD3 | ENSG00000154222 | CC2D1B |
| ENSG00000023191 | RNH1 | ENSG00000101367 | MAPRE1 |
| ENSG00000143621 | ILF2 | ENSG00000188186 | LAMTOR4 |
| ENSG00000112855 | HARS2 | ENSG00000166924 | NYAP1 |
| ENSG00000110536 | PTPMT1 | ENSG00000079805 | DNM2 |
| ENSG00000165629 | ATP5C1 | ENSG00000011260 | UTP18 |
| ENSG00000166847 | DCTN5 | ENSG00000089685 | BIRC5 |
| ENSG00000104852 | SNRNP70 | ENSG00000123908 | AGO2 |
| ENSG00000203814 | HIST2H2BF | ENSG00000057935 | MTA3 |
| ENSG00000009413 | REV3L | ENSG00000100811 | YY1 |
| ENSG00000130772 | MED18 | ENSG00000064102 | ASUN |
| ENSG00000079313 | REXO1 | ENSG00000006025 | OSBPL7 |
| ENSG00000012061 | ERCC1 | ENSG00000107372 | ZFAND5 |
| ENSG00000111642 | CHD4 | ENSG00000172922 | RNASEH2C |
| ENSG00000100462 | PRMT5 | ENSG00000075089 | ACTR6 |
| ENSG00000174100 | MRPL45 | ENSG00000165119 | HNRNPK |
| ENSG00000101421 | CHMP4B | ENSG00000182518 | FAM104B |
| ENSG00000144028 | SNRNP200 | ENSG00000041802 | LSG1 |
| ENSG00000108592 | FTSJ3 | ENSG00000206557 | TRIM71 |
| ENSG00000110048 | OSBP | ENSG00000124140 | SLC12A5 |
| ENSG00000147403 | RPL10 | ENSG00000063046 | EIF4B |
| ENSG00000198783 | ZNF830 | ENSG00000126581 | BECN1 |
| ENSG00000179409 | GEMIN4 | ENSG00000171530 | TBCA |
| ENSG00000147604 | RPL7 | ENSG00000206127 | GOLGA8O |
| ENSG00000136824 | SMC2 | ENSG00000167842 | MIS12 |
| ENSG00000104889 | RNASEH2A | ENSG00000033011 | ALG1 |
| ENSG00000146282 | RARS2 | ENSG00000146670 | CDCA5 |
| ENSG00000068784 | SRBD1 | ENSG00000198856 | OSTC |
| ENSG00000137822 | TUBGCP4 | ENSG00000111605 | CPSF6 |
| ENSG00000059691 | PET112 | ENSG00000087365 | SF3B2 |
| ENSG00000066827 | ZFAT | ENSG00000135845 | PIGC |
| ENSG00000148308 | GTF3C5 | ENSG00000100220 | RTCB |
| ENSG00000170185 | USP38 | ENSG00000131876 | SNRPA1 |
| ENSG00000160201 | U2AF1 | ENSG00000115392 | FANCL |
| ENSG00000141258 | SGSM2 | ENSG00000078618 | NRD1 |
| ENSG00000172660 | TAF15 | ENSG00000025770 | NCAPH2 |
| ENSG00000145833 | DDX46 | ENSG00000117682 | DHDDS |
| ENSG00000104980 | TIMM44 | ENSG00000198844 | ARHGEF15 |
| ENSG00000097046 | CDC7 | ENSG00000132603 | NIP7 |
| ENSG00000131368 | MRPS25 | ENSG00000162377 | SELRC1 |
| ENSG00000204209 | DAXX | ENSG00000137411 | VARS2 |
| ENSG00000129696 | TTI2 | ENSG00000064886 | CHI3L2 |
| ENSG00000108848 | LUC7L3 | ENSG00000137806 | NDUFAF1 |
| ENSG00000013573 | DDX11 | ENSG00000133030 | MPRIP |
| ENSG00000105248 | CCDC94 | ENSG00000136935 | GOLGA1 |
| ENSG00000183598 | HIST2H3D | ENSG00000243927 | MRPS6 |
| ENSG00000224226 | TBC1D3B | ENSG00000046647 | GEMIN8 |
| ENSG00000090470 | PDCD7 | ENSG00000133124 | IRS4 |
| ENSG00000031698 | SARS | ENSG00000255346 | NOX5 |
| ENSG00000108270 | AATF | ENSG00000103275 | UBE2I |
| ENSG00000159111 | MRPL10 | ENSG00000165502 | RPL36AL |
| ENSG00000149806 | FAU | ENSG00000100056 | DGCR14 |
| ENSG00000188739 | RBM34 | ENSG00000167972 | ABCA3 |
| ENSG00000152684 | PELO | ENSG00000053372 | MRTO4 |
| ENSG00000174374 | WBSCR16 | ENSG00000169813 | HNRNPF |
| ENSG00000107036 | KIAA1432 | ENSG00000198258 | UBL5 |
| ENSG00000204619 | PPP1R11 | ENSG00000103245 | NARFL |
| ENSG00000091651 | ORC6 | ENSG00000183513 | COA5 |
| ENSG00000134480 | CCNH | ENSG00000174547 | MRPL11 |
| ENSG00000164151 | KIAA0947 | ENSG00000173457 | PPP1R14B |
| ENSG00000164611 | PTTG1 | ENSG00000088038 | CNOT3 |
| ENSG00000111445 | RFC5 | ENSG00000115539 | PDCL3 |
| ENSG00000127481 | UBR4 | ENSG00000118181 | RPS25 |
| ENSG00000159352 | PSMD4 | ENSG00000160075 | SSU72 |
| ENSG00000137814 | HAUS2 | ENSG00000257949 | TEN1 |
| ENSG00000105220 | GPI | ENSG00000168028 | RPSA |
| ENSG00000140521 | POLG | ENSG00000213066 | FGFR1OP |
| ENSG00000075856 | SART3 | ENSG00000143228 | NUF2 |
| ENSG00000143742 | SRP9 | ENSG00000137413 | TAF8 |
| ENSG00000163029 | SMC6 | ENSG00000124207 | CSE1L |
| ENSG00000162227 | TAF6L | ENSG00000080815 | PSEN1 |
| ENSG00000100129 | EIF3L | ENSG00000132773 | TOE1 |
| ENSG00000170348 | TMED10 | ENSG00000129460 | NGDN |
| ENSG00000182217 | HIST2H4B | ENSG00000188613 | NANOS1 |
| ENSG00000183941 | HIST2H4A | ENSG00000163636 | PSMD6 |
| ENSG00000116221 | MRPL37 | ENSG00000146232 | NFKBIE |
| ENSG00000196235 | SUPT5H | ENSG00000135902 | CHRND |
| ENSG00000161920 | MED11 | ENSG00000143641 | GALNT2 |
| ENSG00000134690 | CDCA8 | ENSG00000073969 | NSF |
| ENSG00000131153 | GINS2 | ENSG00000041982 | TNC |
| ENSG00000138018 | EPT1 | ENSG00000108256 | NUFIP2 |
| ENSG00000173141 | MRP63 | ENSG00000198911 | SREBF2 |
| ENSG00000154727 | GABPA | ENSG00000141385 | AFG3L2 |
| ENSG00000120800 | UTP20 | ENSG00000176108 | CHMP6 |
| ENSG00000114767 | RRP9 | ENSG00000257365 | FNTB |
| ENSG00000174231 | PRPF8 | ENSG00000186487 | MYT1L |
| ENSG00000137547 | MRPL15 | ENSG00000127423 | AUNIP |
| ENSG00000146576 | C7orf26 | ENSG00000112110 | MRPL18 |
| ENSG00000065268 | WDR18 | ENSG00000114650 | SCAP |
| ENSG00000147162 | OGT | ENSG00000178104 | PDE4DIP |
| ENSG00000198917 | C9orf114 | ENSG00000105656 | ELL |
| ENSG00000180822 | PSMG4 | ENSG00000186393 | KRT26 |
| ENSG00000125977 | EIF2S2 | ENSG00000124541 | RRP36 |
| ENSG00000173418 | NAA20 | ENSG00000182108 | DEXI |
| ENSG00000155561 | NUP205 | ENSG00000139133 | ALG10 |
| ENSG00000173545 | ZNF622 | ENSG00000082068 | WDR70 |
| ENSG00000127993 | RBM48 | ENSG00000151388 | ADAMTS12 |
| ENSG00000197102 | DYNC1H1 | ENSG00000172172 | MRPL13 |
| ENSG00000119392 | GLE1 | ENSG00000184979 | USP18 |
| ENSG00000174444 | RPL4 | ENSG00000239857 | GET4 |
| ENSG00000149716 | ORAOV1 | ENSG00000069345 | DNAJA2 |
| ENSG00000155876 | RRAGA | ENSG00000073050 | XRCC1 |
| ENSG00000198841 | KTI12 | ENSG00000070985 | TRPM5 |
| ENSG00000056097 | ZFR | ENSG00000158715 | SLC45A3 |
| ENSG00000227057 | WDR46 | ENSG00000172062 | SMN1 |
| ENSG00000167670 | CHAF1A | ENSG00000205571 | SMN2 |
| ENSG00000127191 | TRAF2 | ENSG00000113141 | IK |
| ENSG00000072506 | HSD17B10 | ENSG00000186105 | LRRC70 |
| ENSG00000215021 | PHB2 | ENSG00000157895 | C12orf43 |
| ENSG00000175467 | SART1 | ENSG00000166441 | RPL27A |
| ENSG00000121073 | SLC35B1 | ENSG00000106346 | USP42 |
| ENSG00000079459 | FDFT1 | ENSG00000185379 | RAD51D |
| ENSG00000143493 | INTS7 | ENSG00000116667 | C1orf21 |
| ENSG00000141543 | EIF4A3 | ENSG00000176444 | CLK2 |
| ENSG00000174197 | MGA | ENSG00000105472 | CLEC11A |
| ENSG00000131269 | ABCB7 | ENSG00000065613 | SLK |
| ENSG00000089009 | RPL6 | ENSG00000005156 | LIG3 |
| ENSG00000197780 | TAF13 | ENSG00000125459 | MSTO1 |
| ENSG00000036549 | ZZZ3 | ENSG00000139146 | FAM60A |
| ENSG00000066135 | KDM4A | ENSG00000060069 | CTDP1 |
| ENSG00000176473 | WDR25 | ENSG00000130935 | NOL11 |
| ENSG00000124614 | RPS10 | ENSG00000115677 | HDLBP |
| ENSG00000107581 | EIF3A | ENSG00000105254 | TBCB |
| ENSG00000084463 | WBP11 | ENSG00000075539 | FRYL |
| ENSG00000137656 | BUD13 | ENSG00000196747 | HIST1H2AI |
| ENSG00000183751 | TBL3 | ENSG00000181513 | ACBD4 |
| ENSG00000119537 | KDSR | ENSG00000153107 | ANAPC1 |
| ENSG00000204220 | PFDN6 | ENSG00000160211 | G6PD |
| ENSG00000170291 | ELP5 | ENSG00000111481 | COPZ1 |
| ENSG00000198563 | DDX39B | ENSG00000070761 | C16orf80 |
| ENSG00000077549 | CAPZB | ENSG00000168924 | LETM1 |
| ENSG00000255529 | POLR2M | ENSG00000105058 | FAM32A |
| ENSG00000100034 | PPM1F | ENSG00000204569 | PPP1R10 |
| ENSG00000196367 | TRRAP | ENSG00000153914 | SREK1 |
| ENSG00000167258 | CDK12 | ENSG00000161509 | GRIN2C |
| ENSG00000039123 | SKIV2L2 | ENSG00000162702 | ZNF281 |
| ENSG00000076043 | REXO2 | ENSG00000004939 | SLC4A1 |
| ENSG00000213676 | ATF6B | ENSG00000139620 | KANSL2 |
| ENSG00000058453 | CROCC | ENSG00000025293 | PHF20 |
| ENSG00000153575 | TUBGCP5 | ENSG00000158545 | ZC3H18 |
| ENSG00000110700 | RPS13 | ENSG00000142546 | NOSIP |
| ENSG00000101181 | MTG2 | ENSG00000143398 | PIP5K1A |
| ENSG00000071539 | TRIP13 | ENSG00000197958 | RPL12 |
| ENSG00000075702 | WDR62 | ENSG00000067225 | PKM |
| ENSG00000171453 | POLR1C | ENSG00000172534 | HCFC1 |
| ENSG00000090989 | EXOC1 | ENSG00000155438 | MKI67IP |
| ENSG00000037897 | METTL1 | ENSG00000166582 | CENPV |
| ENSG00000095139 | ARCN1 | ENSG00000145912 | NHP2 |
| ENSG00000078142 | PIK3C3 | ENSG00000180992 | MRPL14 |
| ENSG00000141030 | COPS3 | ENSG00000118705 | RPN2 |
| ENSG00000126249 | PDCD2L | ENSG00000163161 | ERCC3 |
| ENSG00000117408 | IPO13 | ENSG00000136819 | C9orf78 |
| ENSG00000130725 | UBE2M | ENSG00000124787 | RPP40 |
| ENSG00000175054 | ATR | ENSG00000179104 | TMTC2 |
| ENSG00000149016 | TUT1 | ENSG00000140694 | PARN |
| ENSG00000165060 | FXN | ENSG00000143751 | SDE2 |
| ENSG00000117597 | DIEXF | ENSG00000136997 | MYC |
| ENSG00000185085 | INTS5 | ENSG00000147274 | RBMX |
| ENSG00000113595 | TRIM23 | ENSG00000084693 | AGBL5 |
| ENSG00000040633 | PHF23 | ENSG00000165271 | NOL6 |
| ENSG00000178952 | TUFM | ENSG00000221838 | AP4M1 |
| ENSG00000120539 | MASTL | ENSG00000171444 | MCC |
| ENSG00000103549 | RNF40 | ENSG00000101882 | NKAP |
| ENSG00000119723 | COQ6 | ENSG00000186847 | KRT14 |
| ENSG00000171311 | EXOSC1 | ENSG00000014824 | SLC30A9 |
| ENSG00000106245 | BUD31 | ENSG00000166685 | COG1 |
| ENSG00000118046 | STK11 | ENSG00000108349 | CASC3 |
| ENSG00000125484 | GTF3C4 | ENSG00000175216 | CKAP5 |
| ENSG00000089094 | KDM2B | ENSG00000259494 | MRPL46 |
| ENSG00000121621 | KIF18A | ENSG00000028310 | BRD9 |
| ENSG00000129911 | KLF16 | ENSG00000136450 | SRSF1 |
| ENSG00000102302 | FGD1 | ENSG00000204859 | ZBTB48 |
| ENSG00000135679 | MDM2 | ENSG00000165209 | STRBP |
| ENSG00000185115 | NDNL2 | ENSG00000163466 | ARPC2 |
| ENSG00000140553 | UNC45A | ENSG00000125485 | DDX31 |
| ENSG00000129562 | DAD1 | ENSG00000070778 | PTPN21 |
| ENSG00000100138 | NHP2L1 | ENSG00000126001 | CEP250 |
| ENSG00000111641 | NOP2 | ENSG00000169249 | ZRSR2 |
| ENSG00000173660 | UQCRH | ENSG00000111011 | RSRC2 |
| ENSG00000198677 | TTC37 | ENSG00000139496 | NUPL1 |
| ENSG00000135503 | ACVR1B | ENSG00000131746 | TNS4 |
| ENSG00000180998 | GPR137C | ENSG00000061936 | SFSWAP |
| ENSG00000153187 | HNRNPU | ENSG00000196584 | XRCC2 |
| ENSG00000106459 | NRF1 | ENSG00000168286 | THAP11 |
| ENSG00000156261 | CCT8 | ENSG00000119787 | ATL2 |
| ENSG00000118363 | SPCS2 | ENSG00000182446 | NPLOC4 |
| ENSG00000164134 | NAA15 | ENSG00000071462 | WBSCR22 |
| ENSG00000060642 | PIGV | ENSG00000213397 | HAUS7 |
| ENSG00000090889 | KIF4A | ENSG00000178028 | DMAP1 |
| ENSG00000101361 | NOP56 | ENSG00000067596 | DHX8 |
| ENSG00000167792 | NDUFV1 | ENSG00000198015 | MRPL42 |
| ENSG00000184162 | NR2C2AP | ENSG00000133706 | LARS |
| ENSG00000128524 | ATP6V1F | ENSG00000149635 | OCSTAMP |
| ENSG00000100387 | RBX1 | ENSG00000117505 | DR1 |
| ENSG00000110906 | KCTD10 | ENSG00000155868 | MED7 |
| ENSG00000147457 | CHMP7 | ENSG00000129197 | RPAIN |
| ENSG00000124570 | SERPINB6 | ENSG00000065978 | YBX1 |
| ENSG00000186468 | RPS23 | ENSG00000260238 | PMF1-BGLAP |
| ENSG00000136122 | BORA | ENSG00000178988 | MRFAP1L1 |
| ENSG00000047249 | ATP6V1H | ENSG00000168005 | C11orf84 |
| ENSG00000127804 | METTL16 | ENSG00000162408 | NOL9 |
| ENSG00000104412 | EMC2 | ENSG00000140350 | ANP32A |
| ENSG00000173726 | TOMM20 | ENSG00000261796 | ISY1-RAB43 |
| ENSG00000138777 | PPA2 | ENSG00000174405 | LIG4 |
| ENSG00000170043 | TRAPPC1 | ENSG00000197414 | GOLGA6L1 |
| ENSG00000124486 | USP9X | ENSG00000116062 | MSH6 |
| ENSG00000105705 | SUGP1 | ENSG00000116906 | GNPAT |
| ENSG00000223501 | VPS52 | ENSG00000134597 | RBMX2 |
| ENSG00000107815 | C10orf2 | ENSG00000071994 | PDCD2 |
| ENSG00000100109 | TFIP11 | ENSG00000112742 | TTK |
| ENSG00000136271 | DDX56 | ENSG00000106636 | YKT6 |
| ENSG00000146830 | GIGYF1 | ENSG00000101773 | RBBP8 |
| ENSG00000198382 | UVRAG | ENSG00000103061 | SLC7A6OS |
| ENSG00000160285 | LSS | ENSG00000140259 | MFAP1 |
| ENSG00000137770 | CTDSPL2 | ENSG00000197077 | KIAA1671 |
| ENSG00000116670 | MAD2L2 | ENSG00000204435 | CSNK2B |
| ENSG00000165280 | VCP | ENSG00000055130 | CUL1 |
| ENSG00000183963 | SMTN | ENSG00000100209 | HSCB |
| ENSG00000164961 | KIAA0196 | ENSG00000113048 | MRPS27 |
| ENSG00000157216 | SSBP3 | ENSG00000189403 | HMGB1 |
| ENSG00000129932 | DOHH | ENSG00000173011 | TADA2B |
| ENSG00000167721 | TSR1 | ENSG00000169836 | TACR3 |
| ENSG00000188352 | FOCAD | ENSG00000133816 | MICAL2 |
| ENSG00000104853 | CLPTM1 | ENSG00000141452 | C18orf8 |
| ENSG00000185883 | ATP6V0C | ENSG00000006715 | VPS41 |
| ENSG00000100519 | PSMC6 | ENSG00000136518 | ACTL6A |
| ENSG00000110107 | PRPF19 | ENSG00000100297 | MCM5 |
| ENSG00000184203 | PPP1R2 | ENSG00000165898 | ISCA2 |
| ENSG00000148824 | MTG1 | ENSG00000156384 | SFR1 |
| ENSG00000113810 | SMC4 | ENSG00000145414 | NAF1 |
| ENSG00000121152 | NCAPH | ENSG00000101972 | STAG2 |
| ENSG00000241127 | YAE1D1 | ENSG00000112658 | SRF |
| ENSG00000139197 | PEX5 | ENSG00000162736 | NCSTN |
| ENSG00000101464 | PIGU | ENSG00000103266 | STUB1 |
| ENSG00000132676 | DAP3 | ENSG00000008018 | PSMB1 |
| ENSG00000135972 | MRPS9 | ENSG00000149506 | ZP1 |
| ENSG00000089157 | RPLP0 | ENSG00000111530 | CAND1 |
| ENSG00000138035 | PNPT1 | ENSG00000027001 | MIPEP |
| ENSG00000171824 | EXOSC10 | ENSG00000152266 | PTH |
| ENSG00000153179 | RASSF3 | ENSG00000154174 | TOMM70A |
| ENSG00000110713 | NUP98 | ENSG00000164045 | CDC25A |
| ENSG00000100865 | CINP | ENSG00000164758 | MED30 |
| ENSG00000136045 | PWP1 | ENSG00000160401 | C9orf117 |
| ENSG00000167526 | RPL13 | ENSG00000155959 | VBP1 |
| ENSG00000088766 | CRLS1 | ENSG00000105409 | ATP1A3 |
| ENSG00000103510 | KAT8 | ENSG00000175106 | TVP23C |
| ENSG00000143368 | SF3B4 | ENSG00000185950 | IRS2 |
| ENSG00000156697 | UTP14A | ENSG00000149256 | TENM4 |
| ENSG00000176248 | ANAPC2 | ENSG00000116957 | TBCE |
| ENSG00000188786 | MTF1 | ENSG00000154719 | MRPL39 |
| ENSG00000175756 | AURKAIP1 | ENSG00000105364 | MRPL4 |
| ENSG00000140395 | WDR61 | ENSG00000198218 | QRICH1 |
| ENSG00000113368 | LMNB1 | ENSG00000013503 | POLR3B |
| ENSG00000060339 | CCAR1 | ENSG00000126756 | UXT |
| ENSG00000162385 | MAGOH | ENSG00000184988 | TMEM106A |
| ENSG00000105372 | RPS19 | ENSG00000186432 | KPNA4 |
| ENSG00000083312 | TNPO1 | ENSG00000156304 | SCAF4 |
| ENSG00000100142 | POLR2F | ENSG00000090565 | RAB11FIP3 |
| ENSG00000204560 | DHX16 | ENSG00000163508 | EOMES |
| ENSG00000197771 | MCMBP | ENSG00000147003 | TMEM27 |
| ENSG00000099817 | POLR2E | ENSG00000198730 | CTR9 |
| ENSG00000161980 | POLR3K | ENSG00000105321 | CCDC9 |
| ENSG00000117133 | RPF1 | ENSG00000120333 | MRPS14 |
| ENSG00000125901 | MRPS26 | ENSG00000121680 | PEX16 |
| ENSG00000168827 | GFM1 | ENSG00000088205 | DDX18 |
| ENSG00000161513 | FDXR | ENSG00000132432 | SEC61G |
| ENSG00000137818 | RPLP1 | ENSG00000186329 | TMEM212 |
| ENSG00000150990 | DHX37 | ENSG00000094804 | CDC6 |
| ENSG00000061794 | MRPS35 | ENSG00000169084 | DHRSX |
| ENSG00000143155 | TIPRL | ENSG00000107618 | RBP3 |
| ENSG00000253626 | EIF5AL1 | ENSG00000146426 | TIAM2 |
| ENSG00000231500 | RPS18 | ENSG00000198925 | ATG9A |
| ENSG00000188076 | SCGB1C1 | ENSG00000168242 | HIST1H2BI |
| ENSG00000174442 | ZWILCH | ENSG00000254772 | EEF1G |
| ENSG00000242028 | HYPK | ENSG00000090971 | NAT14 |
| ENSG00000124217 | MOCS3 | ENSG00000144381 | HSPD1 |
| ENSG00000134186 | PRPF38B | ENSG00000127774 | EMC6 |
| ENSG00000105849 | TWISTNB | ENSG00000126259 | KIRREL2 |
| ENSG00000137337 | MDC1 | ENSG00000111364 | DDX55 |
| ENSG00000132207 | SLX1A | ENSG00000100749 | VRK1 |
| ENSG00000181625 | SLX1B | ENSG00000159063 | ALG8 |
| ENSG00000110717 | NDUFS8 | ENSG00000163795 | ZNF513 |
| ENSG00000132341 | RAN | ENSG00000068394 | GPKOW |
| ENSG00000014123 | UFL1 | ENSG00000112659 | CUL9 |
| ENSG00000101191 | DIDO1 | ENSG00000187257 | RSBN1L |
| ENSG00000125952 | MAX | ENSG00000172167 | MTBP |
| ENSG00000163714 | U2SURP | ENSG00000176177 | ENTHD1 |
| ENSG00000253710 | ALG11 | ENSG00000166783 | KIAA0430 |
| ENSG00000104356 | POP1 | ENSG00000165006 | UBAP1 |
| ENSG00000130826 | DKC1 | ENSG00000188958 | UTS2B |
| ENSG00000198780 | FAM169A | ENSG00000136247 | ZDHHC4 |
| ENSG00000116688 | MFN2 | ENSG00000196363 | WDR5 |
| ENSG00000166166 | TRMT61A | ENSG00000116661 | FBXO2 |
| ENSG00000214517 | PPME1 | ENSG00000113013 | HSPA9 |
| ENSG00000077235 | GTF3C1 | ENSG00000090061 | CCNK |
| ENSG00000152240 | HAUS1 | ENSG00000051596 | THOC3 |
| ENSG00000063177 | RPL18 | ENSG00000140534 | TICRR |
| ENSG00000087157 | PGS1 | ENSG00000100216 | TOMM22 |
| ENSG00000100567 | PSMA3 | ENSG00000104613 | INTS10 |
| ENSG00000169371 | SNUPN | ENSG00000183474 | GTF2H2C |
| ENSG00000197651 | CCER1 | ENSG00000159128 | IFNGR2 |
| ENSG00000198900 | TOP1 | ENSG00000243725 | TTC4 |
| ENSG00000213551 | DNAJC9 | ENSG00000102898 | NUTF2 |
| ENSG00000152464 | RPP38 | ENSG00000170515 | PA2G4 |
| ENSG00000131467 | PSME3 | ENSG00000117036 | ETV3 |
| ENSG00000223510 | CDRT15 | ENSG00000196262 | PPIA |
| ENSG00000115053 | NCL | ENSG00000153037 | SRP19 |
| ENSG00000163041 | H3F3A | ENSG00000135801 | TAF5L |
| ENSG00000154813 | DPH3 | ENSG00000119414 | PPP6C |
| ENSG00000181873 | IBA57 | ENSG00000141013 | GAS8 |
| ENSG00000185591 | SP1 | ENSG00000113845 | TIMMDC1 |
| ENSG00000115355 | CCDC88A | ENSG00000175826 | CTDNEP1 |
| ENSG00000139350 | NEDD1 | ENSG00000117543 | DPH5 |
| ENSG00000108518 | PFN1 | ENSG00000204779 | FOXD4L5 |
| ENSG00000108264 | TADA2A | ENSG00000112249 | ASCC3 |
| ENSG00000134809 | TIMM10 | ENSG00000152256 | PDK1 |
| ENSG00000124383 | MPHOSPH10 | ENSG00000169217 | CD2BP2 |
| ENSG00000126067 | PSMB2 | ENSG00000166246 | C16orf71 |
| ENSG00000060688 | SNRNP40 | ENSG00000184164 | CRELD2 |
| ENSG00000042429 | MED17 | ENSG00000107960 | OBFC1 |
| ENSG00000196655 | TRAPPC4 | ENSG00000102384 | CENPI |
| ENSG00000107185 | RGP1 | ENSG00000079785 | DDX1 |
| ENSG00000124608 | AARS2 | ENSG00000133858 | ZFC3H1 |
| ENSG00000092098 | RNF31 | ENSG00000184110 | EIF3C |
| ENSG00000143569 | UBAP2L | ENSG00000146700 | SRCRB4D |
| ENSG00000233822 | HIST1H2BN | ENSG00000163380 | LMOD3 |
| ENSG00000171848 | RRM2 | ENSG00000116273 | PHF13 |
| ENSG00000183161 | FANCF | ENSG00000178229 | ZNF543 |
| ENSG00000166197 | NOLC1 | ENSG00000109475 | RPL34 |
| ENSG00000064703 | DDX20 | ENSG00000156469 | MTERFD1 |
| ENSG00000176102 | CSTF3 | ENSG00000155827 | RNF20 |
| ENSG00000106028 | SSBP1 | ENSG00000213741 | RPS29 |
| ENSG00000143315 | PIGM | ENSG00000165792 | METTL17 |
| ENSG00000136152 | COG3 | ENSG00000110844 | PRPF40B |
| ENSG00000134697 | GNL2 | ENSG00000100842 | EFS |
| ENSG00000159217 | IGF2BP1 | ENSG00000087495 | PHACTR3 |
| ENSG00000080608 | KIAA0020 | ENSG00000126261 | UBA2 |
| ENSG00000267368 | UPK3BL | ENSG00000136718 | IMP4 |
| ENSG00000130119 | GNL3L | ENSG00000091640 | SPAG7 |
| ENSG00000178950 | GAK | ENSG00000184886 | PIGW |
| ENSG00000205659 | LIN52 | ENSG00000184313 | MROH7 |
| ENSG00000123297 | TSFM | ENSG00000163481 | RNF25 |
| ENSG00000241370 | RPP21 | ENSG00000137054 | POLR1E |
| ENSG00000129351 | ILF3 | ENSG00000213085 | CCDC19 |
| ENSG00000174446 | SNAPC5 | ENSG00000171858 | RPS21 |
| ENSG00000132382 | MYBBP1A | ENSG00000130822 | PNCK |
| ENSG00000100664 | EIF5 | ENSG00000145216 | FIP1L1 |
| ENSG00000131469 | RPL27 | ENSG00000147130 | ZMYM3 |
| ENSG00000185128 | TBC1D3F | ENSG00000008086 | CDKL5 |
| ENSG00000111231 | GPN3 | ENSG00000165282 | PIGO |
| ENSG00000182774 | RPS17L | ENSG00000038358 | EDC4 |
| ENSG00000184779 | RPS17 | ENSG00000134684 | YARS |
| ENSG00000186871 | ERCC6L | ENSG00000153832 | FBXO36 |
| ENSG00000204568 | MRPS18B | ENSG00000140006 | WDR89 |
| ENSG00000108312 | UBTF | ENSG00000104643 | MTMR9 |
| ENSG00000167965 | MLST8 | ENSG00000151779 | NBAS |
| ENSG00000115241 | PPM1G | ENSG00000077348 | EXOSC5 |
| ENSG00000171103 | TRMT61B | ENSG00000131043 | AAR2 |
| ENSG00000116586 | LAMTOR2 | ENSG00000160193 | WDR4 |
| ENSG00000105793 | GTPBP10 | ENSG00000140691 | ARMC5 |
| ENSG00000100348 | TXN2 | ENSG00000141959 | PFKL |
| ENSG00000172757 | CFL1 | ENSG00000112053 | SLC26A8 |
| ENSG00000163634 | THOC7 | ENSG00000197111 | PCBP2 |
| ENSG00000008324 | SS18L2 | ENSG00000145191 | EIF2B5 |
| ENSG00000152404 | CWF19L2 | ENSG00000140988 | RPS2 |
| ENSG00000020129 | NCDN | ENSG00000181472 | ZBTB2 |

The gene symbols used in herein (including in Tables 3 and 4) are based on those found in the Human Gene Naming Committee (HGNC) which is searchable on the world-wide web at www.genenames.org. Ensembl IDs are provided for each gene symbol and are searchable world-wide web at www.ensembl.org.

The genes provided in Tables 3 and 4 are non-limiting examples of essential genes. Although additional essential genes will be apparent to the skilled artisan based on the knowledge in the art, the suitability of a particular gene for use according to the present disclosure can be determined, e.g., as discussed herein. For example, in some embodiments, a particular essential gene can be selected by analysis of potential off-target sites elsewhere in the genome. In some embodiments, only essential genes with one or more gRNA target sites that are unique in the human genome are selected for methods described herein. In some embodiments, only essential genes with one or more gRNA target sites that are found in only one other locus in the human genome are selected for methods described herein. In some embodiments, only essential genes with one or more gRNA target sites found in only two other loci in the human genome are selected for methods described herein.

### Gene Product of Interest

The methods, systems and cells of the present disclosure enable the integration of a gene of interest at an essential gene of a cell. The gene of interest can encode any gene product of interest. In certain embodiments, a gene product of interest comprises an antibody, an antigen, an enzyme, a growth factor, a receptor (e.g., cell surface, cytoplasmic, or nuclear), a hormone, a lymphokine, a cytokine, a chemokine, a reporter, a fusion protein comprising an immunogenic protein and an immunoglobulin domain, a chimeric antigen receptor (CAR), a functional fragment of any of the above, or a combination of any of the above.

In some embodiments, sequence for a gene product of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e., a polypeptide found in nature) or fragment thereof; a variant polypeptide (i.e., a mutant of the native polypeptide having less than 100% sequence identity with the native polypeptide) or fragment thereof; an engineered polypeptide or peptide fragment, a therapeutic peptide or polypeptide, an imaging marker, a selectable marker, a degradation signal, and the like.

In some embodiments, a gene product of interest may be but is not limited to, e.g., a therapeutic protein or a gene product that confers a desired feature to the modified cell. In some embodiments, the transgene encodes a reporter protein, such as a fluorescent protein (e.g., as described herein) and an enzyme (e.g., luciferase and lacZ). In some embodiments, a reporter gene may aid the tracking of therapeutic cells once they are introduced to a subject.

In some embodiments, a gene product of interest may be a therapeutic polypeptide, e.g., an enzyme, an antibody or antigen binding fragment thereof, a receptor, a chimeric antigen receptor, or a cytokine.

In some embodiments, a therapeutic polypeptide is a protein lacking and/or deficient in a patient (e.g., a patient having and/or diagnosed with a genetic disease). In some embodiments, a polypeptide is fibrinogen, prothrombin, tissue factor, Factor V, Factor VII, Factor VIII, Factor IX, Factor X, Factor XI, Factor XII (Hageman factor), Factor XIII (fibrin-stabilizing factor), von Willebrand factor, prekallikrein, high molecular weight kininogen (Fitzgerald factor), fibronectin, antithrombin III, heparin cofactor II, protein C, protein S, protein Z, protein Z-related protease inhibitor, plasminogen, alpha 2-antiplasmin, tissue plasminogen activator, urokinase, plasminogen activator inhibitor-1, plasminogen activator inhibitor-2, angiotensin-converting enzyme 2 (Ace2), glucocerebrosidase (GBA), alpha-galactosidase A (GLA), arylsulfatase A, iduronate sulfatase (IDS), iduronidase (IDUA), acid sphingomyelinase (ASM), acid alpha-glucosidase (GAA), MMAA, MMAB, MMACHC, MMADHC (C2orf25), MTRR, LMBRD1, MTR, propionyl-CoA carboxylase (PCC) (PCCA and/or PCCB subunits), a glucose-6-phosphate transporter (G6PT) protein or glucose-6-phosphatase (G6Pase), an LDL receptor (LDLR), ApoB, LDLRAP-1, a PCSK9, a mitochondrial protein such as NAGS (N-acetylglutamate synthetase), CPS1 (carbamoyl phosphate synthetase I), and OTC (omithine transcarbamylase), ASS (argininosuccinic acid synthetase), ASL (argininosuccinase acid lyase) and/or ARGI (arginase), and/or a solute carrier family 25 (SLC25A13, an aspartate/glutamate carrier) protein, a UGTIAI or UDP glucuronsyltransferase polypeptide A1, a fumarylacetoacetate hydrolyase (FAH), an alanine-glyoxylate aminotransferase (AGXT) protein, a glyoxylate reductase/hydroxypyruvate reductase (GRHPR) protein, a transthyretin gene (TTR) protein, an ATP7B protein, a phenylalanine hydroxylase (PAH) protein, a lipoprotein lyase (LPL) protein, phenylalanine ammonia-lyase (PAL) protein, or glucagon-like peptide-1 (GLP-1), or a portion thereof.

In some embodiments, a gene product of interest is an antibody (e.g., a therapeutic antibody), or a portion thereof. Exemplary antibodies include, e.g., Rituximab, Palivizumab, Infliximab, Trastuzumab, Alemtuzumab, Adalimumab, Ibritumomab tiuxetan, Omalizumab, Cetuximab, Bevacizumab, Natalizumab, Panitumumab, Ranibizumab, Certolizumab pegol, Ustekinumab, Canakinumab, Golimumab, Ofatumumab, Tocilizumab, Denosumab, Belimumab, Ipilimumab, Brentuximab vedotin, Pertuzumab, Trastuzumab emtansine, Obinutuzumab, Siltuximab, Ramucirumab, Vedolizumab, Blinatumomab, Nivolumab, Pembrolizumab, Idarucizumab, Necitumumab, Dinutuximab, Secukinumab, Mepolizumab, Alirocumab, Evolocumab, Daratumumab, Elotuzumab, Ixekizumab, Reslizumab, Olaratumab, Bezlotoxumab, Atezolizumab, Obiltoxaximab, Inotuzumab ozogamicin, Brodalumab, Guselkumab, Dupilumab, Sarilumab, Avelumab, Ocrelizumab, Emicizumab, Benralizumab, Gemtuzumab ozogamicin, Durvalumab, Burosumab, Lanadelumab, Mogamulizumab, Erenumab, Galcanezumab, Tildrakizumab, Cemiplimab, Emapalumab, Fremanezumab, Ibalizumab, Moxetumomab pasudodox, Ravulizumab, Romosozumab, Risankizumab, Polatuzumab vedotin, Brolucizumab, or any combination thereof (see e.g., Lu et al., Development of therapeutic antibodies for the treatment of diseases. Journal of Biomedical Science, 2020). Additional gene products of interest include antibodies (or portions thereof) that bind to CD138, CD38, CD33, CD123, CD72, CD79a, CD79b, mesothelin, PD-1, PD-L1, PSMA, BCMA, ROR1, MUC-16, LlCAM, CD22, CD19, CD20, CD23, CD24, CD37, CD30, CA125, CD56, c-Met, EGFR, GD-3, HPV E6, HPV E7, MUC-1, HER2, folate receptor a, CD97, CD171, CD179a, CD44v6, WT1, VEGF-A, VEGFR1, VEGFR2, IL13RA1, IL13RA2, IL11RA, PSA, FcRH5, NKG2D ligand, NY-ESO-1, TAG-72, CEA, ephrin A2, ephrin B2, Lewis A antigen, Lewis Y antigen, MAGE, MAGE-A1, RAGE-1, folate receptor beta, EGFRvlII, LGR5, SSX2, AKAP-4, FLT3, fucosyl GM1, GM3, o-acetyl-GD2, or GD2.

In some embodiments, a gene product of interest may be a protein involved in immune regulation, or an immunomodulatory protein. In some embodiments, for example, such proteins are, PD-L1, CTLA-4, M-CSF, IL-4, IL-6, IL-10, IL-11, IL-13, TGF-β1, and various isoforms thereof. By way of example, in some embodiments, a gene product of interest may be an isoform of HLA-G (e.g., HLA-G1, -G2, -G3, -G4, -G5, -G6, or -G7) or HLA-E; allogeneic cells expressing such a nonclassical MHC class I molecule may be less immunogenic and better tolerated when transplanted into a human patient who is not the source of the cells, making “universal” cell therapy possible.

In some embodiments, a gene product of interest may be a protein involved in promotion of B cell survival, proliferation, and/or differentiation. In some embodiments, for example, such proteins are BAFF, CD40L, IL-4, and various isoforms thereof.

In some embodiments, a gene product of interest may be a cytokine. In some embodiments, expression of a cytokine from a modified cell generated using a method as described herein allows for localized dosing of the cytokine in vivo (e.g., within a subject in need thereof) and/or avoids a need to systemically administer a high-dose of the cytokine to a subject in need thereof (e.g., a lower dose of the cytokine may be administered). In some embodiments, the risk of dose-limiting toxicities associated with administering a cytokine is reduced while cytokine mediated cell functions are maintained. In some embodiments, to facilitate cell function without the need to additionally administer high-doses of soluble cytokines, a partial or full peptide of one or more of IL2, IL4, IL6, IL7, IL9, IL10, IL11, IL12, IL15, IL18, IL21, IFN-α, IFN-β and/or their respective receptor is introduced to the cell to enable cytokine signaling with or without the expression of the cytokine itself, thereby maintaining or improving cell growth, proliferation, expansion, and/or effector function with reduced risk of cytokine toxicities. In some embodiments, the introduced cytokine and/or its respective native or modified receptor for cytokine signaling are expressed on the cell surface. In some embodiments, the cytokine signaling is constitutively activated. In some embodiments, the activation of the cytokine signaling is inducible. In some embodiments, the activation of the cytokine signaling is transient and/or temporal. In some embodiments, a gene product if interest can be IL2, IL3, IL4, IL6, IL7, IL9, IL10, IL11, IL12, IL13, IL15, IL21, GM-CSF, IFN-α, IFN-b, IFN-g, erythropoietin, and/or the respective cytokine receptor. In some embodiments, a gene product of interest can be CCL3, TNFα, CCL23, IL2RB, IL12RB2, or IRF7.

In some embodiments, a gene product of interest can be a chemokine and/or the respective chemokine receptor. In some embodiments, a chemokine receptor can be, but is not limited to, CCR2, CCR5, CCR8, CX3C1, CX3CR1, CXCR1, CXCR2, CXCR3A, CXCR3B, or CXCR2. In some embodiments, a chemokine can be, but is not limited to, CCL7, CCL19, or CXL14.

In some embodiments, a gene product of interest is a CAR, such as but not limited to a CAR targeting mesothelin, EGFR, HER2 and/or MICA/B. CARs are well-known to those of ordinary skill in the art and include those described in, for example: WO13/063419 (mesothelin), WO15/164594 (EGFR), WO13/063419 (HER2), WO16/154585 (MICA and MICB), the entire contents of each of which are expressly incorporated herein by reference in their entireties. Exemplary CARs (or binders that target to a cell), include, but are not limited to, bi-specific antigen binding CARs, switchable CARs, dimerizable CARs, split CARs, multi-chain CARs, inducible CARs, CARs and binders that bind BCMA, androgen receptor, PSMA, PSCA, Mucd, HPV viral peptides (i.e., E7), EBV viral peptides, WT1, CEA, EGFR, EGFRvIII, IL13Rα2, GD2, CA125, EpCAM, Muc16, carbonic anhydrase IX (CAIX), CCR1, CCR4, carcinoembryonic antigen (CEA), CD3, CD5, CD7, CD10, CD19, CD20, CD22, CD23, CD24, CD26, CD30, CD33, CD34, CD35, CD38 CD41, CD44, CD44V6, CD49f, CD56, CD70, CD92, CD99, CD123, CD133, CD135, CD148, CD150, CD261, CD362, CLEC12A, MDM2, CYP1B, livin, cyclin 1, NKp30, NKp46, DNAM1, NKp44, CA9, PD1, PDL1, an antigen of cytomegalovirus (CMV), epithelial glycoprotein-40 (EGP-40), GPRC5D, receptor tyrosine kinases erb-B2,3,4, EGFIR, ERBB folate binding protein (FBP), fetal acetylcholine receptor (AChR), folate receptor-a, ganglioside G3 (GD3) human Epidermal Growth Factor Receptor 2 (HER-2), human telomerase reverse transcriptase (hTERT), ICAM-1, Integrin B7, Interleukin-13 receptor subunit alpha-2 (IL-13Ra2), K-light chain, kinase insert domain receptor (KDR), Lewis A (CA19.9), Lewis Y (Le Y), L1 cell adhesion molecule (LI-CAM), LILRB2, melanoma antigen family A 1 (MAGE-A1), MICA/B, Mucin 16 (Muc-16), NKCSI, NKG2D ligands, c-Met, cancer-testis antigen NYES0-1, oncofetal antigen (h5T4), PRAME, prostate stem cell antigen (PSCA), PRAME prostate-specific membrane antigen (PSMA), tumor-associated glycoprotein 72 (TAG-72), TIM-3, TRBCI, TRBC2, vascular endothelial growth factor R2 (VEGF-R2), Wilms tumor protein (WT-1), a pathogen antigen, or any suitable combination thereof. Additional suitable CARs and binders include those described in FIG. 3 of Davies and Maher, Adoptive T-cell Immunotherapy of Cancer Using Chimeric Antigen Receptor-Grafted T Cells, Archivum Immunologiae et Therapiae Experimentalis 58(3):165-78 (2010), the entire contents of which are incorporated herein by reference. Additional CARs suitable for methods described herein include: CD171-specific CARs (Park et al., Mol Ther (2007) 15(4):825-833), EGFRvIII-specific CARs (Morgan et al, Hum Gene Ther (2012) 23(10): 1043-1053), EGF-R-specific CARs (Kobold et al, J Natl Cancer Inst (2014)107(1):364), carbonic anhydrase K-specific CARs (Lamers et al., Biochem Soc Trans (2016) 44(3):951-959), FR-a-specific CARs (Kershaw et al., Clin Cancer Res (2006) 12(20):6106-6015), HER2-specific CARs (Ahmed et al., J Clin Oncol (2015) 33(15)1688-1696; Nakazawa et al., Mol Ther (2011) 19(12):2133-2143; Ahmed et al., Mol Ther (2009) 17(10): 1779-1787; Luo et al., Cell Res (2016) 26(7):850-853; Morgan et al., Mol Ther (2010) 18(4):843-85 1; Grada et al., Mol Ther Nucleic Acids (2013) 9(2):32), CEA-specific CARs (Katz et al., Clin Cancer Res (2015) 21 (14):3149-3159), IL13Ra2-specific CARs (Brown et al., Clin Cancer Res (2015) 21(18):4062-4072), GD2-specific CARs (Louis et al., Blood (2011) 118(23):6050-6056; Caruana et al., Nat Med (2015) 21(5):524-529), ErbB2-specific CARs (Wilkie et al., J Clin Immunol (2012) 32(5): 1059-1070), VEGF-R-specific CARs (Chinnasamy et al., Cancer Res (2016) 22(2):436-447), FAP-specific CARs (Wang et al., Cancer Immunol Res (2014) 2(2): 154-166), MSLN-specific CARs (Moon et al., Clin Cancer Res (2011) 17(14):4719-30), CD19-specific CARs (Axicabtagene ciloleucel (Yescarta®) and Tisagenlecleucel (Kymriah®). See also, Li et al., J Hematol and Oncol (2018) 11(22), reviewing clinical trials of tumor-specific CARs.

In some embodiments, the gene product of interest comprises a protein or polypeptide whose expression within a cell, e.g., a cell modified as described herein, enables the cell to inhibit or evade immune rejection after transplant or engraftment into a subject. In some embodiments, the gene product of interest is HLA-E, HLA-G, CTL4, CD47, or an associated ligand.

In some embodiments, a gene product of interest comprises a chimeric switch receptor (see e.g., WO2018094244A1—TGFBeta Signal Converter; Ankri et al., Human T cells Engineered to express a programmed death 1/28 costimulatory retargeting molecule display enhanced antitumor activity, The Journal of Immunology, Oct. 15, 2013, 191; Roth et al., Pooled knockin targeting for genome engineering of cellular immunotherapies, Cell. 2020 Apr. 30; 181(3):728-744.e21; and Boyerinas et al., A Novel TGF-β2/Interleukin Receptor Signal Conversion Platform That Protects CAR/TCR T Cells from TGF-β2-Mediated Immune Suppression and Induces T Cell Supportive Signaling Networks, Blood, 2017). In some embodiments, chimeric switch receptors are engineered cell-surface receptors comprising an extracellular domain from an endogenous cell-surface receptor and a heterologous intracellular signaling domain, such that ligand recognition by the extracellular domain results in activation of a different signaling cascade than that activated by the wild type form of the cell-surface receptor. In some embodiments, a chimeric switch receptor comprises an extracellular domain of an inhibitory cell-surface receptor fused to an intracellular domain that leads to the transmission of an activating signal rather than the inhibitory signal normally transduced by the inhibitory cell-surface receptor. In some embodiments, extracellular domains derived from cell-surface receptors known to inhibit immune effector cell activation can be fused to activating intracellular domains. In such an embodiment, engagement of the corresponding ligand may then activate signaling cascades that increase, rather than inhibit, the activation of the immune effector cell. For example, in some embodiments, a gene product of interest is a PD1-CD28 switch receptor, wherein the extracellular domain of PD1 is fused to the intracellular signaling domain of CD28 (See e.g., Liu et al., Cancer Res 76:6 (2016), 1578-1590 and Moon et al., Molecular Therapy 22 (2014), S201). In some embodiments, encoding gene product of interest is or comprises the extracellular domain of CD200R and the intracellular signaling domain of CD28 (See Oda et al., Blood 130:22 (2017), 2410-2419).

In some embodiments, a gene product of interest is a reporter gene (e.g., GFP, mCherry, etc.). In some embodiments, a reporter gene is utilized to confirm the suitability of a knock-in cassette's expression capacity. In certain embodiments, a gene product of interest may be a colored or fluorescent protein such as: blue/UV proteins, e.g. TagBFP, mTagBFP2, Azurite, EBFP2, mKalamal, Sirius, Sapphire, T-Sapphire; cyan proteins, e.g. ECFP, Cerulean, SCFP3A, mTurquoise, mTurquoise2, monomeric Midoriishi-Cyan, TagCFP, mTFPI; green proteins, e.g. EGFP, Emerald, Superfolder GFP, Monomeric Azami Green, TagGFP2, mUKG, m Wasabi, Clover, mNeonGreen; yellow proteins, e.g. EYFP, Citrine, Venus, SYFP2, TagYFP; orange proteins, e.g. Monomeric Kusabira-Orange, mKOK, mKO2, mOrange, mOrange2; red proteins, e.g. mRaspberry, mStrawberry, mTangerine, tdTomato, TagRFP, TagRFP-T, mApple, mRuby, mRuby2; far-red proteins, e.g. mPlum, HcRed-Tandem, mKate2, mNeptune, NirFP; near-IR proteins, e.g. TagRFP657, IFP1.4, iRFP; long stokes shift proteins, e.g. mKeima Red, LSS-mKate1, LSS-mKate2, mBeRFP; photoactivatible proteins, e.g. PA-GFP, PAmCherryl, PATagRFP; photoconvertible proteins, e.g. Kaede (green), Kaede (red), KikGR1 (green), KikGR1 (red), PS-CFP2, PS-CFP2, mEos2 (green), mEos2 (red), mEos3.2 (green), mEos3.2 (red), PSmOrange, PSmOrange, photoswitchable proteins, e.g. Dronpa, and combinations thereof.

In some embodiments, a gene of interest provided herein can optionally include a sequence encoding a destabilizing domain (“a destabilizing sequence”) for temporal and/or spatial control of protein expression. Non-limiting examples of destabilizing sequences include sequences encoding a FK506 sequence, a dihydrofolate reductase (DHFR) sequence, or other exemplary destabilizing sequences.

In the absence of a stabilizing ligand, a protein sequence operatively linked to a destabilizing sequence is degraded by ubiquitination. In contrast, in the presence of a stabilizing ligand, protein degradation is inhibited, thereby allowing the protein sequence operatively linked to the destabilizing sequence to be actively expressed. As a positive control for stabilization of protein expression, protein expression can be detected by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

Additional examples of destabilizing sequences are known in the art. In some embodiments, the destabilizing sequence is a FK506- and rapamycin-binding protein (FKBP12) sequence, and the stabilizing ligand is Shield-1 (Shld1) (Banaszynski et al. (2012) Cell 126(5): 995-1004, which is incorporated in its entirety herein by reference). In some embodiments, a destabilizing sequence is a DHFR sequence, and a stabilizing ligand is trimethoprim (TMP) (Iwamoto et al. (2010) Chem Biol 17:981-988, which is incorporated in its entirety herein by reference). In some embodiments, a destabilizing domain is small molecule-assisted shutoff (SMASh), where a constitutive degron with a protease and its corresponding cleavage site derived from hepatitis C virus are combined. In some embodiments, a destabilizing domain comprises a HaloTag system, dTag system, and/or nanobody (see e.g., Luh et al., Prey for the proteasome: targeted protein degradation—a medicinal chemist's perspective; Angewandte Chemie, 2020). In some embodiments, a destabilizing sequence can be used to temporally control a cell modified as described herein.

In some embodiments, a coding sequence for a single gene product of interest may be included in a knock-in cassette. In some embodiments, coding sequences for two gene products of interest may be included in a single knock-in cassette; in some embodiments, this may be referred to as a bicistronic or multicistronic construct. In some embodiments, coding sequences for more than two gene products of interest may be included in a single knock-in cassette; in some embodiments, this may be referred to as a multicistronic construct. In some embodiments, when more than one coding sequence for more than one gene product of interest is included in a knock-in cassette, these sequences may have a linker sequence connecting them. Linker sequences are generally known in the art, such as a nucleotide sequence encoding the amino acid sequence SGGGSGGGGSGGGGSGGGGSGGGSLQ. In some embodiments, where more than one coding sequence for more than one gene product of interest is included in a knock-in cassette, these sequences may be connected by a linker sequence, an IRES, and/or 2A element.

### AAV Capsids

In some embodiments, the present disclosure provides one or more polynucleotide constructs (e.g., donor templates) packaged into an AAV capsid. In some embodiments, an AAV capsid is from or derived from an AAV capsid of an AAV2, 3, 4, 5, 6, 7, 8, 9, or 10 serotype, or one or more hybrids thereof. In some embodiments, an AAV capsid is from an AAV ancestral serotype. In some embodiments, an AAV capsid is an ancestral (Anc) AAV capsid. An Anc capsid is created from a construct sequence that is constructed using evolutionary probabilities and evolutionary modeling to determine a probable ancestral sequence. In some embodiments, an AAV capsid has been modified in a manner known in the art (see e.g., Büning and Srivastava, Capsid modifications for targeting and improving the efficacy of AAV vectors, Mol Ther Methods Clin Dev. 2019)

In some embodiments, as provided herein, any combination of AAV capsids and AAV constructs (e.g., comprising AAV ITRs) may be used in recombinant AAV (rAAV) particles of the present disclosure. In some embodiments, an AAV ITR is from or derived from an AAV ITR of AAV2, 3, 4, 5, 6, 7, 8, 9, or 10. For example, wild-type or variant AA6 ITRs and AAV6 capsid, wild-type or variant AAV2 ITRs and AAV6 capsid, etc. In some embodiments of the present disclosure, an AAV particle is wholly comprised of AAV6 components (e.g., capsid and ITRs are AAV6 serotype). In some embodiments, an AAV particle is an AAV6/2, AAV6/8 or AAV6/9 particle (e.g., an AAV2, AAV8 or AAV9 capsid with an AAV construct having AAV6 ITRs).

### Nuclease

Any nuclease that causes a break within an endogenous coding sequence of an essential gene of the cell can be used in the methods of the present disclosure. In some embodiments the nuclease is a DNA nuclease. In some embodiments the nuclease causes a single-strand break (SSB) within an endogenous coding sequence of an essential gene of the cell, e.g., in a “prime editing” system. In some embodiments the nuclease causes a double-strand break (DSB) within an endogenous coding sequence of an essential gene of the cell. In some embodiments the double-strand break is caused by a single nuclease. In some embodiments the double-strand break is caused by two nucleases that each cause a single-strand break on opposing strands, e.g., a dual “nickase” system. In some embodiments the nuclease is a CRISPR/Cas nuclease and the method further comprises contacting the cell with one or more guide molecules for the CRISPR/Cas nuclease. Exemplary CRISPR/Cas nucleases and guide molecules are described in more detail herein. It is to be understood that the nuclease (including a nickase) is not limited in any manner and can also be a zinc finger nuclease (ZFN), a transcription activator-like effector nuclease (TALEN), a meganuclease, or other nuclease known in the art (or a combination thereof). Methods for designing zinc finger nucleases (ZFNs) are well known in the art, e.g., see Urnov et al., Nature Reviews Genetics 2010; 11:636-640 and Paschon et al., Nat. Commun. 2019; 10(1):1133 and references cited therein. Methods for designing transcription activator-like effector nucleases (TALENs) are well known in the art, e.g., see Joung and Sander, Nat. Rev. Mol. Cell Biol. 2013; 14(1):49-55 and references cited therein. Methods for designing meganucleases are also well known in the art, e.g., see Silva et al., Curr. Gene Ther. 2011; 11(1):11-27 and Redel and Prather, Toxicol. Pathol. 2016; 44(3):428-433.

In some embodiments, a nuclease suitable for methods described herein can have an editing efficiency that is greater than about 50%. In some embodiments, a nuclease suitable for methods described herein can have an editing efficiency that is greater than about 55%. In some embodiments, a nuclease suitable for methods described herein can have an editing efficiency that is greater than about 60%. In some embodiments, a nuclease suitable for methods described herein can have an editing efficiency that is greater than about 65%. In some embodiments, a nuclease suitable for methods described herein can have an editing efficiency that is greater than about 70%. In some embodiments, a nuclease suitable for methods described herein can have an editing efficiency that is greater than about 75%. In some embodiments, a nuclease suitable for methods described herein can have an editing efficiency that is greater than about 80%. In some embodiments, a nuclease suitable for methods described herein can have an editing efficiency that is greater than about 85%. In some embodiments, a nuclease suitable for methods described herein can have an editing efficiency that is greater than about 90%. In some embodiments, a nuclease suitable for methods described herein can have an editing efficiency that is greater than about 95%. In some embodiments, a nuclease suitable for methods described herein can have an editing efficiency that is greater than about 96%. In some embodiments, a nuclease suitable for methods described herein can have an editing efficiency that is greater than about 97%. In some embodiments, a nuclease suitable for methods described herein can have an editing efficiency that is greater than about 98%. In some embodiments, a nuclease suitable for methods described herein can have an editing efficiency that is greater than about 99%.

In general, the nuclease can be delivered to the cell as a protein or a nucleic acid encoding the protein, e.g., a DNA molecule or mRNA molecule. The protein or nucleic acid can be combined with other delivery agents, e.g., lipids or polymers in a lipid or polymer nanoparticle and targeting agents such as antibodies or other binding agents with specificity for the cell. The DNA molecule can be a nucleic acid vector, such as a viral genome or circular double-stranded DNA, e.g., a plasmid. Nucleic acid vectors encoding a nuclease can include other coding or non-coding elements. For example, a nuclease can be delivered as part of a viral genome (e.g., in an AAV, adenoviral or lentiviral genome) that includes certain genomic backbone elements (e.g., inverted terminal repeats, in the case of an AAV genome).

A CRISPR/Cas nuclease can be delivered to the cell as a protein or a nucleic acid encoding the protein, e.g., a DNA molecule or mRNA molecule. The guide molecule can be delivered as an RNA molecule or encoded by a DNA molecule. A CRISPR/Cas nuclease can also be delivered with a guide molecule as a ribonucleoprotein (RNP) and introduced into the cell via nucleofection (electroporation).

### CRISPR/Cas Nucleases

CRISPR/Cas nucleases according to the present disclosure include, but are not limited to, naturally-occurring Class 2 CRISPR nucleases such as Cas9, and Cpf1 (Cas12a), as well as other Cas12 nucleases and nucleases derived or obtained therefrom. In functional terms, CRISPR/Cas nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, CRISPR/Cas nucleases can be defined, in broad terms, by their PAM specificity and cleavage activity, even though variations may exist between individual CRISPR/Cas nucleases that share the same PAM specificity or cleavage activity. Skilled artisans will appreciate that some aspects of the present disclosure relate to systems and methods that can be implemented using any suitable CRISPR/Cas nuclease having a certain PAM specificity and/or cleavage activity. For this reason, unless otherwise specified, the term CRISPR/Cas nuclease should be understood as a generic term, and not limited to any particular type (e.g., Cas9 vs. Cpf1), species (e.g., S. pyogenes vs. S. aureus) or variation (e.g., full-length vs. truncated or split; naturally-occurring PAM specificity vs. engineered PAM specificity, etc.) of CRISPR/Cas nuclease.

The PAM sequence takes its name from its sequential relationship to the “protospacer” sequence that is complementary to gRNA targeting domains (or “spacers”). Together with protospacer sequences, PAM sequences define target regions or sequences for specific CRISPR/Cas nuclease and gRNA combinations.

Various CRISPR/Cas nucleases may require different sequential relationships between PAMs and protospacers. In general, Cas9s recognize PAM sequences that are 3′ of the protospacer. Cpf1 (Cas12a), on the other hand, generally recognizes PAM sequences that are 5′ of the protospacer.

In addition to recognizing specific sequential orientations of PAMs and protospacers, CRISPR/Cas nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. F. novicida Cpf1 recognizes a TTN PAM sequence. PAM sequences have been identified for a variety of CRISPR/Cas nucleases, and a strategy for identifying novel PAM sequences has been described by Shmakov et al., Molecular Cell 2015; 60:385-397. It should also be noted that engineered CRISPR/Cas nucleases can have PAM specificities that differ from the PAM specificities of reference molecules (for instance, in the case of an engineered CRISPR/Cas nuclease, the reference molecule may be the naturally occurring variant from which the CRISPR/Cas nuclease is derived, or the naturally occurring variant having the greatest amino acid sequence homology to the engineered CRISPR/Cas nuclease).

In addition to their PAM specificity, CRISPR/Cas nucleases can be characterized by their DNA cleavage activity: naturally-occurring CRISPR/Cas nucleases typically form double-strand breaks (DSBs) in target nucleic acids, but engineered variants called “nickases” have been produced that generate only single-strand breaks (SSBs), e.g., those discussed in Ran et al., Cell 2013; 154(6):1380-1389 (“Ran”), or that that do not cut at all.

### Cas9

Crystal structures have been determined for S. pyogenes Cas9 (Jinek et al., Science 2014; 343(6176):1247997 (“Jinek 2014”), and for S. aureus Cas9 in complex with a unimolecular guide RNA and a target DNA. See Nishimasu et al., Cell 1024; 156:935-949 (“Nishimasu 2014”); Nishimasu et al., Cell 2015; 162:1113-1126 (“Nishimasu 2015”); and Anders et al., Nature 2014; 513(7519):569-73 (“Anders 2014”).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g., a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, while the REC domain is thought to interact with the repeat:anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e., bottom) strand of the target nucleic acid. It may be formed from two or more split RuvC motifs (such as RuvC I, RuvCII, and RuvCIII in S. pyogenes and S. aureus). The HNH domain, meanwhile, is structurally similar to HNN endonuclease motifs, and cleaves the complementary (i.e., top) strand of the target nucleic acid. The PI domain, as its name suggests, contributes to PAM specificity.

While certain functions of Cas9 are linked to (but not necessarily fully determined by) the specific domains set forth above, these and other functions may be mediated or influenced by other Cas9 domains, or by multiple domains on either lobe. For instance, in S. pyogenes Cas9, as described in Nishimasu 2014, the repeat:antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains).

### Cpf1

The crystal structure of Acidaminococcus sp. Cpf1 in complex with crRNA and a dsDNA target including a TTTN PAM sequence has been solved by Yamano et al., Cell. 2016; 165(4):949-962 (“Yamano”). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, -II and -III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

### Nuclease Variants

The CRISPR/Cas nucleases described herein have activities and properties that can be useful in a variety of applications, but the skilled artisan will appreciate that CRISPR/Cas nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cpf1 Nuc domain are described in Ran, Yamano and PCT Publication No. WO 2016/073990A1, the entire contents of each of which are incorporated herein by reference. In general, mutations that reduce or eliminate activity in one of the two nuclease domains result in CRISPR/Cas nucleases with nickase activity, but it should be noted that the type of nickase activity varies depending on which domain is inactivated. As one example, inactivation of a RuvC domain or of a Cas9 HNH domain results in a nickase. Exemplary nickase variants include Cas9 D10A and Cas9 1840A (numbering scheme according to SpCas9 wild-type sequence). Additional suitable nickase variants, including Cas12a variants, will be apparent to the skilled artisan based on the present disclosure and the knowledge in the art. The present disclosure is not limited in this respect. In some embodiments a nickase may be fused to a reverse transcriptase to produce a prime editor (PE), e.g., as described in Anzalone et al., Nature 2019; 576:149-157, the entire contents of which are incorporated herein by reference.

Modifications of PAM specificity relative to naturally occurring Cas9 reference molecules has been described for both S. pyogenes (Kleinstiver et al., Nature 2015; 523(7561):481-5); and S. aureus (Kleinstiver et al., Nat Biotechnol. 2015; 33(12):1293-1298). Modifications that improve the targeting fidelity of Cas9 have also been described (Kleinstiver et al., Nature 2016; 529:490-495). Each of these references is incorporated by reference herein.

CRISPR/Cas nucleases have also been split into two or more parts, as described by Zetsche et al., Nat Biotechnol. 2015; 33(2):139-42, incorporated by reference, and by Fine et al., Sci Rep. 2015; 5:10777, incorporated by reference.

CRISPR/Cas nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger et al., Nature Biotech. 2014; 32:577-582, which is incorporated by reference herein.

CRISPR/Cas nucleases also optionally include a tag, such as, but not limited to, a nuclear localization signal, to facilitate movement of CRISPR/Cas nuclease protein into the nucleus. In certain embodiments, the CRISPR/Cas nuclease can incorporate C- and/or N-terminal nuclear localization signals. Nuclear localization sequences are known in the art.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular CRISPR/Cas nucleases, but it should be understood that the CRISPR/Cas nucleases used may be modified in ways that do not alter their operating principles. Such modifications are within the scope of the present disclosure.

Exemplary suitable nuclease variants include, but are not limited to, AsCpf1 (AsCas12a) variants comprising an M537R substitution, an H800A substitution, and/or an F870L substitution, or any combination thereof (numbering scheme according to AsCpf1 wild-type sequence). In some embodiments, a nuclease variant is a Cas12a variant, e.g., a Cas12a variant comprising 1, 2, or 3 of the amino acid substitutions selected from M537R, F870L, and H800A. In some embodiments, a Cas12a variant comprises an amino acid sequence having at least about 90%, 95%, or 100% identity to an AsCpf1 sequence described herein.

Other suitable modifications of the AsCpf1 amino acid sequence are known to those of ordinary skill in the art. Some exemplary sequences of wild-type AsCpf1 and AsCpf1 variants are provided below:

His-AsCpf1-sNLS-sNLS H800A amino acid sequence
SEQ ID NO: 58
MGHHHHHHGSTQFEGFTNLYQVSKTLRFELIPQGKTLKHIQEQGF
IEEDKARNDHYKELKPIIDRIYKTYADQCLQLVQLDWENLSAAID
SYRKEKTEETRNALIEEQATYRNAIHDYFIGRTDNLTDAINKRHA
EIYKGLFKAELFNGKVLKQLGTVTTTEHENALLRSFDKFTTYFSG
FYENRKNVFSAEDISTAIPHRIVQDNFPKFKENCHIFTRLITAVP
SLREHFENVKKAIGIFVSTSIEEVFSFPFYNQLLTQTQIDLYNQL
LGGISREAGTEKIKGLNEVLNLAIQKNDETAHIIASLPHRFIPLF
KQILSDRNTLSFILEEFKSDEEVIQSFCKYKILLRNENVLETAEA
LFNELNSIDLTHIFISHKKLETISSALCDHWDTLRNALYERRISE
LTGKITKSAKEKVQRSLKHEDINLQEIISAAGKELSEAFKQKTSE
ILSHAHAALDQPLPTTLKKQEEKEILKSQLDSLLGLYHLLDWFAV
DESNEVDPEFSARLTGIKLEMEPSLSFYNKARNYATKKPYSVEKF
KLNFQMPTLASGWDVNKEKNNGAILFVKNGLYYLGIMPKQKGRYK
ALSFEPTEKTSEGFDKMYYDYFPDAAKMIPKCSTQLKAVTAHFQT
HTTPILLSNNFIEPLEITKEIYDLNNPEKEPKKFQTAYAKKTGDQ
KGYREALCKWIDFTRDFLSKYTKTTSIDLSSLRPSSQYKDLGEYY
AELNPLLYHISFQRIAEKEIMDAVETGKLYLFQIYNKDFAKGHHG
KPNLHTLYWTGLFSPENLAKTSIKLNGQAELFYRPKSRMKRMAAR
LGEKMLNKKLKDQKTPIPDTLYQELYDYVNHRLSHDLSDEARALL
PNVITKEVSHEIIKDRRFTSDKFFFHVPITLNYQAANSPSKFNQR
VNAYLKEHPETPIIGIDRGERNLIYITVIDSTGKILEQRSLNTIQ
QFDYQKKLDNREKERVAARQAWSVVGTIKDLKQGYLSQVIHEIVD
LMIHYQAVVVLENLNFGFKSKRTGIAEKAVYQQFEKMLIDKLNCL
VLKDYPAEKVGGVLNPYQLTDQFTSFAKMGTQSGFLFYVPAPYTS
KIDPLTGFVDPFVWKTIKNHESRKHFLEGFDFLHYDVKTGDFILH
FKMNRNLSFQRGLPGFMPAWDIVFEKNETQFDAKGTPFIAGKRIV
PVIENHRFTGRYRDLYPANELIALLEEKGIVERDGSNILPKLLEN
DDSHAIDTMVALIRSVLQMRNSNAATGEDYINSPVRDLNGVCFDS
RFQNPEWPMDADANGAYHIALKGQLLLNHLKESKDLKLQNGISNQ
DWLAYIQELRNGSPKKKRKVGSPKKKRKV
Cpf1 variant 1 amino acid sequence
SEQ ID NO: 59
MTQFEGFTNLYQVSKILRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKITSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFLFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLIGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RNGRSSDDEATADSQHAAPPKKKRKVGGSGGSGGSGGSGGSGGSG
GSGGSLEHHHHHH
Cpf1 variant 2 amino acid sequence
SEQ ID NO: 60
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQMPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKITSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFFFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RNGRSSDDEATADSQHAAPPKKKRKVGGSGGSGGSGGSGGSGGSG
GSGGSLEHHHHHH
Cpf1 variant 3 amino acid sequence
SEQ ID NO: 61
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAARLGEKMLNKK
LKDQKTPIPDTLYQELYDYVNHRLSHDLSDEARALLPNVITKEVS
HEIIKDRRFTSDKFLFHVPITLNYQAANSPSKENQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RNGRSSDDEATADSQHAAPPKKKRKVGGSGGSGGSGGSGGSGGSG
GSGGSLEHHHHHH
Cpf1 variant 4 amino acid sequence
SEQ ID NO: 62
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKITSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAARLGEKMLNKK
LKDQKTPIPDTLYQELYDYVNHRLSHDLSDEARALLPNVITKEVS
HEIIKDRRFTSDKFLFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RNGRSSDDEATADSQHAAPPKKKRKV
Cpf1 variant 5 amino acid sequence
SEQ ID NO: 63
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRIDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKILLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKITSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFLFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRIGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RNGRSSDDEATADSQHAAPPKKKRKV
Cpf1 variant 6 amino acid sequence
SEQ ID NO: 64
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFLFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RNGRSSDDEATADSQHAAPPKKKRKVGGSGGSGGSGGSGGSGGSG
GSGGSLEHHHHHH
Cpf1 variant 7 amino acid sequence
SEQ ID NO: 65
MGRDPGKPIPNPLLGLDSTAPKKKRKVGIHGVPAATQFEGFTNLY
QVSKTLRFELIPQGKTLKHIQEQGFIEEDKARNDHYKELKPIIDR
IYKTYADQCLQLVQLDWENLSAAIDSYRKEKTEETRNALIEEQAT
YRNAIHDYFIGRTDNLTDAINKRHAEIYKGLFKAELFNGKVLKQL
GTVTTTEHENALLRSFDKFTTYFSGFYENRKNVFSAEDISTAIPH
RIVQDNFPKFKENCHIFTRLITAVPSLREHFENVKKAIGIFVSTS
IEEVFSFPFYNQLLTQTQIDLYNQLLGGISREAGTEKIKGLNEVL
NLAIQKNDETAHIIASLPHRFIPLFKQILSDRNILSFILEEFKSD
EEVIQSFCKYKTLLRNENVLETAEALFNELNSIDLTHIFISHKKL
ETISSALCDHWDTLRNALYERRISELTGKITKSAKEKVQRSLKHE
DINLQEIISAAGKELSEAFKQKTSEILSHAHAALDQPLPTTLKKQ
EEKEILKSQLDSLLGLYHLLDWFAVDESNEVDPEFSARLTGIKLE
MEPSLSFYNKARNYATKKPYSVEKFKLNFQMPTLASGWDVNKEKN
NGAILFVKNGLYYLGIMPKQKGRYKALSFEPTEKTSEGFDKMYYD
YFPDAAKMIPKCSTQLKAVTAHFQTHTTPILLSNNFIEPLEITKE
IYDLNNPEKEPKKFQTAYAKKTGDQKGYREALCKWIDFTRDFLSK
YTKITSIDLSSLRPSSQYKDLGEYYAELNPLLYHISFQRIAEKEI
MDAVETGKLYLFQIYNKDFAKGHHGKPNLHTLYWTGLFSPENLAK
TSIKLNGQAELFYRPKSRMKRMAHRLGEKMLNKKLKDQKTPIPDT
LYQELYDYVNHRLSHDLSDEARALLPNVITKEVSHEIIKDRRFTS
DKFFFHVPITLNYQAANSPSKFNQRVNAYLKEHPETPIIGIDRGE
RNLIYITVIDSTGKILEQRSLNTIQQFDYQKKLDNREKERVAARQ
AWSVVGTIKDLKQGYLSQVIHEIVDLMIHYQAVVVLENLNFGFKS
KRTGIAEKAVYQQFEKMLIDKLNCLVLKDYPAEKVGGVLNPYQLT
DQFTSFAKMGTQSGFLFYVPAPYTSKIDPLTGFVDPFVWKTIKNH
ESRKHFLEGFDFLHYDVKTGDFILHFKMNRNLSFQRGLPGFMPAW
DIVFEKNETQFDAKGTPFIAGKRIVPVIENHRFTGRYRDLYPANE
LIALLEEKGIVERDGSNILPKLLENDDSHAIDTMVALIRSVLQMR
NSNAATGEDYINSPVRDLNGVCFDSRFQNPEWPMDADANGAYHIA
LKGQLLLNHLKESKDLKLQNGISNQDWLAYIQELRNPKKKRKVKL
AAALEHHHHHH
Exemplary AsCpf1 wild-type amino acid sequence
SEQ ID NO: 66
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKILLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQMPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFFFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RN

Additional suitable nucleases and nuclease variants will be apparent to the skilled artisan based on the present disclosure in view of the knowledge in the art. Exemplary suitable nucleases may include, but are not limited to those provided in Table 5.

TABLE 5
Exemplary Suitable CRISPR/Cas Nucleases

| | Length | | |
|---|---|---|---|
| Nuclease | (A.A.) | PAM | Reference |
| SpCas9 | 1368 | NGG | Cong et al., Science 2013; 339(6121): 819-23 |
| SaCas9 | 1053 | NNGRRT | Ran et al., Nature 2015; 520(7546): 186-91. |
| (KKH) | 1067 | NNNRRT | Kleinstiver et al., Nat Biotechnol. 2015; |
| SaCas9 | | | 33(12): 1293-1298 |
| AsCpf1 | 1353 | TTTV | Zetsche et al., Nat Biotechnol. 2017; 35(1): 31-34. |
| (AsCas12a) | | | |
| LbCpf1 | 1274 | TTTV | Zetsche et al., Cell 2015; 163(3): 759-71. |
| (LbCas12a) | | | |
| CasX | 980 | TTC | Burstein et al., Nature 2017; 542(7640): 237-241. |
| CasY | 1200 | TA | Burstein et al., Nature 2017; 542(7640): 237-241. |
| Cas12h1 | 870 | RTR | Yan et al., Science 2019; 363(6422): 88-91. |
| Cas12i1 | 1093 | TTN | Yan et al., Science 2019; 363(6422): 88-91. |
| Cas12i2 | 1054 | TTN | Yan et al., Science 2019; 363(6422): 88-91. |
| Cas12c1 | unknown | TG | Yan et al., Science 2019; 363(6422): 88-91. |
| Cas12c2 | unknown | TN | Yan et al., Science 2019; 363(6422): 88-91. |
| eSpCas9 | 1423 | NGG | Chen et al., Nature 2017; 550(7676): 407-410. |
| Cas9-HF1 | 1367 | NGG | Chen et al., Nature 2017; 550(7676): 407-410. |
| HypaCas9 | 1404 | NGG | Chen et al., Nature 2017; 550(7676): 407-410. |
| dCas9-Fokl | 1623 | NGG | U.S. Pat. No. 9,322,037 |
| Sniper-Cas9 | 1389 | NGG | Lee et al., Nat Commun. 2018; 9(1): 3048. |
| xCas9 | 1786 | NGG, NG, | Hu et al., Nature. 2018 Apr 5; 556(7699): 57-63. |
| | | GAA, GAT | |
| AaCas12b | 1129 | TTN | Teng et al., Cell Discov. 2018; 4: 63. |
| evoCas9 | 1423 | NGG | Casini et al., Nat Biotechnol. 2018; 36(3): 265-271. |
| SpCas9-NG | 1423 | NG | Nishimasu et al., Science 2018; |
| | | | 361(6408): 1259-1262. |
| VRQR | 1368 | NGA | Li et al., The CRISPR Journal, 2018; 01: 01 |
| VRER | 1372 | NGCG | Kleinstiver et al., Nature 2016; 529(7587): 490-5. |
| NmeCas9 | 1082 | NNNNGATT | Amrani et al., Genome Biol. 2018; 19(1): 214. |
| CjCas9 | 984 | NNNNRYAC | Kim et al., Nat Commun. 2017; 8: 14500. |
| BhCas12b | 1108 | ATTN | Strecker et al., Nat Commun. 2019; 10(1): 212. |
| BhCas12b V4 | 1108 | ATTN | Pausch et al., Science 2020; 369(6501): 333-337. |

Guide RNA (gRNA) Molecules

Guide RNAs (gRNAs) of the present disclosure may be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al., Molecular Cell 2014; 56(2):333-339 (“Briner”), and in PCT Publication No. WO2016/073990A1.

In bacteria and archaea, type II CRISPR systems generally comprise an CRISPR/Cas nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g., GAAA) “tetraloop” or “linker” sequence bridging complementary regions of the crRNA (at its 3′ end) and the tracrRNA (at its 5′ end). See Mali et al., Science 2013; 339(6121):823-826 (“Mali”); Jiang et al., Nat Biotechnol. 2013; 31(3):233-239 (“Jiang”); and Jinek et al., Science 2012; 337(6096):816-821 (“Jinek 2012”).

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol. 2013; 31(9):827-832, (“Hsu”)), “complementarity regions” (PCT Publication No. WO2016/073990A1), “spacers” (Briner) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, as discussed below) include a plurality of domains that may influence the formation or activity of gRNA/Cas9 complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes. See Nishimasu 2014 and 2015. It should be noted that the first and/or second complementarity domains may contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. See Nishimasu 2015. A first stem-loop one near the 3′ portion of the second complementarity domain is referred to variously as the “proximal domain,” (PCT Publication No. WO2016/073990A1) “stem loop 1” (Nishimasu 2014 and 2015) and the “nexus” (Briner). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner.

While the foregoing description has focused on gRNAs for use with Cas9, it should be appreciated that other CRISPR/Cas nucleases have been (or may in the future be) discovered or invented which utilize gRNAs that differ in some ways from those described to this point. For instance, Cpf1 (“CRISPR from Prevotella and Franciscella 1”) which is also called Cas12a is a CRISPR/Cas nuclease that does not require a tracrRNA to function (see Zetsche et al., Cell 2015; 163:759-771 (“Zetsche I”)). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Those of skill in the art will appreciate, however, that although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple CRISPR/Cas nucleases. For this reason, unless otherwise specified, the term gRNA should be understood to encompass any suitable gRNA that can be used with any CRISPR/Cas nuclease, and not only those gRNAs that are compatible with a particular species of Cas9 or Cpf1. By way of illustration, the term gRNA can, in certain embodiments, include a gRNA for use with any CRISPR/Cas nuclease occurring in a Class 2 CRISPR system, such as a type II or type V or CRISPR system, or an CRISPR/Cas nuclease derived or adapted therefrom.

In some embodiments a method or system of the present disclosure may use more than one gRNA. In some embodiments, two or more gRNAs may be used to create two or more double strand breaks in the genome of a cell. In some embodiments, a multiplexed editing strategy may be used that targets two or more essential genes at the same time with two or more knock-in cassettes. In some such embodiments, the two or more knock-in cassettes may comprise different exogenous cargo sequences, e.g., different knock-in cassettes may encode different gene products of interest and thus the edited cells will express a plurality of gene products of interest from different knock-in cassettes targeted to different loci.

In some embodiments using more than one gRNA, a double-strand break may be caused by a dual-gRNA paired “nickase” strategy. In some embodiments for selecting gRNAs, including the determination for which gRNAs can be used for the dual-gRNA paired “nickase” strategy, gRNA pairs should be oriented on the DNA such that PAMs are facing out and cutting with the D10A Cas9 nickase will result in 5′ overhangs.

In some embodiments, a method or system of the present disclosure may use a prime editing gRNA (pegRNA) in conjunction with a prime editor (PE). As is well known in the art, a pegRNA is substantially larger than standard gRNAs, e.g., in some embodiments longer than 50, 100, 150 or 250 nucleotides, e.g., as described in Anzalone et al., Nature 2019; 576:149-157, the entire contents of which are incorporated herein by reference. The pegRNA is a gRNA with a primer binding sequence (PBS) and a donor template containing the desired RNA sequence added at one of the termini, e.g., the 3′ end. The PE:pegRNA complex binds to the target DNA, and the nickase domain of the prime editor nicks only one strand, generating a flap. The PBS, located on the pegRNA, binds to the DNA flap and the edited RNA sequence is reverse transcribed using the reverse transcriptase domain of the prime editor. The edited strand is incorporated into the DNA at the end of the nicked flap, and the target DNA is repaired with the new reverse transcribed DNA. The original DNA segment is removed by a cellular endonuclease. This leaves one strand edited, and one strand unedited. In the newest PE systems, e.g., PE3 and PE3b, the unedited strand can be corrected to match the newly edited strand by using an additional standard gRNA. In this case, the unedited strand is nicked by a nickase and the newly edited strand is used as a template to repair the nick, thus completing the edit.

gRNA Design

Methods for selection and validation of target sequences as well as off-target analyses have been described previously, e.g., in Mali; Hsu; Fu et al., Nat Biotechnol 2014; 32(3):279-84, Heigwer et al., Nat methods 2014; 11(2):122-3; Bae et al., Bioinformatics 2014; 30(10):1473-5; and Xiao et al. Bioinformatics 2014; 30(8):1180-1182. As a non-limiting example, gRNA design may involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in PCT Publication No. WO2016/073990A1.

For example, methods for selection and validation of target sequences as well as off-target analyses can be performed using cas-offinder (Bae et al., Bioinformatics 2014; 30:1473-5). Cas-offinder is a tool that can quickly identify all sequences in a genome that have up to a specified number of mismatches to a guide sequence.

As another example, methods for scoring how likely a given sequence is to be an off-target (e.g., once candidate target sequences are identified) can be performed. An exemplary score includes a Cutting Frequency Determination (CFD) score, as described by Doench et al., Nat Biotechnol. 2016; 34:184-91.

gRNA Modifications

In certain embodiments, gRNAs as used herein may be modified or unmodified gRNAs. In certain embodiments, a gRNA may include one or more modifications. In certain embodiments, the one or more modifications may include a phosphorothioate linkage modification, a phosphorodithioate (PS2) linkage modification, a 2′-O-methyl modification, or combinations thereof. In certain embodiments, the one or more modifications may be at the 5′ end of the gRNA, at the 3′ end of the gRNA, or combinations thereof.

In certain embodiments, a gRNA modification may comprise one or more phosphorodithioate (PS2) linkage modifications.

In some embodiments, a gRNA used herein includes one or more or a stretch of deoxyribonucleic acid (DNA) bases, also referred to herein as a “DNA extension.” In some embodiments, a gRNA used herein includes a DNA extension at the 5′ end of the gRNA, the 3′ end of the gRNA, or a combination thereof. In certain embodiments, the DNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 DNA bases long. For example, in certain embodiments, the DNA extension may be 1, 2, 3, 4, 5, 10, 15, 20, or 25 DNA bases long. In certain embodiments, the DNA extension may include one or more DNA bases selected from adenine (A), guanine (G), cytosine (C), or thymine (T). In certain embodiments, the DNA extension includes the same DNA bases. For example, the DNA extension may include a stretch of adenine (A) bases. In certain embodiments, the DNA extension may include a stretch of thymine (T) bases. In certain embodiments, the DNA extension includes a combination of different DNA bases.

Exemplary suitable 5′ extensions for Cpf1 guide RNAs are provided in Table 6 below:

TABLE 6
Exemplary Cpf1 gRNA 5′ Extensions

| SEQ ID | | 5′ |
|---|---|---|
| NO: | 5′ extension sequence | modification |
| N/A | rCrUrUrUrU | +5 RNA |
| 67 | rArArGrArCrCrUrUrUrU | +10 RNA |
| 68 | rArUrGrUrGrUrUrUrUrUrGrUrCrArArArArGrArCr | +25 RNA |
| | CrUrUrUrU | |
| 69 | rArGrGrCrCrArGrCrUrUrGrCrCrGrGrUrUrUrUrUr | +60 RNA |
| | UrArGrUrCrGrUrGrCrUrGrCrUrUrCrArUrGrUrGr | |
| | UrUrUrUrUrGrUrCrArArArArGrArCrCrUrUrUrU | |
| N/A | CTTTT | +5 DNA |
| 70 | AAGACCTTTT | +10 DNA |
| 71 | ATGTGTTTTTGTCAAAAGACCTTTT | +25 DNA |
| 72 | AGGCCAGCTTGCCGGTTTTTTAGTCGTGCTGC | +60 DNA |
| | TTCATGTGTTTTTGTCAAAAGACCTTTT | |
| 73 | TTTTTGTCAAAAGACCTTTT | +20 DNA |
| 74 | GCTTCATGTGTTTTTGTCAAAAGACCTTTT | +30 DNA |
| 75 | GCCGGTTTTTTAGTCGTGCTGCTTCATGTGTT | +50 DNA |
| | TTTGTCAAAAGACCTTTT | |
| 76 | TAGTCGTGCTGCTTCATGTGTTTTTGTCAAAA | +40 DNA |
| | GACCTTTT | |
| 77 | C*C*GAAGTTTTCTTCGGTTTT | +20 DNA + |
| | | 2× PS |
| 78 | T*T*TTTCCGAAGTTTTCTTCGGTTTT | +25 DNA + |
| | | 2× PS |
| 79 | A*A*CGCTTTTTCCGAAGTTTTCTTCGGTTTT | +30 DNA + |
| | | 2× PS |
| 80 | G*C*GTTGTTTTCAACGCTTTTTCCGAAGTTTT | +41 DNA + |
| | CTTCGGTTTT | 2× PS |
| 81 | G*G*CTTCTTTTGAAGCCTTTTTGCGTTGTTTT | +62 DNA + |
| | CAACGCTTTTTCCGAAGTTTTCTTCGGTTTT | 2× PS |
| 82 | A*T*GTGTTTTTGTCAAAAGACCTTTT | +25 DNA + |
| | | 2× PS |
| 83 | AAAAAAAAAAAAAAAAAAAAAAAAA | +25 A |
| 84 | TTTTTTTTTTTTTTTTTTTTTTTTT | +25 T |
| 85 | mA*mU*rGrUrGrUrUrUrUrUrGrUrCrArArArArGr | +25 RNA |
| | ArCrCrUrUrUrU | + 2× PS |
| 86 | mA*mA*rArArArArArArArArArArArArArArArAr | PolyA RNA |
| | ArArArArArArA | + 2× PS |
| 87 | mU*mU*rUrUrUrUrUrUrUrUrUrUrUrUrUrUrUrUr | PolyU RNA |
| | UrUrUrUrUrUrU | + 2× PS |

In certain embodiments, a gRNA used herein includes a DNA extension as well as a chemical modification, e.g., one or more phosphorothioate linkage modifications, one or more phosphorodithioate (PS2) linkage modifications, one or more 2′-O-methyl modifications, or one or more additional suitable chemical gRNA modification disclosed herein, or combinations thereof. In certain embodiments, the one or more modifications may be at the 5′ end of the gRNA, at the 3′ end of the gRNA, or combinations thereof.

Without wishing to be bound by theory, it is contemplated that any DNA extension may be used with any gRNA disclosed herein, so long as it does not hybridize to the target nucleic acid being targeted by the gRNA and it also exhibits an increase in editing at the target nucleic acid site relative to a gRNA which does not include such a DNA extension.

In some embodiments, a gRNA used herein includes one or more or a stretch of ribonucleic acid (RNA) bases, also referred to herein as an “RNA extension.” In some embodiments, a gRNA used herein includes an RNA extension at the 5′ end of the gRNA, the 3′ end of the gRNA, or a combination thereof. In certain embodiments, the RNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 RNA bases long. For example, in certain embodiments, the RNA extension may be 1, 2, 3, 4, 5, 10, 15, 20, or 25 RNA bases long. In certain embodiments, the RNA extension may include one or more RNA bases selected from adenine (rA), guanine (rG), cytosine (rC), or uracil (rU), in which the “r” represents RNA, 2′-hydroxy. In certain embodiments, the RNA extension includes the same RNA bases. For example, the RNA extension may include a stretch of adenine (rA) bases. In certain embodiments, the RNA extension includes a combination of different RNA bases. In certain embodiments, a gRNA used herein includes an RNA extension as well as one or more phosphorothioate linkage modifications, one or more phosphorodithioate (PS2) linkage modifications, one or more 2′-O-methyl modifications, one or more additional suitable gRNA modification, e.g., chemical modification, disclosed herein, or combinations thereof. In certain embodiments, the one or more modifications may be at the 5′ end of the gRNA, at the 3′ end of the gRNA, or combinations thereof. In certain embodiments, a gRNA including an RNA extension may comprise a sequence set forth herein.

It is contemplated that gRNAs used herein may also include an RNA extension and a DNA extension. In certain embodiments, the RNA extension and DNA extension may both be at the 5′ end of the gRNA, the 3′ end of the gRNA, or a combination thereof. In certain embodiments, the RNA extension is at the 5′ end of the gRNA and the DNA extension is at the 3′ end of the gRNA. In certain embodiments, the RNA extension is at the 3′ end of the gRNA and the DNA extension is at the 5′ end of the gRNA.

In some embodiments, a gRNA which includes a modification, e.g., a DNA extension at the 5′ end and/or a chemical modification as disclosed herein, is complexed with a CRISPR/Cas nuclease, e.g., an AsCpf1 nuclease, to form an RNP, which is then employed to edit a target cell, e.g., a pluripotent stem cell or a progeny thereof.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

As one example, the 5′ end of a gRNA can include a eukaryotic mRNA cap structure or cap analog (e.g., a G(5′)ppp(5′)G cap analog, a m7G(5′)ppp(5′)G cap analog, or a 3′-0-Me-m7G(5′)ppp(5′)G anti reverse cap analog (ARCA)), as shown below:

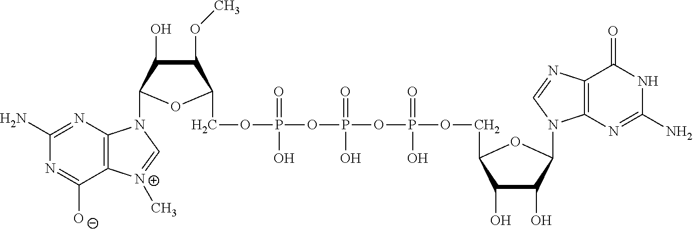

The cap or cap analog can be included during either chemical or enzymatic synthesis of the gRNA.

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another common modification involves the addition, at the 3′ end of a gRNA, of a plurality (e.g., 1-10, 10-20, or 25-200) of adenine (A) residues referred to as a polyA tract. The polyA tract can be added to a gRNA during chemical or enzymatic synthesis, using a polyadenosine polymerase (e.g., E. coli Poly(A)Polymerase).

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

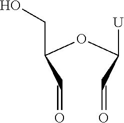

wherein “U” can be an unmodified or modified uridine.

The 3′ terminal U ribose can be modified with a 2′3′ cyclic phosphate as shown below:

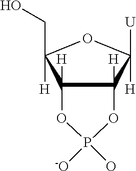

wherein “U” can be an unmodified or modified uridine.

Guide RNAs can contain 3′ nucleotides that can be stabilized against degradation, e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into a gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂, alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, including without limitation methylene, propylene, ether, or amino bridges; O-amino (wherein amino can be, e.g., NH₂, alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂, alkylamino, dialkylamino, heterocyclyl, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into a gRNA. In certain embodiments, 0- and N-alkylated nucleotides, e.g., N6-methyl adenosine, can be incorporated into a gRNA. In certain embodiments, one or more or all of the nucleotides in a gRNA are deoxynucleotides.

Guide RNAs can also include one or more cross-links between complementary regions of the crRNA (at its 3′ end) and the tracrRNA (at its 5′ end) (e.g., within a “tetraloop” structure and/or positioned in any stem loop structure occurring within a gRNA). A variety of linkers are suitable for use. For example, guide RNAs can include common linking moieties including, without limitation, polyvinylether, polyethylene, polypropylene, polyethylene glycol (PEG), polyvinyl alcohol (PVA), polyglycolide (PGA), polylactide (PLA), polycaprolactone (PCL), and copolymers thereof.

In some embodiments, a bifunctional cross-linker is used to link a 5′ end of a first gRNA fragment and a 3′ end of a second gRNA fragment, and the 3′ or 5′ ends of the gRNA fragments to be linked are modified with functional groups that react with the reactive groups of the cross-linker. In general, these modifications comprise one or more of amine, sulfhydryl, carboxyl, hydroxyl, alkene (e.g., a terminal alkene), azide and/or another suitable functional group. Multifunctional (e.g. bifunctional) cross-linkers are also generally known in the art, and may be either heterofunctional or homofunctional, and may include any suitable functional group, including without limitation isothiocyanate, isocyanate, acyl azide, an NHS ester, sulfonyl chloride, tosyl ester, tresyl ester, aldehyde, amine, epoxide, carbonate (e.g., Bis(p-nitrophenyl) carbonate), aryl halide, alkyl halide, imido ester, carboxylate, alkyl phosphate, anhydride, fluorophenyl ester, HOBt ester, hydroxymethyl phosphine, O-methylisourea, DSC, NHS carbamate, glutaraldehyde, activated double bond, cyclic hemiacetal, NHS carbonate, imidazole carbamate, acyl imidazole, methylpyridinium ether, azlactone, cyanate ester, cyclic imidocarbonate, chlorotriazine, dehydroazepine, 6-sulfo-cytosine derivatives, maleimide, aziridine, TNB thiol, Ellman's reagent, peroxide, vinylsulfone, phenylthioester, diazoalkanes, diazoacetyl, epoxide, diazonium, benzophenone, anthraquinone, diazo derivatives, diazirine derivatives, psoralen derivatives, alkene, phenyl boronic acid, etc. In some embodiments, a first gRNA fragment comprises a first reactive group and the second gRNA fragment comprises a second reactive group. For example, the first and second reactive groups can each comprise an amine moiety, which are crosslinked with a carbonate-containing bifunctional crosslinking reagent to form a urea linkage. In other instances, (a) the first reactive group comprises a bromoacetyl moiety and the second reactive group comprises a sulfhydryl moiety, or (b) the first reactive group comprises a sulfhydryl moiety and the second reactive group comprises a bromoacetyl moiety, which are crosslinked by reacting the bromoacetyl moiety with the sulfhydryl moiety to form a bromoacetyl-thiol linkage. These and other cross-linking chemistries are known in the art, and are summarized in the literature, including by Greg T. Hermanson, Bioconjugate Techniques, 3rd Ed. 2013, published by Academic Press.

Additional suitable gRNA modifications will be apparent to those of ordinary skill in the art based on the present disclosure. Suitable gRNA modifications include, for example, those described in PCT Publication No. WO2019070762A1 entitled “MODIFIED CPF1 GUIDE RNA;” in PCT Publication No. WO2016089433A1 entitled “GUIDE RNA WITH CHEMICAL MODIFICATIONS;” in PCT Publication No. WO2016164356A1 entitled “CHEMICALLY MODIFIED GUIDE RNAS FOR CRISPR/CAS-MEDIATED GENE REGULATION;” and in PCT Publication No. WO2017053729A1 entitled “NUCLEASE-MEDIATED GENOME EDITING OF PRIMARY CELLS AND ENRICHMENT THEREOF;” the entire contents of each of which are incorporated herein by reference.

Exemplary gRNAs

Non-limiting examples of guide RNAs suitable for certain embodiments embraced by the present disclosure are provided herein, for example, in the Tables below. Those of ordinary skill in the art will be able to envision suitable guide RNA sequences for a specific nuclease, e.g., a Cas9 or Cpf1 nuclease, from the disclosure of the targeting domain sequence, either as a DNA or RNA sequence. For example, a guide RNA comprising a targeting sequence consisting of RNA nucleotides would include the RNA sequence corresponding to the targeting domain sequence provided as a DNA sequence, and this contain uracil instead of thymidine nucleotides. For example, a guide RNA comprising a targeting domain sequence consisting of RNA nucleotides, and described by the DNA sequence TCTGCAGAAATGTTCCCCGT (SEQ ID NO: 88) would have a targeting domain of the corresponding RNA sequence UCUGCAGAAAUGUUCCCCGU (SEQ ID NO: 89). As will be apparent to the skilled artisan, such a targeting sequence would be linked to a suitable guide RNA scaffold, e.g., a crRNA scaffold sequence or a chimeric crRNA/tracrRNA scaffold sequence. Suitable gRNA scaffold sequences are known to those of ordinary skill in the art. For AsCpf1, for example, a suitable scaffold sequence comprises the sequence UAAUUUCUACUCUUGUAGAU (SEQ ID NO: 90), added to the 5′-terminus of the targeting domain. In the example above, this would result in a Cpf1 guide RNA of the sequence UAAUUUCUACUCUUGUAGAUUCUGCAGAAAUGUUCCCCGU (SEQ ID NO: 91). Those of skill in the art would further understand how to modify such a guide RNA, e.g., by adding a DNA extension (e.g., in the example above, adding a 25-mer DNA extension as described herein would result, for example, in a guide RNA of the sequence ATGTGTTTTTGTCAAAAGACCTTTTrUrArArUrUrUrCrUrArCrUrCrUrUrGrUrArGrArUrUr CrUrGrCrArGrArArArUrGrUrUrCrCrCrCrGrU (SEQ ID NO: 92)). It will be understood that the exemplary targeting sequences provided herein are not limiting, and additional suitable sequences, e.g., variants of the specific sequences disclosed herein, will be apparent to the skilled artisan based on the present disclosure in view of the general knowledge in the art.

Exemplary gRNAs are listed in the following Tables 8 to 13:

TABLE 8
AsCas12 guide RNAs

| SEQ | | Target Domain Sequence |
|---|---|---|
| ID NO | Gene | (DNA) |
| 225 | EIF4G2 | AGGCTTTGGCTGGTTCTTTAG |
| 226 | EIF4G2 | GCTGGTTCTTTAGTCAGCTTC |
| 227 | EIF4G2 | GTCAGCTTCTTCCTCTGATTC |
| 228 | EIF4G2 | TAACCAGGTTAGCCACTGATT |
| 229 | EIF4G2 | ACAAAAGACTTACCTGGAACA |
| 230 | EIF4G2 | CCGGAAACTCTTGGGTTATAT |
| 231 | EIF4G2 | CAAGCCAAGAAAGCTTCTTCT |
| 232 | EIF4G2 | CATGTCATAGAAGTGCACAAA |
| 233 | EIF4G2 | GGAAGTTGCTGTTATAGCAGT |
| 234 | EIF4G2 | TGCATTACTGGCTTGAAAGAT |
| 235 | EIF4G2 | CTGCTCTAACTGTTCTTTGGA |
| 236 | EIF4G2 | GAAGGAGCAGAGGATGAATCT |
| 237 | EIF4G2 | ATCGCTGGGGGGGTTTACTTC |
| 238 | EIF4G2 | CTTCACTAGAAATGTACTGTA |
| 239 | EIF4G2 | TCTACATGAAGTTTGGGAGAG |
| 240 | EIF4G2 | GGAGAGATGTTATCTTTAATC |
| 241 | EIF4G2 | TATATGGTTTGAGGGGATGGA |
| 242 | EIF4G2 | AGGGGATGGATCCAACTTTAT |
| 243 | EIF4G2 | TAGGTGAATCAGTGGCTAACC |
| 244 | EIF4G2 | CAAATCTTAATTTATAGGTGA |
| 245 | EIF4G2 | ATTTACAAATCTTAATTTATA |
| 246 | EIF4G2 | CGGGAAAAGGCAAGGCTTTGT |
| 247 | EIF4G2 | TTGGCTTGGAAAGAAGATATA |
| 248 | EIF4G2 | TGCACTTCTATGACATGGAAA |
| 249 | EIF4G2 | AGGCATGTTACTTCGCTTTTT |
| 250 | EIF4G2 | TTCATGATCACGTTGATCTAC |
| 251 | EIF4G2 | AAGCCAGTAATGCAGAAATTT |
| 252 | EIF4G2 | TAGTGAAGTAAACCCCCCCAG |
| 253 | EIF4G2 | TGTCCAGCTTCTTACAGTACA |
| 254 | EIF4G2 | TGAACATCITAATGACTAGGT |
| 255 | SKP1 | AAGACCTTACCTTTTTTAATA |
| 256 | SKPI | CAATGAACTTACCTTCCAACA |
| 257 | SKP1 | AGCAGGGCAGAATAAAAACCA |
| 258 | SKPI | TTCATAATTTCAGCAGGGCAG |
| 259 | SKP1 | CTTTGTTCATAATTTCAGCAG |
| 260 | SKPI | CAGGCTGCAAACTACTTAGAC |
| 261 | SKP1 | TTGTTGTAGGTCATTCAGTGG |
| 262 | SKP1 | TTAGATTTGGGAATGGATGAT |
| 263 | SKPI | TTCTGGTTTTCTTAGATTTGG |
| 264 | SKPI | GATGCCTTCAATTAAGTTGCA |
| 265 | SKP1 | ATGTCCTTTTTTTTTAGATGC |
| 266 | RPS3 | AAGCTTTATGCTGAAAAGGTG |
| 267 | RPS3 | AAGGGCCTGCTATGGTGTGCT |
| 268 | RPS3 | AAGGAAGCAAGGGATATCCTG |
| 269 | RPS3 | AGCATAAAGCTTTAAAGGAAG |
| 270 | RPS3 | CCAGACACCACAACCTCGCAG |
| 271 | RPS3 | CCAAGCACTCTCAGCTGCTCA |
| 272 | RPS19 | TTCTTCCATCTTTTCCCACAG |
| 273 | RPS19 | CCACAGGTGGCAGCTGCCAAC |
| 274 | RPS19 | TCTGACGTCCCCCATAGATCT |
| 275 | HMGB1 | AGCCCTCTTACCTTCCACCTC |
| 276 | HMGB1 | TGTTCATTTATTGAAGTTCTA |
| 277 | HMGB1 | GTTCGGCCTTCTTCCTCTTCT |
| 278 | HMGB1 | TAGACCATGTCTGCTAAAGAG |
| 279 | HMGB1 | GAAAAATAACTAAACATGGGC |
| 280 | RPL7 | CCCCAAATAGAACCTACCAAG |
| 281 | RPL7 | ACTTCAGGTACCCCAATCTGA |
| 282 | RPL7 | CTTTTTCACTTCAGGTACCCC |
| 283 | RPL7 | TGTTTGCTTTTTCACTTCAGG |
| 284 | RPL7 | ACCACAGTATCAATGGAGTGA |
| 285 | RPL7 | TGGTCCGTTTTCACCACAGTA |
| 286 | RPLP0 | AGGTCAAGGCCTTCTTGGCTG |
| 287 | RPLP0 | ACCACTTCCCCCCTCCTTTCA |
| 288 | G6PD | CTCACCTGCCATAAATATAGG |
| 289 | G6PD | CAGTATGAGGGCACCTACAAG |
| 290 | G6PD | ACCCCACTGCTGCACCAGATT |
| 291 | G6PD | CGCCACGTAGGGGTGCCCTTC |
| 292 | RPL4 | GCTTGTAGTGCCGCTGCTGCA |
| 293 | RPL4 | CCGTGGTGCTCGAAGGGCTCT |
| 294 | RPL4 | TTGCAGCACAAGCTCCGGGTG |
| 295 | RPL4 | TGCCTAATTTGTTGCAGCACA |
| 296 | RPL4 | TAGCAAGAAGATCCATCGCAG |
| 297 | RPL4 | AGTCTTCCCATGCACAAGATG |
| 298 | RPL4 | CCTTTCAGTCTTCCCATGCAC |
| 299 | EEF1G | TCCCCAGCTGAGTCCAGATTG |
| 300 | EEF1G | TTCCTCTTAGTACCTTTGTGT |
| 301 | RPL31 | GATGGCTCCCGCAAAGAAGGG |
| 302 | RPL31 | AATCGTAGGGGCTTCAAGAAG |
| 303 | RPL31 | TTAGGAATGTGCCATACCGAA |
| 304 | RPL31 | CAGATCTACAGACAGICAATG |
| 305 | RPL31 | GCACCTTATTCCTTTGGCCCA |
| 306 | RPL31 | TGGGATGGAGAACTTACTTTT |
| 307 | RPL31 | ATCTGACGATCAGCGATTAGT |
| 308 | ITM2B | ACTGTCTTTTTCATATTTTAG |
| 309 | ITM2B | ATATTTTAGGACCCAGATGAT |
| 310 | ITM2B | GGACCCAGATGATGTGGTACC |
| 311 | ITM2B | GACTAGCATTTATGCTTGCAG |
| 312 | ITM2B | TGCTTGCAGGTGTTATTCTAG |
| 313 | ITM2B | TGAATGTAGGCTGGAACCTAT |
| 314 | ITM2B | CCTCAGTCCTATCTGATTCAT |
| 315 | ITM2B | TTTATTTATCGACTGTGTCAT |
| 316 | ITM2B | TTTATCGACTGTGTCATGACA |
| 317 | ITM2B | TCGACTGTGTCATGACAAGGA |
| 318 | ITM2B | CCTCTCCAACAGGTATTCAGA |
| 319 | ITM2B | GCAATTCGGCATTTTGAAAAC |
| 320 | ITM2B | AAAACAAATTTGCCGTGGAAA |
| 321 | ITM2B | CCGTGGAAACTTTAATTTGTT |
| 322 | ITM2B | GCCAACTGGTACCACATCATC |
| 323 | ITM2B | TACAAGTATGCTCCTCCTAGA |
| 324 | ITM2B | CACTTACTTGAAGTGCAAAAT |
| 325 | ITM2B | AATGCGATCAGTAATAACCAT |
| 326 | ITM2B | CTTGTCATGACACAGTCGATA |
| 327 | ITM2B | TAAGTTTCCTTGTCATGACAC |
| 328 | ITM2B | TCTGCGTTGCAGTTTGTAAGT |
| 329 | ITM2B | ATAGTTTCTCTGCGTTGCAGT |
| 330 | ITM2B | AAAAGTATTACCTTTAATAGT |
| 331 | ITM2B | ATATTTAAAAAGTATTACCTT |
| 332 | ITM2B | AAAATGCCGAATTGCGAAACA |
| 333 | ITM2B | TTTTCAAAATGCCGAATTGCG |
| 334 | ITM2B | CACGGCAAATTTGTTTTCAAA |
| 335 | ITM2B | TTGACTGTTCAAGAACAAATT |
| 336 | RPL23A | CTTTTCTCCCAGCTCCTGCCC |
| 337 | RPL23A | TCCCAGCTCCTGCCCCTCCTA |
| 338 | RPL23A | CCTCTCCCAGGCTTGACCACT |
| 339 | RPL23A | TTTTTCAGATTGGGATCATCT |
| 340 | RPL23A | TAGGAAGGAAACTTACTTTGT |
| 341 | RPL27A | GTCTGGGCTGCCAACATGGTA |
| 342 | RPL27A | TATTCCTGCAGGCAAGCACCG |
| 343 | RPL27A | TCTGTTCTTCTAGGGCTACTA |
| 344 | PCBP2 | CCCTCTGACTCTCTCCCAGTC |
| 345 | PCBP2 | CTCCTTTTGTAGGCCTATACC |
| 346 | PCBP2 | TAGGCCTATACCATTCAAGGA |
| 347 | PCBP2 | CTCCTTGCAGTTGACCAAGCT |
| 348 | PCBP2 | ACTTGTATCTTAACAGGCATT |
| 349 | PCBP2 | GCAGGTTTGGATGCATCTGCT |
| 350 | PCBP2 | TTTCTCCCTTAAGTTGATTGG |
| 351 | PCBP2 | TCCCTTAAGTTGATTGGCTGC |
| 352 | PCBP2 | TGTGTTACAGGCTTTCCTCGG |
| 353 | PCBP2 | AGCATGAGCCTGAGGGCTTAC |
| 354 | PCBP2 | TTACCTGACCACCTGCAAAGA |
| 355 | PCBP2 | ATCATTACCCCAATAGCCTTT |
| 356 | HSPA8 | TCTTCCTCAGACTGCTGAGAA |
| 357 | HSPA8 | CTAGGCCGTTTGAGCAAGGAA |
| 358 | HSPA8 | TTTCCTAGGCCGTTTGAGCAA |
| 359 | HNRNPK | ATCAGCACTGAAACCAACCTG |
| 360 | HNRNPK | AGTTGGCTGGATCTATTATTG |
| 361 | HNRNPK | AAAAATCTTTTCAGTTGGCTG |
| 362 | HNRNPK | AATCAGATTATTCCTATGCAG |
| 363 | HNRNPK | TGTTTTTAGGGTGGCTCCGGA |
| 364 | HNRNPK | TTTCTGTTTTTAGGGTGGCTC |
| 365 | HNRNPK | TCTCTAACAGGTTGGTTTCAG |
| 366 | RPL5 | TCTCTTACTATAGATTGCTTA |
| 367 | RPL5 | CATTGGTTTCTTGAATAGCTT |
| 368 | RPL5 | TTGAATAGCTTCTCAATAGGT |
| 369 | UBL5 | TGTAGCTCCAGCTAGGATGAT |
| 370 | UBL5 | CCTTAACTGCTCTGCGCCCAG |
| 371 | UBL5 | TTAGGTACACGATTTTTAAGG |
| 372 | UBL5 | CTTCAGATGAAATCCACGATG |
| 373 | CST3 | GACAAGGTCATTGTGCCCTGC |
| 374 | CST3 | AGATGTGGCTGGTCATGGAAG |
| 375 | CST3 | TTGTACTCGCCGACGGCAAAG |
| 376 | CST3 | CAGATCTACGCTGTGCCTTGG |
| 377 | CST3 | ACAGAAAGCATTCTGCTCTTT |
| 378 | CST3 | CTTTCACAGAAAGCATTCTGC |
| 379 | CST3 | ACATGTGTAGATCGTAGCTGG |
| 380 | CST3 | CCGTCGGCGAGTACAACAAAG |
| 38 | RPS29 | TCACCAAGAGCGAGAACCCTG |
| 382 | RPS29 | TTACAGTCGTGTCTGTTCAAA |
| 383 | RPS10 | TACTGTACATGCTTCCTTTTT |
| 384 | RPS10 | CAAATGACATTATCTGAGAGC |
| 385 | RPS10 | CTCACGTGGCACAGCACTCCG |
| 386 | RPS10 | TGTGGGAACCATACCTTTAGG |
| 387 | RPS10 | TAAAAAGGAAGCATGTACAGT |
| 388 | RPSIO | TCCTATGGCAGGTCCTCATAG |
| 389 | RPS10 | TAGCTGGTGCCGACAAGAAAG |
| 390 | RPS10 | ACTTTCTAGCTGGTGCCGACA |
| 391 | RPS10 | CATAGGTCTGGAGGGTGAGCG |
| 392 | RPS10 | ATTTACATAGGTCTGGAGGGT |
| 393 | RPS10 | TGCCTTACAGTCTCTCAAGTC |
| 394 | RPL6 | TTACCAGTCACAAGTAATAAG |
| 395 | RPL6 | GAAATATGAGATTACGGAGCA |
| 396 | RPL6 | TTTAGAAATATGAGATTACGG |
| 397 | RPL6 | TCTTTATTTAGAAATATGAGA |
| 398 | RPL6 | ATTTTCTCTTTATTTAGAAAT |
| 399 | RPL6 | CCCCTTAGGACCTCTGGTCCT |
| 400 | RPL6 | ACTTACAGAGGGTGGTTTTCC |
| 401 | RPL6 | TTTTTAACTTACAGAGGGTGG |
| 402 | RPLP2 | TGTAGGTATTGGCAAGCTTGC |
| 403 | ARF1 | ACACTGGCTGCCCGGCAGGCC |
| 404 | RPL15 | TGTGTAGGTTACGTTATATAT |
| 405 | RPL15 | CTATTCTAGGAGCGAGCTGGA |
| 406 | RPL15 | CCTCTGCAACGGACTGAAGGC |
| 407 | FAU | CTGGCCGGTCACCTCGAAGGT |
| 408 | FAU | CCTGTAGGCTCATGTAGCCTC |
| 409 | FAU | CTCAGTCGCCAATATGCAGCT |
| 410 | FAU | TTTACTCAGTCGCCAATATGC |
| 411 | RPL36 | CCCCCTAGCGTCTGACCAAAC |
| 412 | RPL36 | CCCCGTACGAGCGGCGCGCCA |
| 413 | NACA | CTAGTATACCTCTTCCTCTTC |
| 414 | NACA | CTCACCTTGGCTTCCCCAAAA |
| 415 | NACA | AAATCTTACCTTCCGTGCCTT |
| 416 | NACA | TCTGTTACAGGAATTAACAAT |
| 417 | NACA | CCTCTCATCTCTCAGGTCGAT |
| 418 | NACA | TACCCTGTAGATCGAAGATTT |
| 419 | NACA | GGCTATGTCCAAACTGGGTCT |
| 420 | NACA | TCTTCTTTAGGCTATGTCCAA |
| 421 | NACA | TCTTCTTAGCTGGCGGCAGCA |
| 422 | PRDX1 | GACATCAGGCTTGATGGTATC |
| 423 | PRDX1 | CCATGCTAGATGACAGAAGTG |
| 424 | PRDX1 | TTAAATTCTTCTGCCCTATCA |
| 425 | PRDX1 | TCTTGCAGTGTGCCCAGCTGG |
| 426 | PRDX1 | TCATTGATGATAAGGGTATTC |
| 427 | PRDX1 | CCAGGGGCCTTTTTATCATTG |
| 428 | PRDX1 | ATCTCTTTTCCCAGGGGCCTT |
| 429 | PRDX1 | CTTTCATCTCTTTTCCCAGGG |
| 430 | PRDX1 | GTATCAGACCCGAAGCGCACC |
| 431 | PRDX1 | CCATAGGGTCAATACACCTAA |
| 432 | PRDX1 | CCTTTTGCCATAGGGTCAATA |
| 433 | PRDX1 | AGTGATAGGGCAGAAGAATTT |
| 434 | PRDX1 | CCCTCTTGACTTCACCTTTGT |
| 435 | PRDX1 | CCCCCAGGAAAATATGTTGTG |
| 436 | ALDOA | CCTTCTCGGTCACATACTGGC |
| 437 | NCL | GCCCAGTCCAAGGTAACTTTA |
| 438 | NCL | TTTCCATCAATTTCACCGTCT |
| 439 | NCL | CATCAATTTCACCGTCTTCCA |
| 440 | NCL | ACCGTCTTCCATGGCCTCCTT |
| 441 | NCL | GCATCCTCCTCACTGTTGAAG |
| 442 | NCL | GAGGACCCAGTTTCCCGGTCA |
| 443 | NCL | CCGGTCAGTAACTATCCTTGC |
| 444 | NCL | ATGTCTCTTCAGTGGTATCCT |
| 445 | NCL | ACAAACAGAGTTTTGGATGGC |
| 446 | NCL | GTGGCAGAGGCCGGGGAGGCT |
| 447 | NCL | GAGGACGAGGTGGTGGTAGAG |
| 448 | NCL | TAGACTTCAACAGTGAGGAGG |
| 449 | NCL | GTTTTGTAGACTTCAACAGTG |
| 450 | NCL | GTGTTCTAGGTTTGGTTTTGT |
| 451 | NCL | ATTTGGTGTTCTAGGTTTGGT |
| 452 | NCL | ACGGCTCCGTTCGGGCAAGGA |
| 453 | NCL | TCAAAGGCCTGTCTGAGGATA |
| 454 | NCL | CTTCCCAGAGCCATCCAAAAC |
| 455 | BTF3 | TAGATGAAAGAAACAATCATG |
| 456 | BTF3 | CTCTTCTCCCTGACTTTAGGG |
| 457 | BTF3 | GGGAACTGCTCGCAGAAAGAA |
| 458 | BTF3 | TTTTCTTAATAGGTGAATATG |
| 459 | BTF3 | TTAATAGGTGAATATGTTTAC |
| 460 | BTF3 | CATTTTCCTTTCATAGCTGTG |
| 461 | BTF3 | CTTTCATAGCTGTGGATGGAA |
| 462 | BTF3 | ATAGCTGTGGATGGAAAAGCA |
| 463 | BTF3 | TACTCTTTTCCTTTTCCTAGA |
| 464 | BTF3 | CTTTTCCTAGATCTTGTGGAG |
| 465 | BTF3 | CTAGATCTTGTGGAGAATTTT |
| 466 | BTF3 | ATACTTGCCTCTTCAATACCA |
| 467 | E2F4 | GGGGCTATCATTGTAGTGAGT |
| 468 | E2F4 | AGCCCATCAAGGCAGACCCCA |
| 469 | E2F4 | AGTTTTGGAACTCCCCAAAGA |
| 470 | E2F4 | GAACTCCCCAAAGAGCTGTCA |
| 471 | E2F4 | CCCCTCTGCTTCGTCTTTCTC |
| 472 | E2F4 | TCCACCCCCGGGAGACCACGA |
| 473 | E2F4 | ATGTGCCTGTTCTCAACCTCT |
| 474 | E2F4 | TGACAGCTCTTTGGGGAGTTC |
| 475 | KIF11 | ACTAAGCTTAATTGCTTTCTG |
| 476 | KIF11 | TGGAACAGGATCTGAAACTGG |
| 477 | KIF11 | TACCCATCAACACTGGTAAGA |
| 478 | KIF11 | TTCTTTTAGGATGTGGATGTA |
| 479 | KIF11 | GGATGTGGATGTAGAAGAGGC |
| 480 | KIF11 | CCGCCTTAAATCCACAGCATA |
| 481 | KIF11 | ATTAAGTTCTAGATTTTGTGC |
| 482 | KIF11 | TGGTTTCATTAAGTTCTAGAT |
| 483 | KIF11 | AGATCCTGTTCCAGAAAGCAA |
| 484 | KIF11 | AAGTACCTGTTGGGATATCCA |
| 485 | KIF11 | TCTTTTAAAGTACCTGTTGGG |
| 486 | KIF11 | AGCTGATCAAGGAGATGTTCA |
| 487 | KIF11 | CTTTTCAGCTGATCAAGGAGA |
| 488 | KIF11 | GCATCATTAACAGCTCAGGCT |
| 489 | KIF11 | TGAACAGTTTAGCATCATTAA |
| 490 | KIF11 | TTGTTTTCTGAACAGITTAGC |
| 491 | KIF11 | CCGGAATTGTCTCTTCTTTGT |
| 492 | KIF11 | AATTTACCGGAATTGTCTCTT |
| 493 | KIF11 | TCTTTTCCATGTGATTTTTTA |
| 494 | KIF11 | TTTGTCTTTTCCATGTGATTT |
| 495 | KIF11 | GACCTCTCCAGTGTGTTAATG |
| 496 | KIF11 | TTCCACTTTAGACCTCTCCAG |
| 497 | KIF11 | TAACCAAGTGCTCTGTAGTTT |
| 498 | RPL13 | TCTTCTAGGTCTATAAGAAGG |
| 499 | RPL13 | AGTAAGTGTTCACTTACGTTC |
| 500 | PFDN5 | CCTTAATTCTTGCTTCTCAGA |
| 50 | PFDN5 | AGCTGAGCAATGGACGTGGAC |
| 502 | PTMA | AAGGACTTAAAGGAGAAGAAG |
| 503 | PTMA | TGTCGAGGAGAATGAGGAAAA |
| 504 | PTMA | ATTCTCTCCAGGTGAGGAAGA |
| 505 | PTMA | TCTGCTTAGGATGACGATGTC |
| 506 | RPL11 | GCATCCGGAGAAATGAAAAGA |
| 507 | RPL11 | TCCACAGGTGCGGGAGTATGA |
| 508 | RPL11 | AGCATCGCAGACAAGAAGCGC |
| 509 | RPL11 | AGTATGATGGGATCATCCTTC |
| 510 | RPL11 | CGGATGCGAAGTTCCCGCATG |
| 511 | RPL11 | TCCGGATGCCAAAGGATCTGA |
| 512 | RPL11 | ATTTCTCCGGATGCCAAAGGA |
| 513 | RPL11 | GACCCTTCTCCAAGATTTCTT |
| 514 | RPL11 | TTAACTCATACTCCCGCACCT |
| 515 | RPL11 | CCTTCTGCTGGAACCAGCGCA |
| 516 | COX7C | TCTTTTTTTCCAACAGAATTT |
| 517 | COX7C | CAACAGAATTTGCCATTTTCA |
| 518 | RPL8 | TTGAGGCCCTCAGCACTAGTT |
| 519 | RPL8 | CGGCCAGCAGGGGCATCTCTG |
| 520 | RPL8 | TGGGTTACTTACATTCATGGC |
| 521 | RPL8 | TCTGCCTGCAGCCTGTGGAGC |
| 522 | RPL10 | TTCTCCCTACCTAGCCCTGGA |
| 523 | RPL10 | CATTGCTCCTTAGATCCACAT |
| 524 | RPL32 | CCTCCCCAAAAGGAAGAGTTC |
| 525 | TBP | CTGCGGTAATCATGAGGATAA |
| 526 | TBP | AGTTCTGGGAAAATGGTGTGC |
| 527 | TBP | CTTTCCCTAGTGAAGAACAGT |
| 528 | TBP | CCTAGTGAAGAACAGTCCAGA |
| 529 | TBP | CAGCTAAGTTCTTGGACTTCA |
| 530 | TBP | CTATAAGGTTAGAAGGCCTTG |
| 531 | TBP | CAATTTTCCTTCTAGTTATGA |
| 532 | TBP | CTTCTAGTTATGAGCCAGAGT |
| 533 | TBP | CTGGTTTAATCTACAGAATGA |
| 534 | TBP | ATCTACAGAATGATCAAACCC |
| 535 | TBP | TTTCTGGAAAAGTTGTATTAA |
| 536 | TBP | TGGAAAAGTTGTATTAACAGG |
| 537 | TBP | GGTCAAGTTTACAACCAAGAT |
| 538 | TBP | GGGCACGAAGTGCAATGGTCT |
| 539 | TBP | CCAGAACTGAAAATCAGTGCC |
| 540 | TBP | TTACGGCTACCTCTTGGCTCC |
| 541 | TBP | TTGCTGCCAGTCTGGACTGTT |
| 542 | TBP | AGACTTACCTACTAAATTGTT |
| 543 | TBP | ATCATTCTGTAGATTAAACCA |
| 544 | TBP | CAGAAACAAAAATAAGGAGAA |
| 545 | TBP | AAATGCTTCATAAATTTCTGC |
| 546 | CD63 | CTCAGCCAGCCCCCAATCTTC |
| 547 | CD63 | TCCCAATCTGTGTAGTTAGCA |
| 548 | CD63 | GGGTAATTCTCCATCTGCTGC |
| 549 | CD63 | GGAATTGTCTTTGCCTGCTGC |
| 550 | CD63 | CTTCTAGGTTTTGGGAATTGT |
| 551 | CD63 | TGCCTGCCACCTTCAGGGCTG |
| 552 | CD63 | AACGAGAAGGCGATCCATAAG |
| 553 | CD63 | AGTGCTGTGGGGCTGCTAACT |
| 554 | CD63 | TTCCCTCCCCCAGTTTAAGTG |
| 555 | CD63 | ATAACAACTTCCGGCAGCAGA |
| 556 | CD63 | TGTCTCTTATCATGTTGGTGG |
| 557 | CD63 | CCATCTTTCTGTCTCTTATCA |
| 558 | CD63 | CTCCTGCAGTTTGCCATCTTT |
| 559 | CD63 | TGGGCTGCTGCGGGGCCTGCA |
| 560 | RPS24 | TGTTTTCAGAACGACACCGTA |
| 561 | RPS24 | AGAACGACACCGTAACTATCC |
| 562 | RPS24 | GGTCATTGATGTCCTTCACCC |
| 563 | RPS24 | TCATTCAGCATGGCCTGTATG |
| 564 | RPS24 | CCTCTTCTTCTGGATTACAGA |
| 565 | RPS24 | TAGTGCGGATAGTTACGGTGT |
| 566 | RPS24 | CTTAATGAACTATACCTTTTT |
| 567 | RPS23 | GGGCTGTGCCCAAATGAGCTT |
| 568 | RPS23 | TTCCAGGAAAATGATGAAGTT |
| 569 | RPS23 | TACCCAATGACGGTTGCTTGA |
| 570 | RPS23 | AGAGGAGTTGAAGCCAAACAG |
| 571 | RPS23 | TATTTCAGAGGAGTTGAAGCC |
| 572 | RPS23 | GGCAAGTGTCGTGGACTTCGT |
| 573 | RPS23 | ATTTTTAGGCAAGTGTCGTGG |
| 574 | EEF2 | TCCAGGAAGTTGTCCAGGGCA |
| 575 | EEF2 | AGGCCCTTGCGCTTGCGGGTC |
| 576 | EEF2 | ACCACTGGCAGATCCTGCCCG |
| 577 | EEF2 | TGGTCAAGGCCTATCTGCCCG |
| 578 | EEF2 | AACAGGAAGCGGGGCCACGTG |
| 579 | EEF2 | CCTTCTGGCAGTGTCCAGAGC |
| 580 | EEF2 | TTTCCCTTCTGGCAGTGTCCA |
| 581 | CALR | CTTCTCCCTTCTGCAGGGTGA |
| 582 | CALR | GCGTGCTGGGCCTGGACCTCT |
| 583 | CALR | ACAACTTCCTCATCACCAACG |
| 584 | CALR | GCAACGAGACGTGGGGCGTAA |
| 585 | CALR | TGGGTGGATCCAAGTGCCCTT |
| 586 | CALR | CTCCAAGTCTCACCTGCCAGA |
| 587 | CALR | TTACGCCCCACGTCTCGTTGC |
| 588 | CALR | TCCTTCATTTGTTTCTCTGCT |
| 589 | CALR | TTGTCTTCTTCCTCCTCCTTA |
| 590 | CALR | TCCTCATCATCCTCCTTGTCC |
| 591 | RPL36AL | TATGCCCAGGGAAGGAGGCGC |
| 592 | SRP14 | AGGCTTATTCAAACCTCCTTA |
| 593 | SRP14 | AGGTGAGCTCCAAGGAAGTGA |
| 594 | SRP14 | CTTCTTTTTCAGGTGAGCTCC |
| 595 | SRP14 | CTTCAGATGACGGTCGAACCA |
| 596 | SRP14 | CAGAAGTGCCGGACGTCGGGC |
| 597 | SRP14 | CAGTTCCTGACGGAGCTGACC |
| 598 | GABARAP | TTTCGGATCTTCTCGCCCTCA |
| 599 | GABARAP | GGATCTTCTCGCCCTCAGAGC |
| 600 | GABARAP | TCTACATTGCCTACAGTGACG |
| 601 | GABARAP | ATCCCAGGAACACCATGAAGA |
| 602 | GABARAP | TGCTTTCATCCCAGGAACACC |
| 603 | GABARAP | TCAACAATGTCATTCCACCCA |
| 604 | GABARAP | TTTGTCAACAATGTCATTCCA |
| 605 | GABARAP | CAGTTGGTCAGTTCTACTTCT |
| 606 | GABARAP | TTGCATCTTGTATCTTTTGCA |
| 607 | GABARAP | TCAGGTGATAGTAGAAAAGGC |
| 608 | GABARAP | ATCTCTTTATCAGGTGATAGT |
| 609 | RPSA | ATAATCTGCCACTCTTGGCAG |
| 610 | RPSA | TAACCCAGATTGAAAAAGAAG |
| 611 | RPSA | GTATTCTCTTAACAGAAGACT |
| 612 | RPSA | GAGAAGCTTACCTCTTCAGGA |
| 613 | SET | AATTATTTATTACAGTATTTT |
| 614 | SET | TTACAGTATTTTGATGAAAAT |
| 615 | SET | GGATTTGACGAAACGTTCGAG |
| 616 | SET | ACGAAACGTTCGAGTCAAACG |
| 617 | SET | AGGTTCCCGATATGGATGATG |
| 618 | SET | TTTCAGGAGGATGAAGGAGAA |
| 619 | SET | AGGAGGATGAAGGAGAAGATG |
| 620 | SET | TTTTACCTCTCCTTCCTCCCC |
| 621 | SET | GCCAAATTTTCTTTTACCTCT |
| 622 | GAPDH | CAGACCACAGTCCATGCCATC |
| 623 | GAPDH | ATCTTCTAGGTATGACAACGA |
| 624 | RPLP1 | TTTGTTGTAGGAGGATAAGAT |
| 625 | RPLP1 | TTGTAGGAGGATAAGATCAAT |
| 626 | RPLP1 | TAGCTGAGGAGAAGAAAGTGG |
| 627 | RPLP1 | CCACCATCACCTTACCTTTGC |
| 628 | RPLP1 | CTACCTGGAGCAGCAGCAGTG |
| 629 | CFL1 | CTCTTAAGGGGCGCAGACTCG |
| 630 | CFL1 | TAGGGATCAAGCATGAATTGC |
| 631 | CFL1 | TTCTTTATAGGGATCAAGCAT |
| 632 | CFL1 | TGTCCAGGGCCCCCGAGTCTG |
| 633 | RPS15 | CTCTTGGTCTCCCGCAGCCCG |
| 634 | TPT1 | CATTATTTATTTTAACCCACT |
| 635 | TPT1 | TTTTAACCCACTTCCTTGTAC |
| 636 | TPT1 | ACCCACTTCCTTGTACTTACA |
| 637 | TPT1 | CCTGGTAGTTTTTGAAATTAG |
| 638 | TPT1 | GAAATGGAAAAATGTGTAAGT |
| 639 | TPT1 | CTTCCCAAGTTCTTTATTGGT |
| 640 | TPT1 | TTTGCTTCCCAAGTTCTTTAT |
| 641 | TPT1 | GAATCAAAGGGAAACTTGAAG |
| 642 | TPT1 | TTAATGCAGATGGTCAGTAGG |
| 643 | RPL23 | CTACCTTTCATCTCGCCTTTA |
| 644 | RPL23 | TTGTTCACTATGACTCCTGCA |
| 645 | RPL23 | CTCACCCTTTTTTCTGAGCTC |
| 646 | RPL23 | ATGCAGGTTCTGCCATTACAG |
| 647 | RPL23 | TTTTTTTAATGCAGGTTCTGC |
| 648 | RPL23 | TTCTCTCAGTACATCCAGCAG |
| 649 | RPL34 | ACTTTCTAGGTCCCGAACCCC |
| 650 | RPL34 | TAGGTCCCGAACCCCTGGTAA |
| 651 | RPL34 | TTATGCAGGTTCGTGCTGTAA |
| 652 | RPL34 | GTATTTTCCTTTCTAGGATCA |
| 653 | RPL34 | CTTTCTAGGATCAAGCGTGCT |
| 654 | RPL34 | TAGGATCAAGCGTGCTTTCCT |
| 655 | RPL34 | AGAAATACTTACAGCCTAGTT |
| 656 | RPL34 | ACTTACCTGTCACGAACACAT |
| 657 | RPL34 | AGCATTTAACTTACCTGTCAC |
| 658 | COX4I1 | TCTTTCAGAATGTTGGCTACC |
| 659 | COX4I1 | AGAATGTTGGCTACCAGGGTA |
| 660 | COX4I1 | CACCTCTGTGTGTGTACGAGC |
| 661 | COX4I1 | TTCAATATGTTTTTCAGAAAG |
| 662 | COX4I1 | AGAAAGTGTTGTGAAGAGCGA |
| 663 | COX4I1 | GCTCCCAGCTTATATGGATCG |
| 664 | COX4I1 | CTGAGATGAACAGGGGCTCGA |
| 665 | COX4I1 | ACCGCGCTCGTTATCATGTGG |
| 666 | COX4I1 | ACAAAGAGTGGGGGCCAAGC |
| 667 | COX4I1 | TCAAAGCTTTGCGGGAGGGGG |
| 668 | COX4I1 | GTAGTCCCACTTGGAGGCTAA |
| 669 | RPL27 | TCCTTGCTCTCTGCAGAAATG |
| 670 | RPL27 | GAACATTGATGATGGCACCTC |
| 671 | RPL27 | TCCCCAGGTACTCTGTGGATA |
| 672 | RPL27 | CCTTCTAGATACAAGACAGGC |
| 673 | RPL27 | CGTCCGGAGTAGCGTCCAGCC |
| 674 | RPL27 | TCTTTGATCTCTTGGCGATCT |
| 675 | RPL27 | ACAAAAGATTTTATCTTTGAT |
| 676 | EDF1 | GAGGCTTTGTGTTCATTTCGC |
| 677 | EDF1 | TGTTCATTTCGCCCTAGGCCC |
| 678 | EDF1 | GCCCTAGGCCCCTTCTCGATG |
| 679 | EDF1 | CAATGTCCTTTCCCCGGAGCT |
| 680 | EDF1 | CCAAGCACCTGGTTATTGGGT |
| 681 | EDF1 | TTGGAAGTCTCCACATCTTCT |
| 682 | EDF1 | GCCTGGGCGGCCGTAGGGCCC |
| 683 | EDF1 | AGGCCTCAAGCTCCGGGGAAA |
| 684 | EDF1 | GAAAATCAATGAGAAGCCACA |
| 685 | EDF1 | CCTCACACCGACTCCAGGGGC |
| 686 | EDF1 | TAGGCTATCTTAGCGGCACAG |
| 687 | EDF1 | TAATTTTCTAGGCTATCTTAG |
| 688 | TMEM59 | AAAGAAAAATGCTTAAATTTC |
| 689 | TMEM59 | AGAATGAGCAAGATTCACTTT |
| 690 | TMEM59 | TAGGTAGAGGCCCTGCTTCTT |
| 691 | TMEM59 | GATCTAACAACCACAAGAGAA |
| 692 | TMEM59 | GCTTTTGTTCATTCATAAACT |
| 693 | TMEM59 | TTCATTCATAAACTCCAAGTC |
| 694 | TMEM59 | CCTCAGAGGGAACATACTGCT |
| 695 | TMEM59 | TCCATCTTCAAGAAAATTCCT |
| 696 | TMEM59 | CTTAGAGATGATTCTCTCAAA |
| 697 | TMEM59 | TAGGCTCCTGCTCCAAATGTG |
| 698 | TMEM59 | CGTCATCGGCTTGAAGATAAA |
| 699 | TMEM59 | TGAATGAACAAAAGCTAAACA |
| 700 | TMEM59 | CAGAAGCTGAGTATCTATGGT |
| 701 | TMEM59 | TTTTGCAGAAGCTGAGTATCT |
| 702 | TMEM59 | TTGTGCAACTGTTGCTACAGC |
| 703 | TMEM59 | GATTTGTTGTGCAACTGTTGC |
| 704 | TMEM59 | ACTACAACTCTTGTCCTCTCG |
| 705 | TMEM59 | CAGTAACTCTGGGTGGATTTT |
| 706 | TMEM59 | TTGAAGATGGAGAAAGTGATG |
| 707 | TMEM59 | AGCAGATCTGCAAATGAGAAA |
| 708 | TMEM59 | AGAGAATCATCTCTAAGCAAA |
| 709 | TMEM59 | GAGCAGGAGCCTACAAATTTG |
| 710 | TMEM59 | GTCTAAGCCAGAAATCCAGTA |
| 711 | TMEM59 | ATTATTATTTTAGTCTAAGCC |
| 712 | TMEM59 | TCTTCAAGCCGATGACGGAAA |
| 713 | DYNLL1 | TCTTTTCCAGGAATTTGACAA |
| 714 | DYNLL1 | CAGGAATTTGACAAGAAGTAC |
| 715 | DYNLL1 | ATGTGTCACATAACTACCGAA |
| 716 | NME2 | TTTCTTAGGAACATCATTCAT |
| 717 | NME2 | TTAGGAACATCATTCATGGCA |
| 718 | TMBIM6 | GCTGATGGCAACACCTCATAG |
| 719 | TMBIM6 | TGTTTTCTAGGAGTTGGCCTG |
| 720 | TMBIM6 | TAGGAGTTGGCCTGGGCCCTG |
| 721 | TMBIM6 | TATTGCTGTCAACCCCAGGTA |
| 722 | TMBIM6 | TAACAGCATCCTTCCCACTGC |
| 723 | TMBIM6 | ATGGGCACGGCAATGATCTTT |
| 724 | TMBIM6 | CCTGCTTCACCCTCAGTGCAC |
| 725 | TMBIM6 | CTGTGTCTTATAGGTATCTTG |
| 726 | TMBIM6 | TCTTCCCTGGGGAATGTTTTC |
| 727 | TMBIM6 | GATCCATTTGGCTTTTCCAGG |
| 728 | TMBIM6 | TTAGGCAAACCTGTATGTGGG |
| 729 | TMBIM6 | ATACTCAACTCATTATTGAAA |
| 730 | TMBIM6 | AGGCACTGCATTGATCTCTTC |
| 731 | TMBIM6 | ATTACTGTCTTCAGAAAACTC |
| 732 | TMBIM6 | TCCATTTCTAGGATAAGAAGA |
| 733 | TMBIM6 | TAGGATAAGAAGAAAGAGAAG |
| 734 | TMBIM6 | ATGGCTATGAGGTGTTGCCAT |
| 735 | TMBIM6 | TGTTCAGTTTCATGGCTATGA |
| 736 | TMBIM6 | CCAGTTCACACTTACCTCCCA |
| 737 | TMBIM6 | AATAATGAGTTGAGTATCAAA |
| 738 | TMBIM6 | TGAAGACAGTAATGAAATCTA |
| 739 | TMBIM6 | ATTCATGGCCAGGATCATCAT |
| 740 | TMBIM6 | GGTTGTAGGCTAACTAACCTT |
| 741 | RPS7 | TTTAGGAAATTGAAGTTGGTG |
| 742 | RPS7 | GGAAATTGAAGTTGGTGGTGG |
| 743 | RPS7 | CCTTACAGAGGAGAATTCTGC |
| 744 | RPS7 | AACTATTCTTTTAGCCGTACT |
| 745 | RPS7 | GCCGTACTCTGACAGCTGTGC |
| 746 | RPS7 | TTTTCTTGTAGGTTGAAACTT |
| 747 | RPS7 | TTGTAGGTTGAAACTTTTTCT |
| 748 | RPS7 | TGAAACTACTAAAATACTCAC |
| 749 | ACTB | CTTCCCAGGGCGTGATGGTGG |
| 750 | NPM1 | ATTTGTAGTGATGATGATGAT |
| 751 | NPM1 | TAATTGCAGTCTATACGAGAT |
| 752 | NPM1 | GAAATTCATTTCTTTTTCAGG |
| 753 | NPM1 | TTTTTCAGGGACAAGAATCCT |
| 754 | NPM1 | AGGGACAAGAATCCTTCAAGA |
| 755 | NPM1 | TCTTAATAGGGTGGTTCTCTT |
| 756 | NPM1 | CAGGCTATTCAAGATCTCTGG |
| 757 | NPM1 | TAAAATCATACTTACTCTTCA |
| 758 | NPM1 | CTCACTTTTTCTATACTTGCT |
| 759 | RPS6 | TTTTTCTTGGTACGCTGCTTC |
| 760 | RPS6 | GGGCCCAGGCGGCGAGGCACT |
| 761 | RPS6 | GGAGGCTAAGGAGAAGCGCCA |
| 762 | RPS6 | TTTAGGAGGCTAAGGAGAAGC |
| 763 | RPS6 | TTTTGTTTAGGAGGCTAAGGA |
| 764 | RPS6 | GGTAAGAAACCTAGGACCAAA |
| 765 | RPS6 | AATTTTTAGGTAAGAAACCTA |
| 766 | RPS6 | TTCTAAGGAGAGAAGGATATT |
| 767 | RPL12 | CTTAAAGGAACCATTAAAGAG |
| 768 | RPL12 | TTTACTTAAAGGAACCATTAA |
| 769 | RPL12 | CTCTTCTGCAGTTAAACACAG |
| 770 | RPL12 | CTGTTTCCTCTTCTGCAGTTA |
| 771 | RPL12 | TAGTCTCCAAAAAAAGTTGGT |
| 772 | RPL12 | TTTCTAGTCTCCAAAAAAAGT |
| 773 | RPL12 | CCCCAGTATACCTGAGGTGCA |
| 774 | CAPNS1 | AACCTGTTACCCACAGACCCT |
| 775 | CAPNS1 | GCATTGACACATGTCGCAGCA |
| 776 | CAPNS1 | AGGAATTCAAGTACTTGTGGA |
| 777 | CAPNS1 | CAGTAGTGAACTCCCAGGTGC |
| 778 | CAPNS1 | ATGTTGTTCCACAAGTACTTG |
| 779 | CAPNS1 | TACACACCTGCCACCTTTTGA |
| 780 | CAPNSI | AGAGGTTTCTACACACCTGCC |
| 781 | CAPNS1 | ATCTGAGTAGCGTCGGATGAT |
| 782 | CAPNS1 | TCAAGAGATTTGAAGGCACCT |
| 783 | CAPNS1 | TCCAGTGCCATCTTTGTCAAG |
| 784 | RPL3 | CAGGGTGGCTTTGTCCACTAT |
| 785 | RPS13 | TTTATTAGCTTACCTTTCTGT |
| 786 | RPS13 | TTAGCTTACCTTTCTGTTCCT |
| 787 | RPS13 | AGTGAATCATCTACAGCCTCT |
| 788 | RPS13 | TTTTTCAGTGAATCATCTACA |
| 789 | RPS13 | CCCTTTTTTCTTTTTCAGTGA |
| 790 | RPS13 | AGGTGTAATCCTGAGAGATTC |
| 791 | RPS13 | TATTCCATAACAGTGGTTGAA |
| 792 | RPS21 | TCCACAGCTCCGCTAGCAATC |
| 793 | RPS21 | TGACCCTTCTTCTCTTTCTAG |
| 794 | RPS21 | TAGGTTGACAAGGICACAGGC |
| 795 | RPS21 | TTAAGGGTGAGTCAGATGATT |
| 796 | RPS21 | CCCTGGTTCTAGGAACTTTTG |
| 797 | RPS21 | AGACGATGCCATCGGCCTTGG |
| 798 | SERF2 | ATTTTCTTTCCTTAGGCGGTA |
| 799 | SERF2 | TTTCCTTAGGCGGTAACCAGC |
| 800 | SERF2 | CTTAGGCGGTAACCAGCGTGA |
| 801 | SERF2 | TGCTGCCGCCCGCAAGCAGAG |
| 802 | SERF2 | ATATTCTTCTGGCGGGCGAGC |
| 803 | SERF2 | CCTTAACCGAGTCGCTCTGCT |
| 804 | SERF2 | CCTCCCCTCCCTGGGGCTACC |
| 805 | RPL7A | TTTCCCCTCCTGCCTTTTAGG |
| 806 | RPL7A | CCCTCCTGCCTTTTAGGGAAG |
| 807 | RPL7A | GGGAAGACAAAGGCGCTTTGG |
| 808 | RPL7A | TCTTTTCAGATCCGCCGTCAC |
| 809 | RPL7A | AGATCCGCCGTCACTGGGGTG |
| 810 | RPL7A | GGGCCAGGCTGTGTACTTACG |
| 811 | RPL7A | GTGTAAAGCTGCCTCTTACCT |
| 812 | HNRNPA2B1 | TAAATTACCTCCACCATATGG |
| 813 | HNRNPA2B1 | CACTCTTCATTGGACCGTAGT |
| 814 | HNRNPA2B1 | CAAAATCATTGTAATTTCCAC |
| 815 | HNRNPA2B1 | TTACCTCCTCCATAGTTGTCA |
| 816 | HNRNPA2B1 | CACCGCCACCACGTGAATCCC |
| 817 | HNRNPA2B1 | GTGGTAGCAGGAACATGGGGG |
| 818 | HNRNPA2B1 | GAAATTATAACCAGCAACCTT |
| 819 | HNRNPA2B1 | ATAGGAAATTATGGAAGTGGA |
| 820 | HNRNPA2B1 | GAGGTAGCCCCGGTTATGGAG |
| 821 | HNRNPA2B1 | TAATAGGTGGCAATTTTGGAG |
| 822 | HNRNPA2B1 | GGGATGGCTATAATGGGTATG |
| 823 | HNRNPA2B1 | GCCCCTAACAGATGGATATGG |
| 824 | HNRNPA2B1 | GGACCAGGACCAGGAAGTAAC |
| 825 | HNRNPA2B1 | GGGATTCACGTGGTGGCGGTG |
| 826 | HNRNPA2B1 | GCTTTGGGGATTCACGTGGTG |
| 827 | HNRNPA2B1 | TTGTAGGCAACTTTGGCTTTG |
| 828 | HNRNPA2B1 | TCTAGACAAGAAATGCAGGAA |
| 829 | RPL13A | TCTAACAGAAAAAGCGGATGG |
| 830 | RPL13A | GCATAGCTCACCTTGTCGTAG |
| 831 | ENO1 | AGCAGGAGGCAGTTGCAGGAC |
| 832 | ENO1 | TCCTTCCCAAGAATTGAAGAG |
| 833 | ENO1 | CCTTTCTCCTTCCCAAGAATT |
| 834 | ENO1 | TCCTAGATCAAGACTGGTGCC |
| 835 | ENO1 | TTTTCTCCTAGATCAAGACTG |
| 836 | ENO1 | CTTAGTGGTGTCTATCGAAGA |
| 837 | PPIA | CTATATGTTGACAGGGTGGTG |
| 838 | PPIA | AAGGTTGGATGGCAAGCATGT |
| 839 | CD81 | CCTGTGAGGTGGCCGCCGGCA |
| 840 | CD81 | ACCACCTCAGTGCTCAAGAAC |
| 841 | CD81 | TGTCCCTCGGGCAGCAACATC |
| 842 | RPL35 | TTGACAATGCGCCCCTCAGGC |
| 843 | RPL35 | TAGCCGAGTCGTCCGGAAATC |
| 844 | DAD1 | TTCTGTGGGTTGATCTGTATT |
| 845 | DAD1 | CCAGCACCATCCTGCACCTTG |
| 846 | DAD1 | TCTTTGCCAGCACCATCCTGC |
| 847 | DAD1 | CTGATTTTCTCTTTGCCAGCA |
| 848 | DAD1 | CAAGGCATCTCCCCAGAGCGA |
| 849 | DAD1 | CCTGAGAATACAGATCAACCC |
| 850 | DAD1 | CTTCTTGTGCAGTTTGCCTGA |
| 851 | DAD1 | TGTTTTGCTTCTTGTGCAGTT |
| 852 | DAD1 | TCTCGGGCTTCATCTCTTGTG |
| 853 | DAD1 | GCGGTTCTTAGAAGAGTACTT |
| 854 | UBA52 | TGAAGACCCTCACTGGCAAAA |
| 855 | UBA52 | CCAGTGAGGGTCTTCACAAAG |
| 856 | UBA52 | TGGGCAAGCTGGCGGAGAGAA |
| 857 | UBA52 | ACCTTCTTCTTGGGACGCAGG |
| 858 | RPL30 | TAGGTGAAAAGGTTTACTTTT |
| 859 | RPL30 | TGATTTAAAAAGCATACCTGG |
| 860 | RPL30 | AAAAGCATACCTGGATCAATG |
| 861 | RPL30 | GGTGACTCTGACATCATTAGA |
| 862 | RPL30 | TTTTTTAGGTGACTCTGACAT |
| 863 | RPL30 | TTTTTATTTTTTAGGTGACTC |
| 864 | RPL30 | GTTCCCAAAGGAAATCTGAAA |
| 865 | RPL30 | CCCATTTTGGTTCCCAAAGGA |
| 866 | RPL30 | TAGAAAAAGTCGCTGGAGTCG |
| 867 | RPL30 | CTTTGTAGAAAAAGTCGCTGG |
| 868 | RPL30 | ATGTTTGCTTTGTAGAAAAAG |
| 869 | RNASEK | CGCCTGCCGCCCCCGGATGGG |
| 870 | RNASEK | TCCCACCGCTTTCCGAGCCCG |
| 871 | RNASEK | CGAGCCCGCTTGCACCTCGGC |
| 872 | RNASEK | TGGCGTCGCTCCTGTGCTGTG |
| 873 | RPL38 | TGTTGCAGCCTCGGAAAATTG |
| 874 | RPL38 | TCTCTTTCCCTCTAGGTTTGG |
| 875 | RPL38 | CCTCTAGGTTTGGCAGTGAAG |
| 876 | RPL38 | GTCGGGCTGTGAGCAGGAAGT |
| 877 | MYL12B | TTCTTTCTATTGTCTTCCAGG |
| 878 | MYL12B | TATTGTCTTCCAGGCACCATT |
| 879 | MYL12B | GCTAAAGTTCTTTCAGTCATC |
| 880 | PFN1 | CCCATCAGCAGGACTAGCGCT |
| 881 | PFN1 | CTCCTCCTCCAGCGCTAGTCC |
| 882 | PFN1 | TCTTTCCTCCTCCTCCAGCGC |
| 883 | PFN1 | GCATGGATCTTCGTACCAAGA |
| 884 | RPS11 | TCCTCATAATCTGTAGACTGA |
| 885 | RPS11 | TCTTTCCTATCCTTTCAGGCT |
| 886 | RPS11 | CTATCCTTTCAGGCTATTGAG |
| 887 | RPS11 | AGGCTATTGAGGGCACCTACA |
| 888 | RPS11 | TTCTGAGGTTCCCCGCACCTC |

TABLE 9
Cas12b guide RNAs

| SEQ | | | SEQ | | |
|---|---|---|---|---|---|
| ID NO | Gene | Target Domain Sequence (DNA) | ID NO | Gene | Target Domain Sequence (DNA) |
| 889 | GAPDH | CCCAGCTCTCATACCATGAGTCC | 917 | E2F4 | CCAGAGTGCATGAGCTCGGAGCT |
| 890 | TBP | TATCCACAGTGAATCTTGGTTGT | 918 | E2F4 | TATCTACAACCTGGACGAGAGTG |
| 891 | TBP | CACTTCGTGCCCGAAACGCCGAA | 919 | E2F4 | CCTGGACTTCTGCACTGCCAGGG |
| 892 | TBP | TCTCTGACCATTGTAGCGGTTTG | 920 | E2F4 | CTGACAGCTCTTTGGGGAGTTCC |
| 893 | TBP | TAGCGGTTTGCTGCGGTAATCAT | 921 | G6PD | AGCTGGAGAAGCCCAAGCCCATC |
| 894 | TBP | TCAGTTCTGGGAAAATGGTGTGC | 922 | G6PD | TCACCCCACTGCTGCACCAGATT |
| 895 | TBP | AGAATATGGTGGGGAGCTGTGAT | 923 | KIF11 | ATGAAGATAAATTGATAGCACAA |
| 896 | TBP | TCCTTCTAGTTATGAGCCAGAGT | 924 | KIF11 | ATAGCACAAAATCTAGAACTTAA |
| 897 | TBP | CCTGGTTTAATCTACAGAATGAT | 925 | KIF11 | GTTTGACTAAGCTTAATTGCTTT |
| 898 | TBP | TTCTCCTTATTTTTGTTTCTGGA | 926 | KIF11 | CTTTCTGGAACAGGATCTGAAAC |
| 899 | TBP | TTGTTTCTGGAAAAGTTGTATTA | 927 | KIF11 | ATACCCATCAACACTGGTAAGAA |
| 900 | TBP | ATGAAGCATTTGAAAACATCTAC | 928 | KIF11 | TTCATCAATTGGCGGGGTTCCAT |
| 901 | TBP | TAAAGGGATTCAGGAAGACGACG | 929 | KIF11 | GCGGGGTTCCATTTTTCCAGGTA |
| 902 | TBP | GGCGTTTCGGGCACGAAGTGCAA | 930 | KIF11 | TCCCGCCTTAAATCCACAGCATA |
| 903 | TBP | TATTCGGCGTTTCGGGCACGAAG | 931 | KIF11 | ACACACTGGAGAGGTCTAAAGTG |
| 904 | TBP | AAATAGATCTAACCTTGGGATTA | 932 | KIF11 | CCTCTGCGAGCCCAGATCAACCT |
| 905 | TBP | TCCCAGAACTGAAAATCAGTGCC | 933 | KIF11 | AGTTCTAGATTTTGTGCTATCAA |
| 906 | TBP | CTTACGGCTACCTCTTGGCTCCT | 934 | KIF11 | TTATGGTTTCATTAAGTTCTAGA |
| 907 | TBP | TCTTGCTGCCAGTCTGGACTGTT | 935 | KIF11 | AGCTTAGTCAAACCAATTTTTAT |
| 908 | TBP | TGAATCTTGAAGTCCAAGAACTT | 936 | KIF11 | CTCTTTTAAAGTACCTGTTGGGA |
| 909 | TBP | TTGGTGGGTGAGCACAAGGCCTT | 937 | KIF11 | TATTTCTCTTTTAAAGTACCTGT |
| 910 | TBP | CAGACTTACCTACTAAATTGTTG | 938 | KIF11 | ACAGCTCAGGCTGTTTCCTTTTC |
| 911 | TBP | AACCAGGAAATAACTCTGGCTCA | 939 | KIF11 | TCTCTTCTTTGTTGTTTTCTGAA |
| 912 | TBP | TGTAGATTAAACCAGGAAATAAC | 940 | KIF11 | ACCGGAATTGTCTCTTCTTTGTT |
| 913 | TBP | TGGGTTTGATCATTCTGTAGATT | 941 | KIF11 | ATGAACAATCCACACCAGCATCT |
| 914 | TBP | CTGCTCTGACTTTAGCACCTAAG | 942 | KIF11 | AAGGTTGATCTGGGCTCGCAGAG |
| 915 | TBP | CGTCGTCTTCCTGAATCCCTTTA | 943 | KIF11 | CCAACCCCCAAGTGAATTAAAGG |
| 916 | E2F4 | TAGTGAGTGGCGGCCCTGGGACT | — | | |

TABLE 10
Cas12e guide RNAs

| SEQ | | | SEQ | | |
|---|---|---|---|---|---|
| ID NO | Gene | Target Domain Sequence (DNA) | ID NO | Gene | Target Domain Sequence (DNA) |
| 944 | GAPDH | TCTTCTAGGTATGACAACGAA | 993 | E2F4 | CTGGACTTCTGCACTGCCAGG |
| 945 | GAPDH | CCAGCTCTCATACCATGAGTC | 994 | E2F4 | GACAGCTCTTTGGGGAGTTCC |
| 946 | TBP | TGCCCGAAACGCCGAATATAA | 995 | E2F4 | GAGGACATCAACTCCTCCAGC |
| 947 | TBP | CTCTGACCATTGTAGCGGTTT | 996 | E2F4 | AGGGCCACCCACCTTCTGAGG |
| 948 | TBP | GTTCTGGGAAAATGGTGTGCA | 997 | E2F4 | CTCTCGTCCAGGTTGTAGATA |
| 949 | TBP | GGGAAAATGGTGTGCACAGGA | 998 | G6PD | CCCACTTGTAGGTGCCCTCAT |
| 950 | TBP | TTTCCCTAGTGAAGAACAGTC | 999 | G6PD | TCAGCTCGTCTGCCTCCGTGG |
| 951 | TBP | CTAGTGAAGAACAGTCCAGAC | 1000 | G6PD | TCACCTGCCATAAATATAGGG |
| 952 | TBP | AGCTAAGTTCTTGGACTTCAA | 1001 | G6PD | CCAGCTCAATCTGGTGCAGCA |
| 953 | TBP | TGGACTTCAAGATTCAGAATA | 1002 | G6PD | CTGTAGGGCACCTTGTATCTG |
| 954 | TBP | AGATTCAGAATATGGTGGGGA | 1003 | G6PD | TGGTCATCATCTTGGTGTACA |
| 955 | TBP | GAATATGGTGGGGAGCTGTGA | 1004 | G6PD | GGGCCTTGCCGCAGCGCAGGA |
| 956 | TBP | TATAAGGTTAGAAGGCCTTGT | 1005 | G6PD | AGTATGAGGGCACCTACAAGT |
| 957 | TBP | TTCTAGTTATGAGCCAGAGTT | 1006 | G6PD | CCCCACTGCTGCACCAGATTG |
| 958 | TBP | AGTTATGAGCCAGAGTTATTT | 1007 | G6PD | GCGGGAGCCAGATGCACTTCG |
| 959 | TBP | TGGTTTAATCTACAGAATGAT | 1008 | G6PD | ACCCCGAGGAGTCGGAGCTGG |
| 960 | TBP | CCTTATTTTTGTTTCTGGAAA | 1009 | G6PD | TCAACCCCGAGGAGTCGGAGC |
| 961 | TBP | GGAAAAGTTGTATTAACAGGT | 1010 | G6PD | ACCAGCAGTGCAAGCGCAACG |
| 962 | TBP | TAGGTGCTAAAGTCAGAGCAG | 1011 | G6PD | ATGATGTGGCCGGCGACATCT |
| 963 | TBP | AAAGGGATTCAGGAAGACGAC | 1012 | G6PD | TCCTGCGCTGCGGCAAGGCCC |
| 964 | TBP | GGCACGAAGTGCAATGGTCTT | 1013 | G6PD | GCCACGTAGGGGTGCCCTTCA |
| 965 | TBP | GCGTTTCGGGCACGAAGTGCA | 1014 | KIF11 | GGAACAGGATCTGAAACTGGA |
| 966 | TBP | TGGCTCTCTTATCCTCATGAT | 1015 | KIF11 | GAAAACAACAAAGAAGAGACA |
| 967 | TBP | CAGAACTGAAAATCAGTGCCG | 1016 | KIF11 | TCTTTTAGGATGTGGATGTAG |
| 968 | TBP | TACGGCTACCTCTTGGCTCCT | 1017 | KIF11 | TTTAGGATGTGGATGTAGAAG |
| 969 | TBP | TGCTGCCAGTCTGGACTGTTC | 1018 | KIF11 | GGGGCAGTATACTGAAGAACC |
| 970 | TBP | GTACAACTCTAGCATATTTTC | 1019 | KIF11 | TCAATTGGCGGGGTTCCATTT |
| 971 | TBP | GAATCTTGAAGTCCAAGAACT | 1020 | KIF11 | CGCCTTAAATCCACAGCATAA |
| 972 | TBP | CATCACAGCTCCCCACCATAT | 1021 | KIF11 | AGATTTTGTGCTATCAATTTA |
| 973 | TBP | AACCTTATAGGAAACTTCACA | 1022 | KIF11 | TTAAGTTCTAGATTTTGTGCT |
| 974 | TBP | GACTTACCTACTAAATTGTTG | 1023 | KIF11 | AGAAAGCAATTAAGCTTAGTC |
| 975 | TBP | GTAGATTAAACCAGGAAATAA | 1024 | KIF11 | GATCCTGTTCCAGAAAGCAAT |
| 976 | TBP | GGGTTTGATCATTCTGTAGAT | 1025 | KIF11 | CTTTTAAAGTACCTGTTGGGA |
| 977 | TBP | AGAAACAAAAATAAGGAGAAC | 1026 | KIF11 | ATTTCTCTTTTAAAGTACCTG |
| 978 | TBP | TGTTACAACTTACCTGTTAAT | 1027 | KIF11 | TCTGTGGTGTCGTACCTTTAA |
| 979 | TBP | GCTCTGACTTTAGCACCTAAG | 1028 | KIF11 | TACCAGTGTTGATGGGTATAA |
| 980 | TBP | TAAATTTCTGCTCTGACTTTA | 1029 | KIF11 | GTTCTTACCAGTGTTGATGGG |
| 981 | TBP | AATGCTTCATAAATTTCTGCT | 1030 | KIF11 | CGTGGTTCAGTTCTTACCAGT |
| 982 | TBP | TGAATCCCTTTAGAATAGGGT | 1031 | KIF11 | GCTGATCAAGGAGATGTTCAC |
| 983 | E2F4 | CTCCCACTGGGCCCAACAACA | 1032 | KIF11 | TTTTCAGCTGATCAAGGAGAT |
| 984 | E2F4 | GCCCTGCTGGACAGCAGCAGC | 1033 | KIF11 | GAACAGTTTAGCATCATTAAC |
| 985 | E2F4 | TCCGGACCCAACCCTTCTACC | 1034 | KIF11 | TTGTTGTTTTCTGAACAGTTT |
| 986 | E2F4 | ACCTCCTTTGAGCCCATCAAG | 1035 | KIF11 | GTATACTGCCCCAGAACTGCC |
| 987 | E2F4 | TGTTTTTCAGTTTTGGAACTC | 1036 | KIF11 | TCAGTATACTGCCCCAGAACT |
| 988 | E2F4 | GTTTTGGAACTCCCCAAAGAG | 1037 | KIF11 | ATGTGATTTTTTATGCTGTGG |
| 989 | E2F4 | CAGAGTGCATGAGCTCGGAGC | 1038 | KIF11 | TTGTCTTTTCCATGTGATTTT |
| 990 | E2F4 | TCTTTCTCCACCCCCGGGAGA | 1039 | KIF11 | ACTTTAGACCTCTCCAGTGTG |
| 991 | E2F4 | CCACCCCCGGGAGACCACGAT | 1040 | KIF11 | TCCACTTTAGACCTCTCCAGT |
| 992 | E2F4 | GCACTGCCAGGGACAGCAGTG | | | |

TABLE 11
Cas-Phi guide RNAs

| SEQ | | | SEQ | | |
|---|---|---|---|---|---|
| ID NO | Gene | Target Domain Sequence (DNA) | ID NO | Gene | Target Domain Sequence (DNA) |
| 1041 | GAPDH | TGCAGACCACAGTCCATGCCA | 1192 | E2F4 | ATGGGCTCAAAGGAGGTAGAA |
| 1042 | GAPDH | GCAGACCACAGTCCATGCCAT | 1193 | E2F4 | TGACAGCTCTTTGGGGAGTTC |
| 1043 | GAPDH | CAGACCACAGTCCATGCCATC | 1194 | E2F4 | CTGACAGCTCTTTGGGGAGTT |
| 1044 | GAPDH | TCATCTTCTAGGTATGACAAC | 1195 | E2F4 | TGAGGACATCAACTCCTCCAG |
| 1045 | GAPDH | CATCTTCTAGGTATGACAACG | 1196 | E2F4 | CAGGGCCACCCACCTTCTGAG |
| 1046 | GAPDH | ATCTTCTAGGTATGACAACGA | 1197 | E2F4 | TAGATATAATCGTGGTCTCCC |
| 1047 | GAPDH | TAGGTATGACAACGAATTTGG | 1198 | E2F4 | ACTCTCGTCCAGGTTGTAGAT |
| 1048 | GAPDH | CCCAGCTCTCATACCATGAGT | 1199 | G6PD | TGGGGGTTCACCCACTTGTAG |
| 1049 | TBP | TATCCACAGTGAATCTTGGTT | 1200 | G6PD | ACCCACTTGTAGGTGCCCTCA |
| 1050 | TBP | GTTGTAAACTTGACCTAAAGA | 1201 | G6PD | TAGGTGCCCTCATACTGGAAA |
| 1051 | TBP | TAAACTTGACCTAAAGACCAT | 1202 | G6PD | ATCAGCTCGTCTGCCTCCGTG |
| 1052 | TBP | ACCTAAAGACCATTGCACTTC | 1203 | G6PD | CCTCACCTGCCATAAATATAG |
| 1053 | TBP | CACTTCGTGCCCGAAACGCCG | 1204 | G6PD | CTCACCTGCCATAAATATAGG |
| 1054 | TBP | GTGCCCGAAACGCCGAATATA | 1205 | G6PD | GGCTTCTCCAGCTCAATCTGG |
| 1055 | TBP | TCTCTGACCATTGTAGCGGTT | 1206 | G6PD | TCCAGCTCAATCTGGTGCAGC |
| 1056 | TBP | TAGCGGTTTGCTGCGGTAATC | 1207 | G6PD | TCTGTAGGGCACCTTGTATCT |
| 1057 | TBP | GCTGCGGTAATCATGAGGATA | 1208 | G6PD | TATCTGTTGCCGTAGGTCAGG |
| 1058 | TBP | CTGCGGTAATCATGAGGATAA | 1209 | G6PD | CCGTAGGTCAGGTCCAGCTCC |
| 1059 | TBP | TCAGTTCTGGGAAAATGGTGT | 1210 | G6PD | AAGAACATGCCCGGCTTCTTG |
| 1060 | TBP | CAGTTCTGGGAAAATGGTGTG | 1211 | G6PD | TTGGTCATCATCTTGGTGTAC |
| 1061 | TBP | AGTTCTGGGAAAATGGTGTGC | 1212 | G6PD | GTCATCATCTTGGTGTACACG |
| 1062 | TBP | TGGGAAAATGGTGTGCACAGG | 1213 | G6PD | GTGTACACGGCCTCGTTGGGC |
| 1063 | TBP | TTTCCTTTCCCTAGTGAAGAA | 1214 | G6PD | GGCTGCACGCGGATCACCAGC |
| 1064 | TBP | TTCCTTTCCCTAGTGAAGAAC | 1215 | G6PD | CGCTTGCACTGCTGGTGGAAG |
| 1065 | TBP | TCCTTTCCCTAGTGAAGAACA | 1216 | G6PD | CACTGCTGGTGGAAGATGTCG |
| 1066 | TBP | CCTTTCCCTAGTGAAGAACAG | 1217 | G6PD | CGCTCGTTCAGGGCCTTGCCG |
| 1067 | TBP | CTTTCCCTAGTGAAGAACAGT | 1218 | G6PD | AGGGCCTTGCCGCAGCGCAGG |
| 1068 | TBP | CCCTAGTGAAGAACAGTCCAG | 1219 | G6PD | CCGCAGCGCAGGATGAAGGGC |
| 1069 | TBP | CCTAGTGAAGAACAGTCCAGA | 1220 | G6PD | CAGTATGAGGGCACCTACAAG |
| 1070 | TBP | TACAGAAGTTGGGTTTTCCAG | 1221 | G6PD | CCAGTATGAGGGCACCTACAA |
| 1071 | TBP | GGTTTTCCAGCTAAGTTCTTG | 1222 | G6PD | AGCTGGAGAAGCCCAAGCCCA |
| 1072 | TBP | TCCAGCTAAGTTCTTGGACTT | 1223 | G6PD | ACCCCACTGCTGCACCAGATT |
| 1073 | TBP | CCAGCTAAGTTCTTGGACTTC | 1224 | G6PD | CACCCCACTGCTGCACCAGAT |
| 1074 | TBP | CAGCTAAGTTCTTGGACTTCA | 1225 | G6PD | TCACCCCACTGCTGCACCAGA |
| 1075 | TBP | TTGGACTTCAAGATTCAGAAT | 1226 | G6PD | TGCGGGAGCCAGATGCACTTC |
| 1076 | TBP | GACTTCAAGATTCAGAATATG | 1227 | G6PD | AACCCCGAGGAGTCGGAGCTG |
| 1077 | TBP | AAGATTCAGAATATGGTGGGG | 1228 | G6PD | TTCAACCCCGAGGAGTCGGAG |
| 1078 | TBP | AGAATATGGTGGGGAGCTGTG | 1229 | G6PD | CACCAGCAGTGCAAGCGCAAC |
| 1079 | TBP | CCTATAAGGTTAGAAGGCCTT | 1230 | G6PD | CATGATGTGGCCGGCGACATC |
| 1080 | TBP | CTATAAGGTTAGAAGGCCTTG | 1231 | G6PD | ATCCTGCGCTGCGGCAAGGCC |
| 1081 | TBP | TGCTCACCCACCAACAATTTA | 1232 | G6PD | CGCCACGTAGGGGTGCCCTTC |
| 1082 | TBP | TTGCAATTTTCCTTCTAGTTA | 1233 | G6PD | CCGCCACGTAGGGGTGCCCTT |
| 1083 | TBP | TGCAATTTTCCTTCTAGTTAT | 1234 | KIF11 | ATGAAGATAAATTGATAGCAC |
| 1084 | TBP | GCAATTTTCCTTCTAGTTATG | 1235 | KIF11 | ATAGCACAAAATCTAGAACTT |
| 1085 | TBP | CAATTTTCCTTCTAGTTATGA | 1236 | KIF11 | ATGAAACCATAAAAATTGGTT |
| 1086 | TBP | TCCTTCTAGTTATGAGCCAGA | 1237 | KIF11 | GTTTGACTAAGCTTAATTGCT |
| 1087 | TBP | CCTTCTAGTTATGAGCCAGAG | 1238 | KIF11 | GACTAAGCTTAATTGCTTTCT |
| 1088 | TBP | CTTCTAGTTATGAGCCAGAGT | 1239 | KIF11 | ACTAAGCTTAATTGCTTTCTG |
| 1089 | TBP | TAGTTATGAGCCAGAGTTATT | 1240 | KIF11 | ATTGCTTTCTGGAACAGGATC |
| 1090 | TBP | TGAGCCAGAGTTATTTCCTGG | 1241 | KIF11 | CTTTCTGGAACAGGATCTGAA |
| 1091 | TBP | CCTGGTTTAATCTACAGAATG | 1242 | KIF11 | CTGGAACAGGATCTGAAACTG |
| 1092 | TBP | CTGGTTTAATCTACAGAATGA | 1243 | KIF11 | TGGAACAGGATCTGAAACTGG |
| 1093 | TBP | AATCTACAGAATGATCAAACC | 1244 | KIF11 | TCTAATGTCCGTTAAAGGTAC |
| 1094 | TBP | ATCTACAGAATGATCAAACCC | 1245 | KIF11 | AAGGTACGACACCACAGAGGA |
| 1095 | TBP | TTCTCCTTATTTTTGTTTCTG | 1246 | KIF11 | TTTATACCCATCAACACTGGT |
| 1096 | TBP | TCCTTATTTTTGTTTCTGGAA | 1247 | KIF11 | ATACCCATCAACACTGGTAAG |
| 1097 | TBP | TTTTTGTTTCTGGAAAAGTTG | 1248 | KIF11 | TACCCATCAACACTGGTAAGA |
| 1098 | TBP | TTGTTTCTGGAAAAGTTGTAT | 1249 | KIF11 | ATCAGCTGAAAAGGAAACAGC |
| 1099 | TBP | TGTTTCTGGAAAAGTTGTATT | 1250 | KIF11 | ATGATGCTAAACTGTTCAGAA |
| 1100 | TBP | GTTTCTGGAAAAGTTGTATTA | 1251 | KIF11 | AGAAAACAACAAAGAAGAGAC |
| 1101 | TBP | TTTCTGGAAAAGTTGTATTAA | 1252 | KIF11 | CTTCTTTTAGGATGTGGATGT |
| 1102 | TBP | CTGGAAAAGTTGTATTAACAG | 1253 | KIF11 | TTCTTTTAGGATGTGGATGTA |
| 1103 | TBP | TGGAAAAGTTGTATTAACAGG | 1254 | KIF11 | TTTTAGGATGTGGATGTAGAA |
| 1104 | TBP | TCTTCTTAGGTGCTAAAGTCA | 1255 | KIF11 | TAGGATGTGGATGTAGAAGAG |
| 1105 | TBP | TTAGGTGCTAAAGTCAGAGCA | 1256 | KIF11 | AGGATGTGGATGTAGAAGAGG |
| 1106 | TBP | GGTGCTAAAGTCAGAGCAGAA | 1257 | KIF11 | GGATGTGGATGTAGAAGAGGC |
| 1107 | TBP | TAAAGGGATTCAGGAAGACGA | 1258 | KIF11 | TGGGGCAGTATACTGAAGAAC |
| 1108 | TBP | GGTCAAGTTTACAACCAAGAT | 1259 | KIF11 | TTCATCAATTGGCGGGGTTCC |
| 1109 | TBP | AGGTCAAGTTTACAACCAAGA | 1260 | KIF11 | ATCAATTGGCGGGGTTCCATT |
| 1110 | TBP | GGGCACGAAGTGCAATGGTCT | 1261 | KIF11 | GCGGGGTTCCATTTTTCCAGG |
| 1111 | TBP | CGGGCACGAAGTGCAATGGTC | 1262 | KIF11 | TCCCGCCTTAAATCCACAGCA |
| 1112 | TBP | GGCGTTTCGGGCACGAAGTGC | 1263 | KIF11 | CCCGCCTTAAATCCACAGCAT |
| 1113 | TBP | TATTCGGCGTTTCGGGCACGA | 1264 | KIF11 | CCGCCTTAAATCCACAGCATA |
| 1114 | TBP | GGATTATATTCGGCGTTTCGG | 1265 | KIF11 | AATCCACAGCATAAAAAATCA |
| 1115 | TBP | AAATAGATCTAACCTTGGGAT | 1266 | KIF11 | ACACACTGGAGAGGTCTAAAG |
| 1116 | TBP | TCCTCATGATTACCGCAGCAA | 1267 | KIF11 | GTTACAAAGAGCAGATTACCT |
| 1117 | TBP | GTGGCTCTCTTATCCTCATGA | 1268 | KIF11 | CAAAGAGCAGATTACCTCTGC |
| 1118 | TBP | CCAGAACTGAAAATCAGTGCC | 1269 | KIF11 | CCTCTGCGAGCCCAGATCAAC |
| 1119 | TBP | CCCAGAACTGAAAATCAGTGC | 1270 | KIF11 | TAGATTTTGTGCTATCAATTT |
| 1120 | TBP | TCCCAGAACTGAAAATCAGTG | 1271 | KIF11 | AGTTCTAGATTTTGTGCTATC |
| 1121 | TBP | GCTCCTGTGCACACCATTTTC | 1272 | KIF11 | ATTAAGTTCTAGATTTTGTGC |
| 1122 | TBP | CGGCTACCTCTTGGCTCCTGT | 1273 | KIF11 | CATTAAGTTCTAGATTTTGTG |
| 1123 | TBP | TTACGGCTACCTCTTGGCTCC | 1274 | KIF11 | TGGTTTCATTAAGTTCTAGAT |
| 1124 | TBP | CTTACGGCTACCTCTTGGCTC | 1275 | KIF11 | ATGGTTTCATTAAGTTCTAGA |
| 1125 | TBP | CTGCCAGTCTGGACTGTTCTT | 1276 | KIF11 | TATGGTTTCATTAAGTTCTAG |
| 1126 | TBP | TTGCTGCCAGTCTGGACTGTT | 1277 | KIF11 | TTATGGTTTCATTAAGTTCTA |
| 1127 | TBP | CTTGCTGCCAGTCTGGACTGT | 1278 | KIF11 | GTCAAACCAATTTTTATGGTT |
| 1128 | TBP | TCTTGCTGCCAGTCTGGACTG | 1279 | KIF11 | AGCTTAGTCAAACCAATTTTT |
| 1129 | TBP | TGTACAACTCTAGCATATTTT | 1280 | KIF11 | CAGAAAGCAATTAAGCTTAGT |
| 1130 | TBP | GCTGGAAAACCCAACTTCTGT | 1281 | KIF11 | AGATCCTGTTCCAGAAAGCAA |
| 1131 | TBP | AAGTCCAAGAACTTAGCTGGA | 1282 | KIF11 | CAGATCCTGTTCCAGAAAGCA |
| 1132 | TBP | TGAATCTTGAAGTCCAAGAAC | 1283 | KIF11 | GGATATCCAGTTTCAGATCCT |
| 1133 | TBP | ACATCACAGCTCCCCACCATA | 1284 | KIF11 | AAGTACCTGTTGGGATATCCA |
| 1134 | TBP | TAACCTTATAGGAAACTTCAC | 1285 | KIF11 | AAAGTACCTGTTGGGATATCC |
| 1135 | TBP | GTGGGTGAGCACAAGGCCTTC | 1286 | KIF11 | TAAAGTACCTGTTGGGATATC |
| 1136 | TBP | TTGGTGGGTGAGCACAAGGCC | 1287 | KIF11 | TCTTTTAAAGTACCTGTTGGG |
| 1137 | TBP | CCTACTAAATTGTTGGTGGGT | 1288 | KIF11 | CTCTTTTAAAGTACCTGTTGG |
| 1138 | TBP | AGACTTACCTACTAAATTGTT | 1289 | KIF11 | TATTTCTCTTTTAAAGTACCT |
| 1139 | TBP | CAGACTTACCTACTAAATTGT | 1290 | KIF11 | CTCTGTGGTGTCGTACCTTTA |
| 1140 | TBP | AACCAGGAAATAACTCTGGCT | 1291 | KIF11 | CCTCTGTGGTGTCGTACCTTT |
| 1141 | TBP | TGTAGATTAAACCAGGAAATA | 1292 | KIF11 | TCCTCTGTGGTGTCGTACCTT |
| 1142 | TBP | ATCATTCTGTAGATTAAACCA | 1293 | KIF11 | ATGGGTATAAATAACTTTTCC |
| 1143 | TBP | GATCATTCTGTAGATTAAACC | 1294 | KIF11 | CCAGTGTTGATGGGTATAAAT |
| 1144 | TBP | TGGGTTTGATCATTCTGTAGA | 1295 | KIF11 | TTACCAGTGTTGATGGGTATA |
| 1145 | TBP | CAGAAACAAAAATAAGGAGAA | 1296 | KIF11 | AGTTCTTACCAGTGTTGATGG |
| 1146 | TBP | CCAGAAACAAAAATAAGGAGA | 1297 | KIF11 | ACGTGGTTCAGTTCTTACCAG |
| 1147 | TBP | TCCAGAAACAAAAATAAGGAG | 1298 | KIF11 | AGCTGATCAAGGAGATGTTCA |
| 1148 | TBP | ATACAACTTTTCCAGAAACAA | 1299 | KIF11 | CAGCTGATCAAGGAGATGTTC |
| 1149 | TBP | CCTGTTAATACAACTTTTCCA | 1300 | KIF11 | TCAGCTGATCAAGGAGATGTT |
| 1150 | TBP | CAACTTACCTGTTAATACAAC | 1301 | KIF11 | CTTTTCAGCTGATCAAGGAGA |
| 1151 | TBP | CTGTTACAACTTACCTGTTAA | 1302 | KIF11 | CCTTTTCAGCTGATCAAGGAG |
| 1152 | TBP | TGCTCTGACTTTAGCACCTAA | 1303 | KIF11 | ACAGCTCAGGCTGTTTCCTTT |
| 1153 | TBP | CTGCTCTGACTTTAGCACCTA | 1304 | KIF11 | GCATCATTAACAGCTCAGGCT |
| 1154 | TBP | ATAAATTTCTGCTCTGACTTT | 1305 | KIF11 | AGCATCATTAACAGCTCAGGC |
| 1155 | TBP | AAATGCTTCATAAATTTCTGC | 1306 | KIF11 | TGAACAGTTTAGCATCATTAA |
| 1156 | TBP | CAAATGCTTCATAAATTTCTG | 1307 | KIF11 | CTGAACAGTTTAGCATCATTA |
| 1157 | TBP | TCAAATGCTTCATAAATTTCT | 1308 | KIF11 | TCTGAACAGTTTAGCATCATT |
| 1158 | TBP | CTGAATCCCTTTAGAATAGGG | 1309 | KIF11 | TTTTCTGAACAGTTTAGCATC |
| 1159 | TBP | CGTCGTCTTCCTGAATCCCTT | 1310 | KIF11 | TTGTTTTCTGAACAGTTTAGC |
| 1160 | E2F4 | GGGGGCTATCATTGTAGTGAG | 1311 | KIF11 | TTTGTTGTTTTCTGAACAGTT |
| 1161 | E2F4 | GGGGCTATCATTGTAGTGAGT | 1312 | KIF11 | TCTCTTCTTTGTTGTTTTCTG |
| 1162 | E2F4 | TAGTGAGTGGCGGCCCTGGGA | 1313 | KIF11 | CCGGAATTGTCTCTTCTTTGT |
| 1163 | E2F4 | ACTCCCACTGGGCCCAACAAC | 1314 | KIF11 | ACCGGAATTGTCTCTTCTTTG |
| 1164 | E2F4 | TGCCCTGCTGGACAGCAGCAG | 1315 | KIF11 | AATTTACCGGAATTGTCTCTT |
| 1165 | E2F4 | GTCCGGACCCAACCCTTCTAC | 1316 | KIF11 | AAATTTACCGGAATTGTCTCT |
| 1166 | E2F4 | TACCTCCTTTGAGCCCATCAA | 1317 | KIF11 | AGTATACTGCCCCAGAACTGC |
| 1167 | E2F4 | GAGCCCATCAAGGCAGACCCC | 1318 | KIF11 | TTCAGTATACTGCCCCAGAAC |
| 1168 | E2F4 | AGCCCATCAAGGCAGACCCCA | 1319 | KIF11 | GAGGTTCTTCAGTATACTGCC |
| 1169 | E2F4 | CTTGTTTTTCAGTTTTGGAAC | 1320 | KIF11 | ACTTAGAGGTTCTTCAGTATA |
| 1170 | E2F4 | TTTTTCAGTTTTGGAACTCCC | 1321 | KIF11 | ATGAACAATCCACACCAGCAT |
| 1171 | E2F4 | TTCAGTTTTGGAACTCCCCAA | 1322 | KIF11 | TCTGATATGACATACCTGGAA |
| 1172 | E2F4 | TCAGTTTTGGAACTCCCCAAA | 1323 | KIF11 | CATGTGATTTTTTATGCTGTG |
| 1173 | E2F4 | CAGTTTTGGAACTCCCCAAAG | 1324 | KIF11 | CCATGTGATTTTTTATGCTGT |
| 1174 | E2F4 | AGTTTTGGAACTCCCCAAAGA | 1325 | KIF11 | TCCATGTGATTTTTTATGCTG |
| 1175 | E2F4 | TGGAACTCCCCAAAGAGCTGT | 1326 | KIF11 | TCTTTTCCATGTGATTTTTTA |
| 1176 | E2F4 | GGAACTCCCCAAAGAGCTGTC | 1327 | KIF11 | GTCTTTTCCATGTGATTTTTT |
| 1177 | E2F4 | CCAGAGTGCATGAGCTCGGAG | 1328 | KIF11 | TTTGTCTTTTCCATGTGATTT |
| 1178 | E2F4 | GCCCCTCTGCTTCGTCTTTCT | 1329 | KIF11 | CTTTGTCTTTTCCATGTGATT |
| 1179 | E2F4 | CCCCTCTGCTTCGTCTTTCTC | 1330 | KIF11 | TCTTTGTCTTTTCCATGTGAT |
| 1180 | E2F4 | GTCTTTCTCCACCCCCGGGAG | 1331 | KIF11 | ATGCCTCTGTTTTCTTTGTCT |
| 1181 | E2F4 | CTCCACCCCCGGGAGACCACG | 1332 | KIF11 | GACCTCTCCAGTGTGTTAATG |
| 1182 | E2F4 | TCCACCCCCGGGAGACCACGA | 1333 | KIF11 | AGACCTCTCCAGTGTGTTAAT |
| 1183 | E2F4 | TATCTACAACCTGGACGAGAG | 1334 | KIF11 | CACTTTAGACCTCTCCAGTGT |
| 1184 | E2F4 | GATGTGCCTGTTCTCAACCTC | 1335 | KIF11 | TTCCACTTTAGACCTCTCCAG |
| 1185 | E2F4 | ATGTGCCTGTTCTCAACCTCT | 1336 | KIF11 | CTTCCACTTTAGACCTCTCCA |
| 1186 | E2F4 | TGCACTGCCAGGGACAGCAGT | 1337 | KIF11 | TAACCAAGTGCTCTGTAGTTT |
| 1187 | E2F4 | CCTGGACTTCTGCACTGCCAG | 1338 | KIF11 | GTAACCAAGTGCTCTGTAGTT |
| 1188 | E2F4 | CTATCAGTCCCAGGGCCGCCA | 1339 | KIF11 | ATCTGGGCTCGCAGAGGTAAT |
| 1189 | E2F4 | GGCCCAGTGGGAGTGAACTGA | 1340 | KIF11 | AAGGTTGATCTGGGCTCGCAG |
| 1190 | E2F4 | TTGGGCCCAGTGGGAGTGAAC | 1341 | KIF11 | CCAACCCCCAAGTGAATTAAA |
| 1191 | E2F4 | GGTCCGGACGAACTGCTGCTG | | | |

TABLE 12
Mad7 guide RNAs

| SEQ | | | SEQ | | |
|---|---|---|---|---|---|
| ID NO | Gene | Target Domain Sequence (DNA) | ID NO | Gene | Target Domain Sequence (DNA) |
| 1342 | GAPDH | TGCAGACCACAGTCCATGCCA | 1489 | E2F4 | TTGGGCCCAGTGGGAGTGAAC |
| 1343 | GAPDH | GCAGACCACAGTCCATGCCAT | 1490 | E2F4 | GGTCCGGACGAACTGCTGCTG |
| 1344 | GAPDH | CAGACCACAGTCCATGCCATC | 1491 | E2F4 | ATGGGCTCAAAGGAGGTAGAA |
| 1345 | GAPDH | TCATCTTCTAGGTATGACAAC | 1492 | E2F4 | TGACAGCTCTTTGGGGAGTTC |
| 1346 | GAPDH | CATCTTCTAGGTATGACAACG | 1493 | E2F4 | CTGACAGCTCTTTGGGGAGTT |
| 1347 | GAPDH | ATCTTCTAGGTATGACAACGA | 1494 | E2F4 | TGAGGACATCAACTCCTCCAG |
| 1348 | GAPDH | TAGGTATGACAACGAATTTGG | 1495 | E2F4 | CAGGGCCACCCACCTTCTGAG |
| 1349 | GAPDH | CCCAGCTCTCATACCATGAGT | 1496 | E2F4 | TAGATATAATCGTGGTCTCCC |
| 1350 | TBP | TATCCACAGTGAATCTTGGTT | 1497 | E2F4 | ACTCTCGTCCAGGTTGTAGAT |
| 1351 | TBP | GTTGTAAACTTGACCTAAAGA | 1498 | G6PD | TGGGGGTTCACCCACTTGTAG |
| 1352 | TBP | TAAACTTGACCTAAAGACCAT | 1499 | G6PD | ACCCACTTGTAGGTGCCCTCA |
| 1353 | TBP | ACCTAAAGACCATTGCACTTC | 1500 | G6PD | TAGGTGCCCTCATACTGGAAA |
| 1354 | TBP | CACTTCGTGCCCGAAACGCCG | 1501 | G6PD | ATCAGCTCGTCTGCCTCCGTG |
| 1355 | TBP | GTGCCCGAAACGCCGAATATA | 1502 | G6PD | CCTCACCTGCCATAAATATAG |
| 1356 | TBP | TCTCTGACCATTGTAGCGGTT | 1503 | G6PD | CTCACCTGCCATAAATATAGG |
| 1357 | TBP | TAGCGGTTTGCTGCGGTAATC | 1504 | G6PD | GGCTTCTCCAGCTCAATCTGG |
| 1358 | TBP | GCTGCGGTAATCATGAGGATA | 1505 | G6PD | TCCAGCTCAATCTGGTGCAGC |
| 1359 | TBP | CTGCGGTAATCATGAGGATAA | 1506 | G6PD | TCTGTAGGGCACCTTGTATCT |
| 1360 | TBP | TCAGTTCTGGGAAAATGGTGT | 1507 | G6PD | TATCTGTTGCCGTAGGTCAGG |
| 1361 | TBP | CAGTTCTGGGAAAATGGTGTG | 1508 | G6PD | CCGTAGGTCAGGTCCAGCTCC |
| 1362 | TBP | AGTTCTGGGAAAATGGTGTGC | 1509 | G6PD | AAGAACATGCCCGGCTTCTTG |
| 1363 | TBP | TGGGAAAATGGTGTGCACAGG | 1510 | G6PD | TTGGTCATCATCTTGGTGTAC |
| 1364 | TBP | TTTCCTTTCCCTAGTGAAGAA | 1511 | G6PD | GTCATCATCTTGGTGTACACG |
| 1365 | TBP | TTCCTTTCCCTAGTGAAGAAC | 1512 | G6PD | GTGTACACGGCCTCGTTGGGC |
| 1366 | TBP | TCCTTTCCCTAGTGAAGAACA | 1513 | G6PD | GGCTGCACGCGGATCACCAGC |
| 1367 | TBP | CCTTTCCCTAGTGAAGAACAG | 1514 | G6PD | CGCTTGCACTGCTGGTGGAAG |
| 1368 | TBP | CTTTCCCTAGTGAAGAACAGT | 1515 | G6PD | CACTGCTGGTGGAAGATGTCG |
| 1369 | TBP | CCCTAGTGAAGAACAGTCCAG | 1516 | G6PD | CGCTCGTTCAGGGCCTTGCCG |
| 1370 | TBP | CCTAGTGAAGAACAGTCCAGA | 1517 | G6PD | AGGGCCTTGCCGCAGCGCAGG |
| 1371 | TBP | TACAGAAGTTGGGTTTTCCAG | 1518 | G6PD | CCGCAGCGCAGGATGAAGGGC |
| 1372 | TBP | GGTTTTCCAGCTAAGTTCTTG | 1519 | G6PD | CAGTATGAGGGCACCTACAAG |
| 1373 | TBP | TCCAGCTAAGTTCTTGGACTT | 1520 | G6PD | CCAGTATGAGGGCACCTACAA |
| 1374 | TBP | CCAGCTAAGTTCTTGGACTTC | 1521 | G6PD | AGCTGGAGAAGCCCAAGCCCA |
| 1375 | TBP | CAGCTAAGTTCTTGGACTTCA | 1522 | G6PD | ACCCCACTGCTGCACCAGATT |
| 1376 | TBP | TTGGACTTCAAGATTCAGAAT | 1523 | G6PD | CACCCCACTGCTGCACCAGAT |
| 1377 | TBP | GACTTCAAGATTCAGAATATG | 1524 | G6PD | TCACCCCACTGCTGCACCAGA |
| 1378 | TBP | AAGATTCAGAATATGGTGGGG | 1525 | G6PD | TGCGGGAGCCAGATGCACTTC |
| 1379 | TBP | AGAATATGGTGGGGAGCTGTG | 1526 | G6PD | AACCCCGAGGAGTCGGAGCTG |
| 1380 | TBP | CCTATAAGGTTAGAAGGCCTT | 1527 | G6PD | TTCAACCCCGAGGAGTCGGAG |
| 1381 | TBP | CTATAAGGTTAGAAGGCCTTG | 1528 | G6PD | CACCAGCAGTGCAAGCGCAAC |
| 1382 | TBP | TGCTCACCCACCAACAATTTA | 1529 | G6PD | CATGATGTGGCCGGCGACATC |
| 1383 | TBP | TTGCAATTTTCCTTCTAGTTA | 1530 | G6PD | ATCCTGCGCTGCGGCAAGGCC |
| 1384 | TBP | TGCAATTTTCCTTCTAGTTAT | 1531 | G6PD | CGCCACGTAGGGGTGCCCTTC |
| 1385 | TBP | GCAATTTTCCTTCTAGTTATG | 1532 | G6PD | CCGCCACGTAGGGGTGCCCTT |
| 1386 | TBP | CAATTTTCCTTCTAGTTATGA | 1533 | KIF11 | ATGAAGATAAATTGATAGCAC |
| 1387 | TBP | TCCTTCTAGTTATGAGCCAGA | 1534 | KIF11 | ATAGCACAAAATCTAGAACTT |
| 1388 | TBP | CCTTCTAGTTATGAGCCAGAG | 1535 | KIF11 | ATGAAACCATAAAAATTGGTT |
| 1389 | TBP | CTTCTAGTTATGAGCCAGAGT | 1536 | KIF11 | GTTTGACTAAGCTTAATTGCT |
| 1390 | TBP | TAGTTATGAGCCAGAGTTATT | 1537 | KIF11 | GACTAAGCTTAATTGCTTTCT |
| 1391 | TBP | TGAGCCAGAGTTATTTCCTGG | 1538 | KIF11 | ACTAAGCTTAATTGCTTTCTG |
| 1392 | TBP | CCTGGTTTAATCTACAGAATG | 1539 | KIF11 | ATTGCTTTCTGGAACAGGATC |
| 1393 | TBP | CTGGTTTAATCTACAGAATGA | 1540 | KIF11 | CTTTCTGGAACAGGATCTGAA |
| 1394 | TBP | AATCTACAGAATGATCAAACC | 1541 | KIF11 | CTGGAACAGGATCTGAAACTG |
| 1395 | TBP | ATCTACAGAATGATCAAACCC | 1542 | KIF11 | TGGAACAGGATCTGAAACTGG |
| 1396 | TBP | TTCTCCTTATTTTTGTTTCTG | 1543 | KIF11 | TCTAATGTCCGTTAAAGGTAC |
| 1397 | TBP | TCCTTATTTTTGTTTCTGGAA | 1544 | KIF11 | AAGGTACGACACCACAGAGGA |
| 1398 | TBP | TTTTTGTTTCTGGAAAAGTTG | 1545 | KIF11 | TTTATACCCATCAACACTGGT |
| 1399 | TBP | TTGTTTCTGGAAAAGTTGTAT | 1546 | KIF11 | ATACCCATCAACACTGGTAAG |
| 1400 | TBP | TGTTTCTGGAAAAGTTGTATT | 1547 | KIF11 | TACCCATCAACACTGGTAAGA |
| 1401 | TBP | GTTTCTGGAAAAGTTGTATTA | 1548 | KIF11 | ATCAGCTGAAAAGGAAACAGC |
| 1402 | TBP | TTTCTGGAAAAGTTGTATTAA | 1549 | KIF11 | ATGATGCTAAACTGTTCAGAA |
| 1403 | TBP | CTGGAAAAGTTGTATTAACAG | 1550 | KIF11 | AGAAAACAACAAAGAAGAGAC |
| 1404 | TBP | TGGAAAAGTTGTATTAACAGG | 1551 | KIF11 | CTTCTTTTAGGATGTGGATGT |
| 1405 | TBP | TCTTCTTAGGTGCTAAAGTCA | 1552 | KIF11 | TTCTTTTAGGATGTGGATGTA |
| 1406 | TBP | TTAGGTGCTAAAGTCAGAGCA | 1553 | KIF11 | TTTTAGGATGTGGATGTAGAA |
| 1407 | TBP | GGTGCTAAAGTCAGAGCAGAA | 1554 | KIF11 | TAGGATGTGGATGTAGAAGAG |
| 1408 | TBP | TAAAGGGATTCAGGAAGACGA | 1555 | KIF11 | AGGATGTGGATGTAGAAGAGG |
| 1409 | TBP | GGTCAAGTTTACAACCAAGAT | 1556 | KIF11 | GGATGTGGATGTAGAAGAGGC |
| 1410 | TBP | AGGTCAAGTTTACAACCAAGA | 1557 | KIF11 | TGGGGCAGTATACTGAAGAAC |
| 1411 | TBP | GGGCACGAAGTGCAATGGTCT | 1558 | KIF11 | TTCATCAATTGGCGGGGTTCC |
| 1412 | TBP | CGGGCACGAAGTGCAATGGTC | 1559 | KIF11 | ATCAATTGGCGGGGTTCCATT |
| 1413 | TBP | GGCGTTTCGGGCACGAAGTGC | 1560 | KIF11 | GCGGGGTTCCATTTTTCCAGG |
| 1414 | TBP | TATTCGGCGTTTCGGGCACGA | 1561 | KIF11 | TCCCGCCTTAAATCCACAGCA |
| 1415 | TBP | GGATTATATTCGGCGTTTCGG | 1562 | KIF11 | CCCGCCTTAAATCCACAGCAT |
| 1416 | TBP | AAATAGATCTAACCTTGGGAT | 1563 | KIF11 | CCGCCTTAAATCCACAGCATA |
| 1417 | TBP | TCCTCATGATTACCGCAGCAA | 1564 | KIF11 | AATCCACAGCATAAAAAATCA |
| 1418 | TBP | GTGGCTCTCTTATCCTCATGA | 1565 | KIF11 | ACACACTGGAGAGGTCTAAAG |
| 1419 | TBP | CCAGAACTGAAAATCAGTGCC | 1566 | KIF11 | GTTACAAAGAGCAGATTACCT |
| 1420 | TBP | CCCAGAACTGAAAATCAGTGC | 1567 | KIF11 | CAAAGAGCAGATTACCTCTGC |
| 1421 | TBP | TCCCAGAACTGAAAATCAGTG | 1568 | KIF11 | CCTCTGCGAGCCCAGATCAAC |
| 1422 | TBP | GCTCCTGTGCACACCATTTTC | 1569 | KIF11 | TAGATTTTGTGCTATCAATTT |
| 1423 | TBP | CGGCTACCTCTTGGCTCCTGT | 1570 | KIF11 | AGTTCTAGATTTTGTGCTATC |
| 1424 | TBP | TTACGGCTACCTCTTGGCTCC | 1571 | KIF11 | ATTAAGTTCTAGATTTTGTGC |
| 1425 | TBP | CTTACGGCTACCTCTTGGCTC | 1572 | KIF11 | CATTAAGTTCTAGATTTTGTG |
| 1426 | TBP | CTGCCAGTCTGGACTGTTCTT | 1573 | KIF11 | TGGTTTCATTAAGTTCTAGAT |
| 1427 | TBP | TTGCTGCCAGTCTGGACTGTT | 1574 | KIF11 | ATGGTTTCATTAAGTTCTAGA |
| 1428 | TBP | CTTGCTGCCAGTCTGGACTGT | 1575 | KIF11 | TATGGTTTCATTAAGTTCTAG |
| 1429 | TBP | TCTTGCTGCCAGTCTGGACTG | 1576 | KIF11 | TTATGGTTTCATTAAGTTCTA |
| 1430 | TBP | TGTACAACTCTAGCATATTTT | 1577 | KIF11 | GTCAAACCAATTTTTATGGTT |
| 1431 | TBP | GCTGGAAAACCCAACTTCTGT | 1578 | KIF11 | AGCTTAGTCAAACCAATTTTT |
| 1432 | TBP | AAGTCCAAGAACTTAGCTGGA | 1579 | KIF11 | CAGAAAGCAATTAAGCTTAGT |
| 1433 | TBP | TGAATCTTGAAGTCCAAGAAC | 1580 | KIF11 | AGATCCTGTTCCAGAAAGCAA |
| 1434 | TBP | ACATCACAGCTCCCCACCATA | 1581 | KIF11 | CAGATCCTGTTCCAGAAAGCA |
| 1435 | TBP | TAACCTTATAGGAAACTTCAC | 1582 | KIF11 | GGATATCCAGTTTCAGATCCT |
| 1436 | TBP | GTGGGTGAGCACAAGGCCTTC | 1583 | KIF11 | AAGTACCTGTTGGGATATCCA |
| 1437 | TBP | TTGGTGGGTGAGCACAAGGCC | 1584 | KIF11 | AAAGTACCTGTTGGGATATCC |
| 1438 | TBP | CCTACTAAATTGTTGGTGGGT | 1585 | KIF11 | TAAAGTACCTGTTGGGATATC |
| 1439 | TBP | AGACTTACCTACTAAATTGTT | 1586 | KIF11 | TCTTTTAAAGTACCTGTTGGG |
| 1440 | TBP | CAGACTTACCTACTAAATTGT | 1587 | KIF11 | CTCTTTTAAAGTACCTGTTGG |
| 1441 | TBP | AACCAGGAAATAACTCTGGCT | 1588 | KIF11 | TATTTCTCTTTTAAAGTACCT |
| 1442 | TBP | TGTAGATTAAACCAGGAAATA | 1589 | KIF11 | ATGGGTATAAATAACTTTTCC |
| 1443 | TBP | ATCATTCTGTAGATTAAACCA | 1590 | KIF11 | CCAGTGTTGATGGGTATAAAT |
| 1444 | TBP | GATCATTCTGTAGATTAAACC | 1591 | KIF11 | TTACCAGTGTTGATGGGTATA |
| 1445 | TBP | TGGGTTTGATCATTCTGTAGA | 1592 | KIF11 | AGTTCTTACCAGTGTTGATGG |
| 1446 | TBP | CAGAAACAAAAATAAGGAGAA | 1593 | KIF11 | ACGTGGTTCAGTTCTTACCAG |
| 1447 | TBP | CCAGAAACAAAAATAAGGAGA | 1594 | KIF11 | AGCTGATCAAGGAGATGTTCA |
| 1448 | TBP | TCCAGAAACAAAAATAAGGAG | 1595 | KIF11 | CAGCTGATCAAGGAGATGTTC |
| 1449 | TBP | ATACAACTTTTCCAGAAACAA | 1596 | KIF11 | TCAGCTGATCAAGGAGATGTT |
| 1450 | TBP | CCTGTTAATACAACTTTTCCA | 1597 | KIF11 | CTTTTCAGCTGATCAAGGAGA |
| 1451 | TBP | CAACTTACCTGTTAATACAAC | 1598 | KIF11 | CCTTTTCAGCTGATCAAGGAG |
| 1452 | TBP | CTGTTACAACTTACCTGTTAA | 1599 | KIF11 | ACAGCTCAGGCTGTTTCCTTT |
| 1453 | TBP | ATAAATTTCTGCTCTGACTTT | 1600 | KIF11 | GCATCATTAACAGCTCAGGCT |
| 1454 | TBP | AAATGCTTCATAAATTTCTGC | 1601 | KIF11 | AGCATCATTAACAGCTCAGGC |
| 1455 | TBP | CAAATGCTTCATAAATTTCTG | 1602 | KIF11 | TGAACAGTTTAGCATCATTAA |
| 1456 | TBP | TCAAATGCTTCATAAATTTCT | 1603 | KIF11 | CTGAACAGTTTAGCATCATTA |
| 1457 | TBP | CTGAATCCCTTTAGAATAGGG | 1604 | KIF11 | TCTGAACAGTTTAGCATCATT |
| 1458 | TBP | CGTCGTCTTCCTGAATCCCTT | 1605 | KIF11 | TTTTCTGAACAGTTTAGCATC |
| 1459 | E2F4 | GGGGGCTATCATTGTAGTGAG | 1606 | KIF11 | TTGTTTTCTGAACAGTTTAGC |
| 1460 | E2F4 | GGGGCTATCATTGTAGTGAGT | 1607 | KIF11 | TTTGTTGTTTTCTGAACAGTT |
| 1461 | E2F4 | TAGTGAGTGGCGGCCCTGGGA | 1608 | KIF11 | TCTCTTCTTTGTTGTTTTCTG |
| 1462 | E2F4 | ACTCCCACTGGGCCCAACAAC | 1609 | KIF11 | CCGGAATTGTCTCTTCTTTGT |
| 1463 | E2F4 | TGCCCTGCTGGACAGCAGCAG | 1610 | KIF11 | ACCGGAATTGTCTCTTCTTTG |
| 1464 | E2F4 | GTCCGGACCCAACCCTTCTAC | 1611 | KIF11 | AATTTACCGGAATTGTCTCTT |
| 1465 | E2F4 | TACCTCCTTTGAGCCCATCAA | 1612 | KIF11 | AAATTTACCGGAATTGTCTCT |
| 1466 | E2F4 | GAGCCCATCAAGGCAGACCCC | 1613 | KIF11 | AGTATACTGCCCCAGAACTGC |
| 1467 | E2F4 | AGCCCATCAAGGCAGACCCCA | 1614 | KIF11 | TTCAGTATACTGCCCCAGAAC |
| 1468 | E2F4 | CTTGTTTTTCAGTTTTGGAAC | 1615 | KIF11 | GAGGTTCTTCAGTATACTGCC |
| 1469 | E2F4 | TTTTTCAGTTTTGGAACTCCC | 1616 | KIF11 | ACTTAGAGGTTCTTCAGTATA |
| 1470 | E2F4 | TTCAGTTTTGGAACTCCCCAA | 1617 | KIF11 | ATGAACAATCCACACCAGCAT |
| 1471 | E2F4 | TCAGTTTTGGAACTCCCCAAA | 1618 | KIF11 | TCTGATATGACATACCTGGAA |
| 1472 | E2F4 | CAGTTTTGGAACTCCCCAAAG | 1619 | KIF11 | TCTTTTCCATGTGATTTTTTA |
| 1473 | E2F4 | AGTTTTGGAACTCCCCAAAGA | 1620 | KIF11 | GTCTTTTCCATGTGATTTTTT |
| 1474 | E2F4 | TGGAACTCCCCAAAGAGCTGT | 1621 | KIF11 | TTTGTCTTTTCCATGTGATTT |
| 1475 | E2F4 | GGAACTCCCCAAAGAGCTGTC | 1622 | KIF11 | CTTTGTCTTTTCCATGTGATT |
| 1476 | E2F4 | CCAGAGTGCATGAGCTCGGAG | 1623 | KIF11 | TCTTTGTCTTTTCCATGTGAT |
| 1477 | E2F4 | GCCCCTCTGCTTCGTCTTTCT | 1624 | KIF11 | ATGCCTCTGTTTTCTTTGTCT |
| 1478 | E2F4 | CCCCTCTGCTTCGTCTTTCTC | 1625 | KIF11 | GACCTCTCCAGTGTGTTAATG |
| 1479 | E2F4 | GTCTTTCTCCACCCCCGGGAG | 1626 | KIF11 | AGACCTCTCCAGTGTGTTAAT |
| 1480 | E2F4 | CTCCACCCCCGGGAGACCACG | 1627 | KIF11 | CACTTTAGACCTCTCCAGTGT |
| 1481 | E2F4 | TCCACCCCCGGGAGACCACGA | 1628 | KIF11 | TTCCACTTTAGACCTCTCCAG |
| 1482 | E2F4 | TATCTACAACCTGGACGAGAG | 1629 | KIF11 | CTTCCACTTTAGACCTCTCCA |
| 1483 | E2F4 | GATGTGCCTGTTCTCAACCTC | 1630 | KIF11 | TAACCAAGTGCTCTGTAGTTT |
| 1484 | E2F4 | ATGTGCCTGTTCTCAACCTCT | 1631 | KIF11 | GTAACCAAGTGCTCTGTAGTT |
| 1485 | E2F4 | TGCACTGCCAGGGACAGCAGT | 1632 | KIF11 | ATCTGGGCTCGCAGAGGTAAT |
| 1486 | E2F4 | CCTGGACTTCTGCACTGCCAG | 1633 | KIF11 | AAGGTTGATCTGGGCTCGCAG |
| 1487 | E2F4 | CTATCAGTCCCAGGGCCGCCA | 1634 | KIF11 | CCAACCCCCAAGTGAATTAAA |
| 1488 | E2F4 | GGCCCAGTGGGAGTGAACTGA | | | |

TABLE 13
SpyCas9 guide

| SEQ | | | SEQ | | |
|---|---|---|---|---|---|
| ID NO | Gene | Target Domain Sequence (DNA) | ID NO | Gene | Target Domain Sequence (DNA) |
| 1635 | GAPDH | TCTAGGTATGACAACGAATT | 1761 | G6PD | GTGGGGGTTCACCCACTTGT |
| 1636 | GAPDH | AGCCCCAGCGTCAAAGGTGG | 1762 | G6PD | ACTTGTAGGTGCCCTCATAC |
| 1637 | TBP | ATTGTATCCACAGTGAATCT | 1763 | G6PD | CATCAGCTCGTCTGCCTCCG |
| 1638 | TBP | AAACGCCGAATATAATCCCA | 1764 | G6PD | ATCAGCTCGTCTGCCTCCGT |
| 1639 | TBP | ACCATTGTAGCGGTTTGCTG | 1765 | G6PD | TCAGCTCGTCTGCCTCCGTG |
| 1640 | TBP | GGTTTGCTGCGGTAATCATG | 1766 | G6PD | CGTCTGCCTCCGTGGGGCCT |
| 1641 | TBP | GATAAGAGAGCCACGAACCA | 1767 | G6PD | TGCCTCCGTGGGGCCTCGGC |
| 1642 | TBP | ACGGCACTGATTTTCAGTTC | 1768 | G6PD | TCCTCACCTGCCATAAATAT |
| 1643 | TBP | CGGCACTGATTTTCAGTTCT | 1769 | G6PD | CCTCACCTGCCATAAATATA |
| 1644 | TBP | GATTTTCAGTTCTGGGAAAA | 1770 | G6PD | CTCACCTGCCATAAATATAG |
| 1645 | TBP | TCTGGGAAAATGGTGTGCAC | 1771 | G6PD | CCTGCCATAAATATAGGGGA |
| 1646 | TBP | TGGTGTGCACAGGAGCCAAG | 1772 | G6PD | CTGCCATAAATATAGGGGAT |
| 1647 | TBP | TAGTGAAGAACAGTCCAGAC | 1773 | G6PD | ATAAATATAGGGGATGGGCT |
| 1648 | TBP | TGCTAGAGTTGTACAGAAGT | 1774 | G6PD | TAAATATAGGGGATGGGCTT |
| 1649 | TBP | GCTAGAGTTGTACAGAAGTT | 1775 | G6PD | TGGGCTTCTCCAGCTCAATC |
| 1650 | TBP | GGGTTTTCCAGCTAAGTTCT | 1776 | G6PD | AGCTCAATCTGGTGCAGCAG |
| 1651 | TBP | GGACTTCAAGATTCAGAATA | 1777 | G6PD | GCTCAATCTGGTGCAGCAGT |
| 1652 | TBP | CTTCAAGATTCAGAATATGG | 1778 | G6PD | CTCAATCTGGTGCAGCAGTG |
| 1653 | TBP | TTCAAGATTCAGAATATGGT | 1779 | G6PD | CAGTGGGGTGAAAATACGCC |
| 1654 | TBP | TCAAGATTCAGAATATGGTG | 1780 | G6PD | TGAAAATACGCCAGGCCTCA |
| 1655 | TBP | GTGATGTGAAGTTTCCTATA | 1781 | G6PD | CCTCACGGAGCTCGTCGCTG |
| 1656 | TBP | AAGTTTCCTATAAGGTTAGA | 1782 | G6PD | ACCTGCGCACGAAGTGCATC |
| 1657 | TBP | TCACCCACCAACAATTTAGT | 1783 | G6PD | GGCTCCCGCAGAAGACGTCC |
| 1658 | TBP | TATGAGCCAGAGTTATTTCC | 1784 | G6PD | CGCAGAAGACGTCCAGGATG |
| 1659 | TBP | GTTCTCCTTATTTTTGTTTC | 1785 | G6PD | GTCCAGGATGAGGCGCTCAT |
| 1660 | TBP | TCTGGAAAAGTTGTATTAAC | 1786 | G6PD | ATGAGGCGCTCATAGGCGTC |
| 1661 | TBP | AAACATCTACCCTATTCTAA | 1787 | G6PD | TGAGGCGCTCATAGGCGTCA |
| 1662 | TBP | ACCCTATTCTAAAGGGATTC | 1788 | G6PD | CACCTTGTATCTGTTGCCGT |
| 1663 | TBP | GATTCAGGAAGACGACGTAA | 1789 | G6PD | TGTATCTGTTGCCGTAGGTC |
| 1664 | TBP | CACGAAGTGCAATGGTCTTT | 1790 | G6PD | CAGGTCCAGCTCCGACTCCT |
| 1665 | TBP | GTTTCGGGCACGAAGTGCAA | 1791 | G6PD | AGGTCCAGCTCCGACTCCTC |
| 1666 | TBP | GGGATTATATTCGGCGTTTC | 1792 | G6PD | GGTCCAGCTCCGACTCCTCG |
| 1667 | TBP | TGGGATTATATTCGGCGTTT | 1793 | G6PD | TCGGGGTTGAAGAACATGCC |
| 1668 | TBP | TCTAACCTTGGGATTATATT | 1794 | G6PD | GAAGAACATGCCCGGCTTCT |
| 1669 | TBP | ATTAAAATAGATCTAACCTT | 1795 | G6PD | CGGCTTCTTGGTCATCATCT |
| 1670 | TBP | AAAATCAGTGCCGTGGTTCG | 1796 | G6PD | GGTCATCATCTTGGTGTACA |
| 1671 | TBP | AGAACTGAAAATCAGTGCCG | 1797 | G6PD | CTTGGTGTACACGGCCTCGT |
| 1672 | TBP | AATTTCTTACGGCTACCTCT | 1798 | G6PD | TTGGTGTACACGGCCTCGTT |
| 1673 | TBP | AGTCTGGACTGTTCTTCACT | 1799 | G6PD | CGGCCTCGTTGGGCTGCACG |
| 1674 | TBP | ATATTTTCTTGCTGCCAGTC | 1800 | G6PD | GCTCGTTGCGCTTGCACTGC |
| 1675 | TBP | TTGAAGTCCAAGAACTTAGC | 1801 | G6PD | CGTTGCGCTTGCACTGCTGG |
| 1676 | TBP | ACAAGGCCTTCTAACCTTAT | 1802 | G6PD | CTGCTGGTGGAAGATGTCGC |
| 1677 | TBP | ATTGTTGGTGGGTGAGCACA | 1803 | G6PD | AGATGTCGCCGGCCACATCA |
| 1678 | TBP | TTACCTACTAAATTGTTGGT | 1804 | G6PD | ATGGAACTGCAGCCTCACCT |
| 1679 | TBP | CTTACCTACTAAATTGTTGG | 1805 | G6PD | CCTCGGCCTTGCGCTCGTTC |
| 1680 | TBP | AGACTTACCTACTAAATTGT | 1806 | G6PD | CTCGGCCTTGCGCTCGTTCA |
| 1681 | TBP | ATTAAACCAGGAAATAACTC | 1807 | G6PD | TCAGGGCCTTGCCGCAGCGC |
| 1682 | TBP | ATCATTCTGTAGATTAAACC | 1808 | G6PD | CTTGCCGCAGCGCAGGATGA |
| 1683 | TBP | AAAATAAGGAGAACAATTCT | 1809 | G6PD | TTGCCGCAGCGCAGGATGAA |
| 1684 | TBP | CTTTTCCAGAAACAAAAATA | 1810 | G6PD | GTATGAGGGCACCTACAAGT |
| 1685 | TBP | TCCTGAATCCCTTTAGAATA | 1811 | G6PD | AGTATGAGGGCACCTACAAG |
| 1686 | TBP | TTCCTGAATCCCTTTAGAAT | 1812 | G6PD | AGAGTGGGTTTCCAGTATGA |
| 1687 | E2F4 | CTCACTCCCACTGCTGTCCC | 1813 | G6PD | GAGAGTGGGTTTCCAGTATG |
| 1688 | E2F4 | CCCTGGCAGTGCAGAAGTCC | 1814 | G6PD | GACGAGCTGATGAAGAGAGT |
| 1689 | E2F4 | CCTGGCAGTGCAGAAGTCCA | 1815 | G6PD | AGACGAGCTGATGAAGAGAG |
| 1690 | E2F4 | CAGTGCAGAAGTCCAGGGAA | 1816 | G6PD | CTCCAGCCGAGGCCCCACGG |
| 1691 | E2F4 | GCAGAAGTCCAGGGAATGGC | 1817 | G6PD | CACCCGTCACTCTCCAGCCG |
| 1692 | E2F4 | GGCCCAGCAGCTGAGATCAC | 1818 | G6PD | CCATCCCCTATATTTATGGC |
| 1693 | E2F4 | GGGGCTATCATTGTAGTGAG | 1819 | G6PD | AAGCCCATCCCCTATATTTA |
| 1694 | E2F4 | GCTATCATTGTAGTGAGTGG | 1820 | G6PD | ACTGCTGCACCAGATTGAGC |
| 1695 | E2F4 | ATTGTAGTGAGTGGCGGCCC | 1821 | G6PD | GCGACGAGCTCCGTGAGGCC |
| 1696 | E2F4 | TTGTAGTGAGTGGCGGCCCT | 1822 | G6PD | CCTCAGCGACGAGCTCCGTG |
| 1697 | E2F4 | CGGCCCTGGGACTGATAGCA | 1823 | G6PD | GCCAGATGCACTTCGTGCGC |
| 1698 | E2F4 | GGGACTGATAGCAAGGACAG | 1824 | G6PD | TCATCCTGGACGTCTTCTGC |
| 1699 | E2F4 | TGAGCTCAGTTCACTCCCAC | 1825 | G6PD | CTCATCCTGGACGTCTTCTG |
| 1700 | E2F4 | GAGCTCAGTTCACTCCCACT | 1826 | G6PD | CGCCTATGAGCGCCTCATCC |
| 1701 | E2F4 | CCCACTGGGCCCAACAACAC | 1827 | G6PD | GACCTACGGCAACAGATACA |
| 1702 | E2F4 | GCCCAACAACACTGGACACC | 1828 | G6PD | TCGGAGCTGGACCTGACCTA |
| 1703 | E2F4 | ACTGCAGTCTTCTGCCCTGC | 1829 | G6PD | CAACCCCGAGGAGTCGGAGC |
| 1704 | E2F4 | AGTAACAGCAGCAGTTCGTC | 1830 | G6PD | GTTCTTCAACCCCGAGGAGT |
| 1705 | E2F4 | TACCTCCTTTGAGCCCATCA | 1831 | G6PD | GGGCATGTTCTTCAACCCCG |
| 1706 | E2F4 | CCCATCAAGGCAGACCCCAC | 1832 | G6PD | AAGATGATGACCAAGAAGCC |
| 1707 | E2F4 | ATCAAGGCAGACCCCACAGG | 1833 | G6PD | CAAGATGATGACCAAGAAGC |
| 1708 | E2F4 | GAAATCTTTGATCCCACACG | 1834 | G6PD | GATCCGCGTGCAGCCCAACG |
| 1709 | E2F4 | TCTTTGATCCCACACGAGGT | 1835 | G6PD | GCAGTGCAAGCGCAACGAGC |
| 1710 | E2F4 | ATTCCCAGAGTGCATGAGCT | 1836 | G6PD | CTGCAGTTCCATGATGTGGC |
| 1711 | E2F4 | GTGCATGAGCTCGGAGCTGC | 1837 | G6PD | GAGGCTGCAGTTCCATGATG |
| 1712 | E2F4 | GAGGAGTTGATGTCCTCAGA | 1838 | G6PD | ACGAGCGCAAGGCCGAGGTG |
| 1713 | E2F4 | GAGTTGATGTCCTCAGAAGG | 1839 | G6PD | CCTGAACGAGCGCAAGGCCG |
| 1714 | E2F4 | AGTTGATGTCCTCAGAAGGT | 1840 | G6PD | CAAGGCCCTGAACGAGCGCA |
| 1715 | E2F4 | GCTTCGTCTTTCTCCACCCC | 1841 | G6PD | CTTCATCCTGCGCTGCGGCA |
| 1716 | E2F4 | CTTCGTCTTTCTCCACCCCC | 1842 | G6PD | GTGCCCTTCATCCTGCGCTG |
| 1717 | E2F4 | CCACGATTATATCTACAACC | 1843 | G6PD | AGAATGAGAGGTGGGATGGT |
| 1718 | E2F4 | TACAACCTGGACGAGAGTGA | 1844 | G6PD | GTGGAGAATGAGAGGTGGGA |
| 1719 | E2F4 | GCACTGCCAGGGACAGCAGT | 1845 | KIF11 | CTTAATGAAACCATAAAAAT |
| 1720 | E2F4 | TGCACTGCCAGGGACAGCAG | 1846 | KIF11 | GACTAAGCTTAATTGCTTTC |
| 1721 | E2F4 | CCTGGACTTCTGCACTGCCA | 1847 | KIF11 | GCTTAATTGCTTTCTGGAAC |
| 1722 | E2F4 | CCCTGGACTTCTGCACTGCC | 1848 | KIF11 | TCTGGAACAGGATCTGAAAC |
| 1723 | E2F4 | CTGCTGGGCCAGCCATTCCC | 1849 | KIF11 | CTGAAACTGGATATCCCAAC |
| 1724 | E2F4 | TGTCCTTGCTATCAGTCCCA | 1850 | KIF11 | TTAAAGGTACGACACCACAG |
| 1725 | E2F4 | CTGTCCTTGCTATCAGTCCC | 1851 | KIF11 | TTATTTATACCCATCAACAC |
| 1726 | E2F4 | CCAGTGTTGTTGGGCCCAGT | 1852 | KIF11 | ATCTCCTTGATCAGCTGAAA |
| 1727 | E2F4 | TCCAGTGTTGTTGGGCCCAG | 1853 | KIF11 | CAACAAAGAAGAGACAATTC |
| 1728 | E2F4 | GCCGGGTGTCCAGTGTTGTT | 1854 | KIF11 | TTAGGATGTGGATGTAGAAG |
| 1729 | E2F4 | GGCCGGGTGTCCAGTGTTGT | 1855 | KIF11 | GGATGTAGAAGAGGCAGTTC |
| 1730 | E2F4 | AGCAGGGCAGAAGACTGCAG | 1856 | KIF11 | GATGTAGAAGAGGCAGTTCT |
| 1731 | E2F4 | GCTGCTGCTGCTGTCCAGCA | 1857 | KIF11 | ATGTAGAAGAGGCAGTTCTG |
| 1732 | E2F4 | GGAGGTAGAAGGGTTGGGTC | 1858 | KIF11 | CAAGAGCCATCTGTAGATGC |
| 1733 | E2F4 | TGGGCTCAAAGGAGGTAGAA | 1859 | KIF11 | GCCATCTGTAGATGCTGGTG |
| 1734 | E2F4 | ATGGGCTCAAAGGAGGTAGA | 1860 | KIF11 | GGTGTGGATTGTTCATCAAT |
| 1735 | E2F4 | TGCCTTGATGGGCTCAAAGG | 1861 | KIF11 | GTGGATTGTTCATCAATTGG |
| 1736 | E2F4 | GTCTGCCTTGATGGGCTCAA | 1862 | KIF11 | TGGATTGTTCATCAATTGGC |
| 1737 | E2F4 | CCTGTGGGGTCTGCCTTGAT | 1863 | KIF11 | GGATTGTTCATCAATTGGCG |
| 1738 | E2F4 | ACCTGTGGGGTCTGCCTTGA | 1864 | KIF11 | TGGCGGGGTTCCATTTTTCC |
| 1739 | E2F4 | GCAGGTACTCACCACCTGTG | 1865 | KIF11 | CCACAGCATAAAAAATCACA |
| 1740 | E2F4 | GGCAGGTACTCACCACCTGT | 1866 | KIF11 | GGAAAAGACAAAGAAAACAG |
| 1741 | E2F4 | GGGCAGGTACTCACCACCTG | 1867 | KIF11 | AAACAGAGGCATTAACACAC |
| 1742 | E2F4 | AGATTTCTGACAGCTCTTTG | 1868 | KIF11 | GAGGCATTAACACACTGGAG |
| 1743 | E2F4 | AAGATTTCTGACAGCTCTTT | 1869 | KIF11 | CACACTGGAGAGGTCTAAAG |
| 1744 | E2F4 | AAAGATTTCTGACAGCTCTT | 1870 | KIF11 | GGAAGAAACTACAGAGCACT |
| 1745 | E2F4 | TGCAGCAGCCTACCTCGTGT | 1871 | KIF11 | CTTAGTCAAACCAATTTTTA |
| 1746 | E2F4 | ATGCAGCAGCCTACCTCGTG | 1872 | KIF11 | TCTCTTTTAAAGTACCTGTT |
| 1747 | E2F4 | GCTCCGAGCTCATGCACTCT | 1873 | KIF11 | TTCTCTTTTAAAGTACCTGT |
| 1748 | E2F4 | AGCTCCGAGCTCATGCACTC | 1874 | KIF11 | TATAAATAACTTTTCCTCTG |
| 1749 | E2F4 | CCAGGGCCACCCACCTTCTG | 1875 | KIF11 | CAGTTCTTACCAGTGTTGAT |
| 1750 | E2F4 | TGGAGAAAGACGAAGCAGAG | 1876 | KIF11 | TCAGTTCTTACCAGTGTTGA |
| 1751 | E2F4 | GTGGAGAAAGACGAAGCAGA | 1877 | KIF11 | TGATCAAGGAGATGTTCACG |
| 1752 | E2F4 | GGTGGAGAAAGACGAAGCAG | 1878 | KIF11 | GTTTCCTTTTCAGCTGATCA |
| 1753 | E2F4 | TAATCGTGGTCTCCCGGGGG | 1879 | KIF11 | TTTAGCATCATTAACAGCTC |
| 1754 | E2F4 | ATATAATCGTGGTCTCCCGG | 1880 | KIF11 | ACAGATGGCTCTTGACTTAG |
| 1755 | E2F4 | GATATAATCGTGGTCTCCCG | 1881 | KIF11 | TCCACACCAGCATCTACAGA |
| 1756 | E2F4 | AGATATAATCGTGGTCTCCC | 1882 | KIF11 | ATATGACATACCTGGAAAAA |
| 1757 | E2F4 | TAGATATAATCGTGGTCTCC | 1883 | KIF11 | AGGTTGATCTGGGCTCGCAG |
| 1758 | E2F4 | CCAGGTTGTAGATATAATCG | 1884 | KIF11 | AGTGAATTAAAGGTTGATCT |
| 1759 | E2F4 | AGACACCTTCACTCTCGTCC | 1885 | KIF11 | AAGTGAATTAAAGGTTGATC |
| 1760 | E2F4 | TGAGAACAGGCACATCAAAG | | | |

It will be understood that the exemplary gRNAs disclosed herein are provided to illustrate non-limiting embodiments embraced by the present disclosure. Additional suitable gRNA sequences will be apparent to the skilled artisan based on the present disclosure, and the disclosure is not limited in this respect.

### Target Cells

Methods of the disclosure can be used to edit the genome of any cell. In certain embodiments, the target cell is a stem cell, e.g., an iPS or ES cell. In certain embodiments, the target cell is an iPS- or ES-derived cell, where the genetic modification is made at any stage during the reprogramming process from donor cell to iPSC, during the iPSC stage, and/or at any stage of the process of differentiating the iPSC or ESC to a specialized cell, or even up to or at the final specialized cell state. In certain embodiments, the target cell can be an iPSC-derived B cell, where the genetic modification is made at any stage during the reprogramming process from donor cell to iPSC, during the iPSC stage, and/or at any stage of the process of differentiating the iPSC to a B cell.

In certain embodiments, a target cell is one or more of a long-term hematopoietic stem cell, a short term hematopoietic stem cell, a multipotent progenitor cell, a lineage restricted progenitor cell, a lymphoid progenitor cell, an induced pluripotent stem (iPS) cell, an embryonic stem cell, a fibroblast, or a B cell, e.g., a progenitor B cell, a Pre B cell, a Pro B cell, an immature B cell, a transitional B cell, a mature B cell, a naïve B cell, a memory B cell, a marginal zone B cell, a follicular B cell, a germinal center B cell, or a plasma B cell.

In some embodiments, a target cell is a circulating blood cell, e.g., lymphoid progenitor (LP) cell or hematopoietic stem/progenitor cell (HSC). In some embodiments, a target cell is one or more of a bone marrow cell (e.g., LP cell, HSC, multipotent progenitor (MPP) cell, or mesenchymal stem cell). In some embodiments, a target cell is a lymphoid progenitor cell, e.g., a common lymphoid progenitor (CLP) cell. In some embodiments, a target cell is one or more of a hematopoietic stem/progenitor cell (e.g., a long term HSC (LT-HSC), short term HSC (ST-HSC), MPP cell, or lineage restricted progenitor (LRP) cell). In certain embodiments, the target cell is a CD34⁺ cell, CD34⁺CD90⁺ cell, CD34⁺CD38⁻ cell, CD34⁺CD90⁺CD49f⁺CD38⁻CD45RA⁻ cell, CD105⁺ cell, CD31⁺, or CD133⁺ cell, or a CD34⁺CD90⁺CD133⁺ cell. In some embodiments, a target cell is one or more of an umbilical cord blood CD34⁺ HSPC, umbilical cord venous endothelial cell, umbilical cord arterial endothelial cell, amniotic fluid CD34⁺ cell, amniotic fluid endothelial cell, placental endothelial cell, or placental hematopoietic CD34⁺ cell. In some embodiments, a target cell is one or more of a mobilized peripheral blood hematopoietic CD34⁺ cell (after the subject is treated with a mobilization agent, e.g., G-CSF or Plerixafor).

In certain embodiments, a target cell is a primary cell, e.g., a cell isolated from a human subject. In certain embodiments, a target cell is an immune cell, e.g., a primary immune cell isolated from a human subject. In certain embodiments, a target cell is part of a population of cells isolated from a subject, e.g., a human subject. In some embodiments, the population of cells comprises a population of immune cells isolated from a subject. In some embodiments, the population of cells comprises tumor infiltrating lymphocytes (TILs), e.g., TILs isolated from a human subject. In some embodiments, a target cell is isolated from a healthy subject, e.g., a healthy human donor. In some embodiments, a target cell is isolated from a subject having a disease or illness, e.g., a human patient in need of a treatment.

In certain embodiments, a target cell is an immune cell, e.g., a primary immune cell, e.g., a B cell, a progenitor B cell, a Pre B cell, a Pro B cell, an immature B cell, a transitional B cell, a mature B cell, a naïve B cell, a memory B cell, a marginal zone B cell, a follicular B cell, a germinal center B cell, a plasmablast, or a plasma B cell. In some embodiments, a target cell is a CD19⁺ B cell, a CD19⁺ Pro B cell, a CD19⁺ Pre B cell, a CD19⁺ immature B cell, a CD19⁺ transitional B cell, a CD19⁺ mature B cell, a CD19⁺ naïve B cell, a CD19⁺ memory B cell, a CD19⁺ marginal zone B cell, a CD19⁺ follicular B cell, a CD19⁺ germinal center B cell, or a CD19⁺ plasmablast. In some embodiments, a target cell is a CD20⁺ B cell, a CD20⁺ immature B cell, a CD20⁺ transitional B cell, a CD20⁺ mature B cell, a CD20⁺ naïve B cell, a CD20⁺ memory B cell, a CD20⁺ marginal zone B cell, a CD20⁺ follicular B cell, or a CD20⁺ germinal center B cell. In some embodiments, a target cell is a CD40⁺ B cell, a CD40⁺ Pre B cell, a CD40⁺ immature B cell, a CD40⁺ transitional B cell, a CD40⁺ mature B cell, a CD40⁺ naïve B cell, a CD40⁺ memory B cell, a CD40⁺ marginal zone B cell, a CD40⁺ follicular B cell, a CD40⁺ germinal center B cell, or a CD40⁺ plasma B cell.

### Stem Cells

Methods of the disclosure can be used with stem cells. Stem cells are typically cells that have the capacity to produce unaltered daughter cells (self-renewal; cell division produces at least one daughter cell that is identical to the parent cell) and to give rise to specialized cell types (potency). Stem cells include, but are not limited to, embryonic stem (ES) cells, embryonic germ (EG) cells, germline stem (GS) cells, human mesenchymal stem cells (hMSCs), adipose tissue-derived stem cells (ADSCs), multipotent adult progenitor cells (MAPCs), multipotent adult germline stem cells (maGSCs) and unrestricted somatic stem cell (USSCs). Generally, stem cells can divide without limit. After division, the stem cell may remain as a stem cell, become a precursor cell, or proceed to terminal differentiation. A precursor cell is a cell that can generate a fully differentiated functional cell of at least one given cell type. Generally, precursor cells can divide. After division, a precursor cell can remain a precursor cell, or may proceed to terminal differentiation.

Pluripotent stem cells are generally known in the art. The present disclosure provides technologies (e.g., systems, compositions, methods, etc.) related to pluripotent stem cells. In some embodiments, pluripotent stem cells are stem cells that: (a) are capable of inducing teratomas when transplanted in immunodeficient (SCID) mice; (b) are capable of differentiating to cell types of all three germ layers (e.g., can differentiate to ectodermal, mesodermal, and endodermal cell types); and/or (c) express one or more markers of embryonic stem cells (e.g., human embryonic stem cells express Oct-4, alkaline phosphatase, SSEA-3 surface antigen, SSEA-4 surface antigen, nanog, TRA-1-60, TRA-1-81, Sox-2, REX1, etc.). In some aspects, human pluripotent stem cells do not show expression of differentiation markers. In some embodiments, ES cells and/or iPSCs edited using methods of the disclosure maintain their pluripotency, e.g., (a) are capable of inducing teratomas when transplanted in immunodeficient (SCID) mice; (b) are capable of differentiating to cell types of all three germ layers, e.g., can differentiate to ectodermal, mesodermal, and endodermal cell types); and/or (c) express one or more markers of embryonic stem cells.

In some embodiments, ES cells (e.g., human ES cells) can be derived from the inner cell mass of blastocysts or morulae. In some embodiments, ES cells can be isolated from one or more blastomeres of an embryo, e.g., without destroying the remainder of the embryo. In some embodiments, ES cells can be produced by somatic cell nuclear transfer. In some embodiments, ES cells can be derived from fertilization of an egg cell with sperm or DNA, nuclear transfer, parthenogenesis, or by means to generate ES cells, e.g., with homozygosity in the HLA region. In some embodiments, human ES cells can be produced or derived from a zygote, blastomeres, or blastocyst-staged mammalian embryo produced by the fusion of a sperm and egg cell, nuclear transfer, parthenogenesis, or the reprogramming of chromatin and subsequent incorporation of the reprogrammed chromatin into a plasma membrane to produce an embryonic cell. Exemplary human ES cells are known in the art and include, but are not limited to, MAO1, MAO9, ACT-4, No. 3, H1, H7, H9, H14 and ACT30 ES cells. In some embodiments, human ES cells, regardless of their source or the particular method used to produce them, can be identified based on, e.g., (i) the ability to differentiate into cells of all three germ layers, (ii) expression of at least Oct-4 and alkaline phosphatase, and/or (iii) ability to produce teratomas when transplanted into immunocompromised animals. In some embodiments, ES cells have been serially passaged as cell lines.

### iPS Cells

Induced pluripotent stem cells (iPSC) are a type of pluripotent stem cell artificially derived from a non-pluripotent cell, such as an adult somatic cell (e.g., a fibroblast cell or other suitable somatic cell), by inducing expression of certain genes. iPSCs can be derived from any organism, such as a mammal. In some embodiments, iPSCs are produced from mice, rats, rabbits, guinea pigs, goats, pigs, cows, non-human primates or humans. iPSCs are similar to ES cells in many respects, such as the expression of certain stem cell genes and proteins, chromatin methylation patterns, doubling time, embryoid body formation, teratoma formation, viable chimera formation, potency and/or differentiability. Various suitable methods for producing iPSCs are known in the art. In some embodiments, iPSCs can be derived by transfection of certain stem cell-associated genes (such as Oct-3/4 (Pouf51) and Sox-2) into non-pluripotent cells, such as adult fibroblasts. Transfection can be achieved through viral vectors, such as retroviruses, lentiviruses, or adenoviruses. Additional suitable reprogramming methods include the use of vectors that do not integrate into the genome of the host cell, e.g., episomal vectors, or the delivery of reprogramming factors directly via encoding RNA or as proteins has also been described. For example, cells can be transfected with Oct-3/4, Sox-2, Klf4, and/or c-Myc using a retroviral system or with Oct-4, Sox-2, NANOG, and/or LIN28 using a lentiviral system. After 3-4 weeks, small numbers of transfected cells begin to become morphologically and biochemically similar to pluripotent stem cells, and can be isolated through morphological selection, doubling time, or through a reporter gene and antibiotic selection. In one example, iPSCs from adult human cells are generated by the method described by Yu et al., Science 2007; 318(5854):1224 or Takahashi et al., Cell 2007; 131:861-72. Numerous suitable methods for reprogramming are known to those of skill in the art, and the present disclosure is not limited in this respect.

In some embodiments, a target cell for the editing and cargo integration methods described herein is an iPSC, wherein the edited iPSC is then differentiated, e.g., into an iPSC-derived immune cell. In some embodiments, the differentiated cell is an iPSC-derived B cell.

A variety of cell types can be used as a donor cell that can be subjected to reprogramming, differentiation, and/or genetic engineering strategies described herein. For example, the donor cell can be a pluripotent stem cell or a differentiated cell, e.g., a somatic cell. In some embodiments, donor cells are manipulated (e.g., subjected to reprogramming, differentiation, and/or genetic engineering) to generate B cells described herein.

A donor cell can be from any suitable organism. For example, in some embodiments, the donor cell is a mammalian cell, e.g., a human cell or a non-human primate cell. In some embodiments, the donor cell is a somatic cell. In some embodiments, the donor cell is a stem cell or progenitor cell. In certain embodiments, the donor cell is not or was not part of a human embryo and its derivation does not involve destruction of a human embryo.

### Methods of Characterization

Methods of characterizing cells including characterizing cellular phenotype are known to those of skill in the art. In some embodiments, one or more such methods may include, but not be limited to, for example, morphological analyses and flow cytometry. Cellular lineage and identity markers are known to those of skill in the art. One or more such markers may be combined with one or more characterization methods to determine a composition of a cell population or phenotypic identity of one or more cells. For example, in some embodiments, cells of a particular population will be characterized using flow cytometry (for example, see Ye Li et al., Cell Stem Cell. 2018 Aug. 2; 23(2): 181-192.e5). In some such embodiments, a sample of a population of cells will be evaluated for presence and proportion of one or more cell surface markers and/or one or more intracellular markers. As will be understood by those of skill in the art, such cell surface markers may be representative of different lineages. For example, pluripotent cells may be identified by one or more of any number of markers known to be associated with such cells, such as, for example, CD34. Further, in some embodiments, cells may be identified by markers that indicate some degree of differentiation. Such markers will be known to one of skill in the art. For example, in some embodiments, markers of differentiated cells may include those associated with differentiated hematopoietic cells such as, e.g., CD43, CD45 (differentiated hematopoietic cells). In some embodiments, markers of cells may be associated with B cell phenotypes such as, e.g., cluster of differentiation 19 (CD19), cluster of differentiation 20 (CD20), and cluster of differentiation 40 (CD40).

### Methods of Use

A variety of diseases, disorders and/or conditions may be treated through use of cells provided by the present disclosure. For example, in some embodiments, a disease, disorder and/or condition may be treated by introducing genetically modified or engineered cells as described herein (e.g., genetically modified B cells) to a subject. Examples of diseases that may be treated include, but are not limited to, cancer, e.g., solid tumors, e.g., of the brain, prostate, breast, lung, colon, uterus, skin, liver, bone, pancreas, ovary, testes, bladder, kidney, head, neck, stomach, cervix, rectum, larynx, or esophagus; and hematological malignancies, e.g., acute and chronic leukemias, lymphomas, multiple myeloma and myelodysplastic syndromes.

In some embodiments, the present disclosure provides methods of treating a subject in need thereof by administering to the subject a composition comprising any of the cells described herein. In some embodiments, a therapeutic agent or composition may be administered before, during, or after the onset of a disease, disorder, or condition (including, e.g., an injury). In some embodiments, the present disclosure provides any of the cells described herein for use in the preparation of a medicament. In some embodiments, the present disclosure provides any of the cells described herein for use in the treatment of a disease, disorder, or condition, that can be treated by a cell therapy.

In particular embodiments, the subject has a disease, disorder, or condition, that can be treated by a cell therapy. In some embodiments, a subject in need of cell therapy is a subject with a disease, disorder and/or condition, whereby a cell therapy, e.g., a therapy in which a composition comprising a cell described herein, is administered to the subject, whereby the cell therapy treats at least one symptom associated with the disease, disorder, and/or condition. In some embodiments, a subject in need of cell therapy includes, but is not limited to, a candidate for bone marrow or stem cell transplant, a subject who has received chemotherapy or irradiation therapy, a subject who has or is at risk of having cancer, e.g., a cancer of hematopoietic system, a subject having or at risk of developing a tumor, e.g., a solid tumor, and/or a subject who has or is at risk of having a viral infection or a disease associated with a viral infection.

### Pharmaceutical Compositions

In some embodiments, the present disclosure provides pharmaceutical compositions comprising one or more genetically modified or engineered cells described herein, e.g., a genetically modified B cell described herein. In some embodiments, a pharmaceutical composition further comprises a pharmaceutically acceptable excipient.

As one of ordinary skill in the art would understand, both autologous and allogeneic cells can be used in adoptive cell therapies. Autologous cell therapies generally have reduced infection, low probability for GVHD, and rapid immune reconstitution relative to other cell therapies. Allogeneic cell therapies generally have an immune mediated graft-versus-malignancy (GVM) effect, and low rate of relapse relative to other cell therapies. Based on the specific condition(s) of the subject in need of the cell therapy, one of ordinary skill in the art would be able to determine which specific type of therapy(ies) to administer.

In some embodiments, a pharmaceutical composition comprises pluripotent stem cell-derived hematopoietic lineage cells that are allogeneic to a subject. In some embodiments, a pharmaceutical composition comprises pluripotent stem cell-derived hematopoietic lineage cells that are autologous to a subject. For autologous transplantation, the isolated population of pluripotent stem cell-derived hematopoietic lineage cells can be either a complete or partial HLA-match with the subject being treated. In some embodiments, the pluripotent stem cell-derived hematopoietic lineage cells are not HLA-matched to a subject.

In some embodiments, pluripotent stem cell-derived hematopoietic lineage cells can be administered to a subject without being expanded ex vivo or in vitro prior to administration. In particular embodiments, an isolated population of derived hematopoietic lineage cells is modulated and treated ex vivo using one or more agents to obtain immune cells with improved therapeutic potential. In some embodiments, the modulated population of derived hematopoietic lineage cells can be washed to remove the treatment agent(s), and the improved population can be administered to a subject without further expansion of the population in vitro. In some embodiments, an isolated population of derived hematopoietic lineage cells is expanded prior to modulating the isolated population with one or more agents.

### Cancers

Any cancer can be treated using a cell or pharmaceutical composition described herein. Exemplary therapeutic targets of the present disclosure include cancer cells from the bladder, blood, bone, bone marrow, brain, breast, colon, esophagus, eye, gastrointestinal system, gum, head, kidney, liver, lung, nasopharynx, neck, ovary, prostate, skin, stomach, testis, tongue, or uterus. In addition, a cancer may specifically be of the following non-limiting histological type: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer is a breast cancer. In some embodiments, the cancer is colon cancer. In some embodiments, the cancer is gastric cancer. In some embodiments, the cancer is RCC. In another embodiment, the cancer is non-small cell lung cancer (NSCLC).

In some embodiments, solid cancer indications that can be treated with cells described herein (e.g., cells modified using methods of the disclosure, e.g., genetically modified iNK cells), either alone or in combination with one or more additional cancer treatment modality, include: bladder cancer, hepatocellular carcinoma, prostate cancer, ovarian/uterine cancer, pancreatic cancer, mesothelioma, melanoma, glioblastoma, HPV-associated and/or HPV-positive cancers such as cervical and HPV+ head and neck cancer, oral cavity cancer, cancer of the pharynx, thyroid cancer, gallbladder cancer, and soft tissue sarcomas. In some embodiments, hematological cancer indications that can be treated with cells described herein (e.g., cells modified using methods of the disclosure, e.g., genetically modified iNK cells), either alone or in combination with one or more additional cancer treatment modalities, include: ALL, CLL, NHL, DLBCL, AML, CML, and multiple myeloma (MM).

In some embodiments, examples of cellular proliferative and/or differentiative disorders of the lung that can be treated with cells described herein (e.g., cells modified using methods of the disclosure) include, but are not limited to, tumors such as bronchogenic carcinoma, including paraneoplastic syndromes, bronchioloalveolar carcinoma, neuroendocrine tumors, such as bronchial carcinoid, miscellaneous tumors, metastatic tumors, and pleural tumors, including solitary fibrous tumors (pleural fibroma) and malignant mesothelioma.

In some embodiments, examples of cellular proliferative and/or differentiative disorders of the breast that can be treated with cells described herein (e.g., cells modified using methods of the disclosure) include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

In some embodiments, examples of cellular proliferative and/or differentiative disorders involving the colon that can be treated with cells described herein (e.g., cells modified using methods of the disclosure) include, but are not limited to, tumors of the colon, such as non-neoplastic polyps, adenomas, familial syndromes, colorectal carcinogenesis, colorectal carcinoma, and carcinoid tumors.

In some embodiments, examples of cancers or neoplastic conditions, in addition to the ones described above that can be treated with cells described herein (e.g., cells modified using methods of the disclosure), include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

In some embodiments, cells described herein (e.g., cells modified using methods of the disclosure) are used in combination with one or more cancer treatment modalities. In some embodiments, other cancer treatment modalities include, but are not limited to: chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfanide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegall (see, e.g., Agnew, Chem. Intl. Ed. Engl., 1994; 33:183-186); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANET™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; cyclosporine, sirolimus, rapamycin, rapalogs, ibandronate; topoisomerase inhibitor RFS 2000; difluoromethylornithine (DMFO); retinoids such as retinoic acid; CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU, leucovovin; anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); aptamers, described for example in U.S. Pat. No. 6,344,321, which is herein incorporated by reference in its entirety; anti HGF monoclonal antibodies (e.g., AV299 from Aveo, AMG102, from Amgen); truncated mTOR variants (e.g., CGEN241 from Compugen); protein kinase inhibitors that block mTOR induced pathways (e.g., ARQ197 from Arqule, XL880 from Exelexis, SGX523 from SGX Pharmaceuticals, MP470 from Supergen, PF2341066 from Pfizer); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); COX-2 inhibitors such as celecoxib (CELEBREX®; 4-(5-(4-methylphenyl)-3-(trifluoromethyl)-1H-pyrazol-1-yl) benzenesulfonamide; and pharmaceutically acceptable salts, acids or derivatives of any of the above.

In some embodiments, cells described herein (e.g., cells modified using methods of the disclosure) are used in combination with one or more cancer treatment modalities that facilitate the induction of antibody dependent cellular cytotoxicity (ADCC) (see e.g., Janeway's Immunobiology by K. Murphy and C. weaver). In some embodiments, such a cancer treatment modality is an antibody, e.g., an antibody described herein. In some embodiments, cells described herein (e.g., cells modified using methods of the disclosure) are used in combination with one or more cancer treatment modalities that facilitate the induction of antibody dependent cellular cytotoxicity (ADCC), wherein the cancer treatment modality is an antibody or appropriate fragment thereof targeting CD20, TNFα, HER2, CD52, IgE, EGFR, VEGF-A, ITGA4, CTLA-4, CD30, VEGFR2, α4β7 integrin, CD19, CD3, PD-1, GD2, CD38, SLAMF7, PDGFRα, PD-L1, CD22, CD33, IFNγ, CD79β, or any combination thereof.

In some embodiments, cells described herein are utilized in combination with checkpoint inhibitors. Examples of suitable combination therapy checkpoint inhibitors include, but are not limited to, antagonists of PD-1 (Pdcdl, CD279), PDL-1 (CD274), TIM-3 (Havcr2), TIGIT (WUCAM and Vstm3), LAG-3 (Lag3, CD223), CTLA-4 (Ctla4, CD152), 2B4 (CD244), 4-1BB (CD137), 4-1BBL (CD137L), A2aR, BATE, BTLA, CD39 (Entpdl), CD47, CD73 (NT5E), CD94, CD96, CD160, CD200, CD200R, CD274, CEACAM1, CSF-1R, Foxp1, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/HLA-E, inhibitory KIR (for example, 2DL1, 2DL2, 2DL3, 3DL1, and 3DL2), or any suitable combination thereof.

In some embodiments, the antagonist inhibiting any of the above checkpoint molecules is an antibody. In some embodiments, the checkpoint inhibitory antibodies may be murine antibodies, human antibodies, humanized antibodies, a camel Ig, a shark heavychain-only antibody (VNAR), Ig NAR, chimeric antibodies, recombinant antibodies, or antibody fragments thereof. Non-limiting examples of antibody fragments include Fab, Fab′, F(ab)′2, F(ab)′3, Fv, single chain antigen binding fragments (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody, which may be more cost-effective to produce, more easily used, or more sensitive than the whole antibody. In some embodiments, the one, or two, or three, or more checkpoint inhibitors comprise at least one of atezolizumab (anti-PDL1 mAb), avelumab (anti-PDL1 mAb), durvalumab (anti-PDL1 mAb), tremelimumab (anti-CTLA4 mAb), ipilimumab (anti-CTLA4 mAb), IPH4102 (anti-KIR), IPH43 (anti-MICA), IPH33 (anti-TLR3), lirimumab (anti-KIR), monalizumab (anti-NKG2A), nivolumab (anti-PD1 mAb), pembrolizumab (anti-PD 1 mAb), and any derivatives, functional equivalents, or biosimilars thereof.

In some embodiments, the antagonist inhibiting any of the above checkpoint molecules is microRNA-based, as many miRNAs are found as regulators that control the expression of immune checkpoints (Dragomir et al., Cancer Biol Med. 2018, 15(2): 103-115). In some embodiments, the checkpoint antagonistic miRNAs include, but are not limited to, miR-28, miR-15/16, miR-138, miR-342, miR-20b, miR-21, miR-130b, miR-34a, miR-197, miR-200c, miR-200, miR-17-5p, miR-570, miR-424, miR-155, miR-574-3p, miR-513, miR-29c, and/or any suitable combination thereof.

In some embodiments, cells described herein (e.g., cells modified using methods of the disclosure) are used in combination with one or more cancer treatment modalities such as exogenous interleukin (IL) dosing. In some embodiments, an exogenous IL provided to a patient is IL-15. In some embodiments, systemic IL-15 dosing when used in combination with cells described herein is reduced when compared to standard dosing concentrations (see e.g., Waldmann et al., IL-15 in the Combination Immunotherapy of Cancer. Front. Immunology, 2020).

Other compounds that are effective in treating cancer are known in the art and described herein that are suitable for use with the compositions and methods of the present disclosure as additional cancer treatment modalities are described, for example, in the “Physicians' Desk Reference, 62nd edition. Oradell, N.J.: Medical Economics Co., 2008”, Goodman & Gilman's “The Pharmacological Basis of Therapeutics, Eleventh Edition. McGraw-Hill, 2005”, “Remington: The Science and Practice of Pharmacy, 20th Edition. Baltimore, Md.: Lippincott Williams & Wilkins, 2000,” and “The Merck Index, Fourteenth Edition. Whitehouse Station, N.J.: Merck Research Laboratories, 2006”, incorporated herein by reference in relevant parts.

In some embodiments, a gene product of interest described herein is a recombinant polypeptide (e.g., a recombinant antibody, e.g., a therapeutic antibody), and a modified cell described herein can be used to produce such recombinant polypeptide. For example, in some embodiments, the present disclosure provides methods of producing a recombinant polypeptide (e.g., a recombinant antibody, e.g., a therapeutic antibody), comprising providing and/or generating a modified cell described herein (e.g., a cell modified to express a recombinant polypeptide (e.g., a recombinant antibody, e.g., a therapeutic antibody)), culturing the modified cell under conditions suitable for expression of the recombinant polypeptide (e.g., a recombinant antibody, e.g., a therapeutic antibody), and optionally harvesting the recombinant polypeptide (e.g., a recombinant antibody, e.g., a therapeutic antibody).

All publications, patents and patent applications cited herein, whether supra or infra, are hereby incorporated by reference in their entirety.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of is meant including, and limited to, whatever follows the phrase “consisting of:” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially” of indicates that the listed elements are required or mandatory, but that no other elements are optional and may or may not be present depending upon whether or not they affect the activity or action of the listed elements.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. The contents of database entries, e.g., NCBI nucleotide or protein database entries provided herein, are incorporated herein in their entirety. Where database entries are subject to change over time, the contents as of the filing date of the present application are incorporated herein by reference. Aspects of the embodiments can be modified, if necessary to employ concepts of the various patents, applications and publications to provide yet further embodiments.

The disclosure is further illustrated by the following examples. The examples are provided for illustrative purposes only. They are not to be construed as limiting the scope or content of the disclosure in any way.

### EXAMPLES

### Example 1: Screening of Guide RNAs for GAPDH

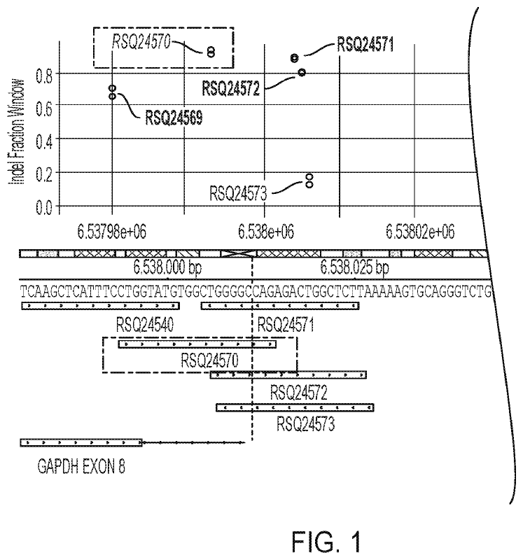

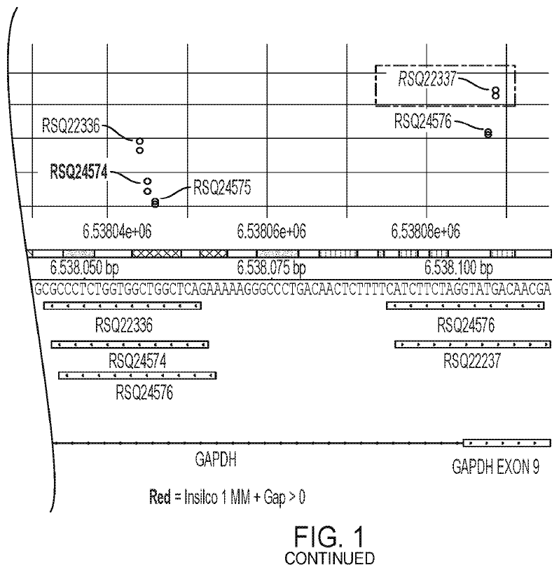

This example describes the screening of AsCpf1 (AsCas12a) guide RNAs that target the housekeeping gene GAPDH. GAPDH encodes Glyceraldehyde-3-Phosphate Dehydrogenase, an essential protein that catalyzes oxidative phosphorylation of glyceraldehyde-3-phosphate in the presence of inorganic phosphate and nicotinamide adenine dinucleotide (NAD), an important energy-yielding step in carbohydrate metabolism. The guide RNAs used in this analysis were all 41-mer RNA molecules with the following design: 5′-UAAUUUCUACUCUUGUAGAU-[21-mer targeting domain sequence]-3′ (SEQ ID NO: 90). For example, the guide RNA denoted RSQ22337 had the following sequence: 5′-UAAUUUCUACUCUUGUAGAUAUCUUCUAGGUAUGACAACGA-3′ (SEQ ID NO: 93) where the 21-mer targeting domain sequence is underlined. The guide RNAs with the targeting domain sequences shown in Table 14 were tested to determine how effective they were at editing GAPDH. Cas12a RNPs (RNPs having an engineered Cas12a (SEQ ID NO: 62)), containing each of these guide RNAs were transfected into iPSCs, and then editing levels were assayed three days after transfection (see e.g., Wong, K. G. et al. CryoPause: A New Method to Immediately Initiate Experiments after Cryopreservation of Pluripotent Stem Cells. Stem Cell Reports 9, 355-365 (2017)). The results are shown in FIG. 1 and FIG. 2. RSQ24570, RSQ24582, RSQ24589, RSQ24585, and RSQ22337 exhibited the greatest levels of measurable editing out of the GAPDH guides tested, editing approximately 70% or more of cells (about 92%, 89%, 88%, 87%, and 70%, respectively). It was observed that cells transfected with gRNAs targeting certain exonic regions yielded much lower amounts of isolatable genomic DNA (gDNA) for analyzing editing efficiency (at day 3 after transfection) when compared to cells transfected with gRNAs targeting intronic regions, indicating that that RNPs with certain exon-targeting gRNAs were cytotoxic to the cells. This suggested that cells edited with gRNAs targeting exonic regions could result in significant cell death due to the introduction of indels within GAPDH leading to expression of a non-functional GAPDH protein or a protein with insufficient function. It was postulated that it might be possible to use a rescue plasmid to repair the gRNA-mediated cleavage site in GAPDH while also knocking in a gene cargo of interest in frame with the repaired GAPDH via HDR, thereby rescuing those cells in which GAPDH is repaired and the cargo of interest is successfully integrated (as shown in FIG. 1 and FIG. 2). Those transfected cells that are edited (the majority of transfected cells, if a highly effective RNA-guided nucleases is used) but do not undergo HDR repair of GAPDH and do not integrate the cargo of interest die over time because they do not have a functioning GAPDH gene. Those cells carrying the cargo of interest would have an advantage due to a fully functioning GAPDH gene as the cells grow and divide, and these cells would be selected for over time. The expected end result would be a population of cells with a very high rate of cargo knock-in within the GAPDH locus.

The data in FIG. 2 suggested that while Cas12a RNP comprising RSQ22337 resulted in an editing level of approximately 70% at 3 days post-transfection, it caused slightly higher levels of toxicity than other exonic guides (RSQ24570, RSQ24582, RSQ24589, and RSQ24585) (see FIG. 2, only about 3.9 ng/μL of gDNA was isolated from edited cells). Thus, the actual editing efficiency was very likely significantly higher than 70%, as many cells had already died by 3 days post-transfection due to the lack of available rescue constructs and NHEJ forming toxic indels. As a result, RSQ22337 was chosen for further testing.

TABLE 14
Guide RNA sequences

| SEQ | | gRNA targeting domain sequence | |
|---|---|---|---|
| ID NO: | Name | (RNA) | Location |
| 94 | RSQ22336 | UGAGCCAGCCACCAGAGGGCG | Intron 8 |
| 95 | RSQ22337 | AUCUUCUAGGUAUGACAACGA | Intron 8/ Exon 9 (cut site |
| | | | in exon 9) |
| 96 | RSQ22338 | GCUACAGCAACAGGGUGGUGG | Exon 9 |
| 97 | RSQ24559 | CCAUAAUUUCCUUUCAAGGUG | Intron 7 |
| 98 | RSQ24560 | CUUUCAAGGUGGGGAGGGAGG | Intron 7 |
| 99 | RSQ24561 | AAGGUGGGGAGGGAGGUAGAG | Intron 7 |
| 100 | RSQ24562 | GCAGACCACAGUCCAUGCCAU | Exon 8 |
| 101 | RSQ24563 | CAGACCACAGUCCAUGCCAUC | Exon 8 |
| 102 | RSQ24564 | CCGGAGGGGCCAUCCACAGUC | Exon 8 |
| 103 | RSQ24565 | UAGACGGCAGGUCAGGUCCAC | Exon 8 |
| 104 | RSQ24566 | CUAGACGGCAGGUCAGGUCCA | Exon 8 |
| 105 | RSQ24567 | UCUAGACGGCAGGUCAGGUCC | Exon 8 |
| 106 | RSQ24568 | GCAGGUUUUUCUAGACGGCAG | Exon 8 |
| 107 | RSQ24569 | UCAAGCUCAUUUCCUGGUAUG | Exon 8 |
| 108 | RSQ24570 | CUGGUAUGUGGCUGGGGCCAG | Exon 8/ Intron 8 (cut site |
| | | | in intron 8) |
| 109 | RSQ24571 | AGAGCCAGUCUCUGGCCCCAG | Intron 8 |
| 110 | RSQ24572 | AAGAGCCAGUCUCUGGCCCCA | Intron 8 |
| 111 | RSQ24573 | UAAGAGCCAGUCUCUGGCCCC | Intron 8 |
| 112 | RSQ24574 | CUGAGCCAGCCACCAGAGGGC | Intron 8 |
| 113 | RSQ24575 | UCUGAGCCAGCCACCAGAGGG | Intron 8 |
| 114 | RSQ24576 | CAUCUUCUAGGUAUGACAACG | Exon 9 |
| 115 | RSQ24578 | UUGAUGGUACAUGACAAGGUG | 1kb_downstream |
| 116 | RSQ24579 | GAGGCCCUACCCUCAGUCUGA | 1kb_downstream |
| 117 | RSQ24580 | CCUCUCCUCGCUCCAGUCCUA | 1kb_downstream |
| 118 | RSQ24581 | CUCUCCUCGCUCCAGUCCUAG | 1kb_downstream |
| 119 | RSQ24582 | GCCAACAGCAGAUAGCCUAGG | 1kb_downstream |
| 120 | RSQ24583 | UGUGCCCUCGUGUCUUAUCUG | 1kb_downstream |
| 121 | RSQ24584 | CCUAGAUGAAUCCUGCUUGAA | 1kb_downstream |
| 122 | RSQ24585 | GGUACUUGGUUUACCUAGAUG | 1kb_downstream |
| 123 | RSQ24586 | AGGUACUUGGUUUACCUAGAU | 1kb_downstream |
| 124 | RSQ24587 | AAACAUUAUAUAGUCCUUACC | 1kb_downstream |
| 125 | RSQ24588 | UAAACAUUAUAUAGUCCUUAC | 1kb_downstream |
| 126 | RSQ24589 | CCGAUUUUUAAACAUUAUAUA | 1kb_downstream |
| 127 | RSQ24590 | ACCGAUUUUUAAACAUUAUAU | 1kb_downstream |
| 128 | RSQ24591 | UACCGAUUUUUAAACAUUAUA | 1kb_downstream |
| 129 | RSQ24592 | AAAAUCGGUAAAAAUGCCCAC | 1kb_downstream |
| 130 | RSQ24593 | GAGGAAGAUGAACUGAGAUGU | 1kb_downstream |
| 131 | RSQ24594 | AGGAAGAUGAACUGAGAUGUG | 1kb_downstream |

### Example 2: Knock-In of Cargo at Essential Gene Locus of B Cells

The present example describes use of the gene editing methods described herein comprising viral vector transduction of a B cell population.

B cells were thawed as known in the art and cultured for 48 hours prior to electroporation. In brief, for electroporation, 1,000,000 B cells were suspended in P3 buffer per well in a Lonza 96-well nucleofector and electroporated with RNP comprising gRNA RSQ22337 (SEQ ID NO: 95) and Cas12a (SEQ ID NO: 62) targeting the GAPDH gene. Appropriate media was added to cells immediately after electroporation and cells were allowed to recover. After plating into a 24 well plate, AAV6 comprising donor template was added at 1.25×10¹⁰ viral genomes (VG)/ml virus. The donor template was designed as described herein, with a 5′ codon-optimized coding portion of GAPDH exon 9 optimized to prevent further binding of the gRNA targeting domain sequence of the guide RNA (RSQ22337)), an in-frame sequence encoding the P2A self-cleaving peptide (“P2A”), an in-frame coding sequence for GFP (“Cargo”), a stop codon, and a polyA signal sequence. Cells were cultured for 7 days and media was refreshed every 2 days to maintain a cell density of 5×10⁵ cells/ml.

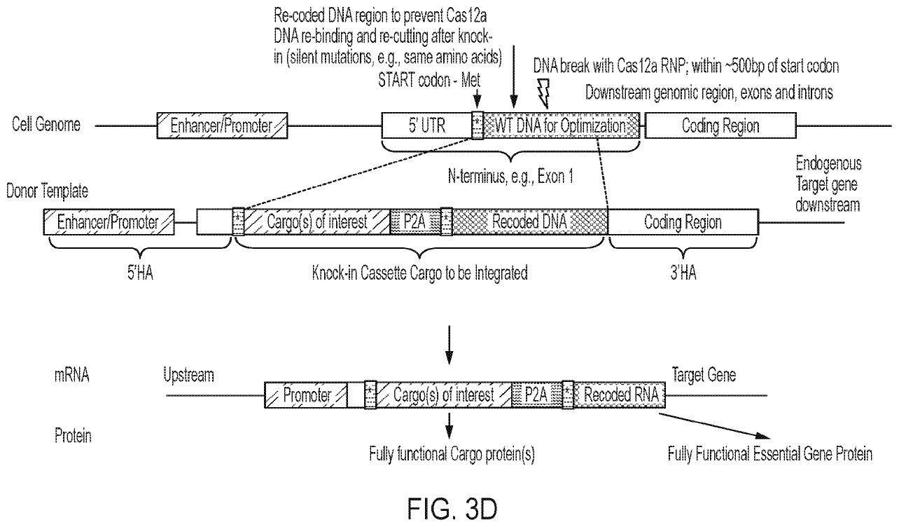

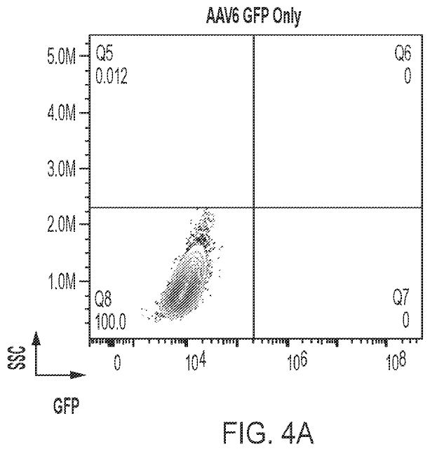

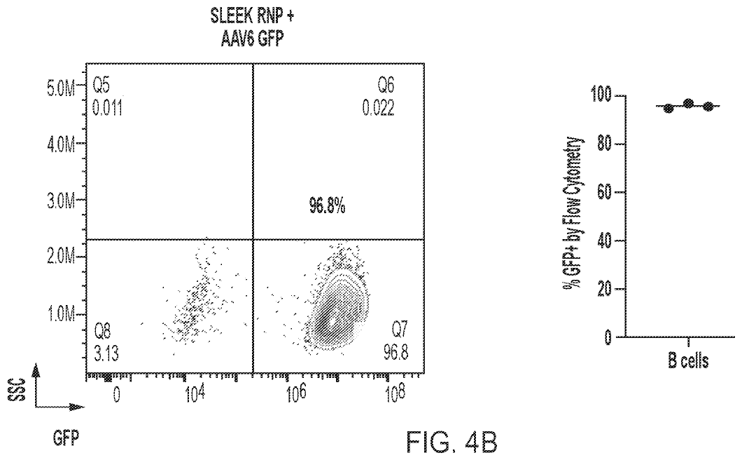

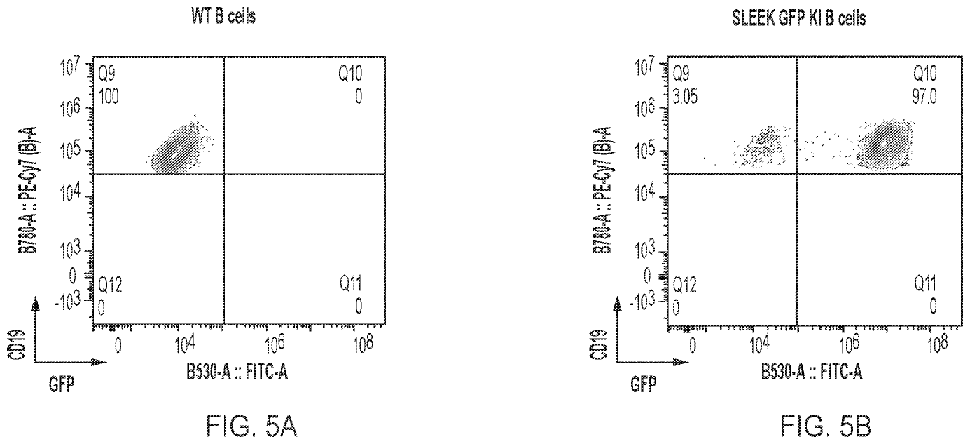

Successful transduction, editing, knock-in cassette integration, and/or expression events were determined using flow cytometry at 7 days post-electroporation, as described herein. Following AAV transduction, a large proportion of the cells are edited at the GAPDH locus by the RNP and have integrated the knock-in cassette via HDR. As seen in FIG. 4A, control cells that received only the AAV6 comprising the donor template (e.g., cells that did not receive the GAPDH targeting RNP) did not display GFP expression. In contrast, as depicted in FIG. 4B, a high proportion (˜96.8%) of cells were observed via flow cytometry to have GFP expression following AAV transduction. Further flow cytometry showed that a high proportion (˜97.0%) of cells expressed both the B cell marker CD19 and GFP (FIG. 5B) while ˜100% of unedited wild-type B cells displayed only CD19 expression and not GFP (FIG. 5A). Thus, the methods described herein can be used to generate and isolate a population of modified B cells that highly express a gene of interest (here represented by GFP) relative to other gene knock-in methods.

### Example 3: Simultaneous Knockout of Genes in B Cells and Knock-In of Cargo at Essential Gene Locus of B Cells

The present example describes use of the gene editing methods described herein comprising viral vector transduction of a B cell population.

CD19+ B cells were thawed using standard methods and cultured for 48 hours prior to electroporation. In brief, for electroporation, 500,000 B cells were suspended in P3 buffer per well in a Lonza 96-well nucleofector and electroporated with RNP comprising a gRNA (SEQ ID NO: 2000) targeting the B2M gene, either gRNA #1 (SEQ ID NO: 2001) or gRNA #2 (SEQ ID NO: 2002) targeting the CIITA gene, and RSQ22337 (SEQ ID NO: 95) targeting the GAPDH gene and Cas12a (SEQ ID NO: 62). Appropriate media was added to cells immediately after electroporation and cells were allowed to recover. After plating into a 12 well plate, AAV6 comprising donor template was added at 1.5×10⁵ MOI. The donor template was designed as described herein, with a 5′ codon-optimized coding portion of GAPDH exon 9 optimized to prevent further binding of the gRNA targeting domain sequence of the guide RNA (RSQ22337)), an in-frame sequence encoding the P2A self-cleaving peptide (“P2A”), an in-frame coding sequence for a HLA-E transgenic polypeptide as described in WO 2022/272292 (“Cargo”), a stop codon, and a polyA signal sequence. Cells were cultured for 7 days and media was refreshed every 2 days.

TABLE 15
Guide RNA sequences

| SEQ | | gRNA targeting |
|---|---|---|
| ID NO: | Gene | domain sequence (RNA) |
| 2000 | B2M | AGTGGGGGTGAATTCAGTGTA |
| 2001 | CIITA | TCTGCAGCCTTCCCAGAGGA |
| 2002 | CIITA | TGCCCAACTTCTGCTGGCATC |

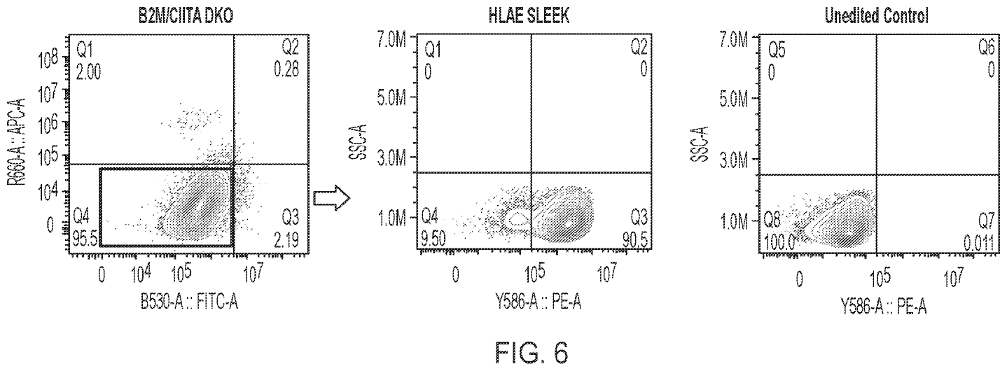

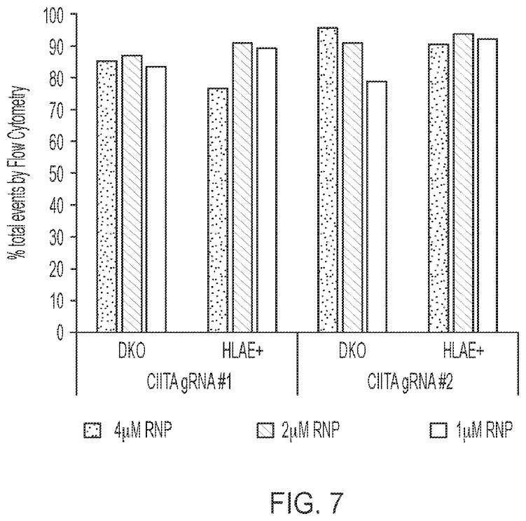

Successful transduction, editing, knock-in cassette integration, and/or expression events were determined using flow cytometry at 7 days post-electroporation, as described herein. Examination of the resulting cells confirmed that the methods described herein were capable of editing a large proportion of B cells. As shown in the left plot in FIG. 6, a high proportion (˜95.5%) of cells were observed via flow cytometry to comprise B2M/CIITA double knockout (DKO). Of these B2M/CIITA DKO B cells, a high proportion (˜90.5%) of cells expressed the HLA-E transgenic polypeptide (center plot, FIG. 6). Meanwhile, unedited control B cells did not display any detectable expression of the HLA-E transgenic polypeptide (right plot, FIG. 6). Additional analysis confirmed robust editing of B cells as up to at least 95% efficiency was achieved for the B2M/CIITA DKO and up to at least 90% efficiency was achieved for the HLA-E transgenic construct knock-in (FIG. 7). Robust editing was observed using two different CIITA gRNAs (SEQ ID NO: 2001 and SEQ ID NO: 2002) and at a range of RNP concentrations (1 uM, 2 uM, 4 uM). These results confirm that methods described herein can be used to simultaneously perform multiple genomic edits—including knockouts and knock-ins—in B cells, and can produce populations of modified B cells exhibiting high expression levels of a gene of interest.

### EQUIVALENTS

It is to be understood that while the disclosure has been described in conjunction with the detailed description thereof, the foregoing description is intended to illustrate and not limit the scope of the present disclosure, which is defined by the scope of the appended claims. Other aspects, advantages, and modifications are within the scope of the following claims.

We claim:

1. A method of editing the genome of a B cell, the method comprising contacting the cell with:
(i) a nuclease that causes a break within an endogenous coding sequence of an essential gene in the B cell, and
(ii) a donor template that comprises a knock-in cassette comprising an exogenous coding sequence for a gene product of interest in frame with and downstream (3′) of an exogenous coding sequence or partial coding sequence of the essential gene, wherein the knock-in cassette is integrated into the genome of the B cell by homology-directed repair (HDR) of the break, resulting in a genome-edited B cell that expresses:
(a) the gene product of interest, and
(b) the gene product encoded by the essential gene, or a functional variant thereof.

2. The method of claim 1, wherein, if the knock-in cassette is not integrated into the genome of the B cell by homology-directed repair (HDR) in the correct position or orientation, the B cell no longer expresses the gene product encoded by the essential gene, or a functional variant thereof.

3. The method of claim 1 or 2, wherein the break is a double-strand break.

4. The method of any one of claims 1-3, wherein the break is located within the last 1000, 500, 400, 300, 200, 100, or 50 base pairs of the endogenous coding sequence of the essential gene.

5. The method of any one of claims 1-4, wherein the break is located within the last exon of the essential gene.

6. The method of any one of claims 1-5, wherein the nuclease is a CRISPR/Cas nuclease and the method further comprises contacting the B cell with a guide molecule for the CRISPR/Cas nuclease.

7. The method of any one of claims 1-5, wherein the nuclease is a zinc finger nuclease (ZFN), a transcription activator-like effector nuclease (TALEN) or a meganuclease.

8. The method of any one of claims 1-7, wherein the donor template is a donor DNA template, optionally wherein the donor DNA template is double-stranded.

9. The method of claim 8, wherein the donor DNA template is a plasmid, optionally wherein the plasmid has not been linearized.

10. The method of any one of claims 1-9, wherein the donor template comprises homology arms on either side of the knock-in cassette.

11. The method of claim 10, wherein the homology arms correspond to sequences located on either side of the break in the genome of the B cell.

12. The method of any one of claims 1-11, wherein the knock-in cassette comprises a regulatory element that enables expression of the gene product encoded by the essential gene and the gene product of interest as separate gene products, optionally, wherein at least one of the gene products is a protein and the regulatory element enables expression of that protein separate from the other gene product.

13. The method of claim 12, wherein the knock-in cassette comprises an IRES or 2A element located between the exogenous coding sequence or partial coding sequence of the essential gene and the exogenous coding sequence for the gene product of interest.

14. The method of claim 13, wherein the 2A element is a T2A element (EGRGSLLTCGDVEENPGP), a P2A element (ATNFSLLKQAGDVEENPGP), a E2A element (QCTNYALLKLAGDVESNPGP), or an F2A element (VKQTLNFDLLKLAGDVESNPGP).

15. The method of claim 13 or 14, wherein the knock-in cassette further comprises a sequence encoding a linker peptide upstream of the 2A element.

16. The method of claim 15, wherein the linker peptide comprises the amino acid sequence GSG.

17. The method of any one of claims 1-16, wherein the knock-in cassette comprises a polyadenylation sequence, and optionally a 3′ UTR sequence, downstream of the exogenous coding sequence for the gene product of interest, wherein, if a 3′UTR sequence is present, the 3′UTR sequence is positioned 3′ of the exogenous coding sequence and 5′ of the polyadenylation sequence.

18. The method of any one of claims 1-17, wherein the exogenous partial coding sequence of the essential gene in the knock-in cassette encodes a C-terminal fragment of a protein encoded by the essential gene.

19. The method of claim 18, wherein the C-terminal fragment is less than 500, 250, 150, 125, 100, 75, 50, 25, 20, 15 or 10 amino acids in length.

20. The method of claim 18 or 19, wherein the C-terminal fragment includes an amino acid sequence that is encoded by a region of the endogenous coding sequence of the essential gene that spans the break.

21. The method of any one of claims 1-20, wherein the exogenous coding sequence or partial coding sequence of the essential gene in the knock-in cassette is less than 100% identical to the corresponding endogenous coding sequence of the essential gene of the B cell.

22. The method of claim 21, wherein the exogenous coding sequence or partial coding sequence of the essential gene in the knock-in cassette has been codon optimized relative to the corresponding endogenous coding sequence of the essential gene of the B cell to prevent further binding of the nuclease to the target site, to reduce the likelihood of recombination after integration of the knock-in cassette into the genome of the B cell, and/or to increase expression of the gene product of the essential gene and/or the gene product of interest after integration of the knock-in cassette into the genome of the B cell.

23. The method of any one of claims 1-22, wherein the essential gene is a housekeeping gene, e.g., a gene listed in Table 3.

24. The method of any one of claims 1-22, wherein the B cell is a progenitor B cell, Pre B cell, Pro B cell, an immature B cell, a transitional B cell, a mature B cell, a naïve B cell, memory B cell, a marginal zone B cell, a follicular B cell, a germinal center B cell, or plasma B cell.

25. The method of any one of claims 1-24, wherein the donor template does not comprise a reporter gene, e.g., a fluorescent reporter gene or an antibiotic resistance gene.

26. A genetically modified B cell comprising a genome with an exogenous coding sequence for a gene product of interest in frame with and downstream (3′) of a coding sequence of an essential gene.

27. An engineered cell comprising a genomic modification, wherein the genomic modification comprises an insertion of an exogenous knock-in cassette within an endogenous coding sequence of an essential gene in the B cell's genome, wherein the knock-in cassette comprises an exogenous coding sequence for a gene product of interest in frame with and downstream (3) of an exogenous coding sequence or partial coding sequence encoding the gene product of the essential gene, or a functional variant thereof, and wherein the B cell expresses the gene product of interest and the gene product encoded by the essential gene, or a functional variant thereof, optionally wherein the gene product of interest and the gene product encoded by the essential gene are expressed from the endogenous promoter of the essential gene.

28. The B cell of claim 26 or 27, wherein the cell's genome comprises a regulatory element that enables expression of the gene product encoded by the essential gene and the gene product of interest as separate gene products, optionally, wherein at least one of the gene products is a protein and the regulatory element enables expression of that protein separate from the other gene product.

29. The cell of claim 28, wherein the B cell's genome comprises an IRES or 2A element located between the coding sequence of the essential gene and the exogenous coding sequence for the gene product of interest.

30. The cell of any one of claims 26-29, wherein the B cell's genome comprises a polyadenylation sequence, and optionally a 3′ UTR sequence, downstream of the exogenous coding sequence for the gene product of interest, wherein, if a 3′UTR sequence is present, the 3′UTR sequence is positioned 3′ of the exogenous coding sequence and 5′ of the polyadenylation sequence.

31. The B cell of any one of claims 26-30, wherein the coding sequence of the essential gene is less than 100% identical to an endogenous coding sequence of the essential gene.

32. The B cell of any one of claims 26-31, wherein the essential gene is a housekeeping gene, e.g., a gene listed in Table 3.

33. The B cell of claim 26-32, wherein the B cell is a progenitor B cell, Pre B cell, Pro B cell, an immature B cell, a transitional B cell, a mature B cell, a naïve B cell, memory B cell, a marginal zone B cell, a follicular B cell, a germinal center B cell, or plasma B cell.

34. The B cell of any one of claims 26-33, wherein the B cell's genome does not comprise a reporter gene, e.g., a fluorescent reporter gene or an antibiotic resistance gene.

35. The B cell of any one of claims 26-34, for use as a medicament.

36. The B cell of any one of claims 26-34, for use in the treatment of a disease, disorder, or condition, e.g., a cancer.

37. A B cell, or population of B cells, produced by the method of any one of claims 1-25 or progeny thereof.

38. A system for editing the genome of a B cell, the system comprising the B cell, a nuclease that causes a break within an endogenous coding sequence of an essential gene of the B cell, and a donor template that comprises a knock-in cassette comprising an exogenous coding sequence for a gene product of interest in frame with and downstream (3′) of an exogenous coding sequence or partial coding sequence of the essential gene.

39. The system of claim 38, wherein the break is a double-strand break.

40. The system of claim 38 or 39, wherein the break is located within the last 1000, 500, 400, 300, 200, 100 or 50 base pairs of the coding sequence of the essential gene.

41. The system of any one of claims 38-40, wherein the break is located within the last exon of the essential gene.

42. The system of any one of claims 38-41, wherein the nuclease is a CRISPR/Cas nuclease and the system further comprises a guide molecule for the CRISPR/Cas nuclease.

43. The system of any one of claims 38-41, wherein the nuclease is a zinc finger nuclease (ZFN), a transcription activator-like effector nuclease (TALEN) or a meganuclease.

44. The system of any one of claims 38-43, wherein the donor template is a donor DNA template, optionally wherein the donor DNA template is double-stranded.

45. The system of claim 44, wherein the donor DNA template is a plasmid, optionally wherein the plasmid has not been linearized.

46. The system of any one of claims 38-45, wherein the donor template comprises homology arms on either side of the knock-in cassette.

47. The system of claim 46, wherein the homology arms correspond to sequences located on either side of the break in the genome of the B cell.

48. The system of any one of claims 38-47, wherein the knock-in cassette comprises a regulatory element that enables expression of the gene product encoded by the essential gene and the gene product of interest as separate gene products, optionally, wherein at least one of the gene products is a protein and the regulatory element enables expression of that protein separate from the other gene product.

49. The system of claim 48, wherein the knock-in cassette comprises an IRES or 2A element located between the exogenous coding sequence or partial coding sequence of the essential gene and the exogenous coding sequence for the gene product of interest.

50. The system of any one of claims 38-49, wherein the knock-in cassette comprises a polyadenylation sequence, and optionally a 3′ UTR sequence, downstream of the exogenous coding sequence for the gene product of interest, wherein, if a 3′UTR sequence is present, the 3′UTR sequence is positioned 3′ of the exogenous coding sequence and 5′ of the polyadenylation sequence.

51. The system of any one of claims 38-50, wherein the exogenous partial coding sequence of the essential gene in the knock-in cassette encodes a C-terminal fragment of a protein encoded by the essential gene.

52. The system of claim 51, wherein the C-terminal fragment is less than 500, 250, 150, 125, 100, 75, 50, 25, 20, 15 or 10 amino acids in length.

53. The system of claim 51 or 52, wherein the C-terminal fragment includes an amino acid sequence that is encoded by a region of the coding sequence of the essential gene that spans the break.

54. The system of any one of claims 38-53, wherein the exogenous coding sequence or partial coding sequence of the essential gene in the knock-in cassette is less than 100% identical to the corresponding endogenous coding sequence of the essential gene of the cell.

55. The system of claim 54, wherein the exogenous coding sequence or partial coding sequence of the essential gene in the knock-in cassette has been codon optimized relative to the corresponding endogenous coding sequence of the essential gene of the B cell to prevent further binding of a nuclease to the target site, to reduce the likelihood of recombination after integration of the knock-in cassette into the genome of the B cell, or to increase expression of the gene product of the essential gene and/or the gene product of interest after integration of the knock-in cassette into the genome of the B cell.

56. The system of claim 55, wherein the exogenous coding sequence or partial coding sequence of the essential gene in the knock-in cassette does not comprise a target site for the nuclease.

57. The system of any one of claims 38-56, wherein the essential gene is a housekeeping gene, e.g., a gene listed in Table 3.

58. The system of any one of claims 38-57, wherein the B cell is progenitor B cell, Pre B cell, Pro B cell, an immature B cell, a transitional B cell, a mature B cell, a naïve B cell, memory B cell, a marginal zone B cell, a follicular B cell, a germinal center B cell, or plasma B cell.

59. The system of any one of claims 38-58, wherein the donor DNA template does not comprise a reporter gene, e.g., a fluorescent reporter gene or an antibiotic resistance gene.
